(19) United States
(12) Patent Application Publication
(10) Pub. No.: US 2024/0238397 A1
(43) Pub. Date: Jul. 18, 2024

(54) KRAS-NEOANTIGEN THERAPIES

(71) Applicant: Gritstone bio, Inc., Emeryville, CA (US)

(72) Inventors: Raphael Rousseau, Los Altos, CA (US); Karin Jooss, San Diego, CA (US); Christine Denise Palmer, Cambridge, MA (US); Amy Rachel Rappaport, Daly City, CA (US)

(21) Appl. No.: 18/607,061

(22) Filed: Mar. 15, 2024

Related U.S. Application Data
(63) Continuation of application No. PCT/US2022/076680, filed on Sep. 19, 2022.
(60) Provisional application No. 63/374,888, filed on Sep. 7, 2022.
(60) Provisional application No. 63/321,587, filed on Mar. 18, 2022.
(60) Provisional application No. 63/281,029, filed on Nov. 18, 2021.
(60) Provisional application No. 63/245,703, filed on Sep. 17, 2021.

(51) Int. Cl. A61K 39/00; A61K 39/395; A61P 35/00; A61P 37/04

(52) U.S. Cl. CPC A61K 39/001164; A61K 39/3955; A61P 35/00; A61P 37/04; A61K 2039/5256; A61K 2039/53; A61K 2039/54; A61K 2039/545

(57) ABSTRACT

Disclosed herein are compositions that include antigen-encoding nucleic acid sequences having multiple iterations of KRAS neoepitope-encoding sequences and/or lacking immunodominant epitopes. Also disclosed are nucleotides, cells, and methods associated with the compositions including their use as vaccines, such as in subjects with cancer that includes (1) a solid tumor expressing the KRAS-associated MHC class I neoepitope, (2) colorectal cancer (CRC), (3) non-small cell lung cancer (NSCLC), and/or (4) pancreatic ductal adenocarcinoma (PDA).

### CROSS REFERENCE TO RELATED APPLICATIONS

This application is a Continuation of International Patent Application No. PCT/US2022/076680, filed Sep. 19, 2022, which claims the benefit of U.S. Provisional Application No. 63/245,703 filed Sep. 17, 2021, U.S. Provisional Application No. 63/281,029 filed Nov. 18, 2021, U.S. Provisional Application No. 63/321,587 filed Mar. 18, 2022, and U.S. Provisional Application No. 63/374,888 filed Sep. 7, 2022, each of which is hereby incorporated in their entirety by reference for all purposes.

### SEQUENCE LISTING

The instant application contains a Sequence Listing which has been submitted electronically in .XML. format and is hereby incorporated by reference in its entirety. Said .XML. copy, created on Feb. 2, 2023, is named GSO-106WO .XML and is 149,971 bytes in size.

### BACKGROUND

Therapeutic vaccines based on tumor-specific antigens hold great promise as a next-generation of personalized cancer immunotherapy. ¹⁻³ For example, cancers with a high mutational burden, such as non-small cell lung cancer (NSCLC) and melanoma, are particularly attractive targets of such therapy given the relatively greater likelihood of neoantigen generation. ^{4, 5} Early evidence shows that neoantigen-based vaccination can elicit T-cell responses⁶ and that neoantigen targeted cell-therapy can cause tumor regression under certain circumstances in selected patients.⁷

One question for antigen vaccine design in both cancer and infectious disease settings is which of the many coding mutations present generate the “best” therapeutic antigens, e.g., antigens that can elicit immunity.

In addition to the challenges of current antigen prediction methods certain challenges also exist with the available vector systems that can be used for antigen delivery in humans, many of which are derived from humans. For example, many humans have pre-existing immunity to human viruses as a result of previous natural exposure, and this immunity can be a major obstacle to the use of recombinant human viruses for antigen delivery in vaccination strategies, such as in cancer treatment or vaccinations against infectious diseases. While some progress has been made in vaccinations strategies addressing the above problems, improvements are still needed, particularly for clinical applications, such as improved vaccine potency and efficacy.

### SUMMARY

Disclosed herein is a method for treating a subject with a disease, wherein the disease is cancer comprising (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula: (Eₓ−(E^{N}ₙ)_{y})_{z}, wherein, E represents a nucleotide sequence a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes the KRAS-associated MHC class I neoepitope.

Also disclosed herein is a method for treating a subject with a disease, wherein the disease is cancer comprising (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding the KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the epitope-encoding nucleic acid sequence encoding the KRAS-associated MHC class I neoepitope.

In some aspects, the epitope-encoding nucleic acid sequence is derived from a tumor of the subject with cancer or from a cell or sample of the infected subject. In some aspects, the epitope-encoding nucleic acid sequence are not derived from a tumor of the subject with cancer or from a cell or sample of the infected subject.

Also disclosed herein is a method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula (Eₓ−(E^{N}ₙ)_{y})_{z}, wherein, E represents a nucleotide sequence comprising a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes the KRAS-associated MHC class I neoepitope.

Also disclosed herein is a method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding the KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of at least one of the epitope-encoding nucleic acid sequences encoding the KRAS-associated MHC class I neoepitope.

In some aspects, the subject expresses at least one HLA allele predicted or known to present the at least one epitope sequence, optionally wherein the at least one epitope sequence predicted or known to be presented comprises the KRAS-associated MHC class I neoepitope. In some aspects, the subject expresses at least one HLA allele predicted or known to present the at least one epitope sequence, and wherein the at least one epitope sequence comprises an epitope known or suspected to be presented by MHC class I on a surface of a cell, optionally wherein the at least one epitope sequence predicted or known to be presented comprises the KRAS-associated MHC class I neoepitope. In some aspects, the surface of the cell is a tumor cell surface.

Also disclosed herein is a method for inducing an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula: (Eₓ−(E^{N}ₙ)_{y})_{z}, wherein, E represents a nucleotide sequence comprising a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes a KRAS-associated MHC class I neoepitope.

Also disclosed herein is a method for inducing an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding the KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, wherein the at least one antigen-encoding nucleic acid sequence comprises at least two iterations of the epitope-encoding nucleic acid sequence encoding the KRAS-associated MHC class I neoepitope, and wherein the subject expresses at least one HLA allele predicted or known to present the at least one KRAS-associated MHC class I neoepitope.

In some aspects, the antigen-based vaccine is administered as a priming dose. In some aspects, the antigen-based vaccine is administered as one or more boosting doses. In some aspects, the boosting dose is different than the priming dose. In some aspects a) the priming dose comprises a chimpanzee adenovirus vector and the boosting dose comprises an alphavirus vector; or b) the priming dose comprises an alphavirus vector and the boosting dose comprises a chimpanzee adenovirus vector. In some aspects, the boosting dose is the same as the priming dose. In some aspects, the injection site of the one or more boosting doses is as close as possible to the injection site of the priming dose.

In some aspects, the method further comprises determining or having determined the HLA-haplotype of the subject.

In some aspects, the antigen-based vaccine is administered intramuscularly (IM), intradermally (ID), subcutaneously (SC), or intravenously (IV). In some aspects, the antigen-based vaccine is administered intramuscularly (IM). In some aspects, the IM administration is administered at separate injection sites. In some aspects, the separate injection sites are in opposing deltoid muscles. In some aspects, the separate injection sites are in gluteus or rectus femoris sites on each side.

In some aspects, the KRAS-associated MHC class I neoepitope or the KRAS mutation comprises a KRAS G12C mutation, a KRAS G12V mutation, a KRAS G12D mutation, or a KRAS Q61H mutation. In some aspects, the KRAS-associated MHC class I neoepitope or the KRAS mutation comprises any one of the amino acid sequence shown in SEQ ID NOs: 75-82. In some aspects, the antigen-encoding cassette comprises each of the amino acid sequence shown in SEQ ID NOs: 75-82. In some aspects, the antigen-encoding cassette comprises two or more iterations of each of the amino acid sequence shown in SEQ ID NOs: 75-82, optionally comprising 4 iterations of each of the amino acid sequence shown in SEQ ID NOs: 75-82. In some aspects, the KRAS-associated MHC class I neoepitope or the KRAS mutation comprises the amino acid sequence shown in SEQ ID NO: 57, SEQ ID NO: 58, SEQ ID NO: 59, or SEQ ID NO: 60. In some aspects, the epitope-encoding nucleic acid sequences comprises two or more distinct epitope-encoding nucleic acid sequences independently encoding a distinct KRAS-associated MHC class I neoepitope or a distinct KRAS mutation. In some aspects, each of the epitope-encoding nucleic acid sequences independently encodes a distinct KRAS-associated MHC class I neoepitope or a distinct KRAS mutation. In some aspects, the epitope-encoding nucleic acid sequences comprises two or more distinct epitope-encoding nucleic acid sequences independently encoding a KRAS G12C mutation, a KRAS G12V mutation, a KRAS G12D mutation, or a KRAS Q61H mutation. In some aspects, the epitope-encoding nucleic acid sequences independently encodes each of a KRAS G12C mutation, a KRAS G12V mutation, and a KRAS G12D mutation, and optionally a KRAS Q61H mutation. In some aspects, the antigen-encoding nucleic acid sequence encodes a peptide comprising the amino acid sequence shown in SEQ ID NO: 64 or SEQ ID NO: 65.

In some aspects, the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response.

In some aspects, the cancer comprises a solid tumor expressing a KRAS-associated and/or a NRAS-associated MHC class I neoepitope. In some aspects, the KRAS-associated and/or the NRAS-associated MHC class I neoepitope comprises a mutation selected from the group consisting of: KRAS_G12C, NRAS_G12C, KRAS_G12D, NRAS_G12D, KRAS_G12V, NRAS_G12V, KRAS_Q61H, and NRAS_Q61H.

In some aspects, the cancer comprises colorectal cancer (CRC). In some aspects, the cancer comprises non-small cell lung cancer (NSCLC). In some aspects, the cancer comprises pancreatic ductal adenocarcinoma (PDA).

In some aspects, the antigen-based vaccine or the one or more boosting doses is administered every 4 weeks (Q4W). In some aspects, the antigen-based vaccine or the one or more boosting doses is administered every 8 weeks (Q8W). In some aspects, the antigen-based vaccine or the one or more boosting doses is administered monthly. In some aspects, the antigen-based vaccine or the one or more boosting doses is administered every two months.

In some aspects, the method comprises administering to the subject a composition for delivery of a self-replicating alphavirus-based expression system and administering to the subject a composition for delivery of a chimpanzee adenovirus (ChAdV)-based expression system, and wherein the composition for delivery of the ChAdV-based expression system is administered as a priming dose and the composition for delivery of the self-replicating alphavirus-based expression system is administered as one or more boosting doses.

In some aspects, two or more boosting doses are administered. In some aspects, 1, 2, 3, 4, 5, 6, 7, or 8 boosting doses are administered.

In some aspects, the ChAdV-based expression system is further administered as a boosting dose. In some aspects, the ChAdV-based boosting dose is only administered as a single boosting dose. In some aspects, the ChAdV-based expression system is administered as the boosting dose on or about day 140 after the priming dose of the ChAdV-based expression system. In some aspects, the ChAdV-based expression system is administered as the boosting dose on or about week 20 after the priming dose of the ChAdV-based expression system. In some aspects, the ChAdV-based expression system is administered as the boosting dose on or about month 5 after the priming dose of the ChAdV-based expression system. In some aspects, the ChAdV-based expression system is administered as the boosting dose on or after day 140 after the priming dose of the ChAdV-based expression system. In some aspects, the ChAdV-based expression system is administered as the boosting dose on or after week 20 after the priming dose of the ChAdV-based expression system. In some aspects, the ChAdV-based expression system is administered as the boosting dose on or after month 5 after the priming dose of the ChAdV-based expression system.

In some aspects, the self-replicating alphavirus-based expression system is administered as at least two boosting doses. In some aspects, the self-replicating alphavirus-based expression system is administered as at least two or more boosting doses at least 28 days apart. In some aspects, the self-replicating alphavirus-based expression system is administered as at least two or more boosting doses at least 4 weeks (Q4W) apart. In some aspects, the self-replicating alphavirus-based expression system is administered as at least two or more boosting doses at least one month apart. In some aspects, the self-replicating alphavirus-based expression system is administered as at least two or more boosting doses at least 56 days apart. In some aspects, the self-replicating alphavirus-based expression system is administered as at least two or more boosting doses at least 8 weeks (Q8W) apart. In some aspects, the self-replicating alphavirus-based expression system is administered as at least two or more boosting doses at least 2 months apart. In some aspects, the self-replicating alphavirus-based expression system is administered as at least two boosting doses on or about days 28 and 84 after the priming dose of the ChAdV-based expression system. In some aspects, the self-replicating alphavirus-based expression system is administered as at least two boosting doses on or about weeks 4 and 12 after the priming dose of the ChAdV-based expression system. In some aspects, the self-replicating alphavirus-based expression system is administered as at least two boosting doses on or about months 1 and 3 after the priming dose of the ChAdV-based expression system.

In some aspects, the self-replicating alphavirus-based expression system is administered as at least four boosting doses. In some aspects, the self-replicating alphavirus-based expression system is administered on or about days 28, 84, 196, and 252 relative to the priming dose of the ChAdV-based expression system. In some aspects, the self-replicating alphavirus-based expression system is administered on or about weeks 4, 12, 28, and 40 relative to the priming dose of the ChAdV-based expression system. In some aspects, the self-replicating alphavirus-based expression system is administered on or about months 1, 3, 7, and 10 relative to the priming dose of the ChAdV-based expression system.

In some aspects, the method further comprises administration of one or more immune modulators, optionally wherein the immune modulator is administered before, concurrently with, or after administration of the composition or pharmaceutical composition. In some aspects, the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the immune modulator is administered intravenously (IV), intramuscularly (IM), intradermally (ID), or subcutaneously (SC). In some aspects, the subcutaneous administration is near the site of the composition or pharmaceutical composition administration or in close proximity to one or more vector or composition draining lymph nodes. In some aspects, the method comprises administration of an anti-CTLA-4 antibody or an antigen-binding fragment thereof only with the priming dose and the first boosting dose. In some aspects, the anti-CTLA-4 antibody comprises ipilimumab. In some aspects, the ipilimumab is administered at a dose of 30 mg subcutaneously. In some aspects, the method comprises administration of an anti-PD-L1 antibody or an antigen-binding fragment thereof every 4 weeks (Q4W), optionally comprising. In some aspects, the anti-PD-L1 antibody comprises atezolizumab or nivolumab. In some aspects, the atezolizumab is administered at a dose of 1680 mg intravenously or the nivolumab is administered at a dose of 480 mg intravenously.

In some aspects, the method comprises at least 2, 3, 4, 5, 6, 7, 8, 9, 10, 11, 12, or 13 administrations of one or more of the immune modulators. In some aspects, the method comprises at least 13 administrations of the anti-PD-L1 antibody. In some aspects, the one or more immune modulators are selected from the group consisting of: atezolizumab, nivolumamb, cemiplimab, and ipilimumab. In some aspects, the KRAS-associated MHC class I neoepitope comprising a KRAS G12C mutation is selected from the group consisting of: VVVGACGVGK (SEQ ID NO: 75), KLVVVGACGV (SEQ ID NO: 76), and GACGVGKSAL (SEQ ID NO: 93); the KRAS-associated MHC class I neoepitope comprising a KRAS G12D mutation is selected from the group consisting of: VVGADGVGK (SEQ ID NO: 77), VVVGADGVGK (SEQ ID NO: 78), KLVVVGADGV (SEQ ID NO: 94), and GADGVGKSAL (SEQ ID NO: 95); the KRAS-associated MHC class I neoepitope comprising a KRAS G12V mutation is selected from the group consisting of: VVGAVGVGK (SEQ ID NO: 79), VVVGAVGVGK (SEQ ID NO: 81), AVGVGKSAL (SEQ ID NO: 80), and GAVGVGKSAL (SEQ ID NO: 96). In some aspects, the KRAS-associated MHC class I neoepitope comprising a KRAS G12C mutation is selected from the group consisting of: VVVGACGVGK (SEQ ID NO: 75), KLVVVGACGV (SEQ ID NO: 76), and GACGVGKSAL (SEQ ID NO: 93). In some aspects, the KRAS-associated MHC class I neoepitope comprising a KRAS G12D mutation is selected from the group consisting of: VVGADGVGK (SEQ ID NO: 77), VVVGADGVGK (SEQ ID NO: 78), KLVVVGADGV (SEQ ID NO: 94), and GADGVGKSAL (SEQ ID NO: 95). In some aspects, the KRAS-associated MHC class I neoepitope comprising a KRAS G12V mutation is selected from the group consisting of: VVGAVGVGK (SEQ ID NO: 79), VVVGAVGVGK (SEQ ID NO: 81), AVGVGKSAL (SEQ ID NO: 80), and GAVGVGKSAL (SEQ ID NO: 96).

In some aspects, the stimulating the immune response comprises stimulating a molecular response. In some aspects, the molecular response comprises a reduction in ctDNA. In some aspects, the reduction in ctDNA is at least a 20%, at least a 30%, at least a 40%, or at least a 50% reduction in ctDNA. In some aspects, the reduction in ctDNA is at least a 30% reduction in ctDNA.

In some aspects, the antigen-encoding cassette, or the polypeptide sequence encoded by the cassette, comprises at least one antigen-encoding nucleic acid sequence described, from 5′ to 3′, by the formula:

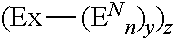

wherein E represents a nucleotide sequence comprising a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes a distinct KRAS-associated MHC class I neoepitope.

In some aspects, the antigen-encoding cassette encodes at least 4 iterations of each of the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82). In some aspects, the KRAS-associated MHC class I neoepitope or the KRAS mutation comprises a KRAS G12C mutation, a KRAS G12V mutation, a KRAS G12D mutation, or a KRAS Q61H mutation. In some aspects, the KRAS-associated MHC class I neoepitope or the KRAS mutation comprises any one of the amino acid sequence shown in SEQ ID NOs: 75-82. In some aspects, the antigen-encoding cassette comprises each of the amino acid sequence shown in SEQ ID NOs: 75-82. In some aspects, the antigen-encoding cassette comprises two or more iterations of each of the amino acid sequence shown in SEQ ID NOs: 75-82. In some aspects, the antigen-encoding cassette comprises 4 iterations of each of the amino acid sequence shown in SEQ ID NOs: 75-82. In some aspects, the KRAS-associated MHC class I neoepitope or the KRAS mutation comprises the amino acid sequence shown in SEQ ID NO: 57, SEQ ID NO: 58, SEQ ID NO: 59, or SEQ ID NO: 60. In some aspects, the epitope-encoding nucleic acid sequences comprises two or more distinct epitope-encoding nucleic acid sequences independently encoding a distinct KRAS-associated MHC class I neoepitope or a distinct KRAS mutation. In some aspects, each of the epitope-encoding nucleic acid sequences independently encodes a distinct KRAS-associated MHC class I neoepitope or a distinct KRAS mutation. In some aspects, the epitope-encoding nucleic acid sequences comprises two or more distinct epitope-encoding nucleic acid sequences independently encoding a KRAS G12C mutation, a KRAS G12V mutation, a KRAS G12D mutation, or a KRAS Q61H mutation. In some aspects, the epitope-encoding nucleic acid sequences independently encodes each of a KRAS G12C mutation, a KRAS G12V mutation, and a KRAS G12D mutation, and optionally a KRAS Q61H mutation. In some aspects, the antigen-encoding nucleic acid sequence encodes a peptide comprising the amino acid sequence shown in SEQ ID NO: 64 or SEQ ID NO: 65. In some aspects, the antigen-encoding nucleic acid sequence encodes a peptide comprising the amino acid sequence shown in SEQ ID NO: 65.

In some aspects, at least two of the distinct epitope-encoding nucleic acid sequences comprising the at least two iterations encode distinct KRAS-associated MHC class I neoepitopes. In some aspects, at least 3, at least 4, at least 5, at least 6, at least 7, or at least 8 of the distinct epitope-encoding nucleic acid sequences comprising the at least two iterations encode distinct KRAS-associated MHC class I neoepitopes. In some aspects, each of the distinct epitope-encoding nucleic acid sequences comprising the at least two iterations encode distinct KRAS-associated MHC class I neoepitopes. In some aspects, one or more of the nucleic acid sequences encoding the KRAS-associated MHC class I neoepitopes comprises at least 2, at least 3, at least 4, at least 5, at least 6, at least 7, or at least 8 iterations. In some aspects, each of the nucleic acid sequences encoding the KRAS-associated MHC class I neoepitopes comprises at least 2, at least 3, at least 4, at least 5, at least 6, at least 7, or at least 8 iterations. In some aspects, one or more of the nucleic acid sequences encoding the distinct KRAS-associated MHC class I neoepitopes comprises at least 4 iterations. In some aspects, each of the nucleic acid sequences encoding the distinct KRAS-associated MHC class I neoepitopes comprises at least 4 iterations. In some aspects, one or more of the distinct KRAS-associated MHC class I neoepitopes independently comprises a KRAS G12C mutation, a KRAS G12V mutation, a KRAS G12D mutation, or a KRAS Q61H mutation.

In some aspects, each E or E^{N} independently comprises a nucleotide sequence described, from 5′ to 3′, by the formula (L5_{b}-N_{c}-L3_{d}), wherein N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. In some aspects, each N encodes an epitope 7-15 amino acids in length, L5 is a native 5′ linker sequence that encodes a native N-terminal amino acid sequence of the epitope, and wherein the 5′ linker sequence encodes a peptide that is at least 2 amino acids in length, and L3 is a native 3′ linker sequence that encodes a native C-terminal amino acid sequence of the epitope, and wherein the 3′ linker sequence encodes a peptide that is at least 32 amino acids in length. In some aspects, the 5′ and/or 3′ linker sequence encodes a peptide that is at least 3 amino acids in length. In some aspects, the 5′ and/or 3′ linker sequence encodes a peptide that is at least 4 amino acids in length. In some aspects, the 5′ and/or 3′ linker sequence encodes a peptide that is at least 5 amino acids in length. In some aspects, the 5′ and/or 3′ linker sequence encodes a peptide that is at least 8 amino acids in length. In some aspects, the 5′ and/or 3′ linker sequence encodes a peptide that is at least 2-8 amino acids in length. In some aspects, the 5′ and/or 3′ linker sequence encodes a peptide that is at least 2-10 amino acids in length.

In some aspects, each E and E^{N} encodes an epitope at least 7 amino acids in length. In some aspects, each E and E^{N} encodes an epitope 7-15 amino acids in length. In some aspects, each E and E^{N} is a nucleotide sequence at least 21 nucleotides in length. In some aspects, each E and E^{N} is a nucleotide sequence 75 nucleotides in length.

In some aspects, the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding a KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the epitope-encoding nucleic acid sequence encoding the KRAS-associated MHC class I neoepitope.

In some aspects, the antigen expression system comprises one or more vectors, the one or more vectors comprising: (a) a vector backbone, wherein the backbone comprises: (i) at least one promoter nucleotide sequence, and (ii) at least one polyadenylation (poly(A)) sequence; and (b) a cassette, wherein the cassette comprises: (i) at least one antigen-encoding nucleic acid sequence, comprising: (I) at least 2, 3, 4, 5, 6, 7, 8, 9, or 10 distinct epitope-encoding nucleic acid sequences linearly linked to each other wherein at least one of the distinct epitope-encoding nucleic acid sequences encodes a KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of at least one of the distinct epitope-encoding nucleic acid sequences encoding the KRAS-associated MHC class I neoepitope.

In some aspects, the at least one antigen-encoding nucleic acid sequence comprises at least 3 distinct epitope-encoding nucleic acid sequences.

In some aspects, the antigen expression system comprises one or more vectors, the one or more vectors comprising: (a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and (b) a cassette, optionally wherein the cassette is integrated between a native promoter nucleotide sequence native to the vector backbone and a poly(A) sequence, optionally wherein the poly(A) sequence is native to the vector backbone, wherein the cassette comprises: (i) at least one antigen-encoding nucleic acid sequence, comprising: (I) an epitope-encoding nucleic acid sequence encoding a KRAS-associated MHC class I neoepitope, optionally comprising at least two distinct epitope-encoding nucleic acid sequences linearly linked to each other, each epitope-encoding nucleic acid sequence optionally comprising: (A) a MHC class I epitope encoding nucleic acid sequence, wherein the MHC class I epitope encoding nucleic acid sequence encodes a MHC class I epitope 7-15 amino acids in length, (B) a 5′ linker sequence, wherein the 5′ linker sequence encodes a native N-terminal amino acid sequence of the MHC class I epitope, and wherein the 5′ linker sequence encodes a peptide that is at least 2 amino acids in length, (C) a 3′ linker sequence, wherein the 3′ linker sequence encodes a native C-terminal acid sequence of the MHC class I epitope, and wherein the 3′ linker sequence encodes a peptide that is at least 2 amino acids in length, and wherein the cassette is operably linked to the native promoter nucleotide sequence, wherein each of the epitope-encoding nucleic acid sequences encodes a polypeptide that is between 13 and 25 amino acids in length, and wherein each 3′ end of each epitope-encoding nucleic acid sequence is linked to the 5′ end of the following epitope-encoding nucleic acid sequence with the exception of the final epitope-encoding nucleic acid sequence in the cassette; and (ii) at least two MHC class II epitope-encoding nucleic acid sequences comprising: (I) a PADRE MHC class II sequence (SEQ ID NO:48), (II) a Tetanus toxoid MHC class II sequence (SEQ ID NO:46), (III) a first nucleic acid sequence encoding a GPGPG amino acid linker sequence (SEQ ID NO:56) linking the PADRE MHC class II sequence and the Tetanus toxoid MHC class II sequence, (IV) a second nucleic acid sequence encoding a GPGPG amino acid linker sequence (SEQ ID NO:56) linking the 5′ end of the at least two MHC class II epitope-encoding nucleic acid sequences to the epitope-encoding nucleic acid sequences, (V) optionally, a third nucleic acid sequence encoding a GPGPG amino acid linker sequence (SEQ ID NO:56) at the 3′ end of the at least two MHC class II epitope-encoding nucleic acid sequences; (iii) optionally, a second promoter nucleotide sequence operably linked to the antigen-encoding nucleic acid sequence; and wherein if the second promoter nucleotide sequence is absent, the antigen-encoding nucleic acid sequence is operably linked to the native promoter nucleotide sequence, and wherein the at least one antigen-encoding nucleic acid sequence comprises at least two iterations of the epitope-encoding nucleic acid sequence encoding the KRAS-associated MHC class I neoepitope.

In some aspects, an ordered sequence of each element of the cassette is described in the formula, from 5′ to 3′, comprising:

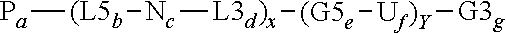

wherein, P comprises the second promoter nucleotide sequence, where a=0 or 1, N comprises one of the distinct epitope-encoding nucleic acid sequences, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO:56), where e=0 or 1, G3 comprises one of the at least one nucleic acid sequences encoding a GPGPG amino acid linker (SEQ ID NO:56), where g=0 or 1, U comprises one of the at least one MHC class II epitope-encoding nucleic acid sequence, where f=1, X=1 to 400, where for each X the corresponding N_{c} is an epitope-encoding nucleic acid sequence, and Y=0, 1, or 2, where for each Y the corresponding U_{f} is an MHC class II epitope-encoding nucleic acid sequence.

In some aspects, for each X the corresponding N_{c} is a distinct epitope-encoding nucleic acid sequence, except for the N_{c} corresponding to the at least two iterations of the distinct epitope-encoding nucleic acid sequence. In some aspects, for each Y the corresponding U_{f} is a distinct MHC class II epitope-encoding nucleic acid sequence. In some aspects, a=0, b=1, d=1, e=1, g=1, h=1, X=16, Y=2, the at least one promoter nucleotide sequence is a single native promoter nucleotide sequence native to the vector backbone, the at least one polyadenylation poly(A) sequence is a poly(A) sequence of at least 80 consecutive A nucleotides provided by the vector backbone, each N encodes an epitope 7-15 amino acids in length, L5 is a native 5′ linker sequence that encodes a native N-terminal amino acid sequence of the epitope, and wherein the 5′ linker sequence encodes a peptide that is at least 2 amino acids in length, L3 is a native 3′ linker sequence that encodes a native C-terminal amino acid sequence of the epitope, and wherein the 3′ linker sequence encodes a peptide that is at least 2 amino acids in length, U is each of a PADRE class II sequence and a Tetanus toxoid MHC class II sequence, the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, optionally wherein the native promoter nucleotide sequence is a subgenomic (e.g., 26S) promoter when the vector backbone comprises an alphavirus vector, and each of the MHC class II epitope-encoding nucleic acid sequences encodes a polypeptide that is between 13 and 25 amino acids in length.

In some aspects, the antigen-encoding cassette encodes at least 4 iterations of each of the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82). In some aspects, the KRAS-associated MHC class I neoepitope or the KRAS mutation comprises a KRAS G12C mutation, a KRAS G12V mutation, a KRAS G12D mutation, or a KRAS Q61H mutation. In some aspects, the KRAS-associated MHC class I neoepitope or the KRAS mutation comprises any one of the amino acid sequence shown in SEQ ID NOs: 75-82. In some aspects, the antigen-encoding cassette comprises each of the amino acid sequence shown in SEQ ID NOs: 75-82. In some aspects, the antigen-encoding cassette comprises two or more iterations of each of the amino acid sequence shown in SEQ ID NOs: 75-82. In some aspects, the antigen-encoding cassette comprises 4 iterations of each of the amino acid sequence shown in SEQ ID NOs: 75-82. In some aspects, the KRAS-associated MHC class I neoepitope or the KRAS mutation comprises the amino acid sequence shown in SEQ ID NO: 57, SEQ ID NO: 58, SEQ ID NO: 59, or SEQ ID NO: 60. In some aspects, the epitope-encoding nucleic acid sequences comprises two or more distinct epitope-encoding nucleic acid sequences independently encoding a distinct KRAS-associated MHC class I neoepitope or a distinct KRAS mutation. In some aspects, each of the epitope-encoding nucleic acid sequences independently encodes a distinct KRAS-associated MHC class I neoepitope or a distinct KRAS mutation. In some aspects, the epitope-encoding nucleic acid sequences comprises two or more distinct epitope-encoding nucleic acid sequences independently encoding a KRAS G12C mutation, a KRAS G12V mutation, a KRAS G12D mutation, or a KRAS Q61H mutation. In some aspects, the epitope-encoding nucleic acid sequences independently encodes each of a KRAS G12C mutation, a KRAS G12V mutation, and a KRAS G12D mutation, and optionally a KRAS Q61H mutation. In some aspects, the antigen-encoding nucleic acid sequence encodes a peptide comprising the amino acid sequence shown in SEQ ID NO: 64 or SEQ ID NO: 65. In some aspects, the antigen-encoding nucleic acid sequence encodes a peptide comprising the amino acid sequence shown in SEQ ID NO: 65.

In some aspects, the at least two iterations is at least 3, at least 4, at least 5, at least 6, at least 7, or at least 8 iterations. In some aspects, the at least two iterations is at least 8 iterations. In some aspects, the at least two iterations is at least 8, at least 9, at least 10, at least 11, at least 12, at least 13, 1 at least 4, at least 15, at least 16, at least 17, at least 18, at least 19, or at least 20 iterations. In some aspects, the at least two iterations is between 2-3, between 2-4, between 2-5, between 2-6, between 2-7 iterations, or between 2-8 iterations. In some aspects, the at least two iterations is 7 iterations or less, 6 iterations or less, 5 iterations or less, 4 iterations or less, or 3 iterations or less.

In some aspects, the at least one antigen-encoding nucleic acid sequence comprises at least two iterations of at least two distinct epitope-encoding nucleic acid sequences. In some aspects, the at least one antigen-encoding nucleic acid sequence comprises at least two iterations of at least 3, at least 4, at least 5, at least 6, at least 7, at least 8, at least 9, or at least 10 distinct epitope-encoding nucleic acid sequences. In some aspects, the at least two iterations are separated by at least one separate distinct epitope-encoding nucleic acid sequence. In some aspects, the at least two iterations are separated by at least 2 separate distinct epitope-encoding nucleic acid sequences. In some aspects, the at least two iterations, inclusive of the optional 5′ linker sequence and/or the optional 3′ linker sequence, are separated by at least 75 nucleotides. In some aspects, the at least two iterations, inclusive of the optional 5′ linker sequence and/or the optional 3′ linker sequence, are separated by at least 150 nucleotides, at least 300 nucleotides, or at least 675 nucleotides. In some aspects, the at least two iterations, inclusive of the optional 5′ linker sequence and/or the optional 3′ linker sequence, are separated by at least 50 nucleotides, at least 100 nucleotides, at least 200 nucleotides, at least 250 nucleotides, at least 350 nucleotides, at least 400 nucleotides, at least 450 nucleotides, at least 500 nucleotides, at least 700 nucleotides, at least 700 nucleotides, at least 750 nucleotides, at least 800 nucleotides, at least 900 nucleotides, or at least 1000 nucleotides. In some aspects, the at least two iterations, inclusive of the optional 5′ linker sequence and/or the optional 3′ linker sequence, are separated by at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, or at least 70 nucleotides.

In some aspects, the at least one antigen-encoding nucleic acid sequence is described, from 5′ to 3′, by the formula:

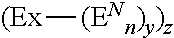

wherein, E represents a nucleotide sequence comprising at least one of the distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof.

In some aspects, the antigen-encoding cassette encodes at least 4 iterations of each of the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82).

In some aspects, the distinct epitope-encoding nucleic acid sequences comprises at least two distinct epitope-encoding nucleic acid sequences each encoding distinct KRAS-associated MHC class I neoepitopes. In some aspects, the distinct epitope-encoding nucleic acid sequences comprises at least 3, at least 4, at least 5, at least 6, at least 7, or at least 8 distinct epitope-encoding nucleic acid sequences each encoding distinct KRAS-associated MHC class I neoepitopes. In some aspects, each of the epitope-encoding nucleic acid sequences of the at least one antigen-encoding nucleic acid sequence encodes a distinct KRAS-associated MHC class I neoepitope. In some aspects, one or more of the nucleic acid sequences encoding the distinct KRAS-associated MHC class I neoepitopes comprises at least 2, at least 3, at least 4, at least 5, at least 6, at least 7, or at least 8 iterations. In some aspects, each of the nucleic acid sequences encoding the distinct KRAS-associated MHC class I neoepitopes comprises at least 2, at least 3, at least 4, at least 5, at least 6, at least 7, or at least 8 iterations. In some aspects, one or more of the nucleic acid sequences encoding the distinct KRAS-associated MHC class I neoepitopes comprises at least 4 iterations. In some aspects, each of the nucleic acid sequences encoding the distinct KRAS-associated MHC class I neoepitopes comprises at least 4 iterations. In some aspects, one or more of the distinct KRAS-associated MHC class I neoepitopes independently comprises a KRAS G12C mutation, a KRAS G12V mutation, a KRAS G12D mutation, or a KRAS Q61H mutation.

In some aspects, the at least two iterations comprises a number of iterations, or z comprises a number, sufficient to stimulate a greater immune response relative to an antigen-encoding nucleic acid sequence comprising a single iteration of the epitope-encoding nucleic acid sequence. In some aspects, the at least two iterations comprises a number of iterations, or z comprises a number, sufficient to stimulate an immune response, and a single iteration of the epitope-encoding nucleic acid sequence is insufficient to stimulate the immune response or insufficient to stimulate a detectable immune response. In some aspects, the immune response is an expansion of epitope-specific T cells following in vivo immunization with the composition for delivery of the antigen expression system. In some aspects, the immune response is increased activation of epitope-specific T cells and/or increased epitope-specific killing by epitope-specific T cells following in vivo immunization with the composition for delivery of the antigen expression system.

Also provided for herein is a composition for delivery of an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least two distinct epitope-encoding nucleic acid sequences, optionally comprising: (1) at least one alteration that makes the encoded epitope sequence distinct from the corresponding peptide sequence encoded by a wild-type nucleic acid sequence, optionally wherein the at least one alteration is a KRAS mutation, or (2) a nucleic acid sequence encoding an infectious disease organism peptide selected from the group consisting of: a pathogen-derived peptide, a virus-derived peptide, a bacteria-derived peptide, a fungus-derived peptide, and a parasite-derived peptide, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response.

Also provided for herein is a composition for delivery of an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least 2, 3, 4, 5, 6, 7, 8, 9, or 10 distinct epitope-encoding nucleic acid sequences linearly linked to each other, optionally comprising: (1) at least one alteration that makes the encoded epitope sequence distinct from the corresponding peptide sequence encoded by a wild-type nucleic acid sequence, optionally wherein the at least one alteration is a KRAS mutation, or (2) a nucleic acid sequence encoding an infectious disease organism peptide selected from the group consisting of: a pathogen-derived peptide, a virus-derived peptide, a bacteria-derived peptide, a fungus-derived peptide, and a parasite-derived peptide, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, and wherein if the second promoter nucleotide sequence is absent, the antigen-encoding nucleic acid sequence is operably linked to the at least one promoter nucleotide sequence, and wherein the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response.

In some aspects, at least one of the distinct epitope-encoding nucleic acid sequences encodes a KRAS-associated MHC class I neoepitope.

Also provided for herein is a composition for delivery of an antigen expression system, comprising: the antigen expression system, wherein the antigen expression system comprises one or more vectors, the one or more vectors comprising: (a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and (b) a cassette, optionally wherein the cassette is integrated between a native promoter nucleotide sequence native to the vector backbone and a poly(A) sequence, optionally wherein the poly(A) sequence is native to the vector backbone, wherein the cassette comprises: (i) at least one antigen-encoding nucleic acid sequence, comprising: (I) at least one epitope-encoding nucleic acid sequence, optionally comprising at least two distinct epitope-encoding nucleic acid sequences linearly linked to each other, each epitope-encoding nucleic acid sequence optionally comprising: (A) a MHC class I epitope encoding nucleic acid sequence, wherein the MHC class I epitope encoding nucleic acid sequence encodes a MHC class I epitope 7-15 amino acids in length, (B) a 5′ linker sequence, wherein the 5′ linker sequence encodes a native N-terminal amino acid sequence of the MHC class I epitope, and wherein the 5′ linker sequence encodes a peptide that is at least 2 amino acids in length, (C) a 3′ linker sequence, wherein the 3′ linker sequence encodes a native C-terminal acid sequence of the MHC class I epitope, and wherein the 3′ linker sequence encodes a peptide that is at least 2 amino acids in length, and wherein the cassette is operably linked to the native promoter nucleotide sequence, wherein each of the epitope-encoding nucleic acid sequences encodes a polypeptide that is between 13 and 25 amino acids in length, and wherein each 3′ end of each epitope-encoding nucleic acid sequence is linked to the 5′ end of the following epitope-encoding nucleic acid sequence with the exception of the final epitope-encoding nucleic acid sequence in the cassette; and (ii) at least two MHC class II epitope-encoding nucleic acid sequences comprising: (I) a PADRE MHC class II sequence (SEQ ID NO:48), (II) a Tetanus toxoid MHC class II sequence (SEQ ID NO:46), (III) a first nucleic acid sequence encoding a GPGPG amino acid linker sequence (SEQ ID NO:56) linking the PADRE MHC class II sequence and the Tetanus toxoid MHC class II sequence, (IV) a second nucleic acid sequence encoding a GPGPG amino acid linker sequence (SEQ ID NO:56) linking the 5′ end of the at least two MHC class II epitope-encoding nucleic acid sequences to the epitope-encoding nucleic acid sequences, (V) optionally, a third nucleic acid sequence encoding a GPGPG amino acid linker sequence (SEQ ID NO:56) at the 3′ end of the at least two MHC class II epitope-encoding nucleic acid sequences, and (iii) optionally, a second promoter nucleotide sequence operably linked to the antigen-encoding nucleic acid sequence; and wherein if the second promoter nucleotide sequence is absent, the antigen-encoding nucleic acid sequence is operably linked to the native promoter nucleotide sequence, and wherein the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response.

In some aspects, the immunodominant MHC class I epitope stimulates a 10-fold, 20-fold, 30-fold, 40-fold, 50-fold, 100-fold, 200-fold, 300-fold, 400-fold, 500-fold, 1000-fold, 2000-fold, 3000-fold, 4000-fold, 5000-fold, or 10,000-fold or greater immune response when administered in a vaccine composition to a subject relative to another MHC class I epitope encoded in the cassette and capable of stimulating an immune response in the subject. In some aspects, the immunodominant MHC class I epitope reduces the immune response of the other MHC class I epitope to below a limit of detection and/or does not stimulate a therapeutically effective response. In some aspects, the subject expresses at least one HLA allele known or predicted to present both the immunodominant MHC class I epitope and the other MHC class I epitope encoded in the cassette.

In some aspects, one or more of the epitope-encoding nucleic acid sequences are derived from a tumor. In some aspects, each of the epitope-encoding nucleic acid sequences are derived from a tumor, an infection, or an infected cell of a subject. In some aspects, one or more of the epitope-encoding nucleic acid sequences are not derived from a tumor, an infection, or an infected cell of a subject. In some aspects, each of the epitope-encoding nucleic acid sequences are not derived from a tumor, an infection, or an infected cell of a subject.

In some aspects, the epitope-encoding nucleic acid sequence encodes an epitope known or suspected to be presented by MHC class I on a surface of a cell, optionally wherein the surface of the cell is a tumor cell surface or an infected cell surface, and optionally wherein the cell is a subject's cell. In some aspects, the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer, or wherein the cell is an infected cell selected from the group consisting of: a pathogen infected cell, a virally infected cell, a bacterially infected cell, a fungally infected cell, and a parasitically infected cell. In some aspects, the virally infected cell is selected from the group consisting of: an HIV infected cell, a Severe acute respiratory syndrome-related coronavirus (SARS) infected cell, a severe acute respiratory syndrome coronavirus 2 (SARS-CoV-2) infected cell, a Ebola infected cell, a Hepatitis B virus (HBV) infected cell, an influenza infected cell, an orthymyxoviridae family virus infected cell, a Human papillomavirus (HPV) infected cell, a Cytomegalovirus (CMV) infected cell, a Chikungunya virus infected cell, a Respiratory syncytial virus (RSV) infected cell, a Dengue virus infected cell, and a Hepatitis C virus (HCV) infected cell.

In some aspects, the composition further comprises a nanoparticulate delivery vehicle. In some aspects, the nanoparticulate delivery vehicle is a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, the cassette is integrated between the at least one promoter nucleotide sequence and the at least one poly(A) sequence. In some aspects, the second promoter is absent and the at least one promoter nucleotide sequence is operably linked to the antigen-encoding nucleic acid sequence.

In some aspects, the one or more vectors comprise one or more +-stranded RNA vectors. In some aspects, the one or more +-stranded RNA vectors comprise a 5′ 7-methylguanosine (m7g) cap. In some aspects, the one or more +-stranded RNA vectors are produced by in vitro transcription. In some aspects, the one or more vectors are self-replicating within a mammalian cell. In some aspects, the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the backbone comprises at least one nucleotide sequence of a Venezuelan equine encephalitis virus. In some aspects, the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the backbone does not encode structural virion proteins capsid, E2 and E1. In some aspects, the cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus.

In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11175. In some aspects, the backbone comprises the sequence set forth in SEQ ID NO:6 or SEQ ID NO:7. In some aspects, the cassette is inserted at position 7544 to replace the deletion between base pairs 7544 and 11175 as set forth in the sequence of SEQ ID NO:3 or SEQ ID NO:5.

In some aspects, the insertion of the cassette provides for transcription of a polycistronic RNA comprising the nsP1-4 genes and the at least one antigen-encoding nucleic acid sequence, wherein the nsP1-4 genes and the at least one antigen-encoding nucleic acid sequence are in separate open reading frames.

In some aspects, the backbone comprises at least one nucleotide sequence of a chimpanzee adenovirus vector. In some aspects, the chimpanzee adenovirus vector is a ChAdV68 vector. In some aspects, the ChAdV68 vector comprises a ChAdV68 vector backbone comprising the sequence set forth in SEQ ID NO:1. In some aspects, the ChAdV68 vector comprises a ChAdV68 vector backbone comprising the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B; (2) E1A, E1B, and E3; or (3) E1A, E1B, E3, and E4 of the sequence set forth in SEQ ID NO:1. In some aspects, the ChAdV68 vector comprises a ChAdV68 vector backbone comprising a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO:1. In some aspects, the ChAdV68 vector comprises a ChAdV68 vector backbone comprising a partially deleted E4 gene comprising a deleted or partially-deleted E4orf2 region and a deleted or partially-deleted E4orf3 region, and optionally a deleted or partially-deleted E4orf4 region. In some aspects, the ChAdV68 vector comprises a ChAdV68 vector backbone comprising at least nucleotides 2 to 36,518 of the sequence set forth in SEQ ID NO:1 and further comprising: (1) an E1 deletion of at least nucleotides 577 to 3403 of the sequence shown in SEQ ID NO:1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion. In some aspects, the ChAdV68 vector comprises a ChAdV68 vector backbone comprising the sequence set forth in SEQ ID NO:68, optionally wherein the antigen cassette is inserted within the E1 deletion. In some aspects, the ChAdV68 vector comprises a ChAdV68 vector backbone comprising one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1. In some aspects, the ChAdV68 vector comprises a ChAdV68 vector backbone comprising one or more deletions between base pair number 3957 and 10346, base pair number 21787 and 23370, and base pair number 33486 and 36193 of the sequence set forth in SEQ ID NO:1. In some aspects, the cassette is inserted in the ChAdV vector backbone at the E1 region, E3 region, and/or any deleted AdV region that allows incorporation of the cassette.

In some aspects, the at least one promoter nucleotide sequence is the native 26S promoter nucleotide sequence encoded by the backbone. In some aspects, the at least one promoter nucleotide sequence is an exogenous RNA promoter. In some aspects, the second promoter nucleotide sequence is a 26S promoter nucleotide sequence. In some aspects, the second promoter nucleotide sequence comprises multiple 26S promoter nucleotide sequences, wherein each 26S promoter nucleotide sequence provides for transcription of one or more of the separate open reading frames.

In some aspects, the one or more vectors are each at least 300nt in size. In some aspects, the one or more vectors are each at least 1 kb in size. In some aspects, the one or more vectors are each 2 kb in size. In some aspects, the one or more vectors are each less than 5 kb in size.

In some aspects, at least one of the at least one antigen-encoding nucleic acid sequences encodes a polypeptide sequence or portion thereof that is presented by MHC class I on a cell surface, optionally a tumor cell surface or an infected cell surface.

In some aspects, each epitope-encoding nucleic acid sequence is linked directly to one another. In some aspects, at least one of the epitope-encoding nucleic acid sequences is linked to a distinct epitope-encoding nucleic acid sequence with a nucleic acid sequence encoding a linker. In some aspects, the linker links two MHC class I sequences or an MHC class I sequence to an MHC class II sequence. In some aspects, the linker is selected from the group consisting of: (1) consecutive glycine residues, at least 2, 3, 4, 5, 6, 7, 8, 9, or 10 residues in length (SEQ ID NO: 99); (2) consecutive alanine residues, at least 2, 3, 4, 5, 6, 7, 8, 9, or 10 residues in length (SEQ ID NO: 100); (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some aspects, the linker links two MHC class II sequences or an MHC class II sequence to an MHC class I sequence. In some aspects, the linker comprises the sequence GPGPG (SEQ ID NO:56). In some aspects, at least one sequence of the epitope-encoding nucleic acid sequences is linked, operably or directly, to a separate or contiguous sequence that enhances the expression, stability, cell trafficking, processing and presentation, and/or immunogenicity of the epitope-encoding nucleic acid sequences of epitope encoded therefrom. In some aspects, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76.

In some aspects, at least one of the epitope-encoding nucleic acid sequences encodes a polypeptide sequence or portion thereof that has increased binding affinity to its corresponding MHC allele relative to the translated, corresponding wild-type nucleic acid sequence. In some aspects, at least one of the epitope-encoding nucleic acid sequences encodes a polypeptide sequence or portion thereof that has increased binding stability to its corresponding MHC allele relative to the translated, corresponding wild-type nucleic acid sequence. In some aspects, at least one of the epitope-encoding nucleic acid sequences encodes a polypeptide sequence or portion thereof that has an increased likelihood of presentation on its corresponding MHC allele relative to the translated, corresponding wild-type nucleic acid sequence. In some aspects, the at least one alteration comprises a point mutation, a frameshift mutation, a non-frameshift mutation, a deletion mutation, an insertion mutation, a splice variant, a genomic rearrangement, or a proteasome-generated spliced antigen.

In some aspects, the tumor is selected from the group consisting of: lung cancer, melanoma, breast cancer, ovarian cancer, prostate cancer, kidney cancer, gastric cancer, colon cancer, testicular cancer, head and neck cancer, pancreatic cancer, bladder cancer, brain cancer, B-cell lymphoma, acute myelogenous leukemia, adult acute lymphoblastic leukemia, chronic myelogenous leukemia, chronic lymphocytic leukemia, T cell lymphocytic leukemia, non-small cell lung cancer, and small cell lung cancer, or the infectious disease organism is selected from the group consisting of: Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, an orthymyxoviridae family virus, and tuberculosis.

In some aspects, the at least one antigen-encoding nucleic acid sequence comprises at least 2-10, 2, 3, 4, 5, 6, 7, 8, 9, or 10 epitope-encoding nucleic acid sequences. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 epitope-encoding nucleic acid sequences. In some aspects, the at least one antigen-encoding nucleic acid sequence comprises at least 2-400 epitope-encoding nucleic acid sequences and wherein at least two of the epitope-encoding nucleic acid sequences encode polypeptide sequences or portions thereof that are presented by MHC class I on a cell surface, optionally a tumor cell surface or an infected cell surface. In some aspects, the at least one antigen-encoding nucleic acid sequence comprises at least 2-10, 2, 3, 4, 5, 6, 7, 8, 9, or 10 antigen-encoding nucleic acid sequences. In some aspects, the at least one antigen-encoding nucleic acid sequence comprises at least 11-20, 15-20, 11-100, 11-200, 11-300, 11-400, 11, 12, 13, 14, 15, 16, 17, 18, 19, 20 or up to 400 antigen-encoding nucleic acid sequences. In some aspects, the at least one antigen-encoding nucleic acid sequence comprises at least 2-400 antigen-encoding nucleic acid sequences and wherein at least two of the antigen-encoding nucleic acid sequences encode polypeptide sequences or portions thereof that are presented by MHC class I on a cell surface, optionally a tumor cell surface or an infected cell surface. In some aspects, at least two of the epitope-encoding nucleic acid sequences encode polypeptide sequences or portions thereof that are presented by MHC class I on a cell surface, optionally a tumor cell surface or an infected cell surface.

In some aspects, when administered to the subject and translated, at least one of the epitopes encoded by the epitope-encoding nucleic acid sequences are presented on antigen presenting cells resulting in an immune response targeting at least one of the antigens on the tumor cell surface or the infected cell surface. In some aspects, the at least one antigen-encoding nucleic acid sequences when administered to the subject and translated, at least one of the MHC class I or class II epitopes are presented on antigen presenting cells resulting in an immune response targeting at least one of the epitopes on a tumor cell surface or the infected cell surface, and optionally wherein the expression of each of the at least one antigen-encoding nucleic acid sequences is driven by the at least one promoter nucleotide sequence.

In some aspects, each epitope-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length.

In some aspects, the at least one MHC class II epitope-encoding nucleic acid sequence is present. In some aspects, the at least one MHC class II epitope-encoding nucleic acid sequence is present and comprises at least one MHC class II epitope-encoding nucleic acid sequence that comprises at least one alteration that makes the encoded peptide sequence distinct from the corresponding peptide sequence encoded by a wild-type nucleic acid sequence. In some aspects, the at least one MHC class II epitope-encoding nucleic acid sequence is 12-20, 12, 13, 14, 15, 16, 17, 18, 19, 20, or 20-40 amino acids in length. In some aspects, the at least one MHC class II epitope-encoding nucleic acid sequence is present and comprises at least one universal MHC class II antigen-encoding nucleic acid sequence, optionally wherein the at least one universal sequence comprises at least one of Tetanus toxoid and PADRE.

In some aspects, the at least one promoter nucleotide sequence or the second promoter nucleotide sequence is inducible. In some aspects, the at least one promoter nucleotide sequence or the second promoter nucleotide sequence is non-inducible.

In some aspects, the at least one poly(A) sequence comprises a poly(A) sequence native to the backbone. In some aspects, the at least one poly(A) sequence comprises a poly(A) sequence exogenous to the backbone. In some aspects, the at least one poly(A) sequence is operably linked to at least one of the at least one antigen-encoding nucleic acid sequences. In some aspects, the at least one poly(A) sequence is at least 20, at least 30, at least 40, at least 50, at least 60, at least 70, at least 80, at least 90, or at least 100 consecutive A nucleotides (SEQ ID NO: 98). In some aspects, the at least one poly(A) sequence is at least 80 consecutive A nucleotides.

In some aspects, the cassette further comprises at least one of: an intron sequence, a woodchuck hepatitis virus posttranscriptional regulatory element (WPRE) sequence, an internal ribosome entry sequence (IRES) sequence, a nucleotide sequence encoding a 2A self-cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences. In some aspects, the cassette further comprises a reporter gene, including but not limited to, green fluorescent protein (GFP), a GFP variant, secreted alkaline phosphatase, luciferase, a luciferase variant, or a detectable peptide or epitope. In some aspects, the detectable peptide or epitope is selected from the group consisting of an HA tag, a Flag tag, a His-tag, or a V5 tag.

In some aspects, the one or more vectors further comprises one or more nucleic acid sequences encoding at least one immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the antibody or antigen-binding fragment thereof is a Fab fragment, a Fab′ fragment, a single chain Fv (scFv), a single domain antibody (sdAb) either as single specific or multiple specificities linked together (e.g., camelid antibody domains), or full-length single-chain antibody (e.g., full-length IgG with heavy and light chains linked by a flexible linker). In some aspects, the heavy and light chain sequences of the antibody are a contiguous sequence separated by either a self-cleaving sequence such as 2A or IRES; or the heavy and light chain sequences of the antibody are linked by a flexible linker such as consecutive glycine residues. In some aspects, the immune modulator is a cytokine. In some aspects, the cytokine is at least one of IL-2, IL-7, IL-12, IL-15, or IL-21 or variants thereof of each.

In some aspects, at least one epitope-encoding nucleic acid sequence is selected by performing the steps of: (a) obtaining at least one of exome, transcriptome, or whole genome nucleotide sequencing data from a tumor, an infected cell, or an infectious disease organism, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a cell surface, optionally a tumor cell surface or an infected cell surface, the set of numerical likelihoods having been identified at least based on received mass spectrometry data; and (c) selecting a subset of the set of antigens based on the set of numerical likelihoods to generate a set of selected antigens which are used to generate the epitope-encoding nucleic acid sequences.

In some aspects, each of the epitope-encoding nucleic acid sequences is selected by performing the steps of: (a) obtaining at least one of exome, transcriptome, or whole genome nucleotide sequencing data from a tumor, an infected cell, or an infectious disease organism, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a cell surface, optionally a tumor cell surface or an infected cell surface, the set of numerical likelihoods having been identified at least based on received mass spectrometry data; and (c) selecting a subset of the set of antigens based on the set of numerical likelihoods to generate a set of selected antigens which are used to generate the at least 20 epitope-encoding nucleic acid sequences. In some aspects, a number of the set of selected antigens is 2-20. In some aspects, the presentation model represents dependence between: (a) presence of a pair of a particular one of the MHC alleles and a particular amino acid at a particular position of a peptide sequence; and (b) likelihood of presentation on a cell surface, optionally a tumor cell surface or an infected cell surface, by the particular one of the MHC alleles of the pair, of such a peptide sequence comprising the particular amino acid at the particular position. In some aspects, selecting the set of selected antigens comprises selecting antigens that have an increased likelihood of being presented on the cell surface relative to unselected antigens based on the presentation model, optionally wherein the selected antigens have been validated as being presented by one or more specific HLA alleles. In some aspects, selecting the set of selected antigens comprises selecting antigens that have an increased likelihood of being capable of inducing a tumor-specific or infectious disease-specific immune response in the subject relative to unselected antigens based on the presentation model. In some aspects, selecting the set of selected antigens comprises selecting antigens that have an increased likelihood of being capable of being presented to naïve T cells by professional antigen presenting cells (APCs) relative to unselected antigens based on the presentation model, optionally wherein the APC is a dendritic cell (DC). In some aspects, selecting the set of selected antigens comprises selecting antigens that have a decreased likelihood of being subject to inhibition via central or peripheral tolerance relative to unselected antigens based on the presentation model. In some aspects, selecting the set of selected antigens comprises selecting antigens that have a decreased likelihood of being capable of inducing an autoimmune response to normal tissue in the subject relative to unselected antigens based on the presentation model. In some aspects, exome or transcriptome nucleotide sequencing data is obtained by performing sequencing on a tumor cell or tissue, an infected cell, or an infectious disease organism. In some aspects, the sequencing is next generation sequencing (NGS) or any massively parallel sequencing approach.

In some aspects, the cassette comprises junctional epitope sequences formed by adjacent sequences in the cassette. In some aspects, at least one or each junctional epitope sequence has an affinity of greater than 500 nM for MHC. In some aspects, each junctional epitope sequence is non-self.

In some aspects, each of the MHC class I epitopes is predicted or validated to be capable of presentation by at least one HLA allele present in at least 5% of a population. In some aspects, each of the MHC class I epitopes is predicted or validated to be capable of presentation by at least one HLA allele, wherein each antigen/HLA pair has an antigen/HLA prevalence of at least 0.01% in a population. In some aspects, each of the MHC class I epitopes is predicted or validated to be capable of presentation by at least one HLA allele, wherein each antigen/HLA pair has an antigen/HLA prevalence of at least 0.1% in a population.

In some aspects, the cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the cassette.

In some aspects, the prediction is based on presentation likelihoods generated by inputting sequences of the non-therapeutic epitopes into a presentation model.

In some aspects, an order of the at least one antigen-encoding nucleic acid sequences in the cassette is determined by a series of steps comprising: (a) generating a set of candidate cassette sequences corresponding to different orders of the at least one antigen-encoding nucleic acid sequences; (b) determining, for each candidate cassette sequence, a presentation score based on presentation of non-therapeutic epitopes in the candidate cassette sequence; and (c) selecting a candidate cassette sequence associated with a presentation score below a predetermined threshold as the cassette sequence for an antigen vaccine.

Also provided for herein is a pharmaceutical composition comprising any of the compositions described herein and a pharmaceutically acceptable carrier. In some aspects, the composition further comprises an adjuvant. In some aspects, the composition further comprises an immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Also provided for herein is an isolated nucleotide sequence or set of isolated nucleotide sequences comprising the cassette of any of the compositions described herein and one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA. In some aspects, the sequence or set of isolated nucleotide sequences comprises the cassette of any of the above composition claims inserted at position 7544 of the sequence set forth in SEQ ID NO:6 or SEQ ID NO:7. In some aspects, the composition further comprises: a) a T7 or SP6 RNA polymerase promoter nucleotide sequence 5′ of the one or more elements obtained from the sequence of SEQ ID NO:3 or SEQ ID NO:5; and b) optionally, one or more restriction sites 3′ of the poly(A) sequence. In some aspects, the cassette of any of the above composition claims is inserted at position 7563 of SEQ ID NO:8 or SEQ ID NO:9.

Also provided for herein is a vector or set of vectors comprising any of the nucleotide sequence described herein.

Also provided for herein is an isolated cell comprising any of the nucleotide sequences or set of isolated nucleotide sequences described herein, optionally wherein the cell is a BHK-21, CHO, HEK293 or variants thereof, 911, HeLa, A549, LP-293, PER.C6, or AE1-2a cell.

Also provided for herein is a kit comprising any of the compositions described herein and instructions for use.

In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of: a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof.

In some aspects, the antigen expression system is fully encapsulated in the LNPs.

In some aspects, the non-lamellar morphology of the LNPs comprises an inverse hexagonal (H_{II}) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs.

In some aspects, the LNP comprises a compound having a structure of Formula I:

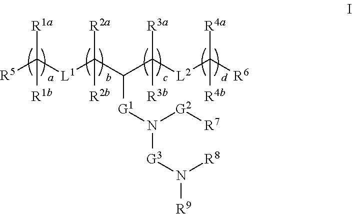

or a pharmaceutically acceptable salt, tautomer, prodrug or stereoisomer thereof, wherein: L¹ and L2 are each independently -0(C=0)-, —(C=0)0-, —C(=0)-, -0-, —S(0)ₓ—, —S—S—, —C(=0)S—, —SC(=0)-, —R^{a}C(=0)-, —C(=0) R^{a}—, —R^{a}C(=0) R^{a}—, -0C(=0) R^{a}—, —R^{a}C(=0)0- or a direct bond; G¹ is C₁-C₂ alkylene, —(C=0)-, -0(C=0)-, —SC(=0)-, —R^{a}C(=0)- or a direct bond: —C(=0)-, —(C=0)0-, —C(=0)S—, —C(=0) R^{a}— or a direct bond; G is C₁-C₆ alkylene; R^{a} is H or C₁-C₁₂ alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R²¹ are, at each occurrence, independently either: (a) H or C₁-C₁₂ alkyl; or (b) R^{2a} is H or C₁-C₁₂ alkyl, and R²¹ together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R⁴¹ are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C₄-C₂₀ alkyl; R⁸ and R⁹ are each independently C₁-C₁₂ alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

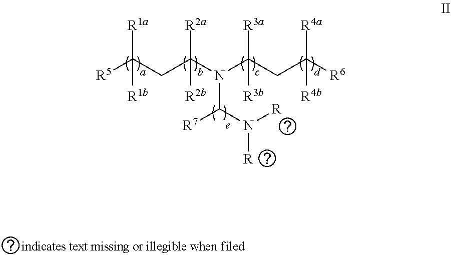

or a pharmaceutically acceptable salt, tautomer, prodrug or stereoisomer thereof, wherein: L¹ and L2 are each independently -0(C=0)-, —(C=0)0- or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C₁-C₁₂ alkyl, or at least one of L¹ or L² is -0(C=0)- or —(C=0)0-; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

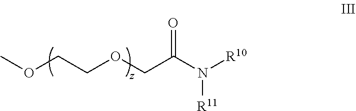

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.
start here

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

Also provided for herein is a method for treating a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject any of the compositions or any of the pharmaceutical compositions described herein. In some aspects, the epitope-encoding nucleic acid sequence is derived from the tumor of the subject with cancer or from a cell or sample of the infected subject. In some aspects, the epitope-encoding nucleic acid sequence are not derived from the tumor of the subject with cancer or from a cell or sample of the infected subject.

Also provided for herein is a method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject any of the compositions or any of the pharmaceutical compositions described herein.

In some aspects, the subject expresses at least one HLA allele predicted or known to present the MHC class I epitope. In some aspects, HLA allele predicted or known to present the MHC class I epitope is A*03:01, A*11:01, A*02:01, A*68:01, B*07:02, C*01:02, C*03:04, C*08:02 and/or A*01:01. In some aspects, HLA allele predicted or known to present the MHC class I epitope is A*03:01. In some aspects, HLA allele predicted or known to present the MHC class I epitope is A*11:01. In some aspects, HLA allele predicted or known to present the MHC class I epitope is A*02:01. In some aspects, HLA allele predicted or known to present the MHC class I epitope is C*01:02. In some aspects, HLA allele predicted or known to present the MHC class I epitope is A*68:01. In some aspects, HLA allele predicted or known to present the MHC class I epitope is B*07:02. In some aspects, HLA allele predicted or known to present the MHC class I epitope is C*03:04. In some aspects, HLA allele predicted or known to present the MHC class I epitope is C*08:02. In some aspects, HLA allele predicted or known to present the MHC class I epitope is A*01:01.

In some aspects, the composition is administered intramuscularly (IM), intradermally (ID), subcutaneously (SC), or intravenously (IV). In some aspects, the composition is administered intramuscularly. In some aspects, the method further comprising administration of one or more immune modulators, optionally wherein the immune modulator is administered before, concurrently with, or after administration of the composition or pharmaceutical composition. In some aspects, the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof. In some aspects, the immune modulator is administered intravenously (IV), intramuscularly (IM), intradermally (ID), or subcutaneously (SC). In some aspects, the subcutaneous administration is near the site of the composition or pharmaceutical composition administration or in close proximity to one or more vector or composition draining lymph nodes. In some aspects, the one or more immune modulators are selected from the group consisting of: nivolumamb, cemiplimab, and ipilimumab. In some aspects, the one or more immune modulators comprises nivolumamb. In some aspects, the one or more immune modulators comprises cemiplimab. In some aspects, the one or more immune modulators comprises ipilimumab.

In some aspects, the method further comprises administering to the subject a second vaccine composition. In some aspects, the second vaccine composition is administered prior to the administration of any of the compositions or the pharmaceutical compositions described herein. In some aspects, the second vaccine composition is administered subsequent to the administration of any of the compositions or the pharmaceutical compositions described herein. In some aspects, the second vaccine composition is the same as any of the compositions or the pharmaceutical compositions described herein. In some aspects, the second vaccine composition is different any of the compositions or the pharmaceutical compositions described herein. In some aspects, the second vaccine composition comprises a chimpanzee adenovirus vector encoding at least one antigen-encoding nucleic acid sequence. In some aspects, the at least one antigen-encoding nucleic acid sequence encoded by the chimpanzee adenovirus vector is the same as the at least one antigen-encoding nucleic acid sequence of any of the above composition claims.

Also provided for herein is a method of manufacturing the one or more vectors of any of the above composition claims, the method comprising: (a) obtaining a linearized DNA sequence comprising the backbone and the cassette; (b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7g cap to the resulting RNA; and (c) isolating the one or more vectors from the in vitro transcription reaction. In some aspects, the linearized DNA sequence is generated by linearizing a DNA plasmid sequence or by amplification using PCR. In some aspects, the DNA plasmid sequence is generated using one of bacterial recombination or full genome DNA synthesis or full genome DNA synthesis with amplification of synthesized DNA in bacterial cells. In some aspects, isolating the one or more vectors from the in vitro transcription reaction involves one or more of phenol chloroform extraction, silica column based purification, or similar RNA purification methods.

Also provided for herein is a method of manufacturing the composition of any of the above composition claims for delivery of the antigen expression system, the method comprising: (a) providing components for the nanoparticulate delivery vehicle; (b) providing the antigen expression system; and (c) providing conditions sufficient for the nanoparticulate delivery vehicle and the antigen expression system to produce the composition for delivery of the antigen expression system. In some aspects, the conditions are provided by microfluidic mixing.

Also provided for herein is a method for treating a subject with a disease, wherein the disease is cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ- (E^{N}ₙ)_{y})_{z}

wherein E represents a nucleotide sequence comprising a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes the distinct KRAS-associated MHC class I neoepitope.

Also provided for herein is a method for treating a subject with a disease, wherein the disease is cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding the KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of the epitope-encoding nucleic acid sequence encoding the KRAS-associated MHC class I neoepitope.

In some aspects, the antigen-encoding cassette encodes at least 4 iterations of each of the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82).

In some aspects, the epitope-encoding nucleic acid sequence is derived from a tumor of the subject with cancer or from a cell or sample of the infected subject. In some aspects, the epitope-encoding nucleic acid sequence are not derived from a tumor of the subject with cancer or from a cell or sample of the infected subject.

Also provided for herein is a method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ-(E^{N}ₙ)_{y})_{z}

wherein E represents a nucleotide sequence comprising a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes the distinct KRAS-associated MHC class I neoepitope.

Also provided for herein is a method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding the KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the at least one antigen-encoding nucleic acid sequence comprises at least two iterations of at least one of the epitope-encoding nucleic acid sequences encoding the KRAS-associated MHC class I neoepitope.

Also provided for herein is a method for treating a subject with a disease, wherein the disease is cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least two distinct epitope-encoding nucleic acid sequences, optionally comprising: (1) at least one alteration that makes the encoded epitope sequence distinct from the corresponding peptide sequence encoded by a wild-type nucleic acid sequence, optionally wherein the at least one alteration is a KRAS mutation, or (2) a nucleic acid sequence encoding an infectious disease organism peptide selected from the group consisting of: a pathogen-derived peptide, a virus-derived peptide, a bacteria-derived peptide, a fungus-derived peptide, and a parasite-derived peptide, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response.

In some aspects, the antigen-encoding cassette encodes at least 4 iterations of each of the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82).

In some aspects, the subject expresses at least one HLA allele predicted or known to present the at least one epitope sequence, optionally wherein the at least one epitope sequence predicted or known to be presented comprises (1) the KRAS-associated MHC class I neoepitope, and/or (2) the immunodominant MHC class I epitope and the other MHC class I epitope encoded in the cassette. In some aspects, the subject expresses at least one HLA allele predicted or known to present the at least one epitope sequence, and wherein the at least one epitope sequence comprises an epitope known or suspected to be presented by MHC class I on a surface of a cell, optionally wherein the at least one epitope sequence predicted or known to be presented comprises (1) the KRAS-associated MHC class I neoepitope, and/or (2) the immunodominant MHC class I epitope and the other MHC class I epitope encoded in the cassette. In some aspects, the surface of the cell is a tumor cell surface. In some aspects, the cell is a tumor cell selected from the group consisting of: lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, and small cell lung cancer. In some aspects, the surface of the cell is an infected cell surface. In some aspects, the cell is an infected cell selected from the group consisting of: a pathogen infected cell, a virally infected cell, a bacterially infected cell, a fungally infected cell, and a parasitically infected cell. In some aspects, the virally infected cell is selected from the group consisting of: an HIV infected cell, a Severe acute respiratory syndrome-related coronavirus (SARS) infected cell, a severe acute respiratory syndrome coronavirus 2 (SARS-CoV-2) infected cell, a Ebola infected cell, a Hepatitis B virus (HBV) infected cell, an influenza infected cell, an orthymyxoviridae family virus infected cell, a Human papillomavirus (HPV) infected cell, a Cytomegalovirus (CMV) infected cell, a Chikungunya virus infected cell, a Respiratory syncytial virus (RSV) infected cell, a Dengue virus infected cell, and a Hepatitis C virus (HCV) infected cell.

Also provided for herein is a method for inducing an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:

(Eₓ- (E^{N}ₙ)_{y})_{z}

wherein E represents a nucleotide sequence comprising a distinct epitope-encoding nucleic acid sequences, n represents the number of separate distinct epitope-encoding nucleic acid sequences and is any integer including 0, E^{N} represents a nucleotide sequence comprising the separate distinct epitope-encoding nucleic acid sequence for each corresponding n, for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and at least one of the distinct epitope-encoding nucleic acid sequences comprising the at least two iterations encodes a distinct KRAS-associated MHC class I neoepitope.

Also provided for herein is a method for inducing an immune response in a subject with cancer, wherein the cancer comprises (1) a solid tumor expressing the KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) an epitope-encoding nucleic acid sequence encoding a KRAS-associated MHC class I neoepitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, wherein the at least one antigen-encoding nucleic acid sequence comprises at least two iterations of the epitope-encoding nucleic acid sequence encoding the KRAS-associated MHC class I neoepitope, and wherein the subject expresses at least one HLA allele predicted or known to present the at least one KRAS-associated MHC class I neoepitope.

Also provided for herein is a method for inducing an immune response in a subject with cancer, wherein the cancer comprises (1) a solid tumor expressing the KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises: an antigen expression system, comprising: the antigen expression system, wherein the antigen expression system comprises one or more vectors, the one or more vectors comprising: (a) a vector backbone, wherein the backbone comprises: (i) at least one promoter nucleotide sequence, and (ii) optionally, at least one polyadenylation (poly(A)) sequence; and (b) a cassette, wherein the cassette comprises: (i) at least one antigen-encoding nucleic acid sequence, comprising: (I) at least two epitope-encoding nucleic acid sequence, optionally comprising: (1) at least one alteration that makes the encoded epitope sequence distinct from the corresponding peptide sequence encoded by a wild-type nucleic acid sequence, optionally wherein the at least one alteration is a KRAS mutation, or (2) a nucleic acid sequence encoding an infectious disease organism peptide selected from the group consisting of: a pathogen-derived peptide, a virus-derived peptide, a bacteria-derived peptide, a fungus-derived peptide, and a parasite-derived peptide, and optionally wherein the epitope-encoding nucleic acid sequence encodes a MHC class I epitope, and wherein each of the epitope-encoding nucleic acid sequences comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II epitope-encoding nucleic acid sequence; (iv) optionally, at least one nucleic acid sequence encoding a GPGPG amino acid linker sequence (SEQ ID NO:56); and (v) optionally, at least one second poly(A) sequence, wherein the second poly(A) sequence is a native poly(A) sequence or an exogenous poly(A) sequence to the vector backbone, wherein if the second promoter nucleotide sequence is absent, the antigen-encoding nucleic acid sequence is operably linked to the at least one promoter nucleotide sequence, and wherein the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response, and wherein the subject expresses at least one HLA allele predicted or known to present both the immunodominant MHC class I epitope and the other MHC class I epitope encoded in the cassette.

In some aspects, the antigen-encoding cassette encodes at least 4 iterations of each of the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82). In some aspects, the KRAS-associated MHC class I neoepitope or the KRAS mutation comprises a KRAS G12C mutation, a KRAS G12V mutation, a KRAS G12D mutation, or a KRAS Q61H mutation. In some aspects, the KRAS-associated MHC class I neoepitope or the KRAS mutation comprises any one of the amino acid sequence shown in SEQ ID NOs: 75-82. In some aspects, the antigen-encoding cassette comprises each of the amino acid sequence shown in SEQ ID NOs: 75-82. In some aspects, the antigen-encoding cassette comprises two or more iterations of each of the amino acid sequence shown in SEQ ID NOs: 75-82. In some aspects, the antigen-encoding cassette comprises 4 iterations of each of the amino acid sequence shown in SEQ ID NOs: 75-82. In some aspects, the KRAS-associated MHC class I neoepitope or the KRAS mutation comprises the amino acid sequence shown in SEQ ID NO: 57, SEQ ID NO: 58, SEQ ID NO: 59, or SEQ ID NO: 60. In some aspects, the epitope-encoding nucleic acid sequences comprises two or more distinct epitope-encoding nucleic acid sequences independently encoding a distinct KRAS-associated MHC class I neoepitope or a distinct KRAS mutation. In some aspects, each of the epitope-encoding nucleic acid sequences independently encodes a distinct KRAS-associated MHC class I neoepitope or a distinct KRAS mutation. In some aspects, the epitope-encoding nucleic acid sequences comprises two or more distinct epitope-encoding nucleic acid sequences independently encoding a KRAS G12C mutation, a KRAS G12V mutation, a KRAS G12D mutation, or a KRAS Q61H mutation. In some aspects, the epitope-encoding nucleic acid sequences independently encodes each of a KRAS G12C mutation, a KRAS G12V mutation, and a KRAS G12D mutation, and optionally a KRAS Q61H mutation. In some aspects, the antigen-encoding nucleic acid sequence encodes a peptide comprising the amino acid sequence shown in SEQ ID NO: 64 or SEQ ID NO: 65. In some aspects, the antigen-encoding nucleic acid sequence encodes a peptide comprising the amino acid sequence shown in SEQ ID NO: 65.

In some aspects, the KRAS-associated MHC class I neoepitope comprising a KRAS G12C mutation is selected from the group consisting of: VVVGACGVGK (SEQ ID NO: 75), KLVVVGACGV (SEQ ID NO: 76), and GACGVGKSAL (SEQ ID NO: 93). In some aspects, the KRAS-associated MHC class I neoepitope comprising a KRAS G12D mutation is selected from the group consisting of: VVGADGVGK (SEQ ID NO: 77), VVVGADGVGK (SEQ ID NO: 78), KLVVVGADGV (SEQ ID NO: 94), and GADGVGKSAL (SEQ ID NO: 95). In some aspects, the KRAS-associated MHC class I neoepitope comprising a KRAS G12V mutation is selected from the group consisting of: VVGAVGVGK (SEQ ID NO: 79), VVVGAVGVGK (SEQ ID NO: 81), AVGVGKSAL (SEQ ID NO: 80), and GAVGVGKSAL (SEQ ID NO: 96).

In some aspects, the cassette does not encode an immunodominant MHC class I epitope that: (1) stimulates a 5-fold or greater immune response when administered in a vaccine composition to a subject relative to another MHC class I epitope encoded in the cassette and capable of stimulating an immune response in the subject, and/or (2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response. In some aspects, the cassette does not encode an immunodominant MHC class I epitope that stimulates a 5-fold or greater immune response when administered in a vaccine composition to a subject relative to a KRAS-associated neoepitope encoded in the cassette and capable of stimulating an immune response in the subject. In some aspects, the cassette does not encode an immunodominant MHC class I epitope that reduces an immune response to another MHC class I epitope encoded in the cassette when administered in a vaccine composition to a subject relative to an immune response when the other MHC class I epitope is administered in the absence of the immunodominant MHC class I epitope. In some aspects, the cassette does not encode an immunodominant MHC class I epitope that reduces an immune response to another MHC class I epitope encoded in the cassette to below a limit of detection when administered in a vaccine composition to a subject relative to an immune response when the other MHC class I epitope is administered in the absence of the immunodominant MHC class I epitope. In some aspects, the cassette does not encode an immunodominant MHC class I epitope that reduces an immune response to another MHC class I epitope encoded in the cassette when administered in a vaccine composition to a subject relative to an immune response when the other MHC class I epitope is administered in the absence of the immunodominant MHC class I epitope, wherein the immune response to the other MHC class I epitope is not a therapeutically effective response.

In some aspects, the immunodominant epitope is a TP53-associated MHC class I neoepitope, optionally wherein the TP53-associated MHC class I neoepitope comprises a S127Y mutation.

In some aspects, the antigen expression system comprises any one of the antigen expression systems described herein. In some aspects, the antigen-based vaccine comprises any one of the pharmaceutical compositions described herein.

In some aspects, the antigen-based vaccine is administered as a priming dose. In some aspects, the antigen-based vaccine is administered as one or more boosting doses. In some aspects, the boosting dose is different than the priming dose. In some aspects, a) the priming dose comprises a chimpanzee adenovirus vector and the boosting dose comprises an alphavirus vector; or b) the priming dose comprises an alphavirus vector and the boosting dose comprises a chimpanzee adenovirus vector. In some aspects, the boosting dose is the same as the priming dose. In some aspects, the injection site of the one or more boosting doses is as close as possible to the injection site of the priming dose.

In some aspects, the method further comprises determining or having determined the HLA-haplotype of the subject.

In some aspects, the antigen-based vaccine is administered intramuscularly (IM), intradermally (ID), subcutaneously (SC), or intravenously (IV). In some aspects, the antigen-based vaccine is administered intramuscularly (IM). In some aspects, the IM administration is administered at separate injection sites. In some aspects, the separate injection sites are in opposing deltoid muscles. In some aspects, the separate injection sites are in gluteus or rectus femoris sites on each side.

Also disclosed herein is a pharmaceutical composition comprising any of the compositions disclosed herein (such as an alphavirus-based or ChAd-based vector disclosed herein) and a pharmaceutically acceptable carrier. In some aspects, the pharmaceutical composition further comprises an adjuvant. In some aspects, the pharmaceutical composition further comprises an immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Also disclosed herein is a vector comprising an isolated nucleotide sequence disclosed herein.

Also disclosed herein is a kit comprising a vector or a composition disclosed herein and instructions for use.

Also disclosed herein is a method for treating a subject with cancer, wherein the cancer comprises (1) a solid tumor expressing the KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject a vector disclosed herein or a pharmaceutical composition disclosed herein. Also disclosed herein is a method for inducing an immune response in a subject with cancer, wherein the cancer comprises (1) a solid tumor expressing the KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject any of the compositions, vectors, or pharmaceutical compositions described herein.

In some aspects, the subject expresses at least one HLA allele predicted or known to present the MHC class I epitope. In some aspects, HLA allele predicted or known to present the MHC class I epitope is A*03:01, A*11:01, A*02:01, A*68:01, B*07:02, C*01:02, C*03:04, C*08:02 and/or A*01:01. In some aspects, HLA allele predicted or known to present the MHC class I epitope is A*03:01. In some aspects, HLA allele predicted or known to present the MHC class I epitope is A*11:01. In some aspects, HLA allele predicted or known to present the MHC class I epitope is A*02:01. In some aspects, HLA allele predicted or known to present the MHC class I epitope is C*01:02. In some aspects, HLA allele predicted or known to present the MHC class I epitope is A*68:01. In some aspects, HLA allele predicted or known to present the MHC class I epitope is B*07:02. In some aspects, HLA allele predicted or known to present the MHC class I epitope is C*03:04. In some aspects, HLA allele predicted or known to present the MHC class I epitope is C*08:02. In some aspects, HLA allele predicted or known to present the MHC class I epitope is A*01:01.

In some aspects, the vector or composition is administered intramuscularly (IM), intradermally (ID), or subcutaneously (SC), or intravenously (IV).

In some aspects, the cancer comprises a solid tumor expressing a KRAS-associated and/or a NRAS-associated MHC class I neoepitope. In some aspects, the KRAS-associated and/or the NRAS-associated MHC class I neoepitope comprises a mutation selected from the group consisting of: KRAS_G12C, NRAS_G12C, KRAS_G12D, NRAS_G12D, KRAS_G12V, NRAS_G12V, KRAS_Q61H, and NRAS_Q61H.

In some aspects, the cancer comprises colorectal cancer (CRC). In some aspects, the the cancer comprises non-small cell lung cancer (NSCLC). In some aspects, the cancer comprises pancreatic ductal adenocarcinoma (PDA).

In some aspects, any one of the compositions, the pharmaceutical compositions, the antigen-based vaccines, or the one or more boosting doses described herein is administered every 4 weeks (Q4W). In some aspects, any one of the compositions, the pharmaceutical compositions, the antigen-based vaccines, or the one or more boosting doses described herein is administered every 8 weeks (Q8W). In some aspects, any one of the compositions, the pharmaceutical compositions, the antigen-based vaccines, or the one or more boosting doses described herein is administered monthly. In some aspects, any one of the compositions, the pharmaceutical compositions, the antigen-based vaccines, or the one or more boosting doses described herein is administered every two months.

In some aspects, stimulating the immune response comprises stimulating a molecular response. In some aspects, the molecular response comprises a reduction in ctDNA. In some aspects, the reduction in ctDNA is at least a 20%, at least a 30%, at least a 40%, or at least a 50% reduction in ctDNA. In some aspects, the reduction in ctDNA is at least a 30% reduction in ctDNA.

Also disclosed herein is a method of manufacturing the one or more vectors of any of the above compositions, the method comprising: obtaining a linearized DNA sequence comprising the backbone and the antigen cassette; in vitro transcribing the linearized DNA sequence by addition of the linearized DNA sequence to an in vitro transcription reaction containing all the necessary components to transcribe the linearized DNA sequence into RNA, optionally further comprising in vitro addition of the m7g cap to the resulting RNA; and isolating the one or more vectors from the in vitro transcription reaction. In some aspects, the linearized DNA sequence is generated by linearizing a DNA plasmid sequence or by amplification using PCR. In some aspects, the DNA plasmid sequence is generated using one of bacterial recombination or full genome DNA synthesis or full genome DNA synthesis with amplification of synthesized DNA in bacterial cells. In some aspects, the isolating the one or more vectors from the in vitro transcription reaction involves one or more of phenol chloroform extraction, silica column based purification, or similar RNA purification methods.

Also disclosed herein is a method of manufacturing any of the compositions disclosed herein, the method comprising: providing components for the nanoparticulate delivery vehicle; providing the antigen expression system; and providing conditions sufficient for the nanoparticulate delivery vehicle and the antigen expression system to produce the composition for delivery of the antigen expression system. In some aspects, the conditions are provided by microfluidic mixing.

Also disclosed herein is a method of manufacturing a adenovirus vector disclosed herein, the method comprising: obtaining a plasmid sequence comprising the at least one promoter sequence and the antigen cassette; transfecting the plasmid sequence into one or more host cells; and isolating the adenovirus vector from the one or more host cells.

In some aspects, isolating comprises: lysing the host cell to obtain a cell lysate comprising the adenovirus vector; and purifying the adenovirus vector from the cell lysate.

In some aspects, the plasmid sequence is generated using one of bacterial recombination or full genome DNA synthesis or full genome DNA synthesis with amplification of synthesized DNA in bacterial cells. In some aspects, the one or more host cells are at least one of CHO, HEK293 or variants thereof, 911, HeLa, A549, LP-293, PER.C6, and AE1-2a cells. In some aspects, purifying the adenovirus vector from the cell lysate involves one or more of chromatographic separation, centrifugation, virus precipitation, and filtration.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A presents an illustration of cassettes featuring either a single copy of KRAS neoepitopes G12C, G12V, G12D, and Q61H (“KRAS 1× (20×1)”; cassette=SEQ ID NO: 63), 2 repeats of the KRAS G12C, G12V, G12D, and Q61H neoepitopes and 2 repeats of additional KRAS neoepitopes (“KRAS 2× (8×2)”; cassette=SEQ ID NO: 64), or 4 repeats of the KRAS neoepitopes (“KRAS 4× (4×4)”; cassette=SEQ ID NO: 65). Numerical identifiers are in reference to the epitope “slot” relative to each cassette respectively and not across cassette designs (e.g., the slot “3” epitope in the 20×1 cassette is not the same as the epitope in slot 3 of the 8×2 cassette).

FIG. 1B demonstrates repeating epitopes increases vaccine induced antigen-specific T-cell response. Shown are ELISpot results for the repeated neoepitope KRAS G12C. Mice engineered to express human HLA-A11:01 were immunized with 8×10¹⁰ VP using the ChAdV68 delivery vectors indicated and splenocytes isolated 14 days post-immunization. The number of antigen-specific T-cells were measured by IFNg ELISpot following overnight stimulation with VVVGACGVGK (SEQ ID NO: 75). Data presented as spot forming colonies (SFC) per 1×10⁶ splenocytes for each animal. Bar represents the median.

FIG. 1C demonstrates repeating epitopes increases vaccine induced antigen-specific T-cell response. Shown are ELISpot results for the repeated neoepitope KRAS G12V. Mice engineered to express human HLA-A11:01 were immunized with 8×10¹⁰ VP using the ChAdV68 delivery vectors indicated and splenocytes isolated 14 days post-immunization. The number of antigen-specific T-cells were measured by IFNg ELISpot following overnight stimulation with VVVGAVGVGK (SEQ ID NO: 81). Data presented as spot forming colonies (SFC) per 1×10⁶ splenocytes for each animal. Bar represents the median. Dashed line represent samples that were too numerous to count (TNTC).

FIG. 1D demonstrates repeating epitopes increases vaccine induced antigen-specific T-cell response. Shown are ELISpot results for the repeated neoepitope KRAS G12D. Mice engineered to express human HLA-A11:01 were immunized with 8×10¹⁰ VP using the ChAdV68 delivery vectors indicated and splenocytes isolated 14 days post-immunization. The number of antigen-specific T-cells were measured by IFNg ELISpot following overnight stimulation with VVVGADGVGK (SEQ ID NO: 78). Data presented as spot forming colonies (SFC) per 1×10⁶ splenocytes for each animal. Bar represents the median.

FIG. 2A presents an illustration of ChAdV68 delivery vectors designed to assess immunodominance of a TP53 epitope, specifically vectors containing only KRAS neoepitopes G12C, G12V, G12D, and Q61H (“KRAS 4×1”; cassette=SEQ ID NO: 66), KRAS neoepitopes in combination with a TP53 R213L neoepitope (“KRAS 4×1+R213L”; cassette=SEQ ID NO: 67), and KRAS neoepitopes in combination with a TP53 S127Y neoepitope (“KRAS 4×1+S127Y”; cassette=SEQ ID NO: 68).

FIG. 2B demonstrates removal of an immunodominant epitope increases vaccine induced antigen-specific T-cell response to KRAS neoepitopes. Shown are ELISpot results for the neoepitope KRAS G12C. Mice engineered to express human HLA-A11:01 were immunized with 5×10¹⁰ VP using the ChAdV68 delivery vectors indicated and splenocytes isolated 14 days post-immunization. The number of antigen-specific T-cells were measured by IFNg ELISpot following overnight stimulation with VVVGACGVGK (SEQ ID NO: 75). Data presented as spot forming colonies (SFC) per 1×10⁶ splenocytes for each animal. Bar represents the median.

FIG. 2C demonstrates removal of an immunodominant epitope increases vaccine induced antigen-specific T-cell response to KRAS neoepitopes. Shown are ELISpot results for the neoepitope KRAS G12D. Mice engineered to express human HLA-A11:01 were immunized with 5×10¹⁰ VP using the ChAdV68 delivery vectors indicated and splenocytes isolated 14 days post-immunization. The number of antigen-specific T-cells were measured by IFNg ELISpot following overnight stimulation with VVVGADGVGK (SEQ ID NO: 78). Data presented as spot forming colonies (SFC) per 1×10⁶ splenocytes for each animal. Bar represents the median.

FIG. 2D demonstrates removal of an immunodominant epitope increases vaccine induced antigen-specific T-cell response to KRAS neoepitopes. Shown are ELISpot results for the neoepitope KRAS G12V. Mice engineered to express human HLA-A11:01 were immunized with 5×10¹⁰ VP using the ChAdV68 delivery vectors indicated and splenocytes isolated 14 days post-immunization. The number of antigen-specific T-cells were measured by IFNg ELISpot following overnight stimulation with VVVGAVGVGK (SEQ ID NO: 81). Data presented as spot forming colonies (SFC) per 1×10⁶ splenocytes for each animal. Bar represents the median.

FIG. 2E demonstrates the immune response of an immunodominant epitope and related control epitope. Shown are ELISpot results for the TP53 neoepitope pools for R213L and S127Y neoepitopes. Mice engineered to express human HLA-A11:01 were immunized with 5×10¹⁰ VP using the ChAdV68 delivery vectors indicated and splenocytes isolated 14 days post-immunization. The number of antigen-specific T-cells were measured by IFNg ELISpot following overnight stimulation. Data presented as spot forming colonies (SFC) per 1×10⁶ splenocytes for each animal. Bar represents the median. Dashed line represent samples that were too numerous to count (TNTC).

FIG. 3 demonstrates repeating epitopes increases vaccine induced antigen-specific T-cell response. Shown are ELISpot results for the repeated neoepitope KRAS G12V (left panel) or KRAS G12D (right panel). Mice engineered to express human HLA-A11:01 were immunized with 5×10¹⁰ VP using the ChAdV68 delivery vectors indicated and splenocytes isolated 14 days post-immunization. The number of antigen-specific T-cells were measured by IFNg ELISpot following overnight stimulation with VVVGAVGVGK (SEQ ID NO: 81) or VVVGADGVGK (SEQ ID NO: 78), respectively. Data presented as spot forming colonies (SFC) per 1×10⁶ splenocytes for each animal. Bar represents the median.

FIG. 4 demonstrates repeating epitopes increases vaccine induced antigen-specific T-cell response. Shown are ELISpot results for the repeated neoepitope KRAS G12V (left panel) or KRAS G12D (right panel). Mice engineered to express human HLA-A11:01 were immunized with 7×10¹⁰ VP using the ChAdV68 delivery vectors indicated and splenocytes isolated 14 days post-immunization. The number of antigen-specific T-cells were measured by IFNg ELISpot following overnight stimulation with VVVGAVGVGK (SEQ ID NO: 81) or VVVGADGVGK (SEQ ID NO: 78), respectively. Data presented as spot forming colonies (SFC) per 1×10⁶ splenocytes for each animal. Bar represents the median.

FIG. 5 demonstrates repeating epitopes increases vaccine induced antigen-specific T-cell response for KRAS Q61H. Shown are ELISpot results for the repeated neoepitope KRAS Q61H for the indicated cassette formats. Mice engineered to express human HLA-A01:01 were immunized with 5×10¹⁰ VP using the ChAdV68 delivery vectors indicated and splenocytes isolated 12 days post-immunization. The number of antigen-specific T-cells were measured by IFNg ELISpot following overnight stimulation with ILDTAGHEEY (SEQ ID NO: 82). Data presented as spot forming colonies (SFC) per 1×10⁶ splenocytes for each animal. Bar represents the median. Dashed line represent samples that were too numerous to count (TNTC).

FIG. 6 demonstrates repeating epitopes increases vaccine induced antigen-specific T-cell response for both ChAdV68 and SAM vector formats. Shown are ELISpot results for the repeated neoepitope KRAS G12V (left panel) or KRAS G12D (right panel). Mice engineered to express human HLA-A11:01 were immunized with 5×10¹⁰ VP using the ChAdV68 delivery vectors indicated or 10 μg the SAM vectors indicated and splenocytes isolated 14 days post-immunization. The number of antigen-specific T-cells were measured by IFNg ELISpot following overnight stimulation with respective peptide pools that contained all possible 38 minimal epitopes that span the 25 mer. Data presented as spot forming colonies (SFC) per 1×10⁶ splenocytes for each animal. Bar represents the median. Columns from left to right are ChAdV68 20×1, SAM 20×1, ChAdV68 4×4, and SAM 4×4.

FIG. 7 demonstrates repeating epitopes increases vaccine induced antigen-specific T-cell response for both ChAdV68 and SAM vector formats. Shown are ELISpot results for the repeated neoepitope KRAS G12V (left panel) or KRAS G12D (right panel). Mice engineered to express human HLA-A11:01 were immunized with 5×10¹⁰ VP using the ChAdV68 delivery vectors indicated or 10 μg the SAM vectors indicated and splenocytes isolated 14 days post-immunization. The number of antigen-specific T-cells were measured by IFNg ELISpot following overnight stimulation with VVGAVGVGK (SEQ ID NO: 79) or VVVGADGVGK (SEQ ID NO: 78). Data presented as spot forming colonies (SFC) per 1×10⁶ splenocytes for each animal. Bar represents the median. Columns from left to right are ChAdV68 20×1, SAM 20×1, ChAdV68 4×4, and SAM 4×4.

FIG. 8 illustrates a Phase 1/2 study designed to assess the dose, safety and tolerability, immunogenicity, and early clinical activity of cancer vaccines encoding the iterated KRAS neoepitope cassettes described herein (“SLATE v2”) administered in combination with immune checkpoint blockade in patients with advanced cancer.

FIG. 9 shows ELISpot CD8 T cell response for SLATE patient S21 administered the SLATE “version 1” (v1) cassette and patient S31 administered the optimized SLATE “version 2” (v2) cassette including iterated KRAS neoepitopes. Shown are overnight stimulations with peptide pool containing 38 minimal epitopes. Timepoint was collected post-second SAM administration.

FIG. 10 shows clinical responses for patient S31 administered the SLATE v2 cassette including iterated KRAS neoepitopes. Top panel: radiological CT scan of tumor lesion. Bottom left panel: quantification of radiological CT scans. Bottom right panel: response assessment through monitoring neoantigen ctDNA.

FIG. 11 illustrates a Phase 1/2 study designed to assess the dose, safety and tolerability, immunogenicity, and early clinical activity of cancer vaccines encoding the iterated KRAS neoepitope cassettes described herein (“SLATE v2”) administered in combination with immune checkpoint blockade in patients with advanced cancer.

FIG. 12 shows a summary of T cells responses assessed by IFNγ ELISpot for the various G12 mutations, alleles, and cassettes indicated.

FIG. 13 shows molecular responses as assessed by monitoring neoantigen ctDNA (left top panel) and standard serum tumor markers CEA and CA 19-9 (left bottom panel), as well as radiological CT scans of tumor lesions (right panels.

FIG. 14 shows overall survival probabilities for subjects with and without molecular responses (reduction in ctDNA≥30%).

FIG. 15 shows a summary of clinical results for patients with NSCLC.

FIG. 16 shows a summary of clinical results for patients with late-stage CRC.

FIG. 17 illustrates a two-month treatment schedule for the Phase 2 trial.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that stimulates an immune response. An antigen can be a neoantigen. An antigen can be a “shared antigen” that is an antigen found among a specific population, e.g., a specific population of cancer patients.

As used herein the term “neoantigen” is an antigen that has at least one alteration that makes it distinct from the corresponding wild-type antigen, e.g., via mutation in a tumor cell or post-translational modification specific to a tumor cell. A neoantigen can include a polypeptide sequence or a nucleotide sequence. A mutation can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. The subject can be identified for administration through the use of various diagnostic methods, e.g., patient selection methods described further below.

As used herein the term “tumor antigen” is an antigen present in a subject's tumor cell or tissue but not in the subject's corresponding normal cell or tissue, or derived from a polypeptide known to or have been found to have altered expression in a tumor cell or cancerous tissue in comparison to a normal cell or tissue.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to stimulate an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, and smoking history.

The term “antigen-encoding nucleic acid sequences derived from a tumor” refers to nucleic acid sequences obtained from the tumor, e.g. via RT-PCR; or sequence data obtained by sequencing the tumor and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native nucleic acid sequence obtained from a tumor.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a subgenomic (e.g., a 26S) promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, a subgenomic promoter sequence (e.g., a 26S subgenomic promoter sequence), a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Antigen Identification

Research methods for NGS analysis of tumor and normal exome and transcriptomes have been described and applied in the antigen identification space. ^{6, 14, 15} Certain optimizations for greater sensitivity and specificity for antigen identification in the clinical setting can be considered. These optimizations can be grouped into two areas, those related to laboratory processes and those related to the NGS data analysis. The research methods described can also be applied to identification of antigens in other settings, such as identification of identifying antigens from an infectious disease organism, an infection in a subject, or an infected cell of a subject. Examples of optimizations are known to those skilled in the art, for example the methods described in more detail in U.S. Pat. No. 10,055,540, US Application Pub. No. US20200010849A1, U.S. application Ser. No. 16/606,577, and international patent application publications WO2020181240A1, WO/2018/195357 and WO/2018/208856, each herein incorporated by reference, in their entirety, for all purposes.

Methods for identifying shared antigens (e.g., neoantigens) include identifying antigens from a tumor of a subject that are likely to be presented on the cell surface of the tumor or immune cells, including professional antigen presenting cells such as dendritic cells, and/or are likely to be immunogenic. As an example, one such method may comprise the steps of: obtaining at least one of exome, transcriptome or whole genome tumor nucleotide sequencing and/or expression data from the tumor cell of the subject, wherein the tumor nucleotide sequencing and/or expression data is used to obtain data representing peptide sequences of each of a set of antigens (e.g., in the case of neoantigens wherein the peptide sequence of each neoantigen comprises at least one alteration that makes it distinct from the corresponding wild-type peptide sequence or in cases of shared antigens without a mutation where peptides are derived from any polypeptide known to or have been found to have altered expression in a tumor cell or cancerous tissue in comparison to a normal cell or tissue); inputting the peptide sequence of each antigen into one or more presentation models to generate a set of numerical likelihoods that each of the antigens is presented by one or more MHC alleles on the tumor cell surface of the tumor cell of the subject or cells present in the tumor, the set of numerical likelihoods having been identified at least based on received mass spectrometry data; and selecting a subset of the set of antigens based on the set of numerical likelihoods to generate a set of selected antigens.

### III. Identification of Tumor Specific Mutations in Neoantigens

Also disclosed herein are methods for the identification of certain mutations (e.g., the variants or alleles that are present in cancer cells). In particular, these mutations can be present in the genome, transcriptome, proteome, or exome of cancer cells of a subject having cancer but not in normal tissue from the subject. Specific methods for identifying neoantigens, including shared neoantigens, that are specific to tumors are known to those skilled in the art, for example the methods described in more detail in U.S. Pat. No. 10,055,540, US Application Pub. No. US20200010849A1, and international patent application publications WO/2018/195357 and WO/2018/208856, each herein incorporated by reference, in their entirety, for all purposes. Examples of shared neoantigens that are specific to tumors are described in more detail in international patent application publication WO2019226941A1, herein incorporated by reference in its entirety, for all purposes. Shared neoantigens include, but are not limited to, KRAS-associated mutations (e.g., KRAS G12C, KRAS G12V, KRAS G12D, and/or KRAS Q61H mutations). For example, KRAS-associated MHC class I neoepitope can include those mutations with reference to wild-type (WT) human KRAS, such as with reference to the following exemplary amino acid sequence:

(SEQ ID NO: 84)
MTEYKLVVVGAGGVGKSALTIQLIQNHFVDEYDPTIEDSY
RKQVVIDGETCLLDILDTAGQEEYSAMRDQYMRTGEGFLC
VFAINNTKSFEDIHHYREQIKRVKDSEDVPMVLVGNKCDL
PSRTVDTKQAQDLARSYGIPFIETSAKTRQRVEDAFYTLV
REIRQYRLKKISKEEKTPGCVKIKKCIIM.

Genetic mutations in tumors can be considered useful for the immunological targeting of tumors if they lead to changes in the amino acid sequence of a protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift mutations or deletions that lead to a new open reading frame with a novel tumor-specific protein sequence. Mutations can also include one or more of non-frameshift indel, missense or nonsense substitution, splice site alteration, genomic rearrangement or gene fusion, or any genomic or expression alteration giving rise to a neoORF.

Peptides with mutations or mutated polypeptides arising from for example, splice-site, frameshift, readthrough, or gene fusion mutations in tumor cells can be identified by sequencing DNA, RNA or protein in tumor versus normal cells.

Also mutations can include previously identified tumor specific mutations. Known tumor mutations can be found at the Catalogue of Somatic Mutations in Cancer (COSMIC) database.

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle includes adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

Any cell type or tissue can be utilized to obtain nucleic acid samples for use in methods described herein. For example, a DNA or RNA sample can be obtained from a tumor or a bodily fluid, e.g., blood, obtained by known techniques (e.g. venipuncture) or saliva. Alternatively, nucleic acid tests can be performed on dry samples (e.g. hair or skin). In addition, a sample can be obtained for sequencing from a tumor and another sample can be obtained from normal tissue for sequencing where the normal tissue is of the same tissue type as the tumor. A sample can be obtained for sequencing from a tumor and another sample can be obtained from normal tissue for sequencing where the normal tissue is of a distinct tissue type relative to the tumor.

Tumors can include one or more of lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, and small cell lung cancer.

Alternatively, protein mass spectrometry can be used to identify or validate the presence of mutated peptides bound to MHC proteins on tumor cells. Peptides can be acid-eluted from tumor cells or from HLA molecules that are immunoprecipitated from tumor, and then identified using mass spectrometry.

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences.

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Specifically, disclosed herein cassettes including iterations of KRAS-associated MHC class I neoepitopes. KRAS-associated MHC class I neoepitopes include, but are not limited to, neoepitopes having KRAS G12 mutations and/or KRAS Q61 mutations. Cassettes can include iterations of KRAS-associated MHC class I neoepitopes having a KRAS G12 mutation. Cassettes can include iterations of KRAS-associated MHC class I neoepitopes having a KRAS Q61 mutation. Cassettes can include iterations of KRAS-associated MHC class I neoepitopes having KRAS G12C, KRAS G12V, KRAS G12D, and/or KRAS Q61H mutations. Cassettes can include iterations of KRAS-associated MHC class I neoepitopes having a KRAS G12C mutation. Cassettes can include iterations of KRAS-associated MHC class I neoepitopes having a KRAS G12V mutation. Cassettes can include iterations of KRAS-associated MHC class I neoepitopes having a KRAS G12D mutation. Cassettes can include iterations of KRAS-associated MHC class I neoepitopes having a KRAS Q61H mutation. Cassettes can include iterations of each of KRAS-associated MHC class I neoepitopes having a KRAS G12C, KRAS G12V, KRAS G12D, and KRAS Q61H mutation. Cassettes can include iterations of at least two distinct KRAS-associated MHC class I neoepitopes selected from the group consisting of: a KRAS G12C, KRAS G12V, KRAS G12D, and KRAS Q61H mutation. Cassettes can include iterations of at least three distinct KRAS-associated MHC class I neoepitopes selected from the group consisting of: a KRAS G12C, KRAS G12V, KRAS G12D, and KRAS Q61H mutation. Cassettes can include iterations only of a single distinct KRAS-associated MHC class I neoepitope. Cassettes can include iterations only of a single distinct KRAS-associated MHC class I neoepitope having a KRAS G12C mutation. Cassettes can include iterations only of a single distinct KRAS-associated MHC class I neoepitope having a KRAS G12D mutation. Cassettes can include iterations only of a single distinct KRAS-associated MHC class I neoepitope having a KRAS G12V mutation. Cassettes can include iterations only of a single distinct KRAS-associated MHC class I neoepitope having a KRAS Q61H mutation.

KRAS-associated MHC class I neoepitopes having a KRAS G12C mutation include VVVGACGVGK (SEQ ID NO: 75), KLVVVGACGV (SEQ ID NO: 76), or GACGVGKSAL (SEQ ID NO: 93). KRAS-associated MHC class I neoepitopes having a KRAS G12D mutation include VVGADGVGK (SEQ ID NO: 77), VVVGADGVGK (SEQ ID NO: 78), KLVVVGADGV (SEQ ID NO: 94), or GADGVGKSAL (SEQ ID NO: 95). KRAS-associated MHC class I neoepitopes having a KRAS G12V mutation include VVGAVGVGK (SEQ ID NO: 79), VVVGAVGVGK (SEQ ID NO: 81), AVGVGKSAL (SEQ ID NO: 80), or GAVGVGKSAL (SEQ ID NO: 96).

Cassettes can include iterations of each of KRAS-associated MHC class I neoepitopes having the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82). Cassettes can include iterations of at least two distinct KRAS-associated MHC class I neoepitopes having the amino acid sequences selected from the group consisting of: VVVGACGVGK (SEQ ID NO: 75), VVVGADGVGK (SEQ ID NO: 78), VVGAVGVGK (SEQ ID NO: 79), and ILDTAGHEEY (SEQ ID NO: 82). Cassettes can include iterations of at least three distinct KRAS-associated MHC class I neoepitopes having the amino acid sequences selected from the group consisting of: VVVGACGVGK (SEQ ID NO: 75), VVVGADGVGK (SEQ ID NO: 78), VVGAVGVGK (SEQ ID NO: 79), and ILDTAGHEEY (SEQ ID NO: 82). Cassettes can include iterations of at least one of KRAS-associated MHC class I neoepitopes having the amino acid sequences VVVGACGVGK (SEQ ID NO: 75), VVVGADGVGK (SEQ ID NO: 78), VVGAVGVGK (SEQ ID NO: 79), and ILDTAGHEEY (SEQ ID NO: 82).

KRAS-associated MHC class I neoepitopes can include native N- and/or C-terminal flanking sequences of the therapeutic vaccine epitope in the context of the native KRAS protein. Illustrative non-limiting examples of KRAS-associated MHC class I neoepitopes are the 25 mers MTEYKLVVVGACGVGKSALTIQLIQ (SEQ ID NO: 57) for KRAS G12C, MTEYKLVVVGADGVGKSALTIQLIQ (SEQ ID NO: 58) for KRAS G12D, MTEYKLVVVGAVGVGKSALTIQLIQ (SEQ ID NO: 59) for KRAS G12V, and ETCLLDILDTAGHEEYSAMRDQYMR (SEQ ID NO: 60) for KRAS Q61H. KRAS-associated MHC class I neoepitopes that include native flanking sequences can be linked (concatenated) to other neoepitopes encoded in a cassette, including other neoepitopes (e.g., other KRAS-associated MHC class I neoepitopes) that include their respective native flanking sequences. An illustrative non-limiting cassette of concatenated KRAS-associated MHC class I neoepitopes that are linked through their native flanking sequences and that includes 4 iterations for each of the KRAS neoepitopes having the mutations KRAS G12C, KRAS G12D, KRAS G12V, and KRAS Q61H is represented by the amino acid sequence shown in SEQ ID NO: 65.

Epitope-encoding nucleic acid sequences that encode KRAS-associated MHC class I neoepitopes, such as those that include native N- and/or C-terminal flanking sequences, can encode multiple known and/or predicted KRAS-associated MHC class I neoepitopes. As an illustrative example, the KRAS G12V 25 mer MTEYKLVVVGAVGVGKSALTIQLIQ (SEQ ID NO: 59) encodes each of the known and/or predicted KRAS-associated MHC class I neoepitopes VVGAVGVGK (SEQ ID NO: 79), VVVGAVGVGK (SEQ ID NO: 81), and AVGVGKSAL (SEQ ID NO: 80).

Epitope-encoding nucleic acid sequences, including those that encode KRAS-associated MHC class I neoepitopes, can be in any order in a cassette. Epitope-encoding nucleic acid sequences, including those that encode KRAS-associated MHC class I neoepitopes, can be in an order that minimizes junctional epitopes, as described further herein. As an illustrative non-limiting example, concatenated KRAS-associated MHC class I neoepitopes linked together to minimize junctional epitopes is represented by the amino acid sequence shown in SEQ ID NO: 65 and has the order: G12C G12D Q61H G12D G12V G12C Q61H G12D G12V G12C Q61H G12D G12V Q61H G12V G12C.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. The peptide contains the tumor specific mutation. Tumor antigens (e.g., shared tumor antigens and tumor neoantigens) can include, but are not limited to, those described in U.S. application Ser. No. 17/058,128, herein incorporated by reference for all purposes. Antigen peptides can be described in the context of their coding sequence where an antigen includes the nucleotide sequence (e.g., DNA or RNA) that codes for the related polypeptide sequence.

Antigens can be selected that are predicted to be presented on the cell surface of a cell, such as a tumor cell or an immune cell, including professional antigen presenting cells such as dendritic cells. Antigens can be selected that are predicted to be immunogenic.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor.

One or more antigens can be immunogenic in a subject having a tumor, e.g., capable of stimulating a T cell response and/or a B cell response in the subject. One or more antigens can be capable of stimulating a B cell response, such as the production of antibodies that recognize the one or more antigens (e.g., antibodies that recognize a tumor). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. Antigens capable of stimulating a B cell response to a tumor can be an antigen found on the surface of tumor cell. Antigens capable of eliciting a B cell response to a tumor can be an intracellular neoantigen expressed in a tumor.

One or more antigens can include a combination of antigens capable of stimulating a T cell response (e.g., peptides including predicted T cell epitope sequences) and distinct antigens capable of stimulating a B cell response (e.g., full-length proteins, protein subunits, protein domains).

One or more antigens that stimulate an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class 115 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would consist of: (3) the entire stretch of novel tumor-specific amino acids—thus bypassing the need for computational or in vitro test-based selection of the strongest HLA-presented shorter peptide. In both cases, use of a longer peptide allows endogenous processing by patient cells and may lead to more effective antigen presentation and stimulation of T cell responses. Longer peptides can also include a full-length protein, a protein subunit, a protein domain, and combinations thereof of a peptide, such as those expressed in a tumor. Longer peptides (e.g., full-length protein, protein subunit, or protein domain) and combinations thereof can be included to stimulate a B cell response.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not stimulate an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. A peptide can include a tumor-specific mutation. Tumor-specific peptides can be derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell). Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. In some aspects the tumor specific mutation is a driver mutation for a particular cancer type. A peptide can include a KRAS mutation (e.g., KRAS G12C, KRAS G12V, KRAS G12D, and/or KRAS Q61H mutations).

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to stimulate CTL activity can be enhanced by linkage to a sequence which contains at least one epitope that is capable of stimulating a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothioate backbone, or combinations thereof and it may or may not contain introns. A polynucleotide sequence encoding an antigen can be sequence-optimized to improve expression, such as through improving transcription, translation, post-transcriptional processing, and/or RNA stability. For example, polynucleotide sequence encoding an antigen can be codon-optimized. “Codon-optimization” herein refers to replacing infrequently used codons, with respect to codon bias of a given organism, with frequently used synonymous codons. Polynucleotide sequences can be optimized to improve post-transcriptional processing, for example optimized to reduce unintended splicing, such as through removal of splicing motifs (e.g., canonical and/or cryptic/non-canonical splice donor, branch, and/or acceptor sequences) and/or introduction of exogenous splicing motifs (e.g., splice donor, branch, and/or acceptor sequences) to bias favored splicing events. Exogenous intron sequences include, but are not limited to, those derived from SV40 (e.g., an SV40 mini-intron) and derived from immunoglobulins (e.g., human β-globin gene). Exogenous intron sequences can be incorporated between a promoter/enhancer sequence and the antigen(s) sequence. Exogenous intron sequences for use in expression vectors are described in more detail in Callendret et al. (Virology. 2007 Jul. 5; 363(2): 288-302), herein incorporated by reference for all purposes. Polynucleotide sequences can be optimized to improve transcript stability, for example through removal of RNA instability motifs (e.g., AU-rich elements and 3′ UTR motifs) and/or repetitive nucleotide sequences. Polynucleotide sequences can be optimized to improve accurate transcription, for example through removal of cryptic transcriptional initiators and/or terminators. Polynucleotide sequences can be optimized to improve translation and translational accuracy, for example through removal of cryptic AUG start codons, premature polyA sequences, and/or secondary structure motifs. Polynucleotide sequences can be optimized to improve nuclear export of transcripts, such as through addition of a Constitutive Transport Element (CTE), RNA Transport Element (RTE), or Woodchuck Posttranscriptional Regulatory Element (WPRE). Nuclear export signals for use in expression vectors are described in more detail in Callendret et al. (Virology. 2007 Jul. 5; 363(2): 288-302), herein incorporated by reference for all purposes. Polynucleotide sequences can be optimized with respect to GC content, for example to reflect the average GC content of a given organism. Sequence optimization can balance one or more sequence properties, such as transcription, translation, post-transcriptional processing, and/or RNA stability. Sequence optimization can generate an optimal sequence balancing each of transcription, translation, post-transcriptional processing, and RNA stability. Sequence optimization algorithms are known to those of skill in the art, such as GeneArt (Thermo Fisher), Codon Optimization Tool (IDT), Cool Tool (University of Singapore), SGI-DNA (La Jolla California). One or more regions of an antigen-encoding protein can be sequence-optimized separately.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### V. Vaccine Compositions

Also disclosed herein is an immunogenic composition, e.g., a vaccine composition, capable of raising a specific immune response, e.g., a tumor-specific immune response. Vaccine compositions typically comprise one or a plurality of antigens, e.g., selected using a method described herein. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences. Antigen-encoding nucleic acid sequences can refer to the antigen encoding portion of an “antigen cassette.” Features of an antigen cassette are described in greater detail herein. An antigen-encoding nucleic acid sequence can contain one or more epitope-encoding nucleic acid sequences (e.g., an antigen-encoding nucleic acid sequence encoding concatenated T cell epitopes).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the T cell epitopes in an antigen-encoding nucleic acid sequence encoding concatenated T cell epitopes.

A vaccine can contain at least two iterations of an epitope-encoding nucleic acid sequence. A used herein, an “iteration” (or interchangeably a “repeat”) refers to two or more identical nucleic acid epitope-encoding nucleic acid sequences (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two iterations of an epitope-encoding nucleic acid sequence. In further non-limiting examples, the antigen-encoding nucleic acid sequence portion of a cassette encodes more than one distinct epitope, and at least one of the distinct epitopes is encoded by at least two iterations of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having iterations of at least one of the distinct epitopes are illustrated by, but is not limited to, the formulas below:

Iteration of One Distinct Epitope (Iteration of Epitope A): E_{A}-E_{B}-E_{C}-E_{A}; or E_{A}-E_{A}-E_{B}-E_{C}
Iteration of Multiple Distinct Epitopes (Iterations of Epitopes a, B, and C): E_{A}-E_{B}-E_{C}-E_{A}E_{B}-E_{C}; or E_{A}-E_{A}-E_{B}-E_{B}-E_{C}-E_{C}
Multiple Iterations of Multiple Distinct Epitopes (Iterations of Epitopes a, B, and C): E_{A}-E_{B}-E_{C}-E_{A}E_{B}-E_{C}-E_{A}-E_{C}; or E_{A}-E_{A}-E_{A}-E_{B}E_{B}-E_{B}-E_{C}-E_{C}-E_{C}

The above examples are not limiting and the antigen-encoding nucleic acid sequences having iterations of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

(Eₓ−(E^{N}ₙ)_{y})_{z}
where E represents a nucleotide sequence including a distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof. In some aspects, at least one of the distinct epitope-encoding nucleic acid sequences with the at least two iterations encodes a KRAS-associated MHC class I neoepitope.

Each E or E^{N} can independently comprise any epitope-encoding nucleic acid sequence described herein (e.g., a peptide encoding an infectious disease T cell epitope and/or a neoantigen epitope). For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein.

Iterations of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Iterations of an epitope-encoding nucleic acid sequences can be separated by one or more additional nucleotides sequences. In general, iterations of an epitope-encoding nucleic acid sequences can be separated by any size nucleotide sequence applicable for the compositions described herein. In one example, iterations of an epitope-encoding nucleic acid sequences can be separated by a separate distinct epitope-encoding nucleic acid sequence (e.g., E_{A}-E_{B}-E_{C}-E_{A} . . . , as illustrated above). In examples where iterations are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEMF VTAPDNLGYMYEVQWPGQTQPQIANCSVYDFFVWLHYYSVRDT (SEQ ID NO:85) encoding iterations of 25 mer antigens Trp1 (VTNTEMFVTAPDNLGYMYEVQWPGQ [SEQ ID NO:86]) and Trp2 (TQPQIANCSVYDFFVWLHYYSVRDT [SEQ ID NO:87]), the iterations of Trp1 are separated by the 25 mer Trp2 and thus the repeats of the Trp1 epitope-encoding nucleic acid sequences are separated the 75 nucleotide Trp2 epitope-encoding nucleic acid sequence. In examples where iterations are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can be capable of stimulating a specific cytotoxic T-cell response and/or a specific helper T-cell response. The vaccine composition can be capable of stimulating a specific cytotoxic T-cell response and a specific helper T-cell response.

The vaccine composition can be capable of stimulating a specific B-cell response (e.g., an antibody response).

The vaccine composition can be capable of stimulating a specific cytotoxic T-cell response, a specific helper T-cell response, and/or a specific B-cell response. The vaccine composition can be capable of stimulating a specific cytotoxic T-cell response and a specific B-cell response. The vaccine composition can be capable of stimulating a specific helper T-cell response and a specific B-cell response. The vaccine composition can be capable of stimulating a specific cytotoxic T-cell response, a specific helper T-cell response, and a specific B-cell response.

A vaccine composition can further comprise an adjuvant and/or a carrier. Examples of useful adjuvants and carriers are given herein below. A composition can be associated with a carrier such as e.g. a protein or an antigen-presenting cell such as, e.g., a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-E_{C}, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example Sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

### V.A. Antigen Cassette

The methods employed for the selection of one or more antigens, the cloning and construction of an “antigen cassette” and its insertion into a viral vector are within the skill in the art given the teachings provided herein. By “antigen cassette” or “cassette” is meant the combination of a selected antigen or plurality of antigens (e.g., antigen-encoding nucleic acid sequences) and the other regulatory elements necessary to transcribe the antigen(s) and express the transcribed product. The selected antigen or plurality of antigens can refer to distinct epitope sequences, e.g., an antigen-encoding nucleic acid sequence in the cassette can encode an epitope-encoding nucleic acid sequence (or plurality of epitope-encoding nucleic acid sequences) such that the epitopes are transcribed and expressed. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. A cassette can include one or more antigens (e.g., one or more KRAS-associated neoepitopes in the vaccine composition, such as any of the KRAS-associated neoepitopes shown in SEQ ID NOs. 75-82). A cassette can have one or more antigen-encoding nucleic acid sequences, such as a cassette containing multiple antigen-encoding nucleic acid sequences each independently operably linked to separate promoters and/or linked together using other multicistonic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjunction with a 2A ribosome skipping sequence element such that the furin protease cleavage site is configured to facilitate removal of the 2A sequence following translation. In a cassette containing more than one antigen-encoding nucleic acid sequences, each antigen-encoding nucleic acid sequence can contain one or more epitope-encoding nucleic acid sequences (e.g., an antigen-encoding nucleic acid sequence encoding concatenated T cell epitopes).

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

Also disclosed herein is a viral vector comprising a cassette with at least one payload sequence operably linked to a regulatable promoter that is a TET promoter system, such as a TET-On system or TET-Off system. Without wishing to be bound by theory, a TET promoter system can be used to minimize transcription of payload nucleic acids encoded in a cassette, such as antigens encoded in a vaccine cassette, during viral production. TET promoter systems are described in detail in international patent application publication WO2020/243719, herein incorporated by reference for all purposes.

A TET promoter system can include a tetracycline (TET) repressor protein (TETr) controlled promoter. Accordingly, also disclosed herein is a viral vector comprising a cassette with at least one payload sequence operably linked to a tetracycline (TET) repressor protein (TETr) controlled promoter. A TETr controlled promoter can include the 19 bp TET operator (TETo) sequence TCCCTATCAGTGATAGAGA (SEQ ID NO: 92). A TETr controlled promoter can include 2, 3, 4, 5, 6, 7, 8, 9, or 10 or more TETo nucleic acid sequences. In TETr controlled promoter have 2 or more TETo nucleic acid sequences, the TETo sequences can be linked together. In TETr controlled promoter have 2 or more TETo nucleic acid sequences, the TETo sequences can be directly linked together. In TETr controlled promoter have 2 or more TETo nucleic acid sequences, the TETo sequences can be linked together with a linker sequence, such as a linker sequence having 1, 2, 3, 4, 5, 6, 7, 8, 9, 10, 11, 12, 13, 14, 15, 16, 17, 18, 19, or 20 or more nucleotides. In general, a TETr controlled promoter can use any promoter sequence desired, such as a SV40, EF-1, RSV, PGK, HSA, MCK or EBV promoter sequence. A TETr controlled promoter can use a CMV promoter sequence. A TETr controlled promoter can use a minimal CMV promoter sequence. TETo sequences can be upstream (5′) of a promoter sequence region where RNA polymerase binds. In an illustrative example, 7 TETo sequences are upstream (5′) of a promoter sequence. A TETr controlled promoter operably linked to the at least one payload nucleic acid sequence with TETo sequence upstream of the promoter sequence region can have an ordered sequence described in the formula, from 5′ to 3′:

(T−L_{Y})ₓ−P−N

where N is a payload nucleic acid sequence, P is a RNA polymerase binding sequence of the promoter sequence operably linked to payload nucleic acid sequence, T is a TETo nucleic acid sequences comprising the nucleotide sequence shown in SEQ ID NO:66, L is a linker sequence, where Y=0 or 1 for each X, and wherein X=1, 2, 3, 4, 5, 6, 7, 8, 9, 10, 11, 12, 13, 14, 15, 16, 17, 18, 19, or 20. In an illustrative example, X=7 and Y=1 for each X describes where 7 TETo sequences are upstream (5′) of the promoter sequence and each TETo sequence is separated by a linker.

A TETo sequences can be downstream (3′) of a promoter sequence region where RNA polymerase binds. In another illustrative example, 2 TETo sequences are downstream (3′) of a promoter sequence. A TETr controlled promoter operably linked to the at least one payload nucleic acid sequence with TETo sequence downstream of the promoter sequence region can have an ordered sequence described in the formula, from 5′ to 3′:

P−(T−L_{Y})ₓ−N

where N is a payload nucleic acid sequence, P is a RNA polymerase binding sequence of the promoter sequence operably linked to payload nucleic acid sequence, T is a TETo nucleic acid sequences comprising the nucleotide sequence shown in SEQ ID NO:66, L is a linker sequence, where Y=0 or 1 for each X, and wherein X=1, 2, 3, 4, 5, 6, 7, 8, 9, 10, 11, 12, 13, 14, 15, 16, 17, 18, 19, or 20. In an illustrative example, X=2 and Y=1 for each X describes where 2 TETo sequences are downstream (3′) of the promoter sequence and each TETo sequence is separated by a linker.

Viral production of vectors with TETr controlled promoters can use any viral production cell line engineered to express a TETr sequence (tTS), such as a 293 cell line or its derivatives (e.g., a 293F cell line) engineered to express tTS. Viral production of vectors with TETr controlled promoters in tTS-expressing cell can improve viral production. Viral production of vectors with TETr controlled promoters in tTS-expressing cell can improve viral infectivity defined as viral particles (VP) per infectious unit (IU). Viral production of vectors with TETr controlled promoters in tTS-expressing cell can improve viral production and/or viral infectivity by at least 1.5, at least 2, at least 2.5, at least 3, at least 3.5, at least 4, at least 4.5, at least 5, at least 6, at least 7, at least 8, at least 9, or at least 10-fold relative to production in a non-tTS-expressing cell. Viral production of vectors with TETr controlled promoters in tTS-expressing cell can improve viral production and/or viral infectivity by at least 15, at least 20, at least 25, at least 30, at least 35, at least 40, at least 45, at least 50, at least 60, at least 70, at least 80, at least 90, or at least 100-fold relative to production in a non-tTS-expressing cell. Viral production of vectors with TETr controlled promoters in tTS-expressing cell can improve viral production and/or viral infectivity by at least 1.5, at least 2, at least 2.5, at least 3, at least 3.5, at least 4, at least 4.5, at least 5, at least 6, at least 7, at least 8, at least 9, or at least 10-fold relative to production of a vector not having a TETr controlled promoter. Viral production of vectors with TETr controlled promoters in tTS-expressing cell can improve viral production and/or viral infectivity by at least 15, at least 20, at least 25, at least 30, at least 35, at least 40, at least 45, at least 50, at least 60, at least 70, at least 80, at least 90, or at least 100-fold relative to production of a vector not having a TETr controlled promoter.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit, Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As described elsewhere herein, the antigen cassette can be located in the site of any selected deletion in a viral vector, such as the deleted structural proteins of a VEE backbone or the site of the E1 gene region deletion or E3 gene region deletion of a ChAd-based vector, among others which may be selected.

The antigen cassette can be described using the following formula to describe the ordered sequence of each element, from 5′ to 3′:

(Pₐ- (L5_{b}- N_{c}- L3_{d})ₓ)_{z}-(P2ₕ-(G5ₑ-U_{f})_{y})_{w}-G3_{g}

wherein P and P2 comprise promoter nucleotide sequences, N comprises an MHC class I epitope-encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding Nc is an epitope encoding nucleic acid sequence, where for each Y the corresponding Uf is a MHC class II epitope-encoding nucleic acid sequence (e.g., universal MHC class II epitope-encoding nucleic acid sequence). A universal sequence can comprise at least one of Tetanus toxoid and PADRE. A universal sequence can comprise a Tetanus toxoid peptide. A universal sequence can comprise a PADRE peptide. A universal sequence can comprise a Tetanus toxoid and PADRE peptides. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, e=1, g=1, h=0, X=10, Y=2, Z=1, and W=1, describing where no additional promoter is present (e.g. only the promoter nucleotide sequence provided by a vector backbone, such as an RNA alphavirus backbone is present), 10 MHC class I epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to the to a vector backbone (e.g., an RNA alphavirus backbone). Examples of linking the 3′ end of the antigen cassette to a vector backbone (e.g., an RNA alphavirus backbone) include linking directly to the 3′ UTR elements provided by the vector backbone, such as a 3′ 19-nt CSE. Examples of linking the 5′ end of the antigen cassette to a vector backbone (e.g., an RNA alphavirus backbone) include linking directly to a promoter or 5′ UTR element of the vector backbone, such as a subgenomic promoter sequence (e.g., a 26S subgenomic promoter sequence), an alphavirus 5′ UTR, a 51-nt CSE, or a 24-nt CSE.

Other examples include: where a=1 describing where a promoter other than the promoter nucleotide sequence provided by a vector backbone (e.g., an RNA alphavirus backbone) is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to a vector backbone (e.g., an RNA alphavirus backbone).

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each antigen that is present can have a 5′ linker, a 3′ linker, neither, or both. In examples where more than one antigen is present in the same antigen cassette, some antigens may have both a 5′ linker and a 3′ linker, while other antigens may have either a 5′ linker, a 3′ linker, or neither. In other examples where more than one antigen is present in the same antigen cassette, some antigens may have either a 5′ linker or a 3′ linker, while other antigens may have either a 5′ linker, a 3′ linker, or neither.

The promoter nucleotide sequences P and/or P2 can be the same as a promoter nucleotide sequence provided by a vector backbone, such as an RNA alphavirus backbone. For example, the promoter sequence provided by the vector backbone, Pn and P2, can each comprise a subgenomic promoter sequence (e.g., a 26S subgenomic promoter sequence) or a CMV promoter. The promoter nucleotide sequences P and/or P2 can be different from the promoter nucleotide sequence provided by a vector backbone (e.g., an RNA alphavirus backbone), as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encode a MHC class I epitope, a MHC class II epitope, an epitope/antigen capable of stimulating a B cell response, or a combination thereof. For each X, each N can encode a combination of a MHC class I epitope, a MHC class II epitope, and an epitope/antigen capable of stimulating a B cell response. For each X, each N can encode a combination of a MHC class I epitope and a MHC class II epitope. For each X, each N can encode a combination of a MHC class I epitope and an epitope/antigen capable of stimulating a B cell response. For each X, each N can encode a combination of a MHC class II epitope and an epitope/antigen capable of stimulating a B cell response. For each X, each N can encode a MHC class II epitope. For each X, each N can encode an epitope/antigen capable of stimulating a B cell response. For each X, each N can encode a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The cassette encoding the one or more antigens can be 700 nucleotides or less. The cassette encoding the one or more antigens can be 700 nucleotides or less and encode 2 distinct epitope-encoding nucleic acid sequences (e.g., encode 2 distinct infectious disease or tumor derived nucleic acid sequences encoding an immunogenic polypeptide). The cassette encoding the one or more antigens can be 700 nucleotides or less and encode at least 2 distinct epitope-encoding nucleic acid sequences. The cassette encoding the one or more antigens can be 700 nucleotides or less and encode 3 distinct epitope-encoding nucleic acid sequences. The cassette encoding the one or more antigens can be 700 nucleotides or less and encode at least 3 distinct epitope-encoding nucleic acid sequences. The cassette encoding the one or more antigens can be 700 nucleotides or less and include 1-10, 1-5, 1, 2, 3, 4, 5, 6, 7, 8, 9, 10, or more antigens.

The cassette encoding the one or more antigens can be between 375-700 nucleotides in length. The cassette encoding the one or more antigens can be between 375-700 nucleotides in length and encode 2 distinct epitope-encoding nucleic acid sequences (e.g., encode 2 distinct infectious disease or tumor derived nucleic acid sequences encoding an immunogenic polypeptide). The cassette encoding the one or more antigens can be between 375-700 nucleotides in length and encode at least 2 distinct epitope-encoding nucleic acid sequences. The cassette encoding the one or more antigens can be between 375-700 nucleotides in length and encode 3 distinct epitope-encoding nucleic acid sequences. The cassette encoding the one or more antigens be between 375-700 nucleotides in length and encode at least 3 distinct epitope-encoding nucleic acid sequences. The cassette encoding the one or more antigens can be between 375-700 nucleotides in length and include 1-10, 1-5, 1, 2, 3, 4, 5, 6, 7, 8, 9, 10, or more antigens.

The cassette encoding the one or more antigens can be 600, 500, 400, 300, 200, or 100 nucleotides in length or less. The cassette encoding the one or more antigens can be 600, 500, 400, 300, 200, or 100 nucleotides in length or less and encode 2 distinct epitope-encoding nucleic acid sequences. The cassette encoding the one or more antigens can be 600, 500, 400, 300, 200, or 100 nucleotides in length or less and encode at least 2 distinct epitope-encoding nucleic acid sequences. The cassette encoding the one or more antigens can be 600, 500, 400, 300, 200, or 100 nucleotides in length or less and encode 3 distinct epitope-encoding nucleic acid sequences. The cassette encoding the one or more antigens can be 600, 500, 400, 300, 200, or 100 nucleotides in length or less and encode at least 3 distinct epitope-encoding nucleic acid sequences. The cassette encoding the one or more antigens can be 600, 500, 400, 300, 200, or 100 nucleotides in length or less and include 1-10, 1-5, 1, 2, 3, 4, 5, 6, 7, 8, 9, 10, or more antigens.

The cassette encoding the one or more antigens can be between 375-600, between 375-500, or between 375-400 nucleotides in length. The cassette encoding the one or more antigens can be between 375-600, between 375-500, or between 375-400 nucleotides in length and encode 2 distinct epitope-encoding nucleic acid sequences. The cassette encoding the one or more antigens can be between 375-600, between 375-500, or between 375-400 nucleotides in length and encode at least 2 distinct epitope-encoding nucleic acid sequences. The cassette encoding the one or more antigens can be between 375-600, between 375-500, or between 375-400 nucleotides in length and encode 3 distinct epitope-encoding nucleic acid sequences. The cassette encoding the one or more antigens can be between 375-600, between 375-500, or between 375-400 nucleotides in length and encode at least 3 distinct epitope-encoding nucleic acid sequences. The cassette encoding the one or more antigens can be between 375-600, between 375-500, or between 375-400 nucleotides in length and include 1-10, 1-5, 1, 2, 3, 4, 5, 6, 7, 8, 9, 10, or more antigens.

In some instances, an antigen or epitope in a cassette encoding additional antigens and/or epitopes may be an immunodominant epitope relative to the others encoded. Immunodominance, in general, is the skewing of an immune response towards only one or a few specific immunogenic peptides. Immunodominance can be assessed as part of an immune monitoring protocol. For example, immunodominance can be assessed through evaluating T cell and/or B cell responses to the encoded antigens.

Immunodominance can be assessed as the impact of an immunodominant antigen's presence on the immune response to one or more other antigens. For example, an immunodominant antigen and its respective immune response (e.g., an immunodominant MHC class I epitope) can reduce the immune response of another antigen relative to the immune response in the absence of the immunodominant antigen. This reduction can be such that the immune response in the presence of the immunodominant antigen is not considered a therapeutically effective response. For example, an MHC class I epitope would generally be considered immunodominant if T cell responses to other antigens are no longer considered therapeutically effective responses compared to responses elicited in the absence of the immunodominant MHC class I epitope. An immune response can also be reduced to below a limit of detection or near the limit of detection. relative to the response in the absence of the immunodominant antigen. For example, an MHC class I epitope would generally be considered immunodominant if T cell responses to other antigens are at or below the limit of detection compared to responses elicited in the absence of the immunodominant MHC class I epitope. In general, the assessment of immunodominance is between two antigens both capable of stimulating an immune response, e.g., between two T cell epitopes in a vaccine composition administered to a subject possessing a cognate MHC allele known or predicted to present each epitope, respectively. Immunodominance can be assessed through evaluating relative immune responses to other antigens in the presence and absence of the suspected immunodominant antigen.

Immunodominance can be assessed as a relative difference in the immune responses between two or more antigens. Immunodominance can refer to a 5-fold, 10-fold, 20-fold, 30-fold, 40-fold, or 50-fold immune response of a specific antigen relative to another antigen encoded in the same cassette. Immunodominance can refer to a 100-fold, 200-fold, 300-fold, 400-fold, or 500-fold immune response of a specific antigen relative to another antigen encoded in the same cassette. Immunodominance can refer to a 1000-fold, 2000-fold, 3000-fold, 4000-fold, or 5000-fold immune response of a specific antigen relative to another antigen encoded in the same cassette. Immunodominance can refer to a 10,000-fold immune response of a specific antigen relative to another antigen encoded in the same cassette.

In some instances, it may be desired to avoid vaccine compositions containing an immunodominant epitope. For example, it may be desired to avoid designing a vaccine cassette encoding an immunodominant epitope. Without wishing to be bound by theory, administering and/or encoding an immunodominant epitope together with additional epitope may reduce the immune response to the additional epitopes, including potentially ultimately reducing vaccine efficacy against the additional epitopes. As an illustrative non-limiting example, vaccine compositions including TP53-associated neoepitopes may have the immune response, e.g., a T cell response, skewed towards the TP53-associated neoepitope negatively impacting (e.g., reducing the immune response to where the immune response is not a therapeutically effective response and/or to below a limit of detection) the immune response to other antigens or epitopes in the vaccine composition (e.g., one or more KRAS-associated neoepitopes in the vaccine composition, such as any of the KRAS-associated neoepitopes shown in SEQ ID NOs. 75-82). Accordingly, vaccine compositions can be designed to not contain an immunodominant epitope, such as designing a vaccine cassette (e.g., a (neo)antigen-encoding cassette) to not encode an immunodominant epitope. For example, the cassette does not encode an epitope that reduces an immune response to another epitope encoded in the cassette when administered in a vaccine composition to a subject relative to an immune response when the other epitope is administered in the absence of the immunodominant MHC class I epitope. In another example, the cassette does not encode an epitope that reduces an immune response to another epitope encoded in the cassette to below a limit of detection when administered in a vaccine composition to a subject relative to an immune response when the other epitope is administered in the absence of the immunodominant MHC class I epitope. In another example, the cassette does not encode an epitope that reduces an immune response to another epitope encoded in the cassette, wherein the immune response is not a therapeutically effective response, when administered in a vaccine composition to a subject relative to an immune response when the other epitope is administered in the absence of the immunodominant MHC class I epitope. In another example, the cassette does not encode an epitope that stimulates a 5-fold, 10-fold, 20-fold, 30-fold, 40-fold, or 50-fold or greater immune response relative to another epitope encoded in the same cassette in a vaccine composition administered to a subject, where each antigen is capable of stimulating an immune response in the subject. In another example, the cassette does not encode an epitope that stimulates a 100-fold, 200-fold, 300-fold, 400-fold, or 500-fold or greater immune response relative to another epitope encoded in the same cassette in a vaccine composition administered to a subject, where each antigen is capable of stimulating an immune response in the subject. In another example, the cassette does not encode an epitope that stimulates a 1000-fold, 2000-fold, 3000-fold, 4000-fold, or 5000-fold or greater immune response relative to another epitope encoded in the same cassette in a vaccine composition administered to a subject, where each antigen is capable of stimulating an immune response in the subject. In another example, the cassette does not encode an epitope that results in a 10,000-fold or greater immune response relative to another epitope encoded in the same cassette in a vaccine composition administered to a subject, where each antigen is capable of stimulating an immune response in the subject.

### V.B. Immune Modulators

Vectors described herein, such as C68 vectors described herein or alphavirus vectors described herein, can comprise a nucleic acid which encodes at least one antigen and the same or a separate vector can comprise a nucleic acid which encodes at least one immune modulator. An immune modulator can include a binding molecule (e.g., an antibody such as an scFv) which binds to and blocks the activity of an immune checkpoint molecule. An immune modulator can include a cytokine, such as IL-2, IL-7, IL-12 (including IL-12 p35, p40, p70, and/or p70-fusion constructs), IL-15, or IL-21. An immune modulator can include a modified cytokine (e.g., pegIL-2). Vectors can comprise an antigen cassette and one or more nucleic acid molecules encoding an immune modulator.

Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8+ (αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker), Nivolumab (anti-PD1 antibody), Cemiplimab (anti-PD1 antibody), CT-011 (anti-PD1 antibody), BY55 monoclonal antibody, AMP224 (anti-PDL1 antibody), BMS-936559 (anti-PDL1 antibody), MPLDL3280A/Atezolizumab (anti-PDL1 antibody), MSB0010718C (anti-PDL1 antibody) and Yervoy/ipilimumab (anti-CTLA-4 checkpoint inhibitor). Antibody-encoding sequences can be engineered into vectors such as C68 using ordinary skill in the art. An exemplary method is described in Fang et al., Stable antibody expression at therapeutic levels using the 2A peptide. Nat Biotechnol. 2005 May; 23(5):584-90. Epub 2005 Apr. 17; herein incorporated by reference for all purposes.

### V.C. Additional Considerations for Vaccine Design and Manufacture

### V.C.1. Determination of a Set of Peptides that Cover All Tumor Subclones

Truncal peptides, meaning those presented by all or most tumor subclones, can be prioritized for inclusion into a vaccine. Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by a vaccine.

### V.C.2. Antigen Prioritization

After all of the above antigen filters are applied, more candidate antigens may still be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that a tumor will escape immune attack via downregulation or mutation of HLA molecules)
7. Coverage of HLA classes (covering both HLA-I and HLA-II may increase the probability of therapeutic response and decrease the probability of tumor escape)

Additionally, optionally, antigens can be deprioritized (e.g., excluded) from the vaccination if they are predicted to be presented by HLA alleles lost or inactivated in either all or part of the patient's tumor. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

### V.D. Alphavirus

### V.D.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### V.D.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, self-amplifying mRNA (SAM) vectors, or samRNA vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly stimulate an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### V.D.3. Alphavirus Production In Vitro

Alphavirus delivery vectors are generally positive-sense RNA polynucleotides. A convenient technique well-known in the art for RNA production is in vitro transcription IVT. In this technique, a DNA template of the desired vector is first produced by techniques well-known to those in the art, including standard molecular biology techniques such as cloning, restriction digestion, ligation, gene synthesis (e.g., chemical and/or enzymatic synthesis), and polymerase chain reaction (PCR). The DNA template contains a RNA polymerase promoter at the 5′ end of the sequence desired to be transcribed into RNA. Promoters include, but are not limited to, bacteriophage polymerase promoters such as T3, T7, or SP6. The DNA template is then incubated with the appropriate RNA polymerase enzyme, buffer agents, and nucleotides (NTPs). The resulting RNA polynucleotide can optionally be further modified including, but limited to, addition of a 5′ cap structure such as 7-methylguanosine or a related structure, and optionally modifying the 3′ end to include a polyadenylate (polyA) tail. The RNA can then be purified using techniques well-known in the field, such as phenol-chloroform extraction or column purification (e.g., chromatography-based purification).

### V.D.4. Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid eliciting immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target affects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

### V.E. Chimpanzee Adenovirus (ChAd)

### V.E.1. Viral Delivery with Chimpanzee Adenovirus

Vaccine compositions for delivery of one or more antigens (e.g., via an antigen cassette and including one or more KRAS-associated neoepitopes, such as any of the KRAS-associated neoepitopes shown in SEQ ID NOs. 75-82) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes. ChAdV68-based vectors and delivery systems are described in detail in US App. Pub. No. US20200197500A1 and international patent application publication WO2020243719A1, each of which is herein incorporated by reference for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for stimulating an immune response in a mammalian host to treat cancer. The method can comprise the step of administering to the host an effective amount of a recombinant chimpanzee adenovirus, such as C68, comprising an antigen cassette that encodes one or more antigens from the tumor against which the immune response is targeted.

Still another aspect provides a method for stimulating an immune response in a mammalian host to treat or prevent a disease in a subject, such as cancer. The method can comprise the step of administering to the host an effective amount of a recombinant chimpanzee adenovirus, such as C68, comprising an antigen cassette that encodes one or more antigens, such as from the cancer/tumor against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus E1A, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of E1A, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO:1, at least a partial deletion of nucleotides 34,952 to 35,305 of the sequence shown in SEQ ID NO:1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less.

A partially deleted E4 gene can be the E4 gene sequence shown in SEQ ID NO:1 that lacks at least nucleotides 34,916 to 35,642 of the sequence shown in SEQ ID NO:1. A partially deleted E4 gene can be the E4 gene sequence shown in SEQ ID NO:1 that lacks the E4 gene sequence shown in SEQ ID NO:1 and that lacks at least nucleotides 34,916 to 34,942, nucleotides 34,952 to 35,305 of the sequence shown in SEQ ID NO:1, and nucleotides 35,302 to 35,642 of the sequence shown in SEQ ID NO:1. A partially deleted E4 gene can be the E4 gene sequence shown in SEQ ID NO:1 and that lacks at least nucleotides 34,980 to 36,516 of the sequence shown in SEQ ID NO:1. A partially deleted E4 gene can be the E4 gene sequence shown in SEQ ID NO:1 and that lacks at least nucleotides 34,979 to 35,642 of the sequence shown in SEQ ID NO:1. The adenovirus vector having A partially deleted E4 gene can have a cassette, wherein the cassette comprises at least one payload nucleic acid sequence, and wherein the cassette comprises at least one promoter sequence operably linked to the at least one payload nucleic acid sequence. The adenovirus vector having A partially deleted E4 gene can have one or more genes or regulatory sequences of the ChAdV68 sequence shown in SEQ ID NO: 1, optionally wherein the one or more genes or regulatory sequences comprise at least one of the chimpanzee adenovirus inverted terminal repeat (ITR), E1A, E1B, E2A, E2B, E3, E4, L1, L2, L3, L4, and L5 genes of the sequence shown in SEQ ID NO: 1. The adenovirus vector having A partially deleted E4 gene can have nucleotides 2 to 34,915 of the sequence shown in SEQ ID NO:1, wherein A partially deleted E4 gene is 3′ of the nucleotides 2 to 34,915, and optionally the nucleotides 2 to 34,915 additionally lack nucleotides 577 to 3403 of the sequence shown in SEQ ID NO:1 corresponding to an E1 deletion and/or lack nucleotides 27,125 to 31,825 of the sequence shown in SEQ ID NO:1 corresponding to an E3 deletion. The adenovirus vector having A partially deleted E4 gene can have nucleotides 35,643 to 36,518 of the sequence shown in SEQ ID NO:1, and wherein A partially deleted E4 gene is 5′ of the nucleotides 35,643 to 36,518. The adenovirus vector having A partially deleted E4 gene can have nucleotides 2 to 34,915 of the sequence shown in SEQ ID NO:1, wherein A partially deleted E4 gene is 3′ of the nucleotides 2 to 34,915, the nucleotides 2 to 34,915 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion. The adenovirus vector having A partially deleted E4 gene can have nucleotides 2 to 34,915 of the sequence shown in SEQ ID NO:1, wherein A partially deleted E4 gene is 3′ of the nucleotides 2 to 34,915, the nucleotides 2 to 34,915 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein A partially deleted E4 gene is 5′ of the nucleotides 35,643 to 36,518.

A partially deleted E4 gene can be the E4 gene sequence shown in SEQ ID NO:1 that lacks at least nucleotides 34,916 to 35,642 of the sequence shown in SEQ ID NO:1, nucleotides 2 to 34,915 of the sequence shown in SEQ ID NO:1, wherein A partially deleted E4 gene is 3′ of the nucleotides 2 to 34,915, the nucleotides 2 to 34,915 additionally lack nucleotides 577 to 3403 of the sequence shown in SEQ ID NO:1 corresponding to an E1 deletion and lack nucleotides 27,125 to 31,825 of the sequence shown in SEQ ID NO:1 corresponding to an E3 deletion, and have nucleotides 35,643 to 36,518 of the sequence shown in SEQ ID NO:1, and wherein A partially deleted E4 gene is 5′ of the nucleotides 35,643 to 36,518.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein, such as a ChAd vector or self-amplifying RNA vector engineered to express an antigen cassette.

Also disclosed herein is a method for producing an antigen comprising introducing a vector disclosed herein into a mammalian cell, culturing the cell under suitable conditions and producing the antigen.

### V.E.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

### V.E.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

### V.E.4. Construction of the Viral Plasmid Vector

The chimpanzee adenovirus C68 vectors useful in this invention include recombinant, defective adenoviruses, that is, chimpanzee adenovirus sequences functionally deleted in the Ela or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

### V.E.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

### V.E.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene Ela and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus Ela and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

### V.E.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

### V.E.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

### V.E.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

### VI. Therapeutic and Manufacturing Methods

Also provided is a method of stimulating a tumor specific immune response in a subject, vaccinating against a tumor, treating and/or alleviating a symptom of cancer in a subject by administering to the subject one or more antigens such as a plurality of antigens identified using methods disclosed herein.

In some aspects, a subject has been diagnosed with cancer or is at risk of developing cancer. A subject can be a human, dog, cat, horse or any animal in which a tumor specific immune response is desired. A tumor can be any solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

An antigen can be administered in an amount sufficient to stimulate a CTL response. An antigen can be administered in an amount sufficient to stimulate a T cell response. An antigen can be administered in an amount sufficient to stimulate a B cell response. An antigen can be administered in an amount sufficient to stimulate both a T cell response and a B cell response

An antigen can be administered alone or in combination with other therapeutic agents. Therapeutic agents can include those that target an infectious disease organism, such as an anti-viral or antibiotic agent.

In addition, a subject can be further administered an anti-immunosuppressive/immunostimulatory agent such as a checkpoint inhibitor. For example, the subject can be further administered an anti-CTLA antibody or anti-PD-1 or anti-PD-L1. Blockade of CTLA-4 or PD-L1 by antibodies can enhance the immune response to cancerous cells in the patient. In particular, CTLA-4 blockade has been shown effective when following a vaccination protocol.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, cancer, infectious disease, and/or patient-specific. For instance, the exact selection of peptides can be guided by expression patterns of the parent proteins in a given tissue or guided by mutation or disease status of a patient. The selection can be dependent on the specific type of cancer, the status of the disease, the goal of the vaccination (e.g., preventative or targeting an ongoing disease), earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for cancer or an infectious disease, antigens with similar normal self-peptides that are expressed in high amounts in normal tissues can be avoided or be present in low amounts in a composition described herein. On the other hand, if it is known that the tumor or infected cell of a patient expresses high amounts of a certain antigen, the respective pharmaceutical composition for treatment of this cancer or infection can be present in high amounts and/or more than one antigen specific for this particularly antigen or pathway of this antigen can be included.

Compositions comprising an antigen can be administered to an individual already suffering from cancer. In therapeutic applications, compositions are administered to a subject in an amount sufficient to stimulate an immune response, such as stimulating an effective CTL response to the tumor antigen and to cure or at least partially arrest symptoms and/or complications. An immune response can include a reduction in tumor size or volume. Reduction in tumor size or volume can include at least a 5%, at least a 10%, at least a 15%, at least a 20%, at least a 25%, at least a 30%, at least a 35%, at least a 40%, at least a 45%, at least a 50%, at least a 55%, at least a 60%, at least a 65%, at least a 70%, at least a 75%, at least a 80%, at least a 85%, at least a 90%, or at least a 95% reduction. Reduction in tumor size or volume can include at least a 15% reduction. Reduction in tumor size or volume can include at least a 20% reduction. An immune response can include stabilization of tumor size or volume. An immune response can result in amelioration of a subject's disease, such a complete response (CR), partial response (PR), or stable disease (SD) (e.g., as assessed by criteria set forth in a clinical study). An amount adequate to accomplish this is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the cancer has metastasized. In such cases, in view of the minimization of extraneous substances and the relative nontoxic nature of an antigen, it is possible and can be felt desirable by the treating physician to administer substantial excesses of these compositions.

For therapeutic use, administration can begin at the detection or surgical removal of tumors. This can be followed by boosting doses until at least symptoms are substantially abated and for a period thereafter, or immunity is considered to be provided (e.g., a memory B cell or T cell population, or antigen specific B cells or antibodies are produced).

Compositions comprising an antigen can be administered as an adjuvant therapy or maintenance therapy as a combination therapy with an additional therapy, such as administered in combination with chemotherapy, immune checkpoint inhibitor therapy, radiation therapy, or combinations thereof. Combination therapies can include fluoropyrimidine, bevacizumab, and/or an immune checkpoint inhibitor therapy. Combination therapies can include fluoropyrimidine and bevacizumab. Combination therapies can include fluoropyrimidine, bevacizumab, and an immune checkpoint inhibitor therapy (e.g., an anti-PD-1 or anti-PD-L1 antibody).

Immune checkpoint inhibitors can include (1) an anti-PD-1 antibody or an antigen-binding fragment thereof, (2) an anti-PD-L1 antibody or an antigen-binding fragment thereof, and/or (3) an anti-CTLA-4 antibody or an antigen-binding fragment thereof. Immune checkpoint inhibitor therapy can include administration of an anti-CTLA-4 antibody or an antigen-binding fragment thereof only with the priming dose and the first boosting dose. Immune checkpoint inhibitor therapy can include the anti-CTLA-4 antibody ipilimumab. Immune checkpoint inhibitor therapy can include ipilimumab administered at a dose of 30 mg subcutaneously. Immune checkpoint inhibitor therapy can include administration of an anti-PD-L1 antibody or an antigen-binding fragment thereof every 4 weeks (Q4W). Immune checkpoint inhibitor therapy can include the anti-PD-L1 antibody atezolizumab or nivolumab. Atezolizumab can be administered at a dose of 1680 mg intravenously. Nivolumab is administered at a dose of 480 mg intravenously.

Immune checkpoint inhibitor therapy can include at least 2, 3, 4, 5, 6, 7, 8, 9, 10, 11, 12, or 13 separate administrations, e.g., at least 2, 3, 4, 5, 6, 7, 8, 9, 10, 11, 12, or 13 separate administrations on or about every 28 days (or every 4 weeks and/or every month). Immune checkpoint inhibitor therapy can include administration of the anti-PD-L1 antibody or an antigen-binding fragment thereof including at least 2, 3, 4, 5, 6, 7, 8, 9, 10, 11, 12, or 13 administrations, e.g., on or about every 28 days (or every 4 weeks and/or every month). Immune checkpoint inhibitor therapy can include at least 13 separate administrations, e.g., on or about every 28 days (or every 4 weeks and/or every month). In some aspects, the administration of the anti-PD-L1 antibody or an antigen-binding fragment thereof comprises at least 13 administrations, e.g., on or about every 28 days (or every 4 weeks and/or every month).

A ChAdV-based expression system can be administered as a boosting dose on or about day 140 after the priming dose of the ChAdV-based expression system. A ChAdV-based expression system can be administered as a boosting dose on or about week 20 after the priming dose of the ChAdV-based expression system. A ChAdV-based expression system can be administered as a boosting dose on or about month 5 after the priming dose of the ChAdV-based expression system. A ChAdV-based expression system can be administered as a boosting dose on or after day 140 after the priming dose of the ChAdV-based expression system. A ChAdV-based expression system can be administered as a boosting dose on or after week 20 after the priming dose of the ChAdV-based expression system. A ChAdV-based expression system can be administered as a boosting dose on or after month 5 after the priming dose of the ChAdV-based expression system.

A self-replicating alphavirus-based expression system can be administered as at least two boosting doses. A self-replicating alphavirus-based expression system can be administered as at least two or more boosting doses at least 28 days apart. A self-replicating alphavirus-based expression system can be administered as at least two or more boosting doses at least 4 weeks (Q4W) apart. A self-replicating alphavirus-based expression system can be administered as at least two or more boosting doses at least one month apart. A self-replicating alphavirus-based expression system can be administered as at least two or more boosting doses at least 56 days apart. A self-replicating alphavirus-based expression system can be administered as at least two or more boosting doses at least 8 weeks (Q8W) apart. A self-replicating alphavirus-based expression system can be administered as at least two or more boosting doses at least 2 months apart.

A self-replicating alphavirus-based expression system can be administered as at least two boosting doses on or about days 28 and 84 after the priming dose of the ChAdV-based expression system. A self-replicating alphavirus-based expression system can be administered as at least two boosting doses on or about weeks 4 and 12 after the priming dose of the ChAdV-based expression system. A self-replicating alphavirus-based expression system can be administered as at least two boosting doses on or about months 1 and 3 after the priming dose of the ChAdV-based expression system.

A self-replicating alphavirus-based expression system can be administered as at least four boosting doses. A self-replicating alphavirus-based expression system can be administered on or about days 28, 84, 196, and 252 relative to the priming dose of the ChAdV-based expression system. A self-replicating alphavirus-based expression system can be administered on or about weeks 4, 12, 28, and 40 relative to the priming dose of the ChAdV-based expression system. A self-replicating alphavirus-based expression system can be administered on or about months 1, 3, 7, and 10 relative to the priming dose of the ChAdV-based expression system.

A ChAdV-based expression system can be administered as a boosting dose on or about, or after day 140 (or week 20 and/or month 5) after the priming dose of the ChAdV-based expression system, and a self-replicating alphavirus-based expression system can be administered as at least two boosting doses. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two or more boosting doses at least 28 days apart. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two or more boosting doses at least 4 weeks (Q4W) apart. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two or more boosting doses at least one month apart. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two or more boosting doses at least 56 days apart. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two or more boosting doses at least 8 weeks (Q8W) apart. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two or more boosting doses at least 2 months apart.

A ChAdV-based expression system can be administered as a boosting dose on or about, or after day 140 (or week 20 and/or month 5) after the priming dose of the ChAdV-based expression system, and a self-replicating alphavirus-based expression system can be administered as at least two boosting doses on or about days 28 and 84 after the priming dose of the ChAdV-based expression system. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two boosting doses on or about weeks 4 and 12 after the priming dose of the ChAdV-based expression system. A ChAdV-based expression system can be administered as a boosting dose on or about, or after day 140 (or week 20 and/or month 5) after the priming dose of the ChAdV-based expression system, and a self-replicating alphavirus-based expression system can be administered as at least two boosting doses on or about months 1 and 3 after the priming dose of the ChAdV-based expression system.

A ChAdV-based expression system can be administered as a boosting dose on or about, or after day 140 (or week 20 and/or month 5) after the priming dose of the ChAdV-based expression system, and a self-replicating alphavirus-based expression system can be administered as at least four boosting doses. A ChAdV-based expression system can be administered as a boosting dose on or about, or after day 140 (or week 20 and/or month 5) after the priming dose of the ChAdV-based expression system, and a self-replicating alphavirus-based expression system can be administered on or about days 28, 84, 196, and 252 relative to the priming dose of the ChAdV-based expression system. A ChAdV-based expression system can be administered as a boosting dose on or about, or after day 140 (or week 20 and/or month 5) after the priming dose of the ChAdV-based expression system, and a self-replicating alphavirus-based expression system can be administered on or about weeks 4, 12, 28, and 40 relative to the priming dose of the ChAdV-based expression system. A ChAdV-based expression system can be administered as a boosting dose on or about, or after day 140 (or week 20 and/or month 5) after the priming dose of the ChAdV-based expression system, and a self-replicating alphavirus-based expression system can be administered on or about months 1, 3, 7, and 10 relative to the priming dose of the ChAdV-based expression system.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered at a site of surgical excision to stimulate a local immune response to a tumor. The compositions can be administered to target specific tissues, organs, and/or cells of a subject. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a vaccine, comprising performing the steps of a method disclosed herein; and producing a vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NSO cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VII. Antigen Use and Administration

A vaccination protocol can be used to dose a subject with one or more antigens. A priming vaccine and a boosting vaccine can be used to dose the subject. Vaccination methods, protocols, and schedules that can be used include, but are not limited to, those described in international application publication WO2021092095, herein incorporated by reference for all purposes.

A priming vaccine, can be based on C68 (e.g., the sequences shown in SEQ ID NO:1 or 2) or samRNA (e.g., the sequences shown in SEQ ID NO:3 or 4). A boosting vaccine can also be based on C68 (e.g., the sequences shown in SEQ ID NO:1 or 2) or samRNA (e.g., the sequences shown in SEQ ID NO:3 or 4).

Each vector in a prime/boost strategy typically includes a cassette that includes antigens. Cassettes can include about 1-50 antigens, separated by spacers such as the natural sequence that normally surrounds each antigen or other non-natural spacer sequences such as AAY. Cassettes can also include MHCII antigens such a tetanus toxoid antigen and PADRE antigen, which can be considered universal class II antigens. Cassettes can also include a targeting sequence such as a ubiquitin targeting sequence. In addition, each vaccine dose can be administered to the subject in conjunction with (e.g., concurrently, before, or after) an immune modulator. Each vaccine dose can be administered to the subject in conjunction with (e.g., concurrently, before, or after) a checkpoint inhibitor (CPI). CPI's can include those that inhibit CTLA4, PD1, and/or PDL1 such as antibodies or antigen-binding portions thereof. Such antibodies can include atezolizumab, ipilimumab, nivolumamb, cemiplimab, tremelimumab or durvalumab. Each vaccine dose can be administered to the subject in conjunction with (e.g., concurrently, before, or after) a cytokine, such as IL-2, IL-7, IL-12 (including IL-12 p35, p40, p70, and/or p70-fusion constructs), IL-15, or IL-21. Each vaccine dose can be administered to the subject in conjunction with (e.g., concurrently, before, or after) a modified cytokine (e.g., pegIL-2).

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 100 ug RNA, in particular 10 or 100 ug can be used.

A vaccine boost (boosting vaccine) can be injected (e.g., intramuscularly) after prime vaccination. A boosting vaccine can be administered about every 1, 2, 3, 4, 5, 6, 7, 8, 9, or 10 weeks, e.g., every 4 weeks and/or 8 weeks after the prime. Bilateral injections per dose can be used. For example, one or more injections of ChAdV68 (C68) can be used (e.g., total dose 1×10¹² viral particles); one or more injections of SAM vectors at low vaccine dose selected from the range 0.001 to 1 ug RNA, in particular 0.1 or 1 ug can be used; or one or more injections of SAM vectors at high vaccine dose selected from the range 1 to 100 ug RNA, in particular 10 or 100 ug can be used.

Anti-CTLA-4 (e.g., tremelimumab) can also be administered to the subject. For example, anti-CTLA4 can be administered subcutaneously near the site of the intramuscular vaccine injection (ChAdV68 prime or samRNA low doses) to ensure drainage into the same lymph node. Tremelimumab is a selective human IgG2 mAb inhibitor of CTLA-4. Target Anti-CTLA-4 (tremelimumab) subcutaneous dose is typically 70-75 mg (in particular 75 mg) with a dose range of, e.g., 1-100 mg or 5-420 mg.

In certain instances an anti-PD-L1 antibody can be used such as durvalumab (MEDI 4736). Durvalumab is a selective, high affinity human IgG1 mAb that blocks PD-L1 binding to PD-1 and CD80. Durvalumab is generally administered at 20 mg/kg i.v. every 4 weeks.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

Immune responses, such as T cell responses and B cell responses, can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA). Antibodies can also be assessed for function, such as assessed for neutralizing ability.

Disease status of a subject can be monitored following administration of any of the vaccine compositions described herein. For example, disease status may be monitored using isolated cell-free DNA (cfDNA) from a subject (also referred to as circulating tumor DNA “ctDNA”). In addition, the efficacy of a vaccine therapy may be monitored using isolated cfDNA from a subject. cfDNA monitoring can include the steps of: a. isolating or having isolated cfDNA from a subject; b. sequencing or having sequenced the isolated cfDNA; c. determining or having determined a frequency of one or more mutations in the cfDNA relative to a wild-type germline nucleic acid sequence of the subject, and d. assessing or having assessed from step (c) the status of a disease in the subject. The method can also include, following step (c) above, d. performing more than one iteration of steps (a)-(c) for the given subject and comparing the frequency of the one or more mutations determined in the more than one iterations; and f. assessing or having assessed from step (d) the status of a disease in the subject. The more than one iterations can be performed at different time points, such as a first iteration of steps (a)-(c) performed prior to administration of the vaccine composition and a second iteration of steps (a)-(c) is performed subsequent to administration of the vaccine composition. Step (c) can include comparing: the frequency of the one or more mutations determined in the more than one iterations, or the frequency of the one or more mutations determined in the first iteration to the frequency of the one or more mutations determined in the second iteration. An increase in the frequency of the one or more mutations determined in subsequent iterations or the second iteration can be assessed as disease progression. A decrease in the frequency of the one or more mutations determined in subsequent iterations or the second iteration can be assessed as a response. In some aspects, the response is a Complete Response (CR) or a Partial Response (PR). A therapy can be administered to a subject following an assessment step, such as where assessment of the frequency of the one or more mutations in the cfDNA indicates the subject has the disease. The cfDNA isolation step can use centrifugation to separate cfDNA from cells or cellular debris. cfDNA can be isolated from whole blood, such as by separating the plasma layer, buffy coat, and red bloods. cfDNA sequencing can use next generation sequencing (NGS), Sanger sequencing, duplex sequencing, whole-exome sequencing, whole-genome sequencing, de novo sequencing, phased sequencing, targeted amplicon sequencing, shotgun sequencing, or combinations thereof, and may include enriching the cfDNA for one or more polynucleotide regions of interest prior to sequencing (e.g., polynucleotides known or suspected to encode the one or more mutations, coding regions, and/or tumor exome polynucleotides). Enriching the cfDNA may include hybridizing one or more polynucleotide probes, which may be modified (e.g., biotinylated), to the one or more polynucleotide regions of interest. In general, any number of mutations may be monitored simultaneously or in parallel.

Response to treatment (i.e., therapeutic response) may be determined by radiological assessment and/or a molecular response by monitoring neoantigen ctDNA (e.g., variant allele frequency “VAF”) as described, for example, in Zhang et al. Cancer Discov. 2020; 10:1842-1853, Parikh et al. Clin Cancer Res. 2020; 26:1877-1885, and Vega et al. JCO Precision Oncology 2022; 6:e2100372 or neoantigen mutated haploid genome equivalents (mutated hGE) as described, for example, in Palmer et al. Nat. Med. 2022; 28:1619-1629 and Chabon et al. Nature 2020; 580(7802): 245-251. Molecular response may be defined as a decrease in ctDNA relative to baseline ctDNA, for example a ≥20%, ≥30%, ≥40%, ≥50%, ≥60%, ≥70%, ≥80%, ≥90%, or ≥95% decrease in ctDNA relative to baseline ctDNA. In some embodiments, molecular response is defined as ≥30% decrease in ctDNA relative to baseline ctDNA. In some embodiments, molecular response is defined as ≥30% decrease in ctDNA relative to baseline ctDNA, wherein the decrease in ctDNA occurs by 24 months, by 18 months, by 12 months, or by 6 months of treatment. In some embodiments, molecular response is defined as ≥30% decrease in ctDNA relative to baseline ctDNA, wherein the decrease in ctDNA occurs by 12 weeks, by 8 weeks, by 4 weeks, or by 2 weeks of treatment. In other embodiments, molecular response is defined as ≥50% decrease in ctDNA relative to baseline ctDNA, wherein the decrease in ctDNA occurs by 24 months, by 18 months, by 12 months, or by 6 months of treatment. In other embodiments, molecular response is defined as ≥50% decrease in ctDNA relative to baseline ctDNA. In some embodiments, molecular response is defined as ≥50% decrease in ctDNA relative to baseline ctDNA, wherein the decrease in ctDNA occurs by 12 weeks, by 8 weeks, by 4 weeks, or by 2 weeks of treatment. Molecular response may be observed during early on-treatment radiological stable disease (SD) and/or in patients with radiological progressive disease (PD).

### VIII. Antigen Identification

### VIII.A. Antigen Candidate Identification

Research methods for NGS analysis of tumor and normal exome and transcriptomes have been described and applied in the antigen identification space. ^{6, 14, 15} Certain optimizations for greater sensitivity and specificity for antigen identification in the clinical setting can be considered. These optimizations can be grouped into two areas, those related to laboratory processes and those related to the NGS data analysis. Examples of optimizations are known to those skilled in the art, for example the methods described in more detail in international patent application publications WO/2017/106638, WO/2018/195357, and WO/2018/208856, each herein incorporated by reference, in their entirety, for all purposes.

### VIII.B. Isolation and Detection of HLA Peptides

Isolation of HLA-peptide molecules was performed using classic immunoprecipitation (IP) methods after lysis and solubilization of the tissue sample (55-58). A clarified lysate was used for HLA specific IP.

Immunoprecipitation was performed using antibodies coupled to beads where the antibody is specific for HLA molecules. For a pan-Class I HLA immunoprecipitation, a pan-Class I CR antibody is used, for Class II HLA-DR, an HLA-DR antibody is used. Antibody is covalently attached to NHS-sepharose beads during overnight incubation. After covalent attachment, the beads were washed and aliquoted for IP. (59, 60) Immunoprecipitations can also be performed with antibodies that are not covalently attached to beads. Typically this is done using sepharose¹²² or magnetic beads coated with Protein A and/or Protein G to hold the antibody to the column. Some antibodies that can be used to selectively enrich MHC/peptide complex are listed below.

| Antibody | |
|---|---|
| Name | Specificity |
| W6/32 | Class I HLA-A, B, C |
| L243 | Class II - HLA-DR |
| Tu36 | Class II - HLA-DR |
| LN3 | Class II - HLA-DR |
| Tu39 | Class II - HLA-DR, DP, DQ |
| SPVL3 | Class II - HLA - DQ |
| B7/21 | Class II - HLA - DP |

The clarified tissue lysate is added to the antibody beads for the immunoprecipitation. After immunoprecipitation, the beads are removed from the lysate and the lysate stored for additional experiments, including additional IPs. The IP beads are washed to remove non-specific binding and the HLA/peptide complex is eluted from the beads using standard techniques. The protein components are removed from the peptides using a molecular weight spin column or C18 fractionation. The resultant peptides are taken to dryness by SpeedVac evaporation and in some instances are stored at −20C prior to MS analysis. HLA IPs can also be performed in 96well plate format using plates that contain filter bottoms. Use of the plates allows for multiple IPs to be performed in tandem.

Dried peptides are reconstituted in an HPLC buffer suitable for reverse phase chromatography and loaded onto a C-18 microcapillary HPLC column for gradient elution in a Fusion Lumos mass spectrometer (Thermo). MS1 spectra of peptide mass/charge (m/z) were collected in the Orbitrap detector at high resolution followed by MS2 low resolution scans collected in the ion trap detector after HCD fragmentation of the selected ion. Additionally, MS2 spectra can be obtained using either CID or ETD fragmentation methods or any combination of the three techniques to attain greater amino acid coverage of the peptide. MS2 spectra can also be measured with high resolution mass accuracy in the Orbitrap detector with targeted method known as parallel reaction monitoring. In targeted PRM, specific peptide precursor ions are isolated in the Orbitrap detector and all resulting HCD fragmentation ions are scanned across the elution of the peptide peak. This enables both peptide identification and quantitation of endogenous peptide in the presence of a co-injected stabile isotopically labeled peptide standard.

MS2 spectra from each analysis are searched against a protein database using Comet (61, 62) and the peptide identification are scored using Percolator (63-65). Additional sequencing is performed using PEAKS studio (Bioinformatics Solutions Inc.) and other search engines or sequencing methods can be used including spectral matching and de novo sequencing (97). Targeted MS1 and MS2 spectra can be processed through Skyline (104).

### VIII.B.1. MS Limit of Detection Studies in Support of Comprehensive HLA Peptide Sequencing

Using the peptide YVYVADVAAK (SEQ ID NO: 88) it was determined what the limits of detection are using different amounts of peptide loaded onto the LC column. The amounts of peptide tested were 1 pmol, 100 fmol, 10 fmol, 1 fmol, and 100 amol. (Table 1) These results indicate that the lowest limit of detection (LoD) is in the attomol¹²³ range (10⁻¹⁸), that the dynamic range spans five orders of magnitude, and that the signal to noise appears sufficient for sequencing at low femtomol¹²³ ranges (10⁻¹⁵).

TABLE 1

| | | |
|---|---|---|
| | Loaded | Copies/Cell |
| Peptide m/z | on Column | in 1e9cells |

| | | | |
|---|---|---|---|
| 566.830 | 1 | pmol | 600 |
| 562.823 | 100 | fmol | 60 |
| 559.816 | 10 | fmol | 6 |
| 556.810 | 1 | fmol | 0.6 |
| 553.802 | 100 | amol | 0.06 |

### IX. Presentation Model

Presentation models can be used to identify likelihoods of peptide presentation in patients. Various presentation models are known to those skilled in the art, for example the presentation models described in more detail in U.S. Pat. No. 10,055,540, US Application Pub. No. US20200010849A1 and US20110293637, and international patent application publications WO/2018/195357, WO/2018/208856, and WO2016187508, each herein incorporated by reference, in their entirety, for all purposes.

### X. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in U.S. Pat. No. 10,055,540, US Application Pub. No. US20200010849A1, and international patent application publications WO/2018/195357 and WO/2018/208856, each herein incorporated by reference, in their entirety, for all purposes. A training module can construct a presentation model to predict presentation likelihoods of peptides on a per-allele basis. A training module can also construct a presentation model to predict presentation likelihoods of peptides in a multiple-allele setting where two or more MHC alleles are present.

### XI. Prediction Module

A prediction module can be used to receive sequence data and select candidate antigens in the sequence data using a presentation model. Specifically, the sequence data may be DNA sequences, RNA sequences, and/or protein sequences extracted from tumor tissue cells of patients. A prediction module may identify candidate neoantigens that are mutated peptide sequences by comparing sequence data extracted from normal tissue cells of a patient with the sequence data extracted from tumor tissue cells of the patient to identify portions containing one or more mutations. A prediction module may identify candidate antigens that have altered expression in a tumor cell or cancerous tissue in comparison to a normal cell or tissue by comparing sequence data extracted from normal tissue cells of a patient with the sequence data extracted from tumor tissue cells of the patient to identify improperly expressed candidate antigens.

A presentation module can apply one or more presentation model to processed peptide sequences to estimate presentation likelihoods of the peptide sequences. Specifically, the prediction module may select one or more candidate antigen peptide sequences that are likely to be presented on tumor HLA molecules by applying presentation models to the candidate antigens. In one implementation, the presentation module selects candidate antigen sequences that have estimated presentation likelihoods above a predetermined threshold. In another implementation, the presentation model selects the N candidate antigen sequences that have the highest estimated presentation likelihoods (where N is generally the maximum number of epitopes that can be delivered in a vaccine). A vaccine including the selected candidate antigens for a given patient can be injected into a subject to stimulate immune responses.

### XI.B. Cassette Design Module

### XI.B.1 Overview

A cassette design module can be used to generate a vaccine cassette sequence based on selected candidate peptides for injection into a patient. Various cassette design modules are known to those skilled in the art, for example the cassette design modules described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜1018 possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

An illustrative non-limiting cassette of concatenated KRAS-associated MHC class I neoepitopes that are linked through their native flanking sequences, includes 4 iterations for each of the KRAS neoepitopes having the mutations KRAS G12C, KRAS G12D, KRAS G12V, and KRAS Q61H, and has been ordered to minimize potential junctional epitopes is represented by the amino acid sequence shown in SEQ ID NO: 65 and having the order of KRAS-associated neoepitopes: G12C G12D Q61H G12D G12V G12C Q61H G12D G12V G12C Q61H G12D G12V Q61H G12V G12C.

Shared (neo)antigen sequences for inclusion in a shared antigen vaccine and appropriate patients for treatment with such vaccine can be chosen by one of skill in the art, e.g., as described in U.S. application Ser. No. 17/058,128, herein incorporated by reference for all purposes. Mass spectrometry (MS) validation of candidate shared (neo)antigens can performed as part of the selection process.

### XIII. Example Computer

A computer can be used for any of the computational methods described herein. One skilled in the art will recognize a computer can have different architectures. Examples of computers are known to those skilled in the art, for example the computers described in more detail U.S. Pat. No. 10,055,540, US Application Pub. No. US20200010849A1, and in international patent application publications WO/2017/106638, WO/2018/195357, and WO/2018/208856, each herein incorporated by reference, in their entirety, for all purposes.

### XIV. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3^{rd} Ed. (Plenum Press) Vols A and B(1992).

### XIV.A. Evaluation of Cassettes with Repeated Neoepitopess and/or Removal of Immunodominant Neoepitopes Overview

Through vaccination, multiple class I MHC restricted neoantigens that stimulate the corresponding cellular immune response(s) can be delivered. Several vaccine cassettes were engineered to encode multiple neoepitopes, specifically multiple distinct KRAS-associated neoepitopes, as a single gene product where the epitopes were embedded within their natural, surrounding peptide sequence. Various cassettes designs feature multiple copies (i.e., iterations) of one or more neoepitopes. Various cassettes designs also feature removing immunodominant neoepitopes.

### XIV.B. Repeated Neoepitopes and/or Removal of Immunodominant Neoepitopes Evaluation Materials and Methods Antigens

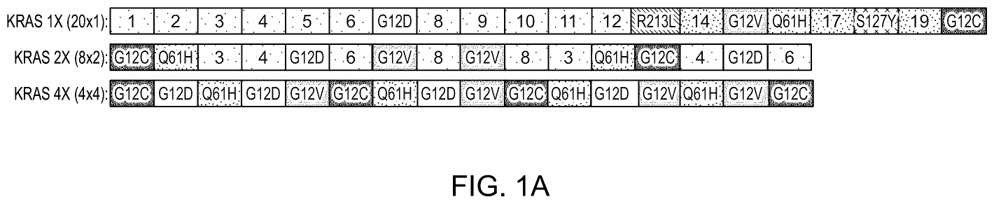

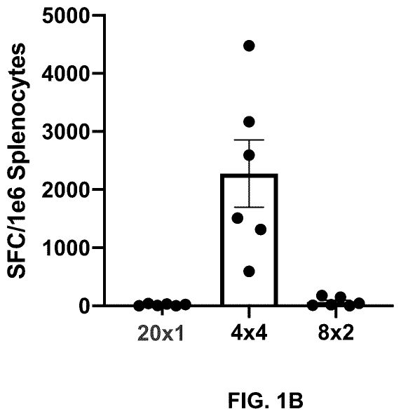

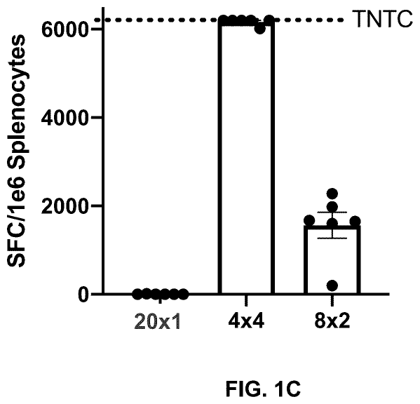

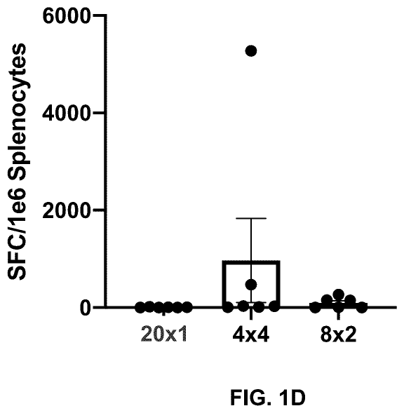

The epitope-containing 25 mer amino acid sequences (i.e., epitope flanked by native N and C terminal amino acid linkers) used in the examples below are presented in Table 2A were used as described below. The antigen-encoding sequences of the cassettes for the various constructs were constructed by directly linking each 25 mer sequence to each other (i.e., no additional amino acids in between consecutive 25 mer sequences) in the order and number described in the examples below, e.g., see FIGS. 1A, 2A, 3A, and 4A. The cassettes containing the full-length antigen-encoding sequences containing the multiple distinct epitopes linked together, as well as universal MHC class II antigens tetanus toxoid and PADRE (bolded sequence), are presented in Table 2B. The complete exogenous nucleotide insertions into the vectors include from 5′ to 3′: a Kozak sequence (GCCACC), nucleotides encoding three amino acids MAG (ATGGCCGGG), one of the cassette sequences of Table 2B, and two stop codons (TAATGA). Further cassettes were generated with 4 iterations of individual KRAS mutations G12V, G12C, and G12D (i.e., separate concatemers of SEQ ID NOs 57, 58, or 59).

TABLE 2A
25mer Neoepitope Containing Sequences

| | | SEQ |
|---|---|---|
| Antigen | | ID |
| Name | 25mer Sequence | NO: |
| KRAS G12C | MTEYKLVVVGACGVGKSALTIQLIQ | 57 |
| KRAS G12D | MTEYKLVVVGADGVGKSALTIQLIQ | 58 |
| KRAS G12V | MTEYKLVVVGAVGVGKSALTIQLIQ | 59 |
| KRAS Q61H | ETCLLDILDTAGHEEYSAMRDQYMR | 60 |
| TP53 R213L | LRVEYLDDRNTFLHSVVVPYEPPEV | 61 |
| TP53 S127Y | HSGTAKSVTCTYYPALNKMFCQLAK | 62 |

TABLE 2B
Full-Length Multi-Neoepitope
Sequences in Cassettes

1X (20x1) Cassette (SEQ ID NO: 63)
MTEYKLVVVGAGDVGKSALTIQLIQETCLLDILDTAGKEE
YSAMRDQYMRMCNSSCMGGMNRMPILTIITLEDSSSGIHS
GATTTAPPLSGKGNPEEEDVSGIHSGATTTAPFLSGKGNP
EEEDVEILDEAYVMAYVMAGVGSPYVSRLLMTEYKLVVVG
ADGVGKSALTIQLIQETCLLDILDTAGREEYSAMRDQYMR
SYLDSGIHSGATATAPSLSGKGNPEKSVTCTYSPALNNMF
CQLAKTCPVQMTEYKLVVVGAAGVGKSALTIQLIQETCLL
DILDTAGLEEYSAMRDQYMRLRVEYLDDRNTFLHSVVVPY
EPPEVDGQITVGQRIGSVSFGTVYKGKWHGMTEYKLVVVG
AVGVGKSALTIQLIQETCLLDILDTAGHEEYSAMRDQYMR
WQQQSYLDSGIHFGATTTAPSLSGKHSGTAKSVTCTYYPA
LNKMFCQLAKKSVTCTYSPALNEMFCQLAKTCPVQMTEYK
LVVVGACGVGKSALTIQLIQGPGPGAKFVAAWTLKAAAGP
GPGQYIKANSKFIGITELGPGPG
KRAS 2X (8x2) Cassette (SEQ ID NO: 64)
MTEYKLVVVGACGVGKSALTIQLIQETCLLDILDTAGHEE
YSAMRDQYMRMTEYKLVVVGAGDVGKSALTIQLIQETCLL
DILDTAGREEYSAMRDQYMRMTEYKLVVVGADGVGKSALT
IQLIQETCLLDILDTAGLEEYSAMRDQYMRMTEYKLVVVG
AVGVGKSALTIQLIQETCLLDILDTAGKEEYSAMRDQYMR
MTEYKLVVVGACGVGKSALTIQLIQETCLLDILDTAGKEE
YSAMRDQYMRMTEYKLVVVGAGDVGKSALTIQLIQETCLL
DILDTAGHEEYSAMRDQYMRMTEYKLVVVGAVGVGKSALT
IQLIQETCLLDILDTAGREEYSAMRDQYMRMTEYKLVVVG
ADGVGKSALTIQLIQETCLLDILDTAGLEEYSAMRDQYMR
GPGPGAKFVAAWTLKAAAGPGPGQYIKANSKFIGITELGP
GPG
KRAS 4X (4x4) Cassette (SEQ ID NO: 65)
MTEYKLVVVGACGVGKSALTIQLIQMTEYKLVVVGADGVG
KSALTIQLIQETCLLDILDTAGHEEYSAMRDQYMRMTEYK
LVVVGADGVGKSALTIQLIQMTEYKLVVVGAVGVGKSALT
IQLIQMTEYKLVVVGACGVGKSALTIQLIQETCLLDILDT
AGHEEYSAMRDQYMRMTEYKLVVVGADGVGKSALTIQLIQ
MTEYKLVVVGAVGVGKSALTIQLIQMTEYKLVVVGACGVG
KSALTIQLIQETCLLDILDTAGHEEYSAMRDQYMRMTEYK
LVVVGADGVGKSALTIQLIQMTEYKLVVVGAVGVGKSALT
IQLIQETCLLDILDTAGHEEYSAMRDQYMRMTEYKLVVVG
AVGVGKSALTIQLIQMTEYKLVVVGACGVGKSALTIQLIQ
GPGPGAKFVAAWTLKAAAGPGPGQYIKANSKFIGITELGP
GPG
KRAS 4x1 Cassette (SEQ ID NO: 66)
MTEYKLVVVGACGVGKSALTIQLIQMTEYKLVVVGADGVG
KSALTIQLIQETCLLDILDTAGHEEYSAMRDQYMRMTEYK
LVVVGAVGVGKSALTIQLIQGPGPGAKFVAAWTLKAAAGP
GPGQYIKANSKFIGITELGPGPG
KRAS 4x1 + R213L Cassette (SEQ ID NO: 67)
MTEYKLVVVGACGVGKSALTIQLIQMTEYKLVVVGADGVG
KSALTIQLIQETCLLDILDTAGHEEYSAMRDQYMRMTEYK
LVVVGAVGVGKSALTIQLIQLRVEYLDDRNTFLHSVVVPY
EPPEVGPGPGAKFVAAWTLKAAAGPGPGQYIKANSKFIGI
TELGPGPG
KRAS 4x1 + S127Y Cassette (SEQ ID NO: 68)
MTEYKLVVVGACGVGKSALTIQLIQMTEYKLVVVGADGVG
KSALTIQLIQETCLLDILDTAGHEEYSAMRDQYMRMTEYK
LVVVGAVGVGKSALTIQLIQHSGTAKSVTCTYYPALNKMF
CQLAKGPGPGAKFVAAWTLKAAAGPGPGQYIKANSKFIGI
TELGPGPG

### Adenoviral Vectors

A modified ChAdV68 vector for the antigen expression system was generated based on AC_000011.1 with E1 (nt 577 to 3403) and E3 (nt 27,125-31,825) sequences deleted and the corresponding ATCC VR-594 (Independently sequenced Full-Length VR-594 C68 SEQ ID NO:10) nucleotides substituted at five positions. The full-length ChAdVC68 AC_000011.1 sequence with corresponding ATCC VR-594 nucleotides substituted at five positions is referred to as “ChAdV68.5WTnt” (SEQ ID NO:1). Antigen cassettes under the control of the CMV promoter/enhancer were inserted in place of deleted E1 sequences. A representative ChAdV68 vector containing 20 model antigens in an antigen cassette is “ChAdV68.5WTnt.MAG25 mer” (SEQ ID NO:2). The vectors featuring antigen cassettes described below having the MAG25 mer cassette can be replaced by the antigen cassettes described above, e.g., in Table 2B.

Adenoviral Production in 293F cells

ChAdV68 virus production was performed in 293F cells grown in 293 FreeStyle™ (ThermoFisher) media in an incubator at 8% CO₂. On the day of infection cells were diluted to 10⁶ cells per mL, with 98% viability and 400 mL were used per production run in 1L Shake flasks (Corning). 4 mL of the tertiary viral stock with a target MOI of >3.3 was used per infection. The cells were incubated for 48-72 h until the viability was <70% as measured by Trypan blue. The infected cells were then harvested by centrifugation, full speed bench top centrifuge and washed in 1×PBS, re-centrifuged and then re-suspended in 20 mL of 10 mM Tris pH7.4. The cell pellet was lysed by freeze thawing 3× and clarified by centrifugation at 4,300×g for 5 minutes.

Adenoviral Purification by CsCl centrifugation

Viral DNA was purified by CsCl centrifugation. Two discontinuous gradient runs were performed. The first to purify virus from cellular components and the second to further refine separation from cellular components and separate defective from infectious particles.

10 mL of 1.2 (26.8g CsCl dissolved in 92 mL of 10 mM Tris pH 8.0) CsCl was added to polyallomer tubes. Then 8 mL of 1.4 CsCl (53g CsCl dissolved in 87 mL of 10 mM Tris pH 8.0) was carefully added using a pipette delivering to the bottom of the tube. The clarified virus was carefully layered on top of the 1.2 layer. If needed more 10 mM Tris was added to balance the tubes. The tubes were then placed in a SW-32Ti rotor and centrifuged for 2 h 30 min at 10° C. The tube was then removed to a laminar flow cabinet and the virus band pulled using an 18 gauge needle and a 10 mL syringe. Care was taken not to remove contaminating host cell DNA and protein. The band was then diluted at least 2× with 10 mM Tris pH 8.0 and layered as before on a discontinuous gradient as described above. The run was performed as described before except that this time the run was performed overnight. The next day the band was pulled with care to avoid pulling any of the defective particle band. The virus was then dialyzed using a Slide-a-Lyzer™ Cassette (Pierce) against ARM buffer (20 mM Tris pH 8.0, 25 mM NaCl, 2.5% Glycerol). This was performed 3×, 1 h per buffer exchange. The virus was then aliquoted for storage at −80° C.

### Adenoviral Viral Assays

VP concentration was performed by using an OD 260 assay based on the extinction coefficient of 1.1×10¹² viral particles (VP) is equivalent to an Absorbance value of 1 at OD260 nm. Two dilutions (1:5 and 1:10) of adenovirus were made in a viral lysis buffer (0.1% SDS, 10 mM Tris pH 7.4, 1 mM EDTA). OD was measured in duplicate at both dilutions and the VP concentration/mL was measured by multiplying the OD260 value X dilution factor X 1.1×10¹²VP.

An infectious unit (IU) titer was calculated by a limiting dilution assay of the viral stock. The virus was initially diluted 100× in DMEM/5% NS/1× PS and then subsequently diluted using 10-fold dilutions down to 1×10⁻⁷. 100 μL of these dilutions were then added to 293A cells that were seeded at least an hour before at 3e5 cells/well of a 24 well plate. This was performed in duplicate. Plates were incubated for 48 h in a CO2 (5%) incubator at 37° C. The cells were then washed with 1×PBS and were then fixed with 100% cold methanol (−20° C.). The plates were then incubated at −20° C. for a minimum of 20 minutes. The wells were washed with 1×PBS then blocked in 1×PBS/0.1% BSA for 1 h at room temperature. A rabbit anti-Ad antibody (Abcam, Cambridge, MA) was added at 1:8,000 dilution in blocking buffer (0.25 ml per well) and incubated for 1 h at room temperature. The wells were washed 4× with 0.5 mL PBS per well. A HRP conjugated Goat anti-Rabbit antibody (Bethyl Labs, Montgomery Texas) diluted 1000× was added per well and incubated for 1 h prior to a final round of washing. 5 PBS washes were performed and the plates were developed using DAB (Diaminobenzidine tetrahydrochloride) substrate in Tris buffered saline (0.67 mg/mL DAB in 50 mM Tris pH 7.5, 150 mM NaCl) with 0.01% H₂O₂. Wells were developed for 5 min prior to counting. Cells were counted under a 10× objective using a dilution that gave between 4-40 stained cells per field of view. The field of view that was used was a 0.32 mm² grid of which there are equivalent to 625 per field of view on a 24 well plate. The number of infectious viruses/mL can be determined by the number of stained cells per grid multiplied by the number of grids per field of view multiplied by a dilution factor 10. Similarly, when working with GFP expressing cells florescent can be used rather than capsid staining to determine the number of GFP expressing virions per mL.

### SAM Vectors

A RNA alphavirus backbone for the antigen expression system was generated from a self-replicating Venezuelan Equine Encephalitis (VEE) virus (Kinney, 1986, Virology 152: 400-413) by deleting the structural proteins of VEE located 3′ of the 26S sub-genomic promoter (VEE sequences 7544 to 11,175 deleted; numbering based on Kinney et al 1986; SEQ ID NO:6). To generate the self-amplifying mRNA (“SAM”) vector, the deleted sequences are replaced by antigen sequences. A representative SAM vector containing 20 model antigens is “VEE-MAG25 mer” (SEQ ID NO:4). The vectors featuring the antigen cassettes described having the MAG25 mer cassette can be replaced by the SARS-CoV-2 cassettes and/or full-length proteins described herein.

### In Vitro Transcription to Generate SAM

For in vivo studies: SAM vectors were generated as “AU-SAM” vectors. A modified T7 RNA polymerase promoter (TAATACGACTCACTATA [SEQ ID NO:89]), which lacks the canonical 3′ dinucleotide GG, was added to the 5′ end of the SAM vector to generate the in vitro transcription template DNA (e.g., the sequence set forth in SEQ ID NO:6; SAM with 7544 to 11,175 deleted without an inserted antigen cassette). Reaction conditions are described below:
1× transcription buffer (40 mM Tris-HCL [pH7.9], 10 mM dithiothreitol, 2 mM spermidine, 0.002% Triton X-100, and 27 mM magnesium chloride) using final concentrations of 1×T7 RNA polymerase mix (E2040S); 0.025 mg/mL DNA transcription template (linearized by restriction digest); 8 mM CleanCap Reagent AU (Cat. No. N-7114) and 10 mM each of ATP, cytidine triphosphate (CTP), GTP, and uridine triphosphate (UTP)
Transcription reactions were incubated at 37° C. for 2 hr and treated with final 2 U DNase I (AM2239)/0.001 mg DNA transcription template in DNase I buffer for 1 hr at 37° C.
SAM was purified by RNeasy Maxi (QIAGEN, 75162)

Alternatively to co-transcriptional addition of a 5′ cap structure, a 7-methylguanosine or a related 5′ cap structure can be enzymatically added following transcription using a vaccinia capping system (NEB Cat. No. M2080) containing mRNA 2′-O-methyltransferase and S-Adenosyl methionine.

### Immunizations

For ChAdV68 vaccines, transgenic mice expressing a chimeric HLA-A11:01 (Taconic Model #9660-[CB6F1-Tg(HLA-A*1101/H2-Kb)A11.01]) were injected with 8×10¹⁰ viral particles (VP) or 5×10¹⁰ VP, as indicated, in 100 μL volume, bilateral intramuscular injection (50 μL per leg).

For SAM vaccines, 10 μg of RNA-LNP complexes in 100 μL volume were administered as a bilateral intramuscular injection (50 μL per leg).

### Splenocyte Dissociation

Splenocytes were isolated 14 days post-immunization. Spleens for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Cytoflex LX (Beckman Coulter) using propidium iodide staining to exclude dead and apoptotic cells. Cell were then adjusted to the appropriate concentration of live cells for subsequent analysis.

### Ex Vivo Enzyme-Linked Immunospot (ELISpot) Analysis

ELISPOT analysis was performed according to ELISPOT harmonization guidelines {DOI: 10.1038/nprot.2015.068} with the mouse IFNg ELISpotPLUS kit (MABTECH). 1×10⁵ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISPOT analysis, wells with saturation>50% were recorded as “too numerous to count”. Samples with deviation of replicate wells >10% were excluded from analysis. Spot counts were then corrected for well confluency using the formula: spot count+2×(spot count×% confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### XIV.C. Repeated Neopitope Evaluation Results

Vaccine efficacy with cassettes having repeated (i.e., iterated) neoepitopes was compared against cassettes having only a single copy of a given neoepitope. Mice engineered to express human HLA-A11:01 were immunized with 8×10¹⁰ VP using the ChAdV68 delivery vectors described below and efficacy was assessed by IFNγ ELISpot.

As illustrated in FIG. 1A, a series of ChAdV68 delivery vectors were designed to assess efficacy of cassettes featuring either a single copy of KRAS neoepitopes G12C, G12V, G12D, and Q61H (“KRAS 1× (20×1)”; cassette=SEQ ID NO: 63), 2 repeats of the KRAS G12C, G12V, G12D, and Q61H neoepitopes and 2 repeats of additional KRAS neoepitopes (“KRAS 2× (8×2)”; cassette=SEQ ID NO: 64), or 4 repeats of the KRAS neoepitopes (“KRAS 4× (4×4)”; cassette=SEQ ID NO: 65). HLA-A11:01 was previously predicted to present KRAS G12C, G12V, and G12D neoepitopes (data not shown).

As shown in FIG. 1B and presented in Table 3, vaccination with vectors featuring multiple repeats of the neoepitopes demonstrated increased spot forming colonies (SFC), indicating repetition of epitope-encoding sequences in a cassette led to an increased antigen-specific immune response against KRAS neoepitope G12C. However, upon further observation that there was a response to the G12C peptide with the G12V 1×4 cassette (only expressing G12V) and none with the G12C 1×4 cassette (only expressing G12C), the observed response to the G12C peptide with the 4×4 cassette was likely driven by the G12V epitope. Mass spec analysis confirmed that the G12C peptide was contaminated with G12V peptide.

As shown in FIG. 1C and FIG. 1D and presented in Table 3A, vaccination with vectors featuring multiple repeats of the neoepitopes demonstrated increased spot forming colonies (SFC), indicating repetition of epitope-encoding sequences in a cassette led to an increased antigen-specific immune response against KRAS neoepitopes, specifically G12V and G12D neoepitopes, using a ChAdV68 delivery system.

TABLE 3
ELISpot data for KRAS Neoepitopes G12C, G12V,
and G12D +/− Repetition

| Antigen | G12C | | G12V | | G12D | |
|---|---|---|---|---|---|---|
| Cassette | Mean | +/−SE | Mean | +/−SE | Mean | +/−SE |
| 20 × 1 | 17 | 7 | 3 | 2 | 5 | 3 |
| 4 × 4 | 2277⁺ | 580 | 6169* | 31 | 970 | 864 |
| 8 × 2 | 66 | 31 | 1563 | 293 | 97 | 45 |

*5/6 samples were too numerous to count (max value 6200)
⁺likely due to contamination with G12V

### XIV.D. Immunodominant Epitope Removal Evaluation Results

Vaccine efficacy with cassettes having a potentially immunodominant TP53 neoepitope capable of stimulating an immune response was compared against cassettes without a TP53 neoepitope capable of stimulating an immune response. Mice engineered to express human HLA-A11:01 were immunized with 5×10¹⁰ VP using the ChAdV68 delivery vectors described below and efficacy was assessed by ELISpot. HLA-A11:01 was previously predicted to present KRAS G12C, G12V, G12D, and TP53 S127Y neoepitopes (data not shown).

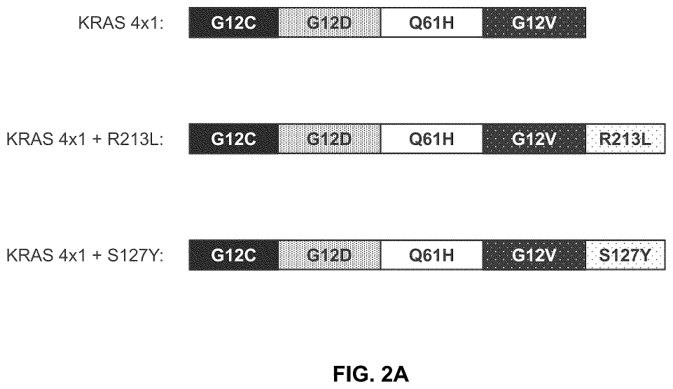

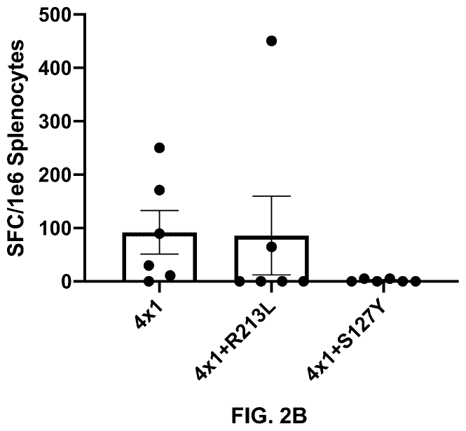

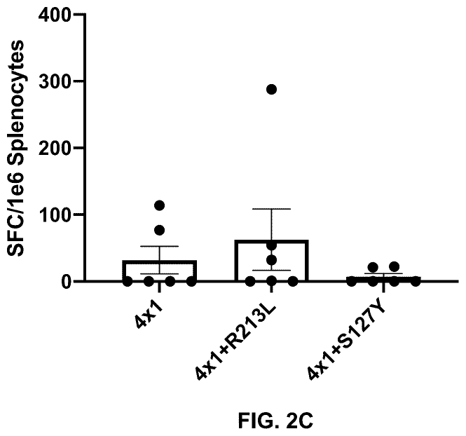

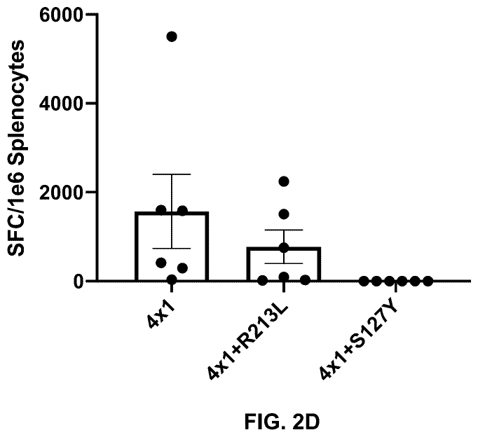

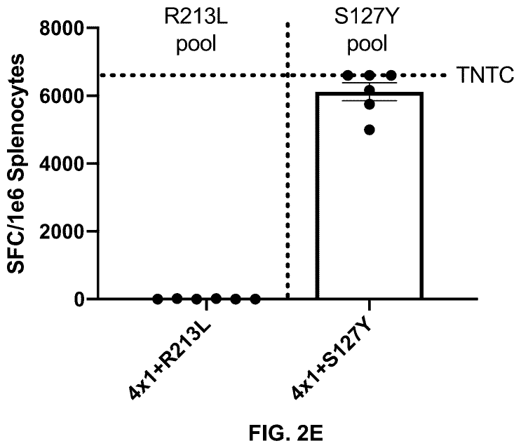

As illustrated in FIG. 2A, a series of ChAdV68 delivery vectors were designed to assess immunodominance of a TP53 epitope. Vector containing only KRAS neoepitopes G12C, G12V, G12D, and Q61H (“KRAS 4×1”; cassette=SEQ ID NO: 66), KRAS neoepitopes in combination with a TP53 R213L neoepitope considered unable to stimulate an immune response in the HLA-A11:01 mouse model as it is predicted to be presented by human HLA-A02:01 and not HLA-A11:01 (“KRAS 4×1+R213L”; cassette=SEQ ID NO: 67), or KRAS neoepitopes in combination with a TP53 S127Y neoepitope predicted to be presented by HLA-A11:01 (“KRAS 4×1+S127Y”; cassette=SEQ ID NO: 68).

As shown in FIG. 2B and presented in Table 4, vaccination with vectors including the immunogenic TP53 S127Y epitope demonstrated reduced spot forming colonies (SFC) for KRAS epitope G12C relative to vectors not including a TP53 neoepitope (“4×1”) or including a non-immunogenic TP53 neoepitope (“4×1+R213L”), indicating presence of the TP53 S127Y neoepitope in the vaccine cassette acts as an immunodominant neoepitope decreasing KRAS-specific T cell responses. However, upon further observation that there was a response to the G12C peptide with the G12V 1×4 cassette (only expressing G12V) and none with the G12C 1×4 cassette (only expressing G12C), the observed response to the G12C peptide with the 4×4 cassette was likely driven by the G12V epitope. Mass spec analysis confirmed that the G12C peptide was contaminated with G12V peptide.

As shown in FIGS. 2C-E and presented in Table 4, vaccination with vectors including the immunogenic TP53 S127Y epitope demonstrated reduced spot forming colonies (SFC) for KRAS epitopes G12V and G12D relative to vectors not including a TP53 neoepitope (“4×1”) or including a non-immunogenic TP53 neoepitope (“4×1+R213L”), indicating presence of the TP53 S127Y neoepitope in the vaccine cassette acts as an immunodominant neoepitope decreasing KRAS-specific T cell responses.

TABLE 4
ELISpot data for KRAS and TP53 Neoepitopes

| Antigen | G12C | | G12D | | G12V | | R213L | | S127Y | |
|---|---|---|---|---|---|---|---|---|---|---|
| Cassette | Mean | +/−SE | Mean | +/−SE | Mean | +/−SE | Mean | +/−SE | Mean | +/−SE |
| 4 × 1 | 92 | 41 | 32 | 21 | 1570 | 833 | ND | ND | ND | ND |
| 4 × 1 + R213L | 86 | 74 | 62 | 46 | 773 | 378 | 5 | 3 | ND | ND |
| 4 × 1 + S127Y | 2 | 1 | 7 | 5 | 0 | 0 | ND | ND | 6118* | 264 |

ND = Not Determined;
*3/6 samples were too numerous to count (max value 6200)

### XIV.E. Evaluation of Presentation of KRAS Neoepitopes by Mass Spectrometry

Mass spectrometry (MS) validation of candidate KRAS epitopes was performed using targeted mass spectrometry methods. Either donor tumor resections (multi-allelic; allele assignment from EDGE prediction) or single allelic K562 lines engineered to express the indicated multi-epitope cassettes (Table 2B) or the indicated neoepitope containing 25 mer sequences in Table 2A were assessed. Mass spectrometry analysis methods are described in more detail in Gillete et al. (Nat Methods. 2013 January; 10(1):28-34), herein incorporated by reference in its entirety for all purposes. Targeted mass spectrometry analysis, including use of K562 single allelic in vitro systems, is described in international application publication WO2021092095, herein incorporated by reference for all purposes. Results are shown in Table 5 demonstrating confirmation of HLA-specific presentation by various KRAS neoepitopes.

TABLE 5
KRAS Neoepitope Mass Spectrometry

| | | | | 20x1 | | | |
|---|---|---|---|---|---|---|---|
| | | | | K562 | 4x4 K562 | 8x2 K562 | SEQ ID 57 |
| | | | | Cell | Cell | Cell | or 59 |
| Mutation | HLA | Sequence | Tumors | Lines | Lines | Lines | 25mer |
| G12C | A*02:01 | KLVVVGACGV | No | No | No | No | Yes |
| | | (SEQ ID NO: 76) | | | | | |
| G12C | A*03:01 | VVVGACGVGK | No | Yes | NA | NA | NA |
| | | (SEQ ID NO: 75) | | | | | |
| G12C | A*11:01 | VVVGACGVGK | No | Yes | Yes | No | NA |
| | | (SEQ ID NO: 75) | | | | | |
| G12C | A*68:01 | VVVGACGVGK | No | Yes | NA | NA | NA |
| | | (SEQ ID NO: 75) | | | | | |
| G12C | B*07:02 | GACGVGKSAL | No | Yes | NA | NA | NA |
| | | (SEQ ID NO: 93) | | | | | |
| G12C | C*03:04 | GACGVGKSAL | No | Yes | NA | NA | NA |
| | | (SEQ ID NO: 93) | | | | | |
| G12D | A*02:01 | KLVVVGADGV | No | No | Yes | No | NA |
| | | (SEQ ID NO: 94) | | | | | |
| G12D | A*03:01 | VVGADGVGK | No | Yes | NA | NA | NA |
| | | (SEQ ID NO: 77) | | | | | |
| G12D | A*03:01 | VVVGADGVGK | No | Yes | NA | NA | NA |
| | | (SEQ ID NO: 78) | | | | | |
| G12D | A*11:01 | VVGADGVGK | Yes | Yes | Yes | No | NA |
| | | (SEQ ID NO: 77) | | | | | |
| G12D | A*11:01 | VVVGADGVGK | Yes | Yes | Yes | Yes | NA |
| | | (SEQ ID NO: 78) | | | | | |
| G12D | A*68:01 | VVVGADGVGK | No | Yes | NA | NA | NA |
| | | (SEQ ID NO: 78) | | | | | |
| G12D | B*07:02 | GADGVGKSAL | No | Yes | NA | NA | NA |
| | | (SEQ ID NO: 95) | | | | | |
| G12D | C*03:04 | GADGVGKSAL | No | Yes | NA | NA | NA |
| | | (SEQ ID NO: 95) | | | | | |
| G12V | A*02:01 | KLVVVGAVGV | No | No | Yes | No | Yes |
| | | (SEQ ID NO: 97) | | | | | |
| G12V | A*03:01 | VVGAVGVGK | No | Yes | NA | NA | NA |
| | | (SEQ ID NO: 79) | | | | | |
| G12V | A*03:01 | VVVGAVGVGK | Yes | Yes | NA | NA | NA |
| | | (SEQ ID NO: 81) | | | | | |
| G12V | A*11:01 | VVGAVGVGK | Yes | Yes | Yes | Yes | Yes |
| | | (SEQ ID NO: 79) | | | | | |
| G12V | A*11:01 | VVVGAVGVGK | Yes | Yes | Yes | Yes | Yes |
| | | (SEQ ID NO: 81) | | | | | |
| G12V | A*68:01 | VVVGAVGVGK | No | Yes | NA | NA | NA |
| | | (SEQ ID NO: 81) | | | | | |
| G12V | B*07:02 | AVGVGKSAL | No | Yes | NA | NA | NA |
| | | (SEQ ID NO: 80) | | | | | |
| G12V | C*01:02 | AVGVGKSAL | Yes | NA | NA | NA | NA |
| | | (SEQ ID NO: 80) | | | | | |
| G12V | C*03:04 | GAVGVGKSAL | No | Yes | NA | NA | NA |
| | | (SEQ ID NO: 96) | | | | | |
| Q61H | A*01:01 | ILDTAGHEEY | NA | Yes | Yes | Yes | NA |
| | | (SEQ ID NO: 82) | | | | | |

NA = not applicable (either tumor or cell line not available/generated to test)

### XIV.F. Iterated KRAS Neoepitope Cassette Evaluation Results

Vaccine efficacy with various cassettes having repeated (i.e., iterated) neoepitopes or a single copy of a given neoepitope were evaluated. Mice engineered to express human HLA-A11:01 for KRAS G12 mutations or human HLA-A01:01 for KRAS Q61H were immunized with 5×10¹⁰ VP using the ChAdV68 delivery vectors described below and efficacy was assessed by IFNγ ELISpot.

A series of ChAdV68 delivery vectors were designed to assess efficacy of cassettes featuring either a single copy of KRAS neoepitopes G12C, G12V, G12D, and Q61H (“KRAS 4×1”; cassette=SEQ ID NO: 66), 4 iterations of each of the KRAS neoepitopes G12C, G12V, G12D, and Q61H (“KRAS 4×(4×4)”; cassette=SEQ ID NO: 65), or 4 iterations of only one of the KRAS neoepitopes G12C, G12V, G12D, or Q61H (“KRAS 1×4”).

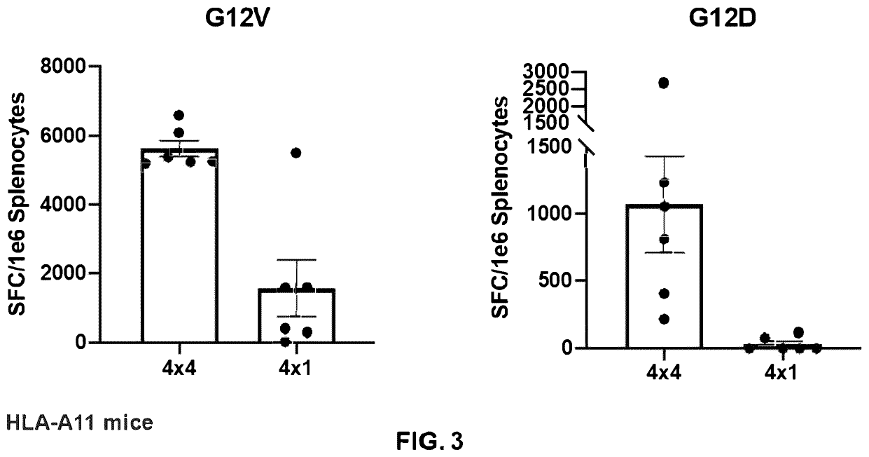

Cassettes featuring either a single copy of KRAS neoepitopes (“4×1”) or 4 iterations of each of the KRAS neoepitopes (“4×4”) were compared. As shown in FIG. 3 and quantified in Table 6, vaccination with vectors featuring multiple iterations of the neoepitopes demonstrated increased spot forming colonies (SFC), indicating repetition of epitope-encoding sequences in a cassette led to an increased antigen-specific immune response against KRAS neoepitopes, specifically G12V and G12D neoepitopes, using a ChAdV68 delivery system.

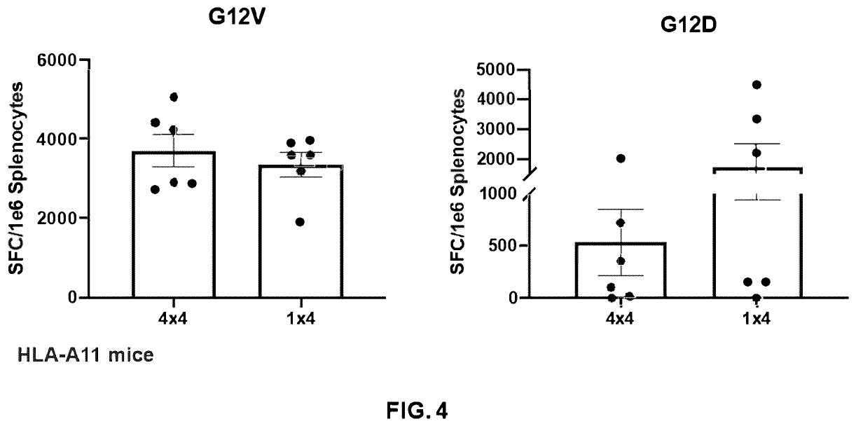

Cassettes featuring either 4 iterations of each of the KRAS neoepitopes (“4×4”) or 4 iterations of only one of the KRAS (“1×4”) were compared. As shown in FIG. 4 and quantified in Table 7, vaccination with vectors featuring multiple iterations of the neoepitopes in either the 4×4 or 1×4 formats generated immune responses by spot forming colonies (SFC) and were comparable across formats, indicating repetition of epitope-encoding sequences in a cassette led to an increased antigen-specific immune response against KRAS neoepitopes, specifically G12V and G12D neoepitopes, using a ChAdV68 delivery system.

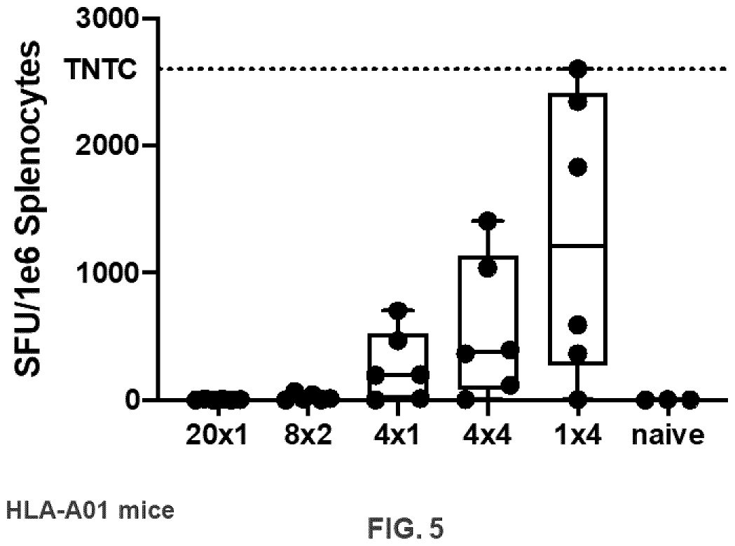

Immune responses for cassette formats for KRAS Q61H were compared, including cassettes featuring either a single copy of KRAS Q61H with 19 other distinct neoepitopes (“20×1”), 2 iterations of the KRAS Q61H (“8×2”), a single copy of KRAS Q61H together with single copies of KRAS G12 neoepitopes (“4×1”), 4 iterations of the KRAS Q61H together with KRAS G12 neoepitope iterations (“4×4”); or 4 iterations of only KRAS Q61H (“1×4”). As shown in FIG. 5 and quantified in Table 8, while vaccination with a vector a single copy of KRAS Q61H together with single copies of KRAS G12 neoepitopes demonstrated a small improvement over larger cassettes (4×1 vs 20×1 or 8×2), vaccination with vectors featuring multiple iterations of the neoepitopes in either the 4×4 or 1×4 formats generated immune responses by spot forming colonies (SFC) with the 1×4 format demonstrating the most robust response, indicating repetition of epitope-encoding sequences in a cassette as well as shorter formats and/or removal of additional distinct neoepitopes led to an increased antigen-specific immune response against the KRAS neoepitopes Q61H, using a ChAdV68 delivery system.

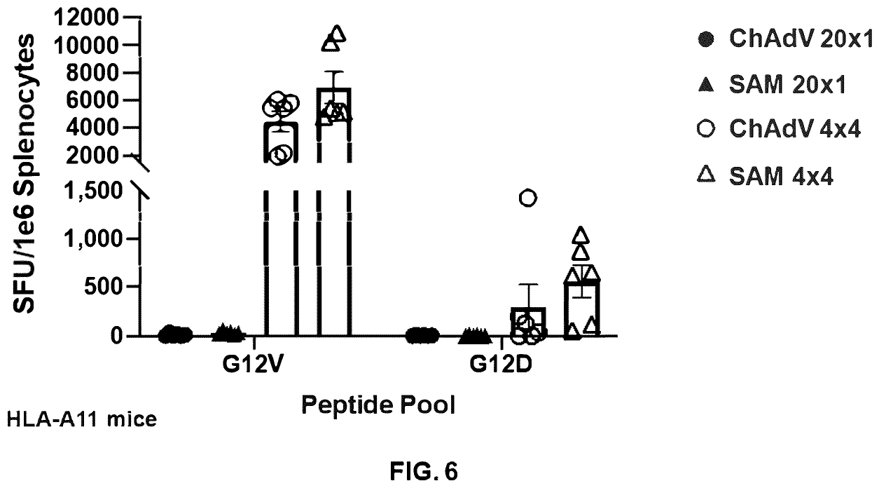

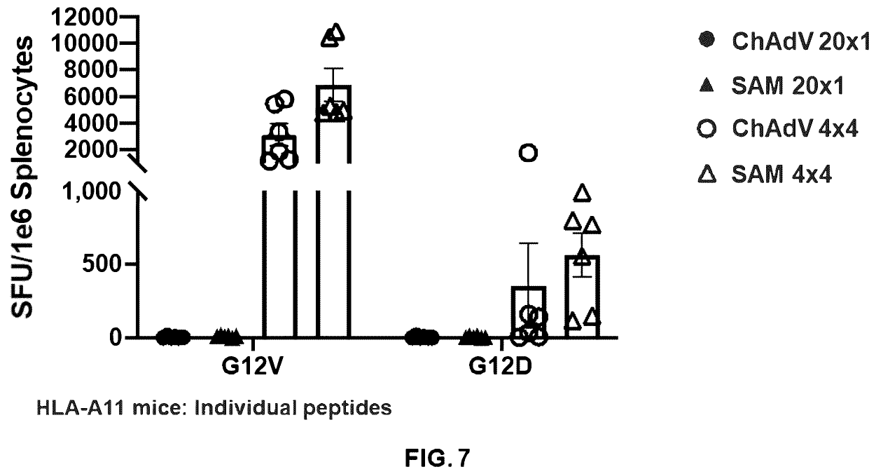

Responses following immunization with either a ChAdV68 delivery system or a SAM delivery system we compared. Cassettes featuring either a single copy of KRAS neoepitopes as well as other neoepitopes (“20×1”) or 4 iterations of each of the KRAS neoepitopes (“4×4”) were assessed. As shown in FIG. 6 and FIG. 7 and quantified in Table 9, vaccination with vectors featuring multiple iterations of the neoepitopes demonstrated increased spot forming colonies (SFC) for both ChAdV68 and SAM formats, indicating repetition of epitope-encoding sequences in a cassette led to an increased antigen-specific immune response against KRAS neoepitopes, specifically G12V and G12D neoepitopes, using either a ChAdV68 or SAM delivery system suggesting the improved response with cassettes featuring iterations of neoepitopes is agnostic of the vector platform used.

TABLE 6
ELISpot data for KRAS Neoepitopes
G12V and G12D +/− Repetition

| | G12V (VVVGAVGGK) | | G12D (VVVGADGVGK) | |
|---|---|---|---|---|
| Antigen | (SEQ ID NO: 91) | | (SEQ ID NO: 78) | |
| Cassette | Mean | +/− SE | Mean | +/− SE |
| 4x4 | 5632 | 237.9 | 1069 | 361.4 |
| 4x1 | 1570 | 833.2 | 32 | 20.6 |

TABLE 7
ELISpot data for KRAS Neoepitopes
G12V and G12D +/− Repetition

| | G12V (VVVGAVGGK) | | G12D (VVVGADGVGK)* | |
|---|---|---|---|---|
| | (SEQ ID NO: 91) | | (SEQ ID NO: 78) | |
| Antigen | | +/− | | +/− |
| Cassette | Mean | SE | Mean | SE |
| 4x4 | 3700 | 404.9 | 536 | 316.1 |
| 1x4 | 3358 | 310.8 | 1726 | 784.7 |

*Results were not significantly different between formats (p = 0.1896)

TABLE 8
ELISpot data for KRAS Neoepitope Q61H +/− Repetition

| | |
|---|---|
| | Q61H (ILDTAGHEEY) |
| Antigen | (SEQ ID NO: 82) |

| Cassette | Mean | +/−SE |
|---|---|---|
| 20 × 1 | 3 | 1.3 |
| 4 × 4 | 554 | 225 |
| 8 × 2 | 20 | 10.8 |
| 4 × 1 | 262 | 112.2 |
| 1 × 4 | 1289 | 451.7 |
| Naive | 1 | 0.6 |

TABLE 9
ELISpot for KRAS G12V and G12D +/−
Repetition in Chad/SAM Formats

| | ChAdV68 20x1 | | | SAM 20x1 | | | ChAdV68 4x4 | | | SAM 4x4 | | |
|---|---|---|---|---|---|---|---|---|---|---|---|---|
| | Mean | SEM | N | Mean | SEM | N | Mean | SEM | N | Mean | SEM | N |
| G12V | 13 | 6 | 6 | 33 | 6 | 6 | 4483 | 767 | 6 | 6939 | 1149 | 6 |
| pool | | | | | | | | | | | | |
| G12D | 5 | 2 | 6 | 6 | 3 | 6 | 297 | 227 | 6 | 560 | 163 | 6 |
| pool | | | | | | | | | | | | |
| G12V | 4 | 2 | 6 | 12 | 3 | 6 | 3167 | 846 | 6 | 6878 | 1216 | 6 |
| (VVGAV | | | | | | | | | | | | |
| GVGK) | | | | | | | | | | | | |
| (SEQ ID | | | | | | | | | | | | |
| NO: 79) | | | | | | | | | | | | |
| G12D | 4 | 3 | 6 | 8 | 3 | 6 | 356 | 288 | 6 | 564 | 148 | 6 |
| (VVVGA | | | | | | | | | | | | |
| DGVGK) | | | | | | | | | | | | |
| (SEQ ID | | | | | | | | | | | | |
| NO: 78) | | | | | | | | | | | | |

### XIV.G. Clinical Assessment of a Neoantigen Vaccine

An open-label, multi-center, multi-dose Phase 1/2 study is performed to assess the dose, safety and tolerability, immunogenicity, and early clinical activity of a heterologous prime/boost vaccination strategy. A vaccine program exploring the iterated KRAS neoepitope cassettes described herein is assessed.

The purpose of this study is to evaluate the dose, safety, immunogenicity and early clinical activity of a ChAdV vector (GRT-C903) and a samRNA vector (GRT-R904), neoantigen-based therapeutic cancer vaccines, in combination with immune checkpoint blockade, in patients with advanced or metastatic non-small cell lung cancer, microsatellite stable colorectal cancer, pancreatic cancer, and shared neoantigen-positive tumors. Tumors harboring non-synonymous deoxyribonucleic acid (DNA) mutations can present peptides containing these mutations as non-self antigens in the context of HLA on the tumor cell surface. A fraction of mutated peptides result in neoantigens capable of generating T-cell responses that exclusively target tumor cells. Some of these tumor-specific neoantigens are known or expected to be common across a subset of patients and are called shared neoantigens. This study targets shared neoantigens using a heterologous prime/boost therapeutic vaccine approach (GRT-C903 first followed by GRT-R904) in combination with checkpoint blockade to stimulate an immune response. This study explores the safety and early clinical activity of this neoantigen-based immunotherapy in inducing T-cell responses specific for the shared neoantigens contained within the therapeutic vaccine. Phase 1 tests multiple doses and combinations with checkpoint blockade and Phase 2 tests for early signs of clinical activity using a vaccine regimen based on Phase 1 data.

A cancer vaccine encoding the iterated KRAS neoepitope cassettes described herein (“SLATE v2” or “SLATE-KRAS”) is administered in combination with immune checkpoint blockade in patients with advanced cancer. The SLATE heterologous prime/boost vaccine regimen involves (1) a ChAdV that is used as a prime vaccination and (2) a samRNA formulated in a LNP that is used for boost vaccinations following the ChAdV vector. Both the ChAdV and samRNA vectors encode the same iterated KRAS neoepitope cassette, which also encodes two universal CD4 T-cell epitopes (PADRE and Tetanus Toxoid). For subject inclusion, tumors are used for whole-exome and transcriptome sequencing to detect somatic mutations, and blood is used for HLA typing.

The ChAdV vector is a replication-defective, E1, E3, E4 open reading frames 2-4 (ORF2-4) deleted adenoviral vector based on chimpanzee adenovirus 68 (C68, 68/SAdV-25, originally designated as Pan 9), which is a subgroup E adenovirus [ChAdV68-Empty-E4deleted; see SEQ ID NO:83 which represents SEQ ID NO:1 with an E1 deletion (577 to 3403), E3 deletion (27,125-31,825), and a partial E4 deletion spanning ORF2-4 (34,916 to 35,642)]. The ChAdV vector is formulated in solution at 5×10¹¹ vp/mL and 1.0 mL is injected IM at each of 2 bilateral vaccine injection sites in opposing deltoid muscles (deltoid muscle preferred, gluteus [dorso or ventro] or rectus femoris on each side may be used).

The samRNA vector is derived from an alphavirus. The samRNA vector encodes the viral proteins and the 5′ and 3′ RNA sequences required for RNA amplification but encoded no structural proteins. The samRNA vector is formulated in LNPs composed of 4 lipids: an ionizable amino lipid, a phosphatidylcholine, cholesterol, and a PEG-based coat lipid to encapsulate the samRNA and form samRNA-LNPs. The samRNA vector contains the same neoantigen expression cassette as used in the ChAdV vector. The samRNA vector is formulated in solution at 1 mg/mL and was injected IM at each of 2 bilateral vaccine injection sites in opposing deltoid muscles (deltoid muscle preferred, gluteus [dorso or ventro] or rectus femoris on each side may be used). The boost vaccination sites are as close to the prime vaccination site as possible. The injection volume is based on the dose to be administered. The dose level amount refers explicitly to the amount of the samRNA vector, i.e., it does not refer to other components, such as the LNP. The ratio of LNP:samRNA is approximately 24:1. Accordingly, the dose of LNP is 720 μg a samRNA vector dose level of 30 μg.

Ipilimumab is a human monoclonal IgG1 antibody that binds to the cytotoxic T-lymphocyte associated antigen 4 (CTLA-4). Ipilimumab is formulated in solution at 5 mg/mL and is injected SC proximally (within ˜2 cm) to each of the bilateral vaccination sites. The SC route of ipilimumab is distinct from the approved IV route of administration. Ipilimumab is administered at a dose of 30 mg in one of two methods listed below:
1. Four 1.5 mL (7.5 mg) injections proximal to the vaccine draining LN at each of the bilateral vaccination sites (i.e., 1.5 mL below the vaccination site and 1.5 mL above the vaccination site on each bilateral side in each deltoid, ventrogluteal, dorsogluteal, or rectus femoris [deltoid preferred, but dependent on clinical site and patient preference]).
2. Six 1 mL (5 mg) injections proximal to the vaccine draining LN at each of the bilateral vaccination sites (i.e., 1 mL below the vaccination site, 1 mL to the side of the vaccination site, and 1 mL above the vaccination site on each bilateral side in each deltoid, ventrogluteal, dorsogluteal, or rectus femoris [deltoid preferred, but dependent on clinical site and patient preference]).

Nivolumab is a human monoclonal IgG4 antibody that blocks the interaction of PD-1 and its ligands, PD-L1 and PD-L2. Nivolumab is formulated in solution at 10 mg/mL and is administered as an IV infusion through a 0.2-micron to 1.2-micron pore size, low-protein binding in-line filter at the protocol-specified doses. It is not administered as an IV push or bolus injection. When the dose is fixed (e.g., 240 mg flat dose), nivolumab injection may be infused undiluted or diluted so as not to exceed a total infusion volume of 160 mL. Nivolumab infusion is promptly followed by a flush of diluent to clear the line. Nivolumab is administered following each vaccination (i.e., each of the samRNA or ChAdV vaccines) with or without ipilimumab on the same day. The dose and route of nivolumab is based on the Food and Drug Administration approved dose and route. Doses of nivolumab may be interrupted, delayed, or discontinued depending on how well the participant tolerates the treatment. Dosing visits are not skipped, only delayed. Vaccination does not occur in the absence of nivolumab unless the Investigator and Sponsor believe treatment with samRNA vectors in the absence of nivolumab is in the best interest of the patient. Cemiplimab may be administered in place of nivolumab, e.g., according to manufacturer's directions and/or appropriate dosing determined as recognized by one skilled in the art. Other PD1 and/or PD-L1 checkpoint inhibitors may alternatively be used.

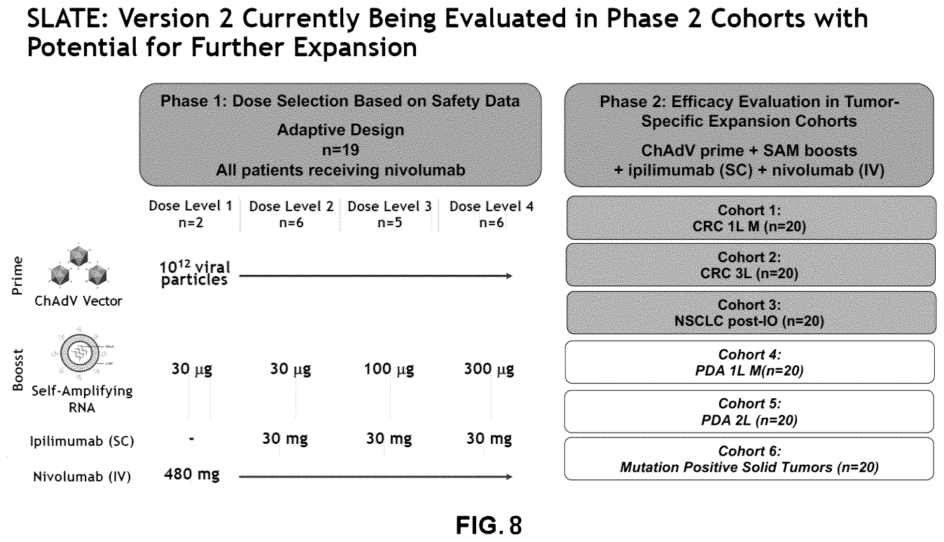

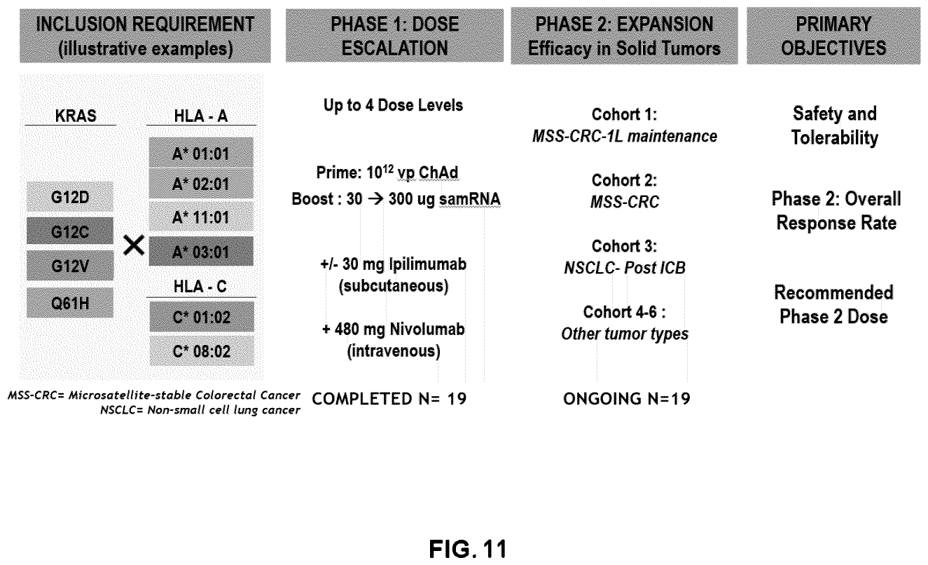

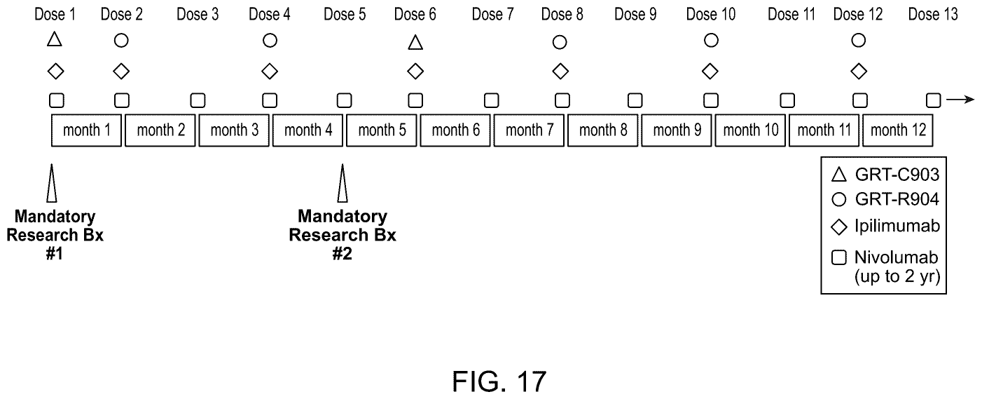

A Phase 1 trial examined prime vaccination with the ChAdV vector followed by multiple dose levels of the samRNA vector with all patients receiving IV nivolumab. Eligible patients included those with advanced or metastatic (1) a solid tumor expressing the KRAS-associated MHC class I neoepitope, (2) colorectal cancer (CRC), including microsatellite-stable CRC (MSS-CRC), (3) non-small cell lung cancer (NSCLC), and/or (4) pancreatic ductal adenocarcinoma (PDA). Following the initial demonstration of the safety and tolerability of Dose Level 1 (30 μg), patients were treated at Dose Level 2 which incorporated SC ipilimumab (30 mg) at the same dose of the samRNA vector as in Dose Level 1 (30 μg). The dose of the SAM vector was escalated to Dose Level 3 (100 μg) followed by Dose Level 4 (300 μg) with all patients continuing to receive SC ipilimumab (30 mg). Phase 2 involves tumor-specific expansion cohorts and a cohort of mutation-positive tumors outside of tumor types already represented by other expansion cohorts. Cohorts 1 and 4 enroll patients who have not experienced disease progression to routine therapy and Cohorts 2, 3, 5, and 6 enroll patients who experienced disease progression on, or after, routine therapy. Patients in Phase 2 receive SC ipilimumab at the dose determined to be well tolerated in Phase 1. Patients receive IV nivolumab at 480 mg Q4W throughout Phase 1 and 2. Details of the Phase 1/2 trials are illustrated in FIG. 8 and FIG. 11. Phase 2 arms include both a monthly treatment schedule or a treatment schedule of every two months. FIG. 17 shows the two-month treatment schedule.

Inclusion criteria specifically include:
1. Patients with the indicated advanced or metastatic solid tumor as follows:
   a. Microsatellite-stable colorectal cancer (MSS-CRC) who are currently receiving systemic treatment with a fluoropyrimidine and oxaliplatin and/or irinotecan that may include a VEGF or EGFR targeting therapy as their 1L therapy for metastatic disease OR who have experienced disease progression following treatment with a fluoropyrimidine, oxaliplatin, and irinotecan that may include a VEGF or EGFR targeting therapy and have not received additional lines of systemic therapy in the metastatic setting.
   b. Non-small cell lung cancer (NSCLC) who are currently receiving systemic treatment with an anti-PD-(L)1 antibody in combination with cytotoxic, platinum-based chemotherapy OR who have experienced disease progression following treatment with an anti-PD-(L)1 antibody in combination with cytotoxic, platinum-based chemotherapy (or anti-PD-(L)1 alone if patient refuses platinum based chemotherapy), and have not received additional lines of systemic therapy in the metastatic setting.
   c. Pancreatic ductal adenocarcinoma (PDA) who are currently receiving systemic cytotoxic chemotherapy as their 1L therapy for metastatic disease OR who have experienced disease progression on 1L systemic cytotoxic chemotherapy and have received no more than 1 prior line of therapy in the metastatic setting.
   d. Any solid tumor histology where the patient has experienced disease progression with all available therapies known to confer clinical benefit; AND/OR
2. Patient's tumor possesses one of the mutations listed below, and is determined to express a HLA allele for antigen presentation of the identified tumor mutation (Table 10): KRAS_G12C or NRAS_G12C; KRAS_G12D or NRAS_G12D; KRAS_G12V or NRAS_G12V; KRAS_Q61H or NRAS_Q61H.

TABLE 10
Neoantigen and HLA Allele Pairs

| | |
|---|---|
| KRAS_G12C | A0201, A0301, A1101, A6801, B0702, C0304 |
| KRAS_G12D | A0201, A0301, A1101, A6801, B0702, C0304, C0802 |
| KRAS_G12V | A0201, A0301, A1101, A6801, B0702, C0102, C0304 |
| KRAS_Q61H | A0101 |

Inclusion criteria also includes: ECOG Performance Status 0 or 1; Measurable disease according to RECIST v1.1; Adequate organ function, as measured by laboratory values.

Exclusion criteria also includes: Tumors with genetic characteristics as follows: (a) For NSCLC, patients with a known genetic driver alteration in EGFR, ALK, ROS1, RET, or TRK; (b) Patients with known MSI-high disease based on institutional standard; Known exposure to chimpanzee adenovirus or any history of anaphylaxis in reaction to a vaccination; Bleeding disorder (e.g., factor deficiency, coagulopathy) or history of significant bruising or bleeding following IM injections or blood draws; History of allogenic/solid organ transplant; Active, known, or suspected autoimmune disease; Active tuberculosis or recent (<2 week) clinically significant infection, or evidence of active hepatitis B or hepatitis C; Known history of positive test for human immunodeficiency (HIV) or known acquired immunodeficiency syndrome (AIDS).

The overall demographics for patients assessed in the Phase 1/2 trial are shown below in Table 11.

TABLE 11
Subject Demographics

| | | SLATE- | |
|---|---|---|---|
| | Version 1 | KRAS | Overall |
| | N = 26 | N = 12 | N = 38 |
| Demographics and Tumor Type | | | |
| Age: mean years (min, max) | 60 | 59 | 60 |
| | (33, 83) | (36, 86) | (33, 86) |
| Gender (Male/Female) | 10/16 | 4/8 | 14/24 |
| Tumor Types | | | |
| Non-small Cell Lung Cancer | 13 | 5 | 18 |
| (NSCLC) | | | |
| No. Prior therapy (median, | 1 | 2 | 1.5 |
| range) | (1-4) | (1-3) | (1-4) |
| Prior anti-PD-(L)1 therapy | 13 | 5 | 18 |
| Microsatellite-Stable (MSS) | 6 | 7 | 13 |
| Colorectal Cancer | | | |
| No. Prior therapy (median, | 1.5 | 1 | 1 |
| range) | (1-2) | (1-2) | (1-2) |
| No. Prior Oxaliplatin/Irinotecan | 6 | 6 | 12 |
| Pancreatic Ductal Adenocarcinoma | 5 | 0 | 5 |
| (PDA) | | | |
| No. Prior therapy (median, range) | 1 | 0 | 1 |
| | (1-3) | | (1-3) |
| Other | 2 | 0 | 2 |
| No. Prior therapy (median, range) | 3 | 0 | 3 |
| | (3) | | (3) |

A summary of adverse events for heterologous prime-boost neoantigen directed VI and SLATE-KRAS was well-tolerated in advanced solid cancer patients, as in shown in Table 12.

TABLE 12
Summary of Adverse Events

| Preferred | V1 (n = 26) | | | KRAS-SLATE (n = 12) | | | Overall (n = 38) | |
|---|---|---|---|---|---|---|---|---|
| Terms; n(%) | Grade 1/2 | | ≥Grade 3 | Grade 1/2 | | ≥Grade 3 | All Grades | |
| Pyrexia | 14 | (53.8) | 0.0 | 3 | (25.0) | 0.0 | 17 | (44.7) |
| Fatigue | 8 | (30.8) | 1(3.8) | 2 | (16.7) | 0.0 | 11 | (28.9) |
| Nausea | 6 | (23.1) | 0.0 | 3 | (25.0) | 0.0 | 9 | (23.7) |
| Vomiting | 7 | (26.9) | 0.0 | 1 | (8.3) | 0.0 | 8 | (21.1) |
| Chills | 3 | (11.5) | 0.0 | 4 | (33.3) | 0.0 | 7 | (18.4) |
| Diarrhoea | 5 | (19.2) | 0.0 | 1 | (8.3) | 0.0 | 6 | (15.8) |
| Myalgia | 2 | (7.7) | 0.0 | 3 | (25.0) | 0.0 | 5 | (13.2) |
| Decreased appetite | 2 | (7.7) | 0.0 | 2 | (16.7) | 0.0 | 4 | (10.5) |
| Injection site pain | 2 | (7.7) | 0.0 | 2 | (16.7) | 0.0 | 4 | (10.5) |

| | | | | | | | |
|---|---|---|---|---|---|---|---|
| Injection site reaction | 4 | (15.4) | 0.0 | 0.00 | 0.0 | 4 | (10.5) |

Treatment-related Grade 3/4 AEs with frequency <10%:
With V1, one patient experienced transient Grade 3 increase in alanine aminotransferase and aspartate aminotransferase
With V1, one patient had Grade 3 confusional state associated with pyrexia

The cancer vaccine encoding the optimized SLATE v2 iterated KRAS neoepitope cassette was administered to a SLATE patient (S31) that was 41-year-old male with metastatic NSCLC identified as having a KRAS G12V mutation as well as haplotyped as A*03:01. S31 had prior treatment with pembrolizumab, carboplatin, and pemetrexed for 16 months with progressive disease. S31 received Dose 1-ChAdV vector in combination with SC ipilimumab (30 mg) and IV nivolumab (480 mg) as the prime vaccination followed by dosing visits every 4 weeks as follows:
Dose 2—samRNA vector (30 μg) in combination with SC ipilimumab (30 mg) and IV nivolumab (480 mg) as a boost vaccination
Dose 3—nivolumab (480 mg) monotherapy
Dose 4—SAM vector in combination with SC ipilimumab (30 mg) and IV nivolumab (480 mg) as a boost vaccination
Dose 5—nivolumab (480 mg) monotherapy
S31 remained on the study treatment for 6 months

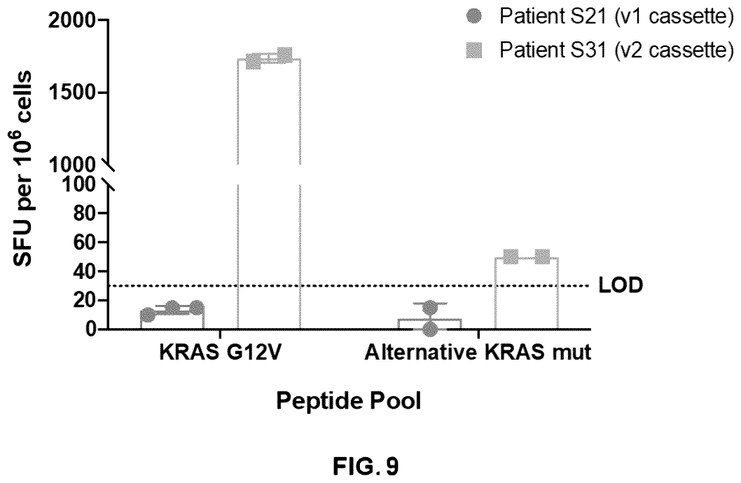

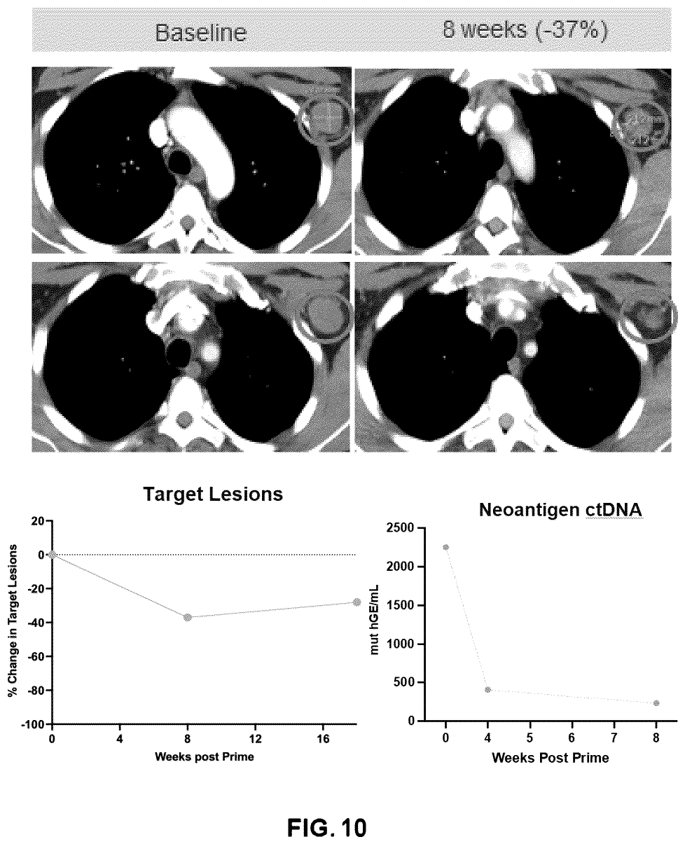

T cell responses were assessed and compared between the patient S31 administered the optimized SLATE v2 cassette that included the iterated KRAS neoepitopes and a SLATE patient (S21) that was administered the prior SLATE “version 1” (v1) cassette. As shown in FIG. 9, patient S31 receiving the SLATE v2 cassette demonstrated an increased response (˜100-fold) relative to the patient receiving the SLATE v1 cassette, in particular for the KRAS G12V peptide. Accordingly, the cassette featuring the iterated KRAS neoepitopes demonstrated an improved immune response stimulatory capacity. Further, as shown in FIG. 10, patient S31 demonstrated a 37% reduction in tumor size at week 8 (FIG. 10 top and bottom left panels) and 28% at week 18 (FIG. 10 bottom left panel), as well as a molecular response as assessed by monitoring neoantigen ctDNA (FIG. 10 right panel), demonstrating potential clinical efficacy at the timepoints assessed here.

The cancer vaccine encoding the optimized SLATE v2 iterated KRAS neoepitope cassette was administered to a SLATE patient (S37) that was 64-year-old female with metastatic CRC identified as having a KRAS G12V mutation as well as haplotyped as A*11:01. S37 had prior treatment with FOLFOX/FOLFIR/bevacizumab for 23 months with progressive disease. S37 received Dose 1-ChAdV vector in combination with SC ipilimumab (30 mg) and IV nivolumab (480 mg) as the prime vaccination followed by dosing visits every 4 weeks (Q4W) as follows (e.g., as illustrated in FIG. 17):
Dose 2—samRNA vector (30 μg) in combination with SC ipilimumab (30 mg) and IV nivolumab (480 mg) as a boost vaccination
Dose 3—nivolumab (480 mg) monotherapy
Dose 4—samRNA vector (30 μg) in combination with SC ipilimumab (30 mg) and IV nivolumab (480 mg) as a boost vaccination
Dose 5—nivolumab (480 mg) monotherapy
Dose 6—ChAdV vector (same dose as prime) in combination with SC ipilimumab (30 mg) and IV nivolumab (480 mg)
Dose 7—nivolumab (480 mg) monotherapy
Dose 8—samRNA vector (30 μg) in combination with SC ipilimumab (30 mg) and IV nivolumab (480 mg) as a boost vaccination
Dose 9—nivolumab (480 mg) monotherapy
Dose 10—samRNA vector (30 μg) in combination with SC ipilimumab (30 mg) and IV nivolumab (480 mg) as a boost vaccination
S37 remained on the study treatment

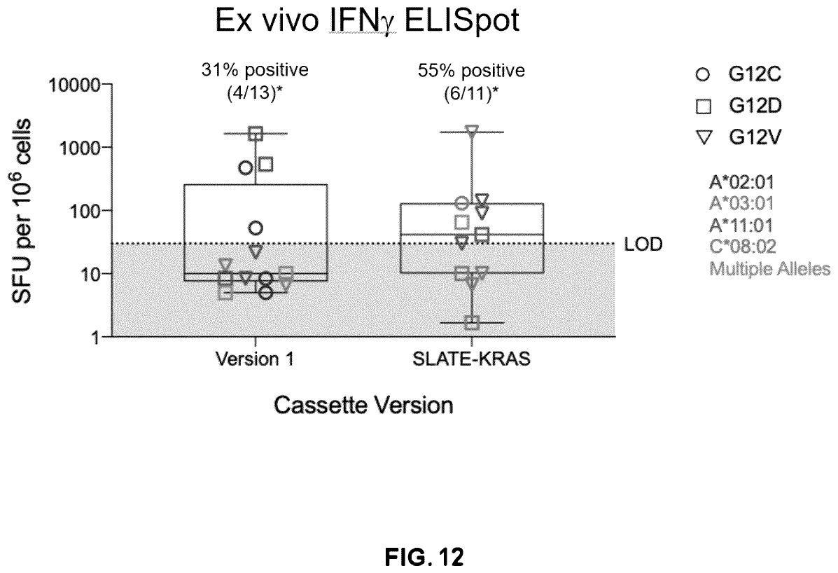

T cell responses were assessed and compared between the patient S37 administered the optimized SLATE v2 cassette that included the iterated KRAS neoepitopes and a SLATE patient (S21) that was administered the prior SLATE “version 1” (v1) cassette. As shown in FIG. 9, patient S31 receiving the SLATE v2 cassette demonstrated an increased response (˜100-fold) relative to the patient receiving the SLATE v1 cassette, in particular for the KRAS G12V peptide. A summary of T cells responses assessed by IFNγ ELISpot for various G12 mutations, alleles, and cassettes in shown in FIG. 12. Accordingly, the cassette featuring the iterated KRAS neoepitopes demonstrated an improved immune response stimulatory capacity.

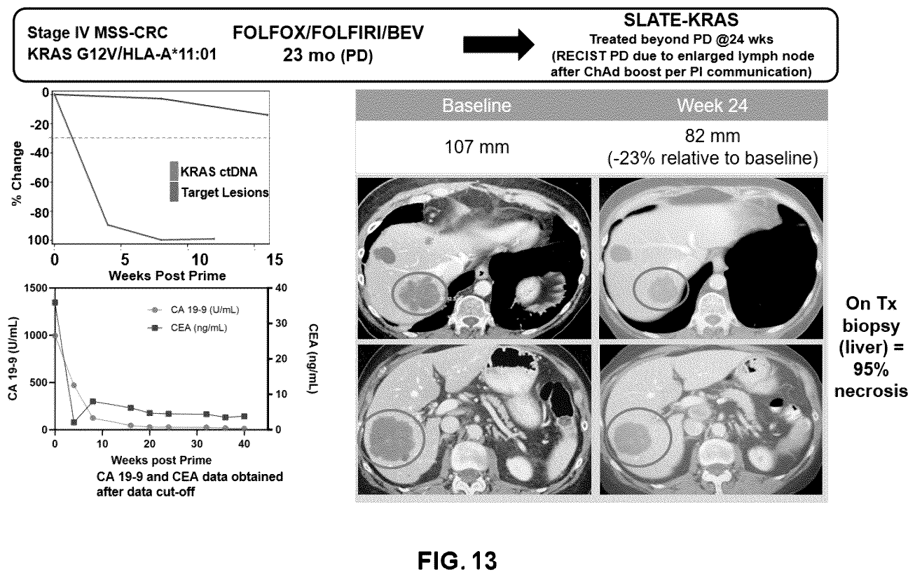

Further, as shown in FIG. 13, patient S37 demonstrated a 23% reduction in tumor size at week 24 (FIG. 13 right panels; week 24), as well as a molecular response as assessed by monitoring neoantigen ctDNA (FIG. 13 left top panel) and standard serum tumor markers CEA and CA 19-9 (FIG. 13 left bottom panel), demonstrating potential clinical efficacy.

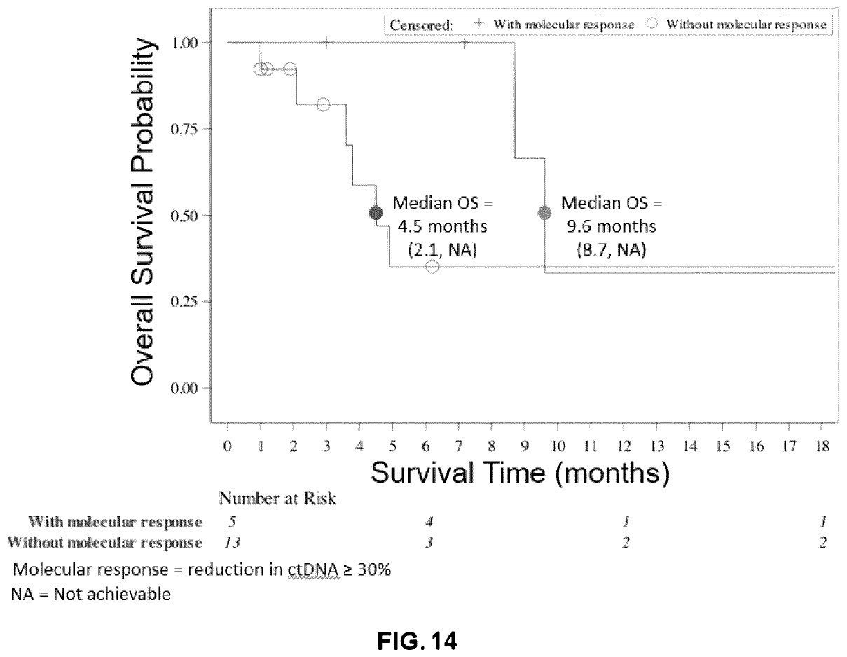

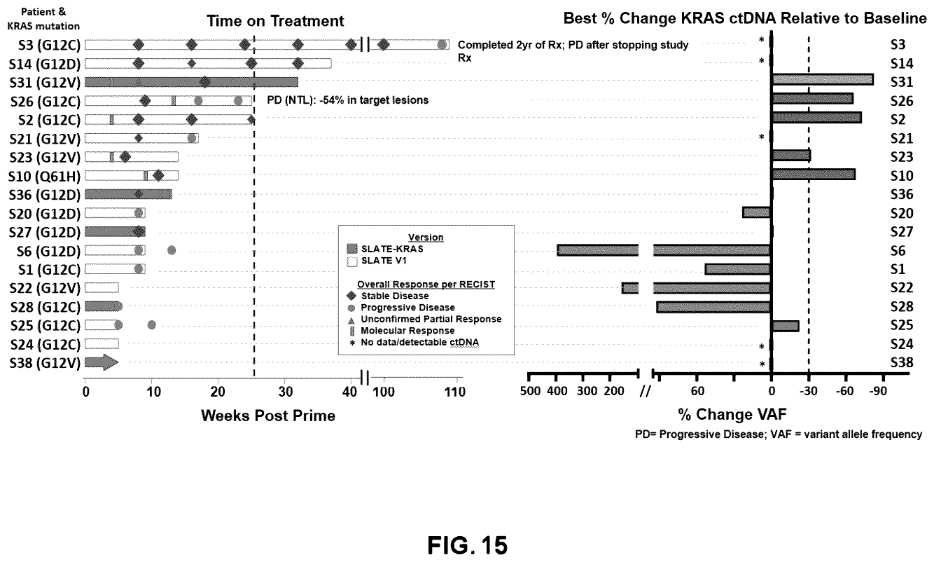

Clinical efficacy was assessed in patients with NSCLC. Molecular responses (reduction in ctDNA>30%) and survival probabilities are shown in FIG. 14, demonstrating a trend of improved overall survival in patients with NSCLC with a molecular response compared to those without. A summary of clinical results for patients with NSCLC is presented FIG. 15, demonstrating early signals of durability and molecular response in NSCLC subjects following immune-checkpoint blockade (anti-PD-(L)1 therapy).

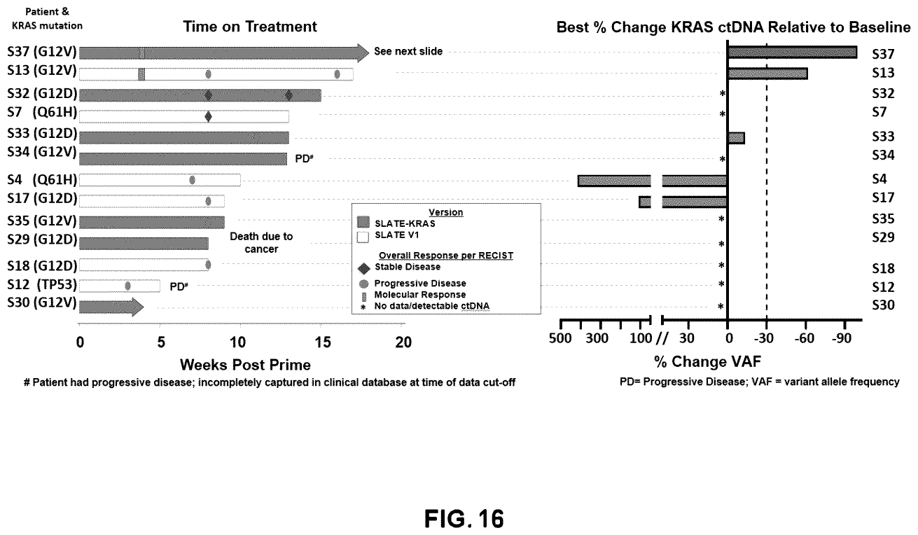

Clinical efficacy was assessed in patients with late-stage CRC. A summary of clinical results for patients with late-stage CRC is presented FIG. 16, demonstrating early signals of durability and molecular response in late-stage CRC.

Overall, a molecular response (reduction in ctDNA) was observed in 31.8% of patients with CRC and NSCLC preceding clinical benefit, including a molecular and clinical benefit in a patient with KRAS G12V mutant CRC who had progressed on 2 prior therapies. Additionally, assessing molecular responses by ctDNA may provided an improved biomarker of benefit versus RECIST radiology with this immunotherapy.

Vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2);
AC_000011.1 with E1 (nt 577 to 3403) and
E3 (nt 27,125-31,825) sequences deleted;
corresponding ATCC VR-594 nucleotides
substituted at five positions; model
neoantigen cassette under the control
of the CMV promoter/enhancer inserted
in place of deleted E1; SV40 polyA
3′ of cassette
Venezuelan equine encephalitis virus
[VEE](SEQ ID NO: 3) GenBank: L01442.2
VEE-MAG25mer (SEQ ID NO: 4); contains
MAG-25merPDTT nucleotide (bases 30-1755)
Venezuelan equine encephalitis virus
strain TC-83 [TC-83](SEQ ID NO: 5)
GenBank: L01443.1
ATAGGCGGCGCATGAGAGAAGCCCAGACCAATTACCTACCCAAAA
TGGAGAAAGTTCACGTTGACATCGAGGAAGACAGCCCATTCCTCA
GAGCTTTGCAGCGGAGCTTCCCGCAGTTTGAGGTAGAAGCCAAGC
AGGTCACTGATAATGACCATGCTAATGCCAGAGCGTTTTCGCATC
TGGCTTCAAAACTGATCGAAACGGAGGTGGACCCATCCGACACGA
TCCTTGACATTGGAAGTGCGCCCGCCCGCAGAATGTATTCTAAGC
ACAAGTATCATTGTATCTGTCCGATGAGATGTGCGGAAGATCCGG
ACAGATTGTATAAGTATGCAACTAAGCTGAAGAAAAACTGTAAGG
AAATAACTGATAAGGAATTGGACAAGAAAATGAAGGAGCTCGCCG
CCGTCATGAGCGACCCTGACCTGGAAACTGAGACTATGTGCCTCC
ACGACGACGAGTCGTGTCGCTACGAAGGGCAAGTCGCTGTTTACC
AGGATGTATACGCGGTTGACGGACCGACAAGTCTCTATCACCAAG
CCAATAAGGGAGTTAGAGTCGCCTACTGGATAGGCTTTGACACCA
CCCCTTTTATGTTTAAGAACTTGGCTGGAGCATATCCATCATACT
CTACCAACTGGGCCGACGAAACCGTGTTAACGGCTCGTAACATAG
GCCTATGCAGCTCTGACGTTATGGAGCGGTCACGTAGAGGGATGT
CCATTCTTAGAAAGAAGTATTTGAAACCATCCAACAATGTTCTAT
TCTCTGTTGGCTCGACCATCTACCACGAGAAGAGGGACTTACTGA
GGAGCTGGCACCTGCCGTCTGTATTTCACTTACGTGGCAAGCAAA
ATTACACATGTCGGTGTGAGACTATAGTTAGTTGCGACGGGTACG
TCGTTAAAAGAATAGCTATCAGTCCAGGCCTGTATGGGAAGCCTT
CAGGCTATGCTGCTACGATGCACCGCGAGGGATTCTTGTGCTGCA
AAGTGACAGACACATTGAACGGGGAGAGGGTCTCTTTTCCCGTGT
GCACGTATGTGCCAGCTACATTGTGTGACCAAATGACTGGCATAC
TGGCAACAGATGTCAGTGCGGACGACGCGCAAAAACTGCTGGTTG
GGCTCAACCAGCGTATAGTCGTCAACGGTCGCACCCAGAGAAACA
CCAATACCATGAAAAATTACCTTTTGCCCGTAGTGGCCCAGGCAT
TTGCTAGGTGGGCAAAGGAATATAAGGAAGATCAAGAAGATGAAA
GGCCACTAGGACTACGAGATAGACAGTTAGTCATGGGGTGTTGTT
GGGCTTTTAGAAGGCACAAGATAACATCTATTTATAAGCGCCCGG
ATACCCAAACCATCATCAAAGTGAACAGCGATTTCCACTCATTCG
TGCTGCCCAGGATAGGCAGTAACACATTGGAGATCGGGCTGAGAA
CAAGAATCAGGAAAATGTTAGAGGAGCACAAGGAGCCGTCACCTC
TCATTACCGCCGAGGACGTACAAGAAGCTAAGTGCGCAGCCGATG
AGGCTAAGGAGGTGCGTGAAGCCGAGGAGTTGCGCGCAGCTCTAC
CACCTTTGGCAGCTGATGTTGAGGAGCCCACTCTGGAAGCCGATG
TCGACTTGATGTTACAAGAGGCTGGGGCCGGCTCAGTGGAGACAC
CTCGTGGCTTGATAAAGGTTACCAGCTACGATGGCGAGGACAAGA
TCGGCTCTTACGCTGTGCTTTCTCCGCAGGCTGTACTCAAGAGTG
AAAAATTATCTTGCATCCACCCTCTCGCTGAACAAGTCATAGTGA
TAACACACTCTGGCCGAAAAGGGCGTTATGCCGTGGAACCATACC
ATGGTAAAGTAGTGGTGCCAGAGGGACATGCAATACCCGTCCAGG
ACTTTCAAGCTCTGAGTGAAAGTGCCACCATTGTGTACAACGAAC
GTGAGTTCGTAAACAGGTACCTGCACCATATTGCCACACATGGAG
GAGCGCTGAACACTGATGAAGAATATTACAAAACTGTCAAGCCCA
GCGAGCACGACGGCGAATACCTGTACGACATCGACAGGAAACAGT
GCGTCAAGAAAGAACTAGTCACTGGGCTAGGGCTCACAGGCGAGC
TGGTGGATCCTCCCTTCCATGAATTCGCCTACGAGAGTCTGAGAA
CACGACCAGCCGCTCCTTACCAAGTACCAACCATAGGGGTGTATG
GCGTGCCAGGATCAGGCAAGTCTGGCATCATTAAAAGCGCAGTCA
CCAAAAAAGATCTAGTGGTGAGCGCCAAGAAAGAAAACTGTGCAG
AAATTATAAGGGACGTCAAGAAAATGAAAGGGCTGGACGTCAATG
CCAGAACTGTGGACTCAGTGCTCTTGAATGGATGCAAACACCCCG
TAGAGACCCTGTATATTGACGAAGCTTTTGCTTGTCATGCAGGTA
CTCTCAGAGCGCTCATAGCCATTATAAGACCTAAAAAGGCAGTGC
TCTGCGGGGATCCCAAACAGTGCGGTTTTTTTAACATGATGTGCC
TGAAAGTGCATTTTAACCACGAGATTTGCACACAAGTCTTCCACA
AAAGCATCTCTCGCCGTTGCACTAAATCTGTGACTTCGGTCGTCT
CAACCTTGTTTTACGACAAAAAAATGAGAACGACGAATCCGAAAG
AGACTAAGATTGTGATTGACACTACCGGCAGTACCAAACCTAAGC
AGGACGATCTCATTCTCACTTGTTTCAGAGGGTGGGTGAAGCAGT
TGCAAATAGATTACAAAGGCAACGAAATAATGACGGCAGCTGCCT
CTCAAGGGCTGACCCGTAAAGGTGTGTATGCCGTTCGGTACAAGG
TGAATGAAAATCCTCTGTACGCACCCACCTCAGAACATGTGAACG
TCCTACTGACCCGCACGGAGGACCGCATCGTGTGGAAAACACTAG
CCGGCGACCCATGGATAAAAACACTGACTGCCAAGTACCCTGGGA
ATTTCACTGCCACGATAGAGGAGTGGCAAGCAGAGCATGATGCCA
TCATGAGGCACATCTTGGAGAGACCGGACCCTACCGACGTCTTCC
AGAATAAGGCAAACGTGTGTTGGGCCAAGGCTTTAGTGCCGGTGC
TGAAGACCGCTGGCATAGACATGACCACTGAACAATGGAACACTG
TGGATTATTTTGAAACGGACAAAGCTCACTCAGCAGAGATAGTAT
TGAACCAACTATGCGTGAGGTTCTTTGGACTCGATCTGGACTCCG
GTCTATTTTCTGCACCCACTGTTCCGTTATCCATTAGGAATAATC
ACTGGGATAACTCCCCGTCGCCTAACATGTACGGGCTGAATAAAG
AAGTGGTCCGTCAGCTCTCTCGCAGGTACCCACAACTGCCTCGGG
CAGTTGCCACTGGAAGAGTCTATGACATGAACACTGGTACACTGC
GCAATTATGATCCGCGCATAAACCTAGTACCTGTAAACAGAAGAC
TGCCTCATGCTTTAGTCCTCCACCATAATGAACACCCACAGAGTG
ACTTTTCTTCATTCGTCAGCAAATTGAAGGGCAGAACTGTCCTGG
TGGTCGGGGAAAAGTTGTCCGTCCCAGGCAAAATGGTTGACTGGT
TGTCAGACCGGCCTGAGGCTACCTTCAGAGCTCGGCTGGATTTAG
GCATCCCAGGTGATGTGCCCAAATATGACATAATATTTGTTAATG
TGAGGACCCCATATAAATACCATCACTATCAGCAGTGTGAAGACC
ATGCCATTAAGCTTAGCATGTTGACCAAGAAAGCTTGTCTGCATC
TGAATCCCGGCGGAACCTGTGTCAGCATAGGTTATGGTTACGCTG
ACAGGGCCAGCGAAAGCATCATTGGTGCTATAGCGCGGCAGTTCA
AGTTTTCCCGGGTATGCAAACCGAAATCCTCACTTGAAGAGACGG
AAGTTCTGTTTGTATTCATTGGGTACGATCGCAAGGCCCGTACGC
ACAATCCTTACAAGCTTTCATCAACCTTGACCAACATTTATACAG
GTTCCAGACTCCACGAAGCCGGATGTGCACCCTCATATCATGTGG
TGCGAGGGGATATTGCCACGGCCACCGAAGGAGTGATTATAAATG
CTGCTAACAGCAAAGGACAACCTGGCGGAGGGGTGTGCGGAGCGC
TGTATAAGAAATTCCCGGAAAGCTTCGATTTACAGCCGATCGAAG
TAGGAAAAGCGCGACTGGTCAAAGGTGCAGCTAAACATATCATTC
ATGCCGTAGGACCAAACTTCAACAAAGTTTCGGAGGTTGAAGGTG
ACAAACAGTTGGCAGAGGCTTATGAGTCCATCGCTAAGATTGTCA
ACGATAACAATTACAAGTCAGTAGCGATTCCACTGTTGTCCACCG
GCATCTTTTCCGGGAACAAAGATCGACTAACCCAATCATTGAACC
ATTTGCTGACAGCTTTAGACACCACTGATGCAGATGTAGCCATAT
ACTGCAGGGACAAGAAATGGGAAATGACTCTCAAGGAAGCAGTGG
CTAGGAGAGAAGCAGTGGAGGAGATATGCATATCCGACGACTCTT
CAGTGACAGAACCTGATGCAGAGCTGGTGAGGGTGCATCCGAAGA
GTTCTTTGGCTGGAAGGAAGGGCTACAGCACAAGCGATGGCAAAA
CTTTCTCATATTTGGAAGGGACCAAGTTTCACCAGGCGGCCAAGG
ATATAGCAGAAATTAATGCCATGTGGCCCGTTGCAACGGAGGCCA
ATGAGCAGGTATGCATGTATATCCTCGGAGAAAGCATGAGCAGTA
TTAGGTCGAAATGCCCCGTCGAAGAGTCGGAAGCCTCCACACCAC
CTAGCACGCTGCCTTGCTTGTGCATCCATGCCATGACTCCAGAAA
GAGTACAGCGCCTAAAAGCCTCACGTCCAGAACAAATTACTGTGT
GCTCATCCTTTCCATTGCCGAAGTATAGAATCACTGGTGTGCAGA
AGATCCAATGCTCCCAGCCTATATTGTTCTCACCGAAAGTGCCTG
CGTATATTCATCCAAGGAAGTATCTCGTGGAAACACCACCGGTAG
ACGAGACTCCGGAGCCATCGGCAGAGAACCAATCCACAGAGGGGA
CACCTGAACAACCACCACTTATAACCGAGGATGAGACCAGGACTA
GAACGCCTGAGCCGATCATCATCGAAGAGGAAGAAGAGGATAGCA
TAAGTTTGCTGTCAGATGGCCCGACCCACCAGGTGCTGCAAGTCG
AGGCAGACATTCACGGGCCGCCCTCTGTATCTAGCTCATCCTGGT
CCATTCCTCATGCATCCGACTTTGATGTGGACAGTTTATCCATAC
TTGACACCCTGGAGGGAGCTAGCGTGACCAGCGGGGCAACGTCAG
CCGAGACTAACTCTTACTTCGCAAAGAGTATGGAGTTTCTGGCGC
GACCGGTGCCTGCGCCTCGAACAGTATTCAGGAACCCTCCACATC
CCGCTCCGCGCACAAGAACACCGTCACTTGCACCCAGCAGGGCCT
GCTCGAGAACCAGCCTAGTTTCCACCCCGCCAGGCGTGAATAGGG
TGATCACTAGAGAGGAGCTCGAGGCGCTTACCCCGTCACGCACTC
CTAGCAGGTCGGTCTCGAGAACCAGCCTGGTCTCCAACCCGCCAG
GCGTAAATAGGGTGATTACAAGAGAGGAGTTTGAGGCGTTCGTAG
CACAACAACAATGACGGTTTGATGCGGGTGCATACATCTTTTCCT
CCGACACCGGTCAAGGGCATTTACAACAAAAATCAGTAAGGCAAA
CGGTGCTATCCGAAGTGGTGTTGGAGAGGACCGAATTGGAGATTT
CGTATGCCCCGCGCCTCGACCAAGAAAAAGAAGAATTACTACGCA
AGAAATTACAGTTAAATCCCACACCTGCTAACAGAAGCAGATACC
AGTCCAGGAAGGTGGAGAACATGAAAGCCATAACAGCTAGACGTA
TTCTGCAAGGCCTAGGGCATTATTTGAAGGCAGAAGGAAAAGTGG
AGTGCTACCGAACCCTGCATCCTGTTCCTTTGTATTCATCTAGTG
TGAACCGTGCCTTTTCAAGCCCCAAGGTCGCAGTGGAAGCCTGTA
ACGCCATGTTGAAAGAGAACTTTCCGACTGTGGCTTCTTACTGTA
TTATTCCAGAGTACGATGCCTATTTGGACATGGTTGACGGAGCTT
CATGCTGCTTAGACACTGCCAGTTTTTGCCCTGCAAAGCTGCGCA
GCTTTCCAAAGAAACACTCCTATTTGGAACCCACAATACGATCGG
CAGTGCCTTCAGCGATCCAGAACACGCTCCAGAACGTCCTGGCAG
CTGCCACAAAAAGAAATTGCAATGTCACGCAAATGAGAGAATTGC
CCGTATTGGATTCGGCGGCCTTTAATGTGGAATGCTTCAAGAAAT
ATGCGTGTAATAATGAATATTGGGAAACGTTTAAAGAAAACCCCA
TCAGGCTTACTGAAGAAAACGTGGTAAATTACATTACCAAATTAA
AAGGACCAAAAGCTGCTGCTCTTTTTGCGAAGACACATAATTTGA
ATATGTTGCAGGACATACCAATGGACAGGTTTGTAATGGACTTAA
AGAGAGACGTGAAAGTGACTCCAGGAACAAAACATACTGAAGAAC
GGCCCAAGGTACAGGTGATCCAGGCTGCCGATCCGCTAGCAACAG
CGTATCTGTGCGGAATCCACCGAGAGCTGGTTAGGAGATTAAATG
CGGTCCTGCTTCCGAACATTCATACACTGTTTGATATGTCGGCTG
AAGACTTTGACGCTATTATAGCCGAGCACTTCCAGCCTGGGGATT
GTGTTCTGGAAACTGACATCGCGTCGTTTGATAAAAGTGAGGACG
ACGCCATGGCTCTGACCGCGTTAATGATTCTGGAAGACTTAGGTG
TGGACGCAGAGCTGTTGACGCTGATTGAGGCGGCTTTCGGCGAAA
TTTCATCAATACATTTGCCCACTAAAACTAAATTTAAATTCGGAG
CCATGATGAAATCTGGAATGTTCCTCACACTGTTTGTGAACACAG
TCATTAACATTGTAATCGCAAGCAGAGTGTTGAGAGAACGGCTAA
CCGGATCACCATGTGCAGCATTCATTGGAGATGACAATATCGTGA
AAGGAGTCAAATCGGACAAATTAATGGCAGACAGGTGCGCCACCT
GGTTGAATATGGAAGTCAAGATTATAGATGCTGTGGTGGGCGAGA
AAGCGCCTTATTTCTGTGGAGGGTTTATTTTGTGTGACTCCGTGA
CCGGCACAGCGTGCCGTGTGGCAGACCCCCTAAAAAGGCTGTTTA
AGCTTGGCAAACCTCTGGCAGCAGACGATGAACATGATGATGACA
GGAGAAGGGCATTGCATGAAGAGTCAACACGCTGGAACCGAGTGG
GTATTCTTTCAGAGCTGTGCAAGGCAGTAGAATCAAGGTATGAAA
CCGTAGGAACTTCCATCATAGTTATGGCCATGACTACTCTAGCTA
GCAGTGTTAAATCATTCAGCTACCTGAGAGGGGCCCCTATAACTC
TCTACGGCTAACCTGAATGGACTACGACATAGTCTAGTCCGCCAA
GATGTTCCCGTTCCAGCCAATGTATCCGATGCAGCCAATGCCCTA
TCGCAACCCGTTCGCGGCCCCGCGCAGGCCCTGGTTCCCCAGAAC
CGACCCTTTTCTGGCGATGCAGGTGCAGGAATTAACCCGCTCGAT
GGCTAACCTGACGTTCAAGCAACGCCGGGACGCGCCACCTGAGGG
GCCATCCGCTAAGAAACCGAAGAAGGAGGCCTCGCAAAAACAGAA
AGGGGGAGGCCAAGGGAAGAAGAAGAAGAACCAAGGGAAGAAGAA
GGCTAAGACAGGGCCGCCTAATCCGAAGGCACAGAATGGAAACAA
GAAGAAGACCAACAAGAAACCAGGCAAGAGACAGCGCATGGTCAT
GAAATTGGAATCTGACAAGACGTTCCCAATCATGTTGGAAGGGAA
GATAAACGGCTACGCTTGTGTGGTCGGAGGGAAGTTATTCAGGCC
GATGCATGTGGAAGGCAAGATCGACAACGACGTTCTGGCCGCGCT
TAAGACGAAGAAAGCATCCAAATACGATCTTGAGTATGCAGATGT
GCCACAGAACATGCGGGCCGATACATTCAAATACACCCATGAGAA
ACCCCAAGGCTATTACAGCTGGCATCATGGAGCAGTCCAATATGA
AAATGGGCGTTTCACGGTGCCGAAAGGAGTTGGGGCCAAGGGAGA
CAGCGGACGACCCATTCTGGATAACCAGGGACGGGTGGTCGCTAT
TGTGCTGGGAGGTGTGAATGAAGGATCTAGGACAGCCCTTTCAGT
CGTCATGTGGAACGAGAAGGGAGTTACCGTGAAGTATACTCCGGA
GAACTGCGAGCAATGGTCACTAGTGACCACCATGTGTCTGCTCGC
CAATGTGACGTTCCCATGTGCTCAACCACCAATTTGCTACGACAG
AAAACCAGCAGAGACTTTGGCCATGCTCAGCGTTAACGTTGACAA
CCCGGGCTACGATGAGCTGCTGGAAGCAGCTGTTAAGTGCCCCGG
AAGGAAAAGGAGATCCACCGAGGAGCTGTTTAATGAGTATAAGCT
AACGCGCCCTTACATGGCCAGATGCATCAGATGTGCAGTTGGGAG
CTGCCATAGTCCAATAGCAATCGAGGCAGTAAAGAGCGACGGGCA
CGACGGTTATGTTAGACTTCAGACTTCCTCGCAGTATGGCCTGGA
TTCCTCCGGCAACTTAAAGGGCAGGACCATGCGGTATGACATGCA
CGGGACCATTAAAGAGATACCACTACATCAAGTGTCACTCTATAC
ATCTCGCCCGTGTCACATTGTGGATGGGCACGGTTATTTCCTGCT
TGCCAGGTGCCCGGCAGGGGACTCCATCACCATGGAATTTAAGAA
AGATTCCGTCAGACACTCCTGCTCGGTGCCGTATGAAGTGAAATT
TAATCCTGTAGGCAGAGAACTCTATACTCATCCCCCAGAACACGG
AGTAGAGCAAGCGTGCCAAGTCTACGCACATGATGCACAGAACAG
AGGAGCTTATGTCGAGATGCACCTCCCGGGCTCAGAAGTGGACAG
CAGTTTGGTTTCCTTGAGCGGCAGTTCAGTCACCGTGACACCTCC
TGATGGGACTAGCGCCCTGGTGGAATGCGAGTGTGGCGGCACAAA
GATCTCCGAGACCATCAACAAGACAAAACAGTTCAGCCAGTGCAC
AAAGAAGGAGCAGTGCAGAGCATATCGGCTGCAGAACGATAAGTG
GGTGTATAATTCTGACAAACTGCCCAAAGCAGCGGGAGCCACCTT
AAAAGGAAAACTGCATGTCCCATTCTTGCTGGCAGACGGCAAATG
CACCGTGCCTCTAGCACCAGAACCTATGATAACCTTCGGTTTCAG
ATCAGTGTCACTGAAACTGCACCCTAAGAATCCCACATATCTAAT
CACCCGCCAACTTGCTGATGAGCCTCACTACACGCACGAGCTCAT
ATCTGAACCAGCTGTTAGGAATTTTACCGTCACCGAAAAAGGGTG
GGAGTTTGTATGGGGAAACCACCCGCCGAAAAGGTTTTGGGCACA
GGAAACAGCACCCGGAAATCCACATGGGCTACCGCACGAGGTGAT
AACTCATTATTACCACAGATACCCTATGTCCACCATCCTGGGTTT
GTCAATTTGTGCCGCCATTGCAACCGTTTCCGTTGCAGCGTCTAC
CTGGCTGTTTTGCAGATCTAGAGTTGCGTGCCTAACTCCTTACCG
GCTAACACCTAACGCTAGGATACCATTTTGTCTGGCTGTGCTTTG
CTGCGCCCGCACTGCCCGGGCCGAGACCACCTGGGAGTCCTTGGA
TCACCTATGGAACAATAACCAACAGATGTTCTGGATTCAATTGCT
GATCCCTCTGGCCGCCTTGATCGTAGTGACTCGCCTGCTCAGGTG
CGTGTGCTGTGTCGTGCCTTTTTTAGTCATGGCCGGCGCCGCAGG
CGCCGGCGCCTACGAGCACGCGACCACGATGCCGAGCCAAGCGGG
AATCTCGTATAACACTATAGTCAACAGAGCAGGCTACGCACCACT
CCCTATCAGCATAACACCAACAAAGATCAAGCTGATACCTACAGT
GAACTTGGAGTACGTCACCTGCCACTACAAAACAGGAATGGATTC
ACCAGCCATCAAATGCTGCGGATCTCAGGAATGCACTCCAACTTA
CAGGCCTGATGAACAGTGCAAAGTCTTCACAGGGGTTTACCCGTT
CATGTGGGGTGGTGCATATTGCTTTTGCGACACTGAGAACACCCA
AGTCAGCAAGGCCTACGTAATGAAATCTGACGACTGCCTTGCGGA
TCATGCTGAAGCATATAAAGCGCACACAGCCTCAGTGCAGGCGTT
CCTCAACATCACAGTGGGAGAACACTCTATTGTGACTACCGTGTA
TGTGAATGGAGAAACTCCTGTGAATTTCAATGGGGTCAAAATAAC
TGCAGGTCCGCTTTCCACAGCTTGGACACCCTTTGATCGCAAAAT
CGTGCAGTATGCCGGGGAGATCTATAATTATGATTTTCCTGAGTA
TGGGGCAGGACAACCAGGAGCATTTGGAGATATACAATCCAGAAC
AGTCTCAAGCTCTGATCTGTATGCCAATACCAACCTAGTGCTGCA
GAGACCCAAAGCAGGAGCGATCCACGTGCCATACACTCAGGCACC
TTCGGGTTTTGAGCAATGGAAGAAAGATAAAGCTCCATCATTGAA
ATTTACCGCCCCTTTCGGATGCGAAATATATACAAACCCCATTCG
CGCCGAAAACTGTGCTGTAGGGTCAATTCCATTAGCCTTTGACAT
TCCCGACGCCTTGTTCACCAGGGTGTCAGAAACACCGACACTTTC
AGCGGCCGAATGCACTCTTAACGAGTGCGTGTATTCTTCCGACTT
TGGTGGGATCGCCACGGTCAAGTACTCGGCCAGCAAGTCAGGCAA
GTGCGCAGTCCATGTGCCATCAGGGACTGCTACCCTAAAAGAAGC
AGCAGTCGAGCTAACCGAGCAAGGGTCGGCGACTATCCATTTCTC
GACCGCAAATATCCACCCGGAGTTCAGGCTCCAAATATGCACATC
ATATGTTACGTGCAAAGGTGATTGTCACCCCCCGAAAGACCATAT
TGTGACACACCCTCAGTATCACGCCCAAACATTTACAGCCGCGGT
GTCAAAAACCGCGTGGACGTGGTTAACATCCCTGCTGGGAGGATC
AGCCGTAATTATTATAATTGGCTTGGTGCTGGCTACTATTGTGGC
CATGTACGTGCTGACCAACCAGAAACATAATTGAATACAGCAGCA
ATTGGCAAGCTGCTTACATAGAACTCGCGGCGATTGGCATGCCGC
CTTAAAATTTTTATTTTATTTTTCTTTTCTTTTCCGAATCGGATT
TTGTTTTTAATATTTC
VEE Delivery Vector (SEQ ID NO: 6); VEE
genome with nucleotides 7544-11175 deleted
[alphavirus structural proteins removed]
ATGggcggcgcatgagagaagcccagaccaattacctacccaaaA
TGGagaaagttcacgttgacatcgaggaagacagcccattcctca
gagctttgcagcggagcttcccgcagtttgaggtagaagccaagc
aggtcactgataatgaccatgctaatgccagagcgttttcgcatc
tggcttcaaaactgatcgaaacggaggtggacccatccgacacga
tccttgacattggaagtgcgcccgcccgcagaatgtattctaagc
acaagtatcattgtatctgtccgatgagatgtgcggaagatccgg
acagattgtataagtatgcaactaagctgaagaaaaactgtaagg
aaataactgataaggaattggacaagaaaatgaaggagctcgccg
ccgtcatgagcgaccctgacctggaaactgagactatgtgcctcc
acgacgacgagtcgtgtcgctacgaagggcaagtcgctgtttacc
aggatgtatacgcggttgacggaccgacaagtctctatcaccaag
ccaataagggagttagagtcgcctactggataggctttgacacca
ccccttttatgtttaagaacttggctggagcatatccatcatact
ctaccaactgggccgacgaaaccgtgttaacggctcgtaacatag
gcctatgcagctctgacgttatggagcggtcacgtagagggatgt
ccattcttagaaagaagtatttgaaaccatccaacaatgttctat
tctctgttggctcgaccatctaccacgagaagagggacttactga
ggagctggcacctgccgtctgtatttcacttacgtggcaagcaaa
attacacatgtcggtgtgagactatagttagttgcgacgggtacg
tcgttaaaagaatagctatcagtccaggcctgtatgggaagcctt
caggctatgctgctacgatgcaccgcgagggattcttgtgctgca
aagtgacagacacattgaacggggagagggtctcttttcccgtgt
gcacgtatgtgccagctacattgtgtgaccaaatgactggcatac
tggcaacagatgtcagtgcggacgacgcgcaaaaactgctggttg
ggctcaaccagcgtatagtcgtcaacggtcgcacccagagaaaca
ccaataccatgaaaaattaccttttgcccgtagtggcccaggcat
ttgctaggtgggcaaaggaatataaggaagatcaagaagatgaaa
ggccactaggactacgagatagacagttagtcatggggtgttgtt
gggcttttagaaggcacaagataacatctatttataagcgcccgg
atacccaaaccatcatcaaagtgaacagcgatttccactcattcg
tgctgcccaggataggcagtaacacattggagatcgggctgagaa
caagaatcaggaaaatgttagaggagcacaaggagccgtcacctc
tcattaccgccgaggacgtacaagaagctaagtgcgcagccgatg
aggctaaggaggtgcgtgaagccgaggagttgcgcgcagctctac
cacctttggcagctgatgttgaggagcccactctggaagccgatg
tcgacttgatgttacaagaggctggggccggctcagtggagacac
ctcgtggcttgataaaggttaccagctacgctggcgaggacaaga
tcggctcttacgctgtgctttctccgcaggctgtactcaagagtg
aaaaattatcttgcatccaccctctcgctgaacaagtcatagtga
taacacactctggccgaaaagggcgttatgccgtggaaccatacc
atggtaaagtagtggtgccagagggacatgcaatacccgtccagg
actttcaagctctgagtgaaagtgccaccattgtgtacaacgaac
gtgagttcgtaaacaggtacctgcaccatattgccacacatggag
gagcgctgaacactgatgaagaatattacaaaactgtcaagccca
gcgagcacgacggcgaatacctgtacgacatcgacaggaaacagt
gcgtcaagaaagaactagtcactgggctagggctcacaggcgagc
tggtggatcctcccttccatgaattcgcctacgagagtctgagaa
cacgaccagccgctccttaccaagtaccaaccataggggtgtatg
gcgtgccaggatcaggcaagtctggcatcattaaaagcgcagtca
ccaaaaaagatctagtggtgagcgccaagaaagaaaactgtgcag
aaattataagggacgtcaagaaaatgaaagggctggacgtcaatg
ccagaactgtggactcagtgctcttgaatggatgcaaacaccccg
tagagaccctgtatattgacgaagcttttgcttgtcatgcaggta
ctctcagagcgctcatagccattataagacctaaaaaggcagtgc
tctgcggggatcccaaacagtgcggtttttttaacatgatgtgcc
tgaaagtgcattttaaccacgagatttgcacacaagtcttccaca
aaagcatctctcgccgttgcactaaatctgtgacttcggtcgtct
caaccttgttttacgacaaaaaaatgagaacgacgaatccgaaag
agactaagattgtgattgacactaccggcagtaccaaacctaagc
aggacgatctcattctcacttgtttcagaggggggtgaagcagtt
gcaaatagattacaaaggcaacgaaataatgacggcagctgcctc
tcaagggctgacccgtaaaggtgtgtatgccgttcggtacaaggt
gaatgaaaatcctctgtacgcacccacctcagaacatgtgaacgt
cctactgacccgcacggaggaccgcatcgtgtggaaaacactagc
cggcgacccatggataaaaacactgactgccaagtaccctgggaa
tttcactgccacgatagaggagtggcaagcagagcatgatgccat
catgaggcacatcttggagagaccggaccctaccgacgtcttcca
gaataaggcaaacgtgtgttgggccaaggctttagtgccggtgct
gaagaccgctggcatagacatgaccactgaacaatggaacactgt
ggattattttgaaacggacaaagctcactcagcagagatagtatt
gaaccaactatgcgtgaggttctttggactcgatctggactccgg
tctattttctgcacccactgttccgttatccattaggaataatca
ctgggataactccccgtcgcctaacatgtacgggctgaataaaga
agtggtccgtcagctctctcgcaggtacccacaactgcctcgggc
agttgccactggaagagtctatgacatgaacactggtacactgcg
caattatgatccgcgcataaacctagtacctgtaaacagaagact
gcctcatgctttagtcctccaccataatgaacacccacagagtga
cttttcttcattcgtcagcaaattgaagggcagaactgtcctggt
ggtcggggaaaagttgtccgtcccaggcaaaatggttgactggtt
gtcagaccggcctgaggctaccttcagagctcggctggatttagg
catcccaggtgatgtgcccaaatatgacataatatttgttaatgt
gaggaccccatataaataccatcactatcagcagtgtgaagacca
tgccattaagcttagcatgttgaccaagaaagcttgtctgcatct
gaatcccggcggaacctgtgtcagcataggttatggttacgctga
cagggccagcgaaagcatcattggtgctatagcgcggcagttcaa
gttttcccgggtatgcaaaccgaaatcctcacttgaagagacgga
agttctgtttgtattcattgggtacgatcgcaaggcccgtacgca
caatccttacaagctttcatcaaccttgaccaacatttatacagg
ttccagactccacgaagccggatgtgcaccctcatatcatgtggt
gcgaggggatattgccacggccaccgaaggagtgattataaatgc
tgctaacagcaaaggacaacctggcggaggggtgtgcggagcgct
gtataagaaattcccggaaagcttcgatttacagccgatcgaagt
aggaaaagcgcgactggtcaaaggtgcagctaaacatatcattca
tgccgtaggaccaaacttcaacaaagtttcggaggttgaaggtga
caaacagttggcagaggcttatgagtccatcgctaagattgtcaa
cgataacaattacaagtcagtagcgattccactgttgtccaccgg
catcttttccgggaacaaagatcgactaacccaatcattgaacca
tttgctgacagctttagacaccactgatgcagatgtagccatata
ctgcagggacaagaaatgggaaatgactctcaaggaagcagtggc
taggagagaagcagtggaggagatatgcatatccgacgactcttc
agtgacagaacctgatgcagagctggtgagggtgcatccgaagag
ttctttggctggaaggaagggctacagcacaagcgatggcaaaac
tttctcatatttggaagggaccaagtttcaccaggcggccaagga
tatagcagaaattaatgccatgtggcccgttgcaacggaggccaa
tgagcaggtatgcatgtatatcctcggagaaagcatgagcagtat
taggtcgaaatgccccgtcgaagagtcggaagcctccacaccacc
tagcacgctgccttgcttgtgcatccatgccatgactccagaaag
agtacagcgcctaaaagcctcacgtccagaacaaattactgtgtg
ctcatcctttccattgccgaagtatagaatcactggtgtgcagaa
gatccaatgctcccagcctatattgttctcaccgaaagtgcctgc
gtatattcatccaaggaagtatctcgtggaaacaccaccggtaga
cgagactccggagccatcggcagagaaccaatccacagaggggac
acctgaacaaccaccacttataaccgaggatgagaccaggactag
aacgcctgagccgatcatcatcgaagaggaagaagaggatagcat
aagtttgctgtcagatggcccgacccaccaggtgctgcaagtcga
ggcagacattcacgggccgccctctgtatctagctcatcctggtc
cattcctcatgcatccgactttgatgtggacagtttatccatact
tgacaccctggagggagctagcgtgaccagcggggcaacgtcagc
cgagactaactcttacttcgcaaagagtatggagtttctggcgcg
accggtgcctgcgcctcgaacagtattcaggaaccctccacatcc
cgctccgcgcacaagaacaccgtcacttgcacccagcagggcctg
ctcgagaaccagcctagtttccaccccgccaggcgtgaatagggt
gatcactagagaggagctcgaggcgcttaccccgtcacgcactcc
tagcaggtcggtctcgagaaccagcctggtctccaacccgccagg
cgtaaatagggtgattacaagagaggagtttgaggcgttcgtagc
acaacaacaatgacggtttgatgcgggtgcatacatcttttcctc
cgacaccggtcaagggcatttacaacaaaaatcagtaaggcaaac
ggtgctatccgaagtggtgttggagaggaccgaattggagatttc
gtatgccccgcgcctcgaccaagaaaaagaagaattactacgcaa
gaaattacagttaaatcccacacctgctaacagaagcagatacca
gtccaggaaggtggagaacatgaaagccataacagctagacgtat
tctgcaaggcctagggcattatttgaaggcagaaggaaaagtgga
gtgctaccgaaccctgcatcctgttcctttgtattcatctagtgt
gaaccgtgccttttcaagccccaaggtcgcagtggaagcctgtaa
cgccatgttgaaagagaactttccgactgtggcttcttactgtat
tattccagagtacgatgcctatttggacatggttgacggagcttc
atgctgcttagacactgccagtttttgccctgcaaagctgcgcag
ctttccaaagaaacactcctatttggaacccacaatacgatcggc
agtgccttcagcgatccagaacacgctccagaacgtcctggcagc
tgccacaaaaagaaattgcaatgtcacgcaaatgagagaattgcc
cgtattggattcggcggcctttaatgtggaatgcttcaagaaata
tgcgtgtaataatgaatattgggaaacgtttaaagaaaaccccat
caggcttactgaagaaaacgtggtaaattacattaccaaattaaa
aggaccaaaagctgctgctctttttgcgaagacacataatttgaa
tatgttgcaggacataccaatggacaggtttgtaatggacttaaa
gagagacgtgaaagtgactccaggaacaaaacatactgaagaacg
gcccaaggtacaggtgatccaggctgccgatccgctagcaacagc
gtatctgtgcggaatccaccgagagctggttaggagattaaatgc
ggtcctgcttccgaacattcatacactgtttgatatgtcggctga
agactttgacgctattatagccgagcacttccagcctggggattg
tgttctggaaactgacatcgcgtcgtttgataaaagtgaggacga
cgccatggctctgaccgcgttaatgattctggaagacttaggtgt
ggacgcagagctgttgacgctgattgaggcggctttcggcgaaat
ttcatcaatacatttgcccactaaaactaaatttaaattcggagc
catgatgaaatctggaatgttcctcacactgtttgtgaacacagt
cattaacattgtaatcgcaagcagagtgttgagagaacggctaac
cggatcaccatgtgcagcattcattggagatgacaatatcgtgaa
aggagtcaaatcggacaaattaatggcagacaggtgcgccacctg
gttgaatatggaagtcaagattatagatgctgtggtgggcgagaa
agcgccttatttctgtggagggtttattttgtgtgactccgtgac
cggcacagcgtgccgtgtggcagaccccctaaaaaggctgtttaa
gcttggcaaacctctggcagcagacgatgaacatgatgatgacag
gagaagggcattgcatgaagagtcaacacgctggaaccgagtggg
tattctttcagagctgtgcaaggcagtagaatcaaggtatgaaac
cgtaggaacttccatcatagttatggccatgactactctagctag
cagtgttaaatcattcagctacctgagaggggcccctataactct
ctacggcTAAcctgaatggactacgacgtatcacgcccaaacatt
tacagccgcggtgtcaaaaaccgcgtggacgtggttaacatccct
gctgggaggatcagccgtaattattataattggcttggtgctggc
tactattgtggccatgtacgtgctgaccaaccagaaacataattg
aatacagcagcaattggcaagctgcttacatagaactcgcggcga
ttggcatgccgccttaaaatttttattttattttttttttctttt
ccgaatcggattttgtttttaatatttcAAAAAAAAAAAAAAAAA
AAAAAAAAAAAAAAAAAAAAAAAAAAAAAAAAAAAAAAAAAAAAA
AAAAAAAAAAAAAAAAAA
TC-83 Delivery Vector(SEQ ID NO: 7); TC-83
genome with nucleotides 7544-11175 deleted
[alphavirus structural proteins removed]
ATAGGCGGCGCATGAGAGAAGCCCAGACCAATTACCTACCCAAAA
TGGAGAAAGTTCACGTTGACATCGAGGAAGACAGCCCATTCCTCA
GAGCTTTGCAGCGGAGCTTCCCGCAGTTTGAGGTAGAAGCCAAGC
AGGTCACTGATAATGACCATGCTAATGCCAGAGCGTTTTCGCATC
TGGCTTCAAAACTGATCGAAACGGAGGTGGACCCATCCGACACGA
TCCTTGACATTGGAAGTGCGCCCGCCCGCAGAATGTATTCTAAGC
ACAAGTATCATTGTATCTGTCCGATGAGATGTGCGGAAGATCCGG
ACAGATTGTATAAGTATGCAACTAAGCTGAAGAAAAACTGTAAGG
AAATAACTGATAAGGAATTGGACAAGAAAATGAAGGAGCTCGCCG
CCGTCATGAGCGACCCTGACCTGGAAACTGAGACTATGTGCCTCC
ACGACGACGAGTCGTGTCGCTACGAAGGGCAAGTCGCTGTTTACC
AGGATGTATACGCGGTTGACGGACCGACAAGTCTCTATCACCAAG
CCAATAAGGGAGTTAGAGTCGCCTACTGGATAGGCTTTGACACCA
CCCCTTTTATGTTTAAGAACTTGGCTGGAGCATATCCATCATACT
CTACCAACTGGGCCGACGAAACCGTGTTAACGGCTCGTAACATAG
GCCTATGCAGCTCTGACGTTATGGAGCGGTCACGTAGAGGGATGT
CCATTCTTAGAAAGAAGTATTTGAAACCATCCAACAATGTTCTAT
TCTCTGTTGGCTCGACCATCTACCACGAGAAGAGGGACTTACTGA
GGAGCTGGCACCTGCCGTCTGTATTTCACTTACGTGGCAAGCAAA
ATTACACATGTCGGTGTGAGACTATAGTTAGTTGCGACGGGTACG
TCGTTAAAAGAATAGCTATCAGTCCAGGCCTGTATGGGAAGCCTT
CAGGCTATGCTGCTACGATGCACCGCGAGGGATTCTTGTGCTGCA
AAGTGACAGACACATTGAACGGGGAGAGGGTCTCTTTTCCCGTGT
GCACGTATGTGCCAGCTACATTGTGTGACCAAATGACTGGCATAC
TGGCAACAGATGTCAGTGCGGACGACGCGCAAAAACTGCTGGTTG
GGCTCAACCAGCGTATAGTCGTCAACGGTCGCACCCAGAGAAACA
CCAATACCATGAAAAATTACCTTTTGCCCGTAGTGGCCCAGGCAT
TTGCTAGGTGGGCAAAGGAATATAAGGAAGATCAAGAAGATGAAA
GGCCACTAGGACTACGAGATAGACAGTTAGTCATGGGGTGTTGTT
GGGCTTTTAGAAGGCACAAGATAACATCTATTTATAAGCGCCCGG
ATACCCAAACCATCATCAAAGTGAACAGCGATTTCCACTCATTCG
TGCTGCCCAGGATAGGCAGTAACACATTGGAGATCGGGCTGAGAA
CAAGAATCAGGAAAATGTTAGAGGAGCACAAGGAGCCGTCACCTC
TCATTACCGCCGAGGACGTACAAGAAGCTAAGTGCGCAGCCGATG
AGGCTAAGGAGGTGCGTGAAGCCGAGGAGTTGCGCGCAGCTCTAC
CACCTTTGGCAGCTGATGTTGAGGAGCCCACTCTGGAAGCCGATG
TCGACTTGATGTTACAAGAGGCTGGGGCCGGCTCAGTGGAGACAC
CTCGTGGCTTGATAAAGGTTACCAGCTACGATGGCGAGGACAAGA
TCGGCTCTTACGCTGTGCTTTCTCCGCAGGCTGTACTCAAGAGTG
AAAAATTATCTTGCATCCACCCTCTCGCTGAACAAGTCATAGTGA
TAACACACTCTGGCCGAAAAGGGCGTTATGCCGTGGAACCATACC
ATGGTAAAGTAGTGGTGCCAGAGGGACATGCAATACCCGTCCAGG
ACTTTCAAGCTCTGAGTGAAAGTGCCACCATTGTGTACAACGAAC
GTGAGTTCGTAAACAGGTACCTGCACCATATTGCCACACATGGAG
GAGCGCTGAACACTGATGAAGAATATTACAAAACTGTCAAGCCCA
GCGAGCACGACGGCGAATACCTGTACGACATCGACAGGAAACAGT
GCGTCAAGAAAGAACTAGTCACTGGGCTAGGGCTCACAGGCGAGC
TGGTGGATCCTCCCTTCCATGAATTCGCCTACGAGAGTCTGAGAA
CACGACCAGCCGCTCCTTACCAAGTACCAACCATAGGGGTGTATG
GCGTGCCAGGATCAGGCAAGTCTGGCATCATTAAAAGCGCAGTCA
CCAAAAAAGATCTAGTGGTGAGCGCCAAGAAAGAAAACTGTGCAG
AAATTATAAGGGACGTCAAGAAAATGAAAGGGCTGGACGTCAATG
CCAGAACTGTGGACTCAGTGCTCTTGAATGGATGCAAACACCCCG
TAGAGACCCTGTATATTGACGAAGCTTTTGCTTGTCATGCAGGTA
CTCTCAGAGCGCTCATAGCCATTATAAGACCTAAAAAGGCAGTGC
TCTGCGGGGATCCCAAACAGTGCGGTTTTTTTAACATGATGTGCC
TGAAAGTGCATTTTAACCACGAGATTTGCACACAAGTCTTCCACA
AAAGCATCTCTCGCCGTTGCACTAAATCTGTGACTTCGGTCGTCT
CAACCTTGTTTTACGACAAAAAAATGAGAACGACGAATCCGAAAG
AGACTAAGATTGTGATTGACACTACCGGCAGTACCAAACCTAAGC
AGGACGATCTCATTCTCACTTGTTTCAGAGGGTGGGTGAAGCAGT
TGCAAATAGATTACAAAGGCAACGAAATAATGACGGCAGCTGCCT
CTCAAGGGCTGACCCGTAAAGGTGTGTATGCCGTTCGGTACAAGG
TGAATGAAAATCCTCTGTACGCACCCACCTCAGAACATGTGAACG
TCCTACTGACCCGCACGGAGGACCGCATCGTGTGGAAAACACTAG
CCGGCGACCCATGGATAAAAACACTGACTGCCAAGTACCCTGGGA
ATTTCACTGCCACGATAGAGGAGTGGCAAGCAGAGCATGATGCCA
TCATGAGGCACATCTTGGAGAGACCGGACCCTACCGACGTCTTCC
AGAATAAGGCAAACGTGTGTTGGGCCAAGGCTTTAGTGCCGGTGC
TGAAGACCGCTGGCATAGACATGACCACTGAACAATGGAACACTG
TGGATTATTTTGAAACGGACAAAGCTCACTCAGCAGAGATAGTAT
TGAACCAACTATGCGTGAGGTTCTTTGGACTCGATCTGGACTCCG
GTCTATTTTCTGCACCCACTGTTCCGTTATCCATTAGGAATAATC
ACTGGGATAACTCCCCGTCGCCTAACATGTACGGGCTGAATAAAG
AAGTGGTCCGTCAGCTCTCTCGCAGGTACCCACAACTGCCTCGGG
CAGTTGCCACTGGAAGAGTCTATGACATGAACACTGGTACACTGC
GCAATTATGATCCGCGCATAAACCTAGTACCTGTAAACAGAAGAC
TGCCTCATGCTTTAGTCCTCCACCATAATGAACACCCACAGAGTG
ACTTTTCTTCATTCGTCAGCAAATTGAAGGGCAGAACTGTCCTGG
TGGTCGGGGAAAAGTTGTCCGTCCCAGGCAAAATGGTTGACTGGT
TGTCAGACCGGCCTGAGGCTACCTTCAGAGCTCGGCTGGATTTAG
GCATCCCAGGTGATGTGCCCAAATATGACATAATATTTGTTAATG
TGAGGACCCCATATAAATACCATCACTATCAGCAGTGTGAAGACC
ATGCCATTAAGCTTAGCATGTTGACCAAGAAAGCTTGTCTGCATC
TGAATCCCGGCGGAACCTGTGTCAGCATAGGTTATGGTTACGCTG
ACAGGGCCAGCGAAAGCATCATTGGTGCTATAGCGCGGCAGTTCA
AGTTTTCCCGGGTATGCAAACCGAAATCCTCACTTGAAGAGACGG
AAGTTCTGTTTGTATTCATTGGGTACGATCGCAAGGCCCGTACGC
ACAATCCTTACAAGCTTTCATCAACCTTGACCAACATTTATACAG
GTTCCAGACTCCACGAAGCCGGATGTGCACCCTCATATCATGTGG
TGCGAGGGGATATTGCCACGGCCACCGAAGGAGTGATTATAAATG
CTGCTAACAGCAAAGGACAACCTGGCGGAGGGGTGTGCGGAGCGC
TGTATAAGAAATTCCCGGAAAGCTTCGATTTACAGCCGATCGAAG
TAGGAAAAGCGCGACTGGTCAAAGGTGCAGCTAAACATATCATTC
ATGCCGTAGGACCAAACTTCAACAAAGTTTCGGAGGTTGAAGGTG
ACAAACAGTTGGCAGAGGCTTATGAGTCCATCGCTAAGATTGTCA
ACGATAACAATTACAAGTCAGTAGCGATTCCACTGTTGTCCACCG
GCATCTTTTCCGGGAACAAAGATCGACTAACCCAATCATTGAACC
ATTTGCTGACAGCTTTAGACACCACTGATGCAGATGTAGCCATAT
ACTGCAGGGACAAGAAATGGGAAATGACTCTCAAGGAAGCAGTGG
CTAGGAGAGAAGCAGTGGAGGAGATATGCATATCCGACGACTCTT
CAGTGACAGAACCTGATGCAGAGCTGGTGAGGGTGCATCCGAAGA
GTTCTTTGGCTGGAAGGAAGGGCTACAGCACAAGCGATGGCAAAA
CTTTCTCATATTTGGAAGGGACCAAGTTTCACCAGGCGGCCAAGG
ATATAGCAGAAATTAATGCCATGTGGCCCGTTGCAACGGAGGCCA
ATGAGCAGGTATGCATGTATATCCTCGGAGAAAGCATGAGCAGTA
TTAGGTCGAAATGCCCCGTCGAAGAGTCGGAAGCCTCCACACCAC
CTAGCACGCTGCCTTGCTTGTGCATCCATGCCATGACTCCAGAAA
GAGTACAGCGCCTAAAAGCCTCACGTCCAGAACAAATTACTGTGT
GCTCATCCTTTCCATTGCCGAAGTATAGAATCACTGGTGTGCAGA
AGATCCAATGCTCCCAGCCTATATTGTTCTCACCGAAAGTGCCTG
CGTATATTCATCCAAGGAAGTATCTCGTGGAAACACCACCGGTAG
ACGAGACTCCGGAGCCATCGGCAGAGAACCAATCCACAGAGGGGA
CACCTGAACAACCACCACTTATAACCGAGGATGAGACCAGGACTA
GAACGCCTGAGCCGATCATCATCGAAGAGGAAGAAGAGGATAGCA
TAAGTTTGCTGTCAGATGGCCCGACCCACCAGGTGCTGCAAGTCG
AGGCAGACATTCACGGGCCGCCCTCTGTATCTAGCTCATCCTGGT
CCATTCCTCATGCATCCGACTTTGATGTGGACAGTTTATCCATAC
TTGACACCCTGGAGGGAGCTAGCGTGACCAGCGGGGCAACGTCAG
CCGAGACTAACTCTTACTTCGCAAAGAGTATGGAGTTTCTGGCGC
GACCGGTGCCTGCGCCTCGAACAGTATTCAGGAACCCTCCACATC
CCGCTCCGCGCACAAGAACACCGTCACTTGCACCCAGCAGGGCCT
GCTCGAGAACCAGCCTAGTTTCCACCCCGCCAGGCGTGAATAGGG
TGATCACTAGAGAGGAGCTCGAGGCGCTTACCCCGTCACGCACTC
CTAGCAGGTCGGTCTCGAGAACCAGCCTGGTCTCCAACCCGCCAG
GCGTAAATAGGGTGATTACAAGAGAGGAGTTTGAGGCGTTCGTAG
CACAACAACAATGACGGTTTGATGCGGGTGCATACATCTTTTCCT
CCGACACCGGTCAAGGGCATTTACAACAAAAATCAGTAAGGCAAA
CGGTGCTATCCGAAGTGGTGTTGGAGAGGACCGAATTGGAGATTT
CGTATGCCCCGCGCCTCGACCAAGAAAAAGAAGAATTACTACGCA
AGAAATTACAGTTAAATCCCACACCTGCTAACAGAAGCAGATACC
AGTCCAGGAAGGTGGAGAACATGAAAGCCATAACAGCTAGACGTA
TTCTGCAAGGCCTAGGGCATTATTTGAAGGCAGAAGGAAAAGTGG
AGTGCTACCGAACCCTGCATCCTGTTCCTTTGTATTCATCTAGTG
TGAACCGTGCCTTTTCAAGCCCCAAGGTCGCAGTGGAAGCCTGTA
ACGCCATGTTGAAAGAGAACTTTCCGACTGTGGCTTCTTACTGTA
TTATTCCAGAGTACGATGCCTATTTGGACATGGTTGACGGAGCTT
CATGCTGCTTAGACACTGCCAGTTTTTGCCCTGCAAAGCTGCGCA
GCTTTCCAAAGAAACACTCCTATTTGGAACCCACAATACGATCGG
CAGTGCCTTCAGCGATCCAGAACACGCTCCAGAACGTCCTGGCAG
CTGCCACAAAAAGAAATTGCAATGTCACGCAAATGAGAGAATTGC
CCGTATTGGATTCGGCGGCCTTTAATGTGGAATGCTTCAAGAAAT
ATGCGTGTAATAATGAATATTGGGAAACGTTTAAAGAAAACCCCA
TCAGGCTTACTGAAGAAAACGTGGTAAATTACATTACCAAATTAA
AAGGACCAAAAGCTGCTGCTCTTTTTGCGAAGACACATAATTTGA
ATATGTTGCAGGACATACCAATGGACAGGTTTGTAATGGACTTAA
AGAGAGACGTGAAAGTGACTCCAGGAACAAAACATACTGAAGAAC
GGCCCAAGGTACAGGTGATCCAGGCTGCCGATCCGCTAGCAACAG
CGTATCTGTGCGGAATCCACCGAGAGCTGGTTAGGAGATTAAATG
CGGTCCTGCTTCCGAACATTCATACACTGTTTGATATGTCGGCTG
AAGACTTTGACGCTATTATAGCCGAGCACTTCCAGCCTGGGGATT
GTGTTCTGGAAACTGACATCGCGTCGTTTGATAAAAGTGAGGACG
ACGCCATGGCTCTGACCGCGTTAATGATTCTGGAAGACTTAGGTG
TGGACGCAGAGCTGTTGACGCTGATTGAGGCGGCTTTCGGCGAAA
TTTCATCAATACATTTGCCCACTAAAACTAAATTTAAATTCGGAG
CCATGATGAAATCTGGAATGTTCCTCACACTGTTTGTGAACACAG
TCATTAACATTGTAATCGCAAGCAGAGTGTTGAGAGAACGGCTAA
CCGGATCACCATGTGCAGCATTCATTGGAGATGACAATATCGTGA
AAGGAGTCAAATCGGACAAATTAATGGCAGACAGGTGCGCCACCT
GGTTGAATATGGAAGTCAAGATTATAGATGCTGTGGTGGGCGAGA
AAGCGCCTTATTTCTGTGGAGGGTTTATTTTGTGTGACTCCGTGA
CCGGCACAGCGTGCCGTGTGGCAGACCCCCTAAAAAGGCTGTTTA
AGCTTGGCAAACCTCTGGCAGCAGACGATGAACATGATGATGACA
GGAGAAGGGCATTGCATGAAGAGTCAACACGCTGGAACCGAGTGG
GTATTCTTTCAGAGCTGTGCAAGGCAGTAGAATCAAGGTATGAAA
CCGTAGGAACTTCCATCATAGTTATGGCCATGACTACTCTAGCTA
GCAGTGTTAAATCATTCAGCTACCTGAGAGGGGCCCCTATAACTC
TCTACGGCTAACCTGAATGGACTACGACATAGTCTAGTCCGCCAA
GATGTTCCCGTTCCAGCCAATGTATCCGATGCAGCCAATGCCCTA
TCGCAACCCGTTCGCGGCCCCGCGCAGGCCCTGGTTCCCCAGAAC
CGACCCTTTTCTGGCGATGCAGGTGCAGGAATTAACCCGCTCGAT
GGCTAACCTGACGTTCAAGCAACGCCGGGACGCGCCACCTGAGGG
GCCATCCGCTAAGAAACCGAAGAAGGAGGCCTCGCAAAAACAGAA
AGGGGGAGGCCAAGGGAAGAAGAAGAAGAACCAAGGGAAGAAGAA
GGCTAAGACAGGGCCGCCTAATCCGAAGGCACAGAATGGAAACAA
GAAGAAGACCAACAAGAAACCAGGCAAGAGACAGCGCATGGTCAT
GAAATTGGAATCTGACAAGACGTTCCCAATCATGTTGGAAGGGAA
GATAAACGGCTACGCTTGTGTGGTCGGAGGGAAGTTATTCAGGCC
GATGCATGTGGAAGGCAAGATCGACAACGACGTTCTGGCCGCGCT
TAAGACGAAGAAAGCATCCAAATACGATCTTGAGTATGCAGATGT
GCCACAGAACATGCGGGCCGATACATTCAAATACACCCATGAGAA
ACCCCAAGGCTATTACAGCTGGCATCATGGAGCAGTCCAATATGA
AAATGGGCGTTTCACGGTGCCGAAAGGAGTTGGGGCCAAGGGAGA
CAGCGGACGACCCATTCTGGATAACCAGGGACGGGTGGTCGCTAT
TGTGCTGGGAGGTGTGAATGAAGGATCTAGGACAGCCCTTTCAGT
CGTCATGTGGAACGAGAAGGGAGTTACCGTGAAGTATACTCCGGA
GAACTGCGAGCAATGGTCACTAGTGACCACCATGTGTCTGCTCGC
CAATGTGACGTTCCCATGTGCTCAACCACCAATTTGCTACGACAG
AAAACCAGCAGAGACTTTGGCCATGCTCAGCGTTAACGTTGACAA
CCCGGGCTACGATGAGCTGCTGGAAGCAGCTGTTAAGTGCCCCGG
AAGGAAAAGGAGATCCACCGAGGAGCTGTTTAATGAGTATAAGCT
AACGCGCCCTTACATGGCCAGATGCATCAGATGTGCAGTTGGGAG
CTGCCATAGTCCAATAGCAATCGAGGCAGTAAAGAGCGACGGGCA
CGACGGTTATGTTAGACTTCAGACTTCCTCGCAGTATGGCCTGGA
TTCCTCCGGCAACTTAAAGGGCAGGACCATGCGGTATGACATGCA
CGGGACCATTAAAGAGATACCACTACATCAAGTGTCACTCTATAC
ATCTCGCCCGTGTCACATTGTGGATGGGCACGGTTATTTCCTGCT
TGCCAGGTGCCCGGCAGGGGACTCCATCACCATGGAATTTAAGAA
AGATTCCGTCAGACACTCCTGCTCGGTGCCGTATGAAGTGAAATT
TAATCCTGTAGGCAGAGAACTCTATACTCATCCCCCAGAACACGG
AGTAGAGCAAGCGTGCCAAGTCTACGCACATGATGCACAGAACAG
AGGAGCTTATGTCGAGATGCACCTCCCGGGCTCAGAAGTGGACAG
CAGTTTGGTTTCCTTGAGCGGCAGTTCAGTCACCGTGACACCTCC
TGATGGGACTAGCGCCCTGGTGGAATGCGAGTGTGGCGGCACAAA
GATCTCCGAGACCATCAACAAGACAAAACAGTTCAGCCAGTGCAC
AAAGAAGGAGCAGTGCAGAGCATATCGGCTGCAGAACGATAAGTG
GGTGTATAATTCTGACAAACTGCCCAAAGCAGCGGGAGCCACCTT
AAAAGGAAAACTGCATGTCCCATTCTTGCTGGCAGACGGCAAATG
CACCGTGCCTCTAGCACCAGAACCTATGATAACCTTCGGTTTCAG
ATCAGTGTCACTGAAACTGCACCCTAAGAATCCCACATATCTAAT
CACCCGCCAACTTGCTGATGAGCCTCACTACACGCACGAGCTCAT
ATCTGAACCAGCTGTTAGGAATTTTACCGTCACCGAAAAAGGGTG
GGAGTTTGTATGGGGAAACCACCCGCCGAAAAGGTTTTGGGCACA
GGAAACAGCACCCGGAAATCCACATGGGCTACCGCACGAGGTGAT
AACTCATTATTACCACAGATACCCTATGTCCACCATCCTGGGTTT
GTCAATTTGTGCCGCCATTGCAACCGTTTCCGTTGCAGCGTCTAC
CTGGCTGTTTTGCAGATCTAGAGTTGCGTGCCTAACTCCTTACCG
GCTAACACCTAACGCTAGGATACCATTTTGTCTGGCTGTGCTTTG
CTGCGCCCGCACTGCCCGGGCCGAGACCACCTGGGAGTCCTTGGA
TCACCTATGGAACAATAACCAACAGATGTTCTGGATTCAATTGCT
GATCCCTCTGGCCGCCTTGATCGTAGTGACTCGCCTGCTCAGGTG
CGTGTGCTGTGTCGTGCCTTTTTTAGTCATGGCCGGCGCCGCAGG
CGCCGGCGCCTACGAGCACGCGACCACGATGCCGAGCCAAGCGGG
AATCTCGTATAACACTATAGTCAACAGAGCAGGCTACGCACCACT
CCCTATCAGCATAACACCAACAAAGATCAAGCTGATACCTACAGT
GAACTTGGAGTACGTCACCTGCCACTACAAAACAGGAATGGATTC
ACCAGCCATCAAATGCTGCGGATCTCAGGAATGCACTCCAACTTA
CAGGCCTGATGAACAGTGCAAAGTCTTCACAGGGGTTTACCCGTT
CATGTGGGGTGGTGCATATTGCTTTTGCGACACTGAGAACACCCA
AGTCAGCAAGGCCTACGTAATGAAATCTGACGACTGCCTTGCGGA
TCATGCTGAAGCATATAAAGCGCACACAGCCTCAGTGCAGGCGTT
CCTCAACATCACAGTGGGAGAACACTCTATTGTGACTACCGTGTA
TGTGAATGGAGAAACTCCTGTGAATTTCAATGGGGTCAAAATAAC
TGCAGGTCCGCTTTCCACAGCTTGGACACCCTTTGATCGCAAAAT
CGTGCAGTATGCCGGGGAGATCTATAATTATGATTTTCCTGAGTA
TGGGGCAGGACAACCAGGAGCATTTGGAGATATACAATCCAGAAC
AGTCTCAAGCTCTGATCTGTATGCCAATACCAACCTAGTGCTGCA
GAGACCCAAAGCAGGAGCGATCCACGTGCCATACACTCAGGCACC
TTCGGGTTTTGAGCAATGGAAGAAAGATAAAGCTCCATCATTGAA
ATTTACCGCCCCTTTCGGATGCGAAATATATACAAACCCCATTCG
CGCCGAAAACTGTGCTGTAGGGTCAATTCCATTAGCCTTTGACAT
TCCCGACGCCTTGTTCACCAGGGTGTCAGAAACACCGACACTTTC
AGCGGCCGAATGCACTCTTAACGAGTGCGTGTATTCTTCCGACTT
TGGTGGGATCGCCACGGTCAAGTACTCGGCCAGCAAGTCAGGCAA
GTGCGCAGTCCATGTGCCATCAGGGACTGCTACCCTAAAAGAAGC
AGCAGTCGAGCTAACCGAGCAAGGGTCGGCGACTATCCATTTCTC
GACCGCAAATATCCACCCGGAGTTCAGGCTCCAAATATGCACATC
ATATGTTACGTGCAAAGGTGATTGTCACCCCCCGAAAGACCATAT
TGTGACACACCCTCAGTATCACGCCCAAACATTTACAGCCGCGGT
GTCAAAAACCGCGTGGACGTGGTTAACATCCCTGCTGGGAGGATC
AGCCGTAATTATTATAATTGGCTTGGTGCTGGCTACTATTGTGGC
CATGTACGTGCTGACCAACCAGAAACATAATTGAATACAGCAGCA
ATTGGCAAGCTGCTTACATAGAACTCGCGGCGATTGGCATGCCGC
CTTAAAATTTTTATTTTATTTTTCTTTTCTTTTCCGAATCGGATT
TTGTTTTTAATATTTC
VEE Production Vector (SEQ ID NO: 8); VEE
genome with nucleotides 7544-11175 deleted,
plus 5′ T7-promoter, plus 3′ restriction
sites
TAATACGACTCACTATAGGATGggcggcgcatgagagaagcccag
accaattacctacccaaaATGGagaaagttcacgttgacatcgag
gaagacagcccattcctcagagctttgcagcggagcttcccgcag
tttgaggtagaagccaagcaggtcactgataatgaccatgctaat
gccagagcgttttcgcatctggcttcaaaactgatcgaaacggag
gtggacccatccgacacgatccttgacattggaagtgcgcccgcc
cgcagaatgtattctaagcacaagtatcattgtatctgtccgatg
agatgtgcggaagatccggacagattgtataagtatgcaactaag
ctgaagaaaaactgtaaggaaataactgataaggaattggacaag
aaaatgaaggagctcgccgccgtcatgagcgaccctgacctggaa
actgagactatgtgcctccacgacgacgagtcgtgtcgctacgaa
gggcaagtcgctgtttaccaggatgtatacgcggttgacggaccg
acaagtctctatcaccaagccaataagggagttagagtcgcctac
tggataggctttgacaccaccccttttatgtttaagaacttggct
ggagcatatccatcatactctaccaactgggccgacgaaaccgtg
ttaacggctcgtaacataggcctatgcagctctgacgttatggag
cggtcacgtagagggatgtccattcttagaaagaagtatttgaaa
ccatccaacaatgttctattctctgttggctcgaccatctaccac
gagaagagggacttactgaggagctggcacctgccgtctgtattt
cacttacgtggcaagcaaaattacacatgtcggtgtgagactata
gttagttgcgacgggtacgtcgttaaaagaatagctatcagtcca
ggcctgtatgggaagccttcaggctatgctgctacgatgcaccgc
gagggattcttgtgctgcaaagtgacagacacattgaacggggag
agggtctcttttcccgtgtgcacgtatgtgccagctacattgtgt
gaccaaatgactggcatactggcaacagatgtcagtgcggacgac
gcgcaaaaactgctggttgggctcaaccagcgtatagtcgtcaac
ggtcgcacccagagaaacaccaataccatgaaaaattaccttttg
cccgtagtggcccaggcatttgctaggtgggcaaaggaatataag
gaagatcaagaagatgaaaggccactaggactacgagatagacag
ttagtcatggggtgttgttgggcttttagaaggcacaagataaca
tctatttataagcgcccggatacccaaaccatcatcaaagtgaac
agcgatttccactcattcgtgctgcccaggataggcagtaacaca
ttggagatcgggctgagaacaagaatcaggaaaatgttagaggag
cacaaggagccgtcacctctcattaccgccgaggacgtacaagaa
gctaagtgcgcagccgatgaggctaaggaggtgcgtgaagccgag
gagttgcgcgcagctctaccacctttggcagctgatgttgaggag
cccactctggaagccgatgtcgacttgatgttacaagaggctggg
gccggctcagtggagacacctcgtggcttgataaaggttaccagc
tacgctggcgaggacaagatcggctcttacgctgtgctttctccg
caggctgtactcaagagtgaaaaattatcttgcatccaccctctc
gctgaacaagtcatagtgataacacactctggccgaaaagggcgt
tatgccgtggaaccataccatggtaaagtagtggtgccagaggga
catgcaatacccgtccaggactttcaagctctgagtgaaagtgcc
accattgtgtacaacgaacgtgagttcgtaaacaggtacctgcac
catattgccacacatggaggagcgctgaacactgatgaagaatat
tacaaaactgtcaagcccagcgagcacgacggcgaatacctgtac
gacatcgacaggaaacagtgcgtcaagaaagaactagtcactggg
ctagggctcacaggcgagctggtggatcctcccttccatgaattc
gcctacgagagtctgagaacacgaccagccgctccttaccaagta
ccaaccataggggtgtatggcgtgccaggatcaggcaagtctggc
atcattaaaagcgcagtcaccaaaaaagatctagtggtgagcgcc
aagaaagaaaactgtgcagaaattataagggacgtcaagaaaatg
aaagggctggacgtcaatgccagaactgtggactcagtgctcttg
aatggatgcaaacaccccgtagagaccctgtatattgacgaagct
tttgcttgtcatgcaggtactctcagagcgctcatagccattata
agacctaaaaaggcagtgctctgcggggatcccaaacagtgcggt
ttttttaacatgatgtgcctgaaagtgcattttaaccacgagatt
tgcacacaagtcttccacaaaagcatctctcgccgttgcactaaa
tctgtgacttcggtcgtctcaaccttgttttacgacaaaaaaatg
agaacgacgaatccgaaagagactaagattgtgattgacactacc
ggcagtaccaaacctaagcaggacgatctcattctcacttgtttc
agaggggggtgaagcagttgcaaatagattacaaaggcaacgaaa
taatgacggcagctgcctctcaagggctgacccgtaaaggtgtgt
atgccgttcggtacaaggtgaatgaaaatcctctgtacgcaccca
cctcagaacatgtgaacgtcctactgacccgcacggaggaccgca
tcgtgtggaaaacactagccggcgacccatggataaaaacactga
ctgccaagtaccctgggaatttcactgccacgatagaggagtggc
aagcagagcatgatgccatcatgaggcacatcttggagagaccgg
accctaccgacgtcttccagaataaggcaaacgtgtgttgggcca
aggctttagtgccggtgctgaagaccgctggcatagacatgacca
ctgaacaatggaacactgtggattattttgaaacggacaaagctc
actcagcagagatagtattgaaccaactatgcgtgaggttctttg
gactcgatctggactccggtctattttctgcacccactgttccgt
tatccattaggaataatcactgggataactccccgtcgcctaaca
tgtacgggctgaataaagaagtggtccgtcagctctctcgcaggt
acccacaactgcctcgggcagttgccactggaagagtctatgaca
tgaacactggtacactgcgcaattatgatccgcgcataaacctag
tacctgtaaacagaagactgcctcatgctttagtcctccaccata
atgaacacccacagagtgacttttcttcattcgtcagcaaattga
agggcagaactgtcctggtggtcggggaaaagttgtccgtcccag
gcaaaatggttgactggttgtcagaccggcctgaggctaccttca
gagctcggctggatttaggcatcccaggtgatgtgcccaaatatg
acataatatttgttaatgtgaggaccccatataaataccatcact
atcagcagtgtgaagaccatgccattaagcttagcatgttgacca
agaaagcttgtctgcatctgaatcccggcggaacctgtgtcagca
taggttatggttacgctgacagggccagcgaaagcatcattggtg
ctatagcgcggcagttcaagttttcccgggtatgcaaaccgaaat
cctcacttgaagagacggaagttctgtttgtattcattgggtacg
atcgcaaggcccgtacgcacaatccttacaagctttcatcaacct
tgaccaacatttatacaggttccagactccacgaagccggatgtg
caccctcatatcatgtggtgcgaggggatattgccacggccaccg
aaggagtgattataaatgctgctaacagcaaaggacaacctggcg
gaggggtgtgcggagcgctgtataagaaattcccggaaagcttcg
atttacagccgatcgaagtaggaaaagcgcgactggtcaaaggtg
cagctaaacatatcattcatgccgtaggaccaaacttcaacaaag
tttcggaggttgaag gtgacaaacagttggcagaggcttatgag
tccatcgctaagattgtcaacgataacaattacaagtcagtagcg
attccactgttgtccaccggcatcttttccgggaacaaagatcga
ctaacccaatcattgaaccatttgctgacagctttagacaccact
gatgcagatgtagccatatactgcagggacaagaaatgggaaatg
actctcaaggaagcagtggctaggagagaagcagtggaggagata
tgcatatccgacgactcttcagtgacagaacctgatgcagagctg
gtgagggtgcatccgaagagttctttggctggaaggaagggctac
agcacaagcgatggcaaaactttctcatatttggaagggaccaag
tttcaccaggggccaaggatatagcagaaattaatgccatgtggc
ccgttgcaacggaggccaatgagcaggtatgcatgtatatcctcg
gagaaagcatgagcagtattaggtcgaaatgccccgtcgaagagt
cggaagcctccacaccacctagcacgctgccttgcttgtgcatcc
atgccatgactccagaaagagtacagcgcctaaaagcctcacgtc
cagaacaaattactgtgtgctcatcctttccattgccgaagtata
gaatcactggtgtgcagaagatccaatgctcccagcctatattgt
tctcaccgaaagtgcctgcgtatattcatccaaggaagtatctcg
tggaaacaccaccggtagacgagactccggagccatcggcagaga
accaatccacagaggggacacctgaacaaccaccacttataaccg
aggatgagaccaggactagaacgcctgagccgatcatcatcgaag
aggaagaagaggatagcataagtttgctgtcagatggcccgaccc
accaggtgctgcaagtcgaggcagacattcacgggccgccctctg
tatctagctcatcctggtccattcctcatgcatccgactttgatg
tggacagtttatccatacttgacaccctggagggagctagcgtga
ccagcggggcaacgtcagccgagactaactcttacttcgcaaaga
gtatggagtttctggcgcgaccggtgcctgcgcctcgaacagtat
tcaggaaccctccacatcccgctccgcgcacaagaacaccgtcac
ttgcacccagcagggcctgctcgagaaccagcctagtttccaccc
cgccaggcgtgaatagggtgatcactagagaggagctcgaggcgc
ttaccccgtcacgcactcctagcaggtcggtctcgagaaccagcc
tggtctccaacccgccaggcgtaaatagggtgattacaagagagg
agtttgaggcgttcgtagcacaacaacaatgacggtttgatgcgg
gtgcatacatcttttcctccgacaccggtcaagggcatttacaac
aaaaatcagtaaggcaaacggtgctatccgaagtggtgttggaga
ggaccgaattggagatttcgtatgccccgcgcctcgaccaagaaa
aagaagaattactacgcaagaaattacagttaaatcccacacctg
ctaacagaagcagataccagtccaggaaggtggagaacatgaaag
ccataacagctagacgtattctgcaaggcctagggcattatttga
aggcagaaggaaaagtggagtgctaccgaaccctgcatcctgttc
ctttgtattcatctagtgtgaaccgtgccttttcaagccccaagg
tcgcagtggaagcctgtaacgccatgttgaaagagaactttccga
ctgtggcttcttactgtattattccagagtacgatgcctatttgg
acatggttgacggagcttcatgctgcttagacactgccagttttt
gccctgcaaagctgcgcagctttccaaagaaacactcctatttgg
aacccacaatacgatcggcagtgccttcagcgatccagaacacgc
tccagaacgtcctggcagctgccacaaaaagaaattgcaatgtca
cgcaaatgagagaattgcccgtattggattcggcggcctttaatg
tggaatgcttcaagaaatatgcgtgtaataatgaatattgggaaa
cgtttaaagaaaaccccatcaggcttactgaagaaaacgtggtaa
attacattaccaaattaaaaggaccaaaagctgctgctctttttg
cgaagacacataatttgaatatgttgcaggacataccaatggaca
ggtttgtaatggacttaaagagagacgtgaaagtgactccaggaa
caaaacatactgaagaacggcccaaggtacaggtgatccaggctg
ccgatccgctagcaacagcgtatctgtgcggaatccaccgagagc
tggttaggagattaaatgcggtcctgcttccgaacattcatacac
tgtttgatatgtcggctgaagactttgacgctattatagccgagc
acttccagcctggggattgtgttctggaaactgacatcgcgtcgt
ttgataaaagtgaggacgacgccatggctctgaccgcgttaatga
ttctggaagacttaggtgtggacgcagagctgttgacgctgattg
aggcggctttcggcgaaatttcatcaatacatttgcccactaaaa
ctaaatttaaattcggagccatgatgaaatctggaatgttcctca
cactgtttgtgaacacagtcattaacattgtaatcgcaagcagag
tgttgagagaacggctaaccggatcaccatgtgcagcattcattg
gagatgacaatatcgtgaaaggagtcaaatcggacaaattaatgg
cagacaggtgcgccacctggttgaatatggaagtcaagattatag
atgctgtggtgggcgagaaagcgccttatttctgtggagggttta
ttttgtgtgactccgtgaccggcacagcgtgccgtgtggcagacc
ccctaaaaaggctgtttaagcttggcaaacctctggcagcagacg
atgaacatgatgatgacaggagaagggcattgcatgaagagtcaa
cacgctggaaccgagtgggtattctttcagagctgtgcaaggcag
tagaatcaaggtatgaaaccgtaggaacttccatcatagttatgg
ccatgactactctagctagcagtgttaaatcattcagctacctga
gaggggcccctataactctctacggcTAAcctgaatggactacga
cgtatcacgcccaaacatttacagccgcggtgtcaaaaaccgcgt
ggacgtggttaacatccctgctgggaggatcagccgtaattatta
taattggcttggtgctggctactattgtggccatgtacgtgctga
ccaaccagaaacataattgaatacagcagcaattggcaagctgct
tacatagaactcgcggcgattggcatgccgccttaaaatttttat
tttattttttcttttcttttccgaatcggattttgtttttaatat
ttcAAAAAAAAAAAAAAAAAAAAAAAAAAAAAAAAAAAAAAAAAA
AAAAAAAAAAAAAAAAAAAAAAAAAAAAAAAAAAAAAA
TC-83 Production Vector(SEQ ID NO: 9);
TC-83 genome with nucleotides 7544-11175
deleted, plus 5′ T7-promoter,
plus 3′ restriction sites
ATAGGCGGCGCATGAGAGAAGCCCAGACCAATTACCTACCCAAAA
TGGAGAAAGTTCACGTTGACATCGAGGAAGACAGCCCATTCCTCA
GAGCTTTGCAGCGGAGCTTCCCGCAGTTTGAGGTAGAAGCCAAGC
AGGTCACTGATAATGACCATGCTAATGCCAGAGCGTTTTCGCATC
TGGCTTCAAAACTGATCGAAACGGAGGTGGACCCATCCGACACGA
TCCTTGACATTGGAAGTGCGCCCGCCCGCAGAATGTATTCTAAGC
ACAAGTATCATTGTATCTGTCCGATGAGATGTGCGGAAGATCCGG
ACAGATTGTATAAGTATGCAACTAAGCTGAAGAAAAACTGTAAGG
AAATAACTGATAAGGAATTGGACAAGAAAATGAAGGAGCTCGCCG
CCGTCATGAGCGACCCTGACCTGGAAACTGAGACTATGTGCCTCC
ACGACGACGAGTCGTGTCGCTACGAAGGGCAAGTCGCTGTTTACC
AGGATGTATACGCGGTTGACGGACCGACAAGTCTCTATCACCAAG
CCAATAAGGGAGTTAGAGTCGCCTACTGGATAGGCTTTGACACCA
CCCCTTTTATGTTTAAGAACTTGGCTGGAGCATATCCATCATACT
CTACCAACTGGGCCGACGAAACCGTGTTAACGGCTCGTAACATAG
GCCTATGCAGCTCTGACGTTATGGAGCGGTCACGTAGAGGGATGT
CCATTCTTAGAAAGAAGTATTTGAAACCATCCAACAATGTTCTAT
TCTCTGTTGGCTCGACCATCTACCACGAGAAGAGGGACTTACTGA
GGAGCTGGCACCTGCCGTCTGTATTTCACTTACGTGGCAAGCAAA
ATTACACATGTCGGTGTGAGACTATAGTTAGTTGCGACGGGTACG
TCGTTAAAAGAATAGCTATCAGTCCAGGCCTGTATGGGAAGCCTT
CAGGCTATGCTGCTACGATGCACCGCGAGGGATTCTTGTGCTGCA
AAGTGACAGACACATTGAACGGGGAGAGGGTCTCTTTTCCCGTGT
GCACGTATGTGCCAGCTACATTGTGTGACCAAATGACTGGCATAC
TGGCAACAGATGTCAGTGCGGACGACGCGCAAAAACTGCTGGTTG
GGCTCAACCAGCGTATAGTCGTCAACGGTCGCACCCAGAGAAACA
CCAATACCATGAAAAATTACCTTTTGCCCGTAGTGGCCCAGGCAT
TTGCTAGGTGGGCAAAGGAATATAAGGAAGATCAAGAAGATGAAA
GGCCACTAGGACTACGAGATAGACAGTTAGTCATGGGGTGTTGTT
GGGCTTTTAGAAGGCACAAGATAACATCTATTTATAAGCGCCCGG
ATACCCAAACCATCATCAAAGTGAACAGCGATTTCCACTCATTCG
TGCTGCCCAGGATAGGCAGTAACACATTGGAGATCGGGCTGAGAA
CAAGAATCAGGAAAATGTTAGAGGAGCACAAGGAGCCGTCACCTC
TCATTACCGCCGAGGACGTACAAGAAGCTAAGTGCGCAGCCGATG
AGGCTAAGGAGGTGCGTGAAGCCGAGGAGTTGCGCGCAGCTCTAC
CACCTTTGGCAGCTGATGTTGAGGAGCCCACTCTGGAAGCCGATG
TCGACTTGATGTTACAAGAGGCTGGGGCCGGCTCAGTGGAGACAC
CTCGTGGCTTGATAAAGGTTACCAGCTACGATGGCGAGGACAAGA
TCGGCTCTTACGCTGTGCTTTCTCCGCAGGCTGTACTCAAGAGTG
AAAAATTATCTTGCATCCACCCTCTCGCTGAACAAGTCATAGTGA
TAACACACTCTGGCCGAAAAGGGCGTTATGCCGTGGAACCATACC
ATGGTAAAGTAGTGGTGCCAGAGGGACATGCAATACCCGTCCAGG
ACTTTCAAGCTCTGAGTGAAAGTGCCACCATTGTGTACAACGAAC
GTGAGTTCGTAAACAGGTACCTGCACCATATTGCCACACATGGAG
GAGCGCTGAACACTGATGAAGAATATTACAAAACTGTCAAGCCCA
GCGAGCACGACGGCGAATACCTGTACGACATCGACAGGAAACAGT
GCGTCAAGAAAGAACTAGTCACTGGGCTAGGGCTCACAGGCGAGC
TGGTGGATCCTCCCTTCCATGAATTCGCCTACGAGAGTCTGAGAA
CACGACCAGCCGCTCCTTACCAAGTACCAACCATAGGGGTGTATG
GCGTGCCAGGATCAGGCAAGTCTGGCATCATTAAAAGCGCAGTCA
CCAAAAAAGATCTAGTGGTGAGCGCCAAGAAAGAAAACTGTGCAG
AAATTATAAGGGACGTCAAGAAAATGAAAGGGCTGGACGTCAATG
CCAGAACTGTGGACTCAGTGCTCTTGAATGGATGCAAACACCCCG
TAGAGACCCTGTATATTGACGAAGCTTTTGCTTGTCATGCAGGTA
CTCTCAGAGCGCTCATAGCCATTATAAGACCTAAAAAGGCAGTGC
TCTGCGGGGATCCCAAACAGTGCGGTTTTTTTAACATGATGTGCC
TGAAAGTGCATTTTAACCACGAGATTTGCACACAAGTCTTCCACA
AAAGCATCTCTCGCCGTTGCACTAAATCTGTGACTTCGGTCGTCT
CAACCTTGTTTTACGACAAAAAAATGAGAACGACGAATCCGAAAG
AGACTAAGATTGTGATTGACACTACCGGCAGTACCAAACCTAAGC
AGGACGATCTCATTCTCACTTGTTTCAGAGGGTGGGTGAAGCAGT
TGCAAATAGATTACAAAGGCAACGAAATAATGACGGCAGCTGCCT
CTCAAGGGCTGACCCGTAAAGGTGTGTATGCCGTTCGGTACAAGG
TGAATGAAAATCCTCTGTACGCACCCACCTCAGAACATGTGAACG
TCCTACTGACCCGCACGGAGGACCGCATCGTGTGGAAAACACTAG
CCGGCGACCCATGGATAAAAACACTGACTGCCAAGTACCCTGGGA
ATTTCACTGCCACGATAGAGGAGTGGCAAGCAGAGCATGATGCCA
TCATGAGGCACATCTTGGAGAGACCGGACCCTACCGACGTCTTCC
AGAATAAGGCAAACGTGTGTTGGGCCAAGGCTTTAGTGCCGGTGC
TGAAGACCGCTGGCATAGACATGACCACTGAACAATGGAACACTG
TGGATTATTTTGAAACGGACAAAGCTCACTCAGCAGAGATAGTAT
TGAACCAACTATGCGTGAGGTTCTTTGGACTCGATCTGGACTCCG
GTCTATTTTCTGCACCCACTGTTCCGTTATCCATTAGGAATAATC
ACTGGGATAACTCCCCGTCGCCTAACATGTACGGGCTGAATAAAG
AAGTGGTCCGTCAGCTCTCTCGCAGGTACCCACAACTGCCTCGGG
CAGTTGCCACTGGAAGAGTCTATGACATGAACACTGGTACACTGC
GCAATTATGATCCGCGCATAAACCTAGTACCTGTAAACAGAAGAC
TGCCTCATGCTTTAGTCCTCCACCATAATGAACACCCACAGAGTG
ACTTTTCTTCATTCGTCAGCAAATTGAAGGGCAGAACTGTCCTGG
TGGTCGGGGAAAAGTTGTCCGTCCCAGGCAAAATGGTTGACTGGT
TGTCAGACCGGCCTGAGGCTACCTTCAGAGCTCGGCTGGATTTAG
GCATCCCAGGTGATGTGCCCAAATATGACATAATATTTGTTAATG
TGAGGACCCCATATAAATACCATCACTATCAGCAGTGTGAAGACC
ATGCCATTAAGCTTAGCATGTTGACCAAGAAAGCTTGTCTGCATC
TGAATCCCGGCGGAACCTGTGTCAGCATAGGTTATGGTTACGCTG
ACAGGGCCAGCGAAAGCATCATTGGTGCTATAGCGCGGCAGTTCA
AGTTTTCCCGGGTATGCAAACCGAAATCCTCACTTGAAGAGACGG
AAGTTCTGTTTGTATTCATTGGGTACGATCGCAAGGCCCGTACGC
ACAATCCTTACAAGCTTTCATCAACCTTGACCAACATTTATACAG
GTTCCAGACTCCACGAAGCCGGATGTGCACCCTCATATCATGTGG
TGCGAGGGGATATTGCCACGGCCACCGAAGGAGTGATTATAAATG
CTGCTAACAGCAAAGGACAACCTGGCGGAGGGGTGTGCGGAGCGC
TGTATAAGAAATTCCCGGAAAGCTTCGATTTACAGCCGATCGAAG
TAGGAAAAGCGCGACTGGTCAAAGGTGCAGCTAAACATATCATTC
ATGCCGTAGGACCAAACTTCAACAAAGTTTCGGAGGTTGAAGGTG
ACAAACAGTTGGCAGAGGCTTATGAGTCCATCGCTAAGATTGTCA
ACGATAACAATTACAAGTCAGTAGCGATTCCACTGTTGTCCACCG
GCATCTTTTCCGGGAACAAAGATCGACTAACCCAATCATTGAACC
ATTTGCTGACAGCTTTAGACACCACTGATGCAGATGTAGCCATAT
ACTGCAGGGACAAGAAATGGGAAATGACTCTCAAGGAAGCAGTGG
CTAGGAGAGAAGCAGTGGAGGAGATATGCATATCCGACGACTCTT
CAGTGACAGAACCTGATGCAGAGCTGGTGAGGGTGCATCCGAAGA
GTTCTTTGGCTGGAAGGAAGGGCTACAGCACAAGCGATGGCAAAA
CTTTCTCATATTTGGAAGGGACCAAGTTTCACCAGGCGGCCAAGG
ATATAGCAGAAATTAATGCCATGTGGCCCGTTGCAACGGAGGCCA
ATGAGCAGGTATGCATGTATATCCTCGGAGAAAGCATGAGCAGTA
TTAGGTCGAAATGCCCCGTCGAAGAGTCGGAAGCCTCCACACCAC
CTAGCACGCTGCCTTGCTTGTGCATCCATGCCATGACTCCAGAAA
GAGTACAGCGCCTAAAAGCCTCACGTCCAGAACAAATTACTGTGT
GCTCATCCTTTCCATTGCCGAAGTATAGAATCACTGGTGTGCAGA
AGATCCAATGCTCCCAGCCTATATTGTTCTCACCGAAAGTGCCTG
CGTATATTCATCCAAGGAAGTATCTCGTGGAAACACCACCGGTAG
ACGAGACTCCGGAGCCATCGGCAGAGAACCAATCCACAGAGGGGA
CACCTGAACAACCACCACTTATAACCGAGGATGAGACCAGGACTA
GAACGCCTGAGCCGATCATCATCGAAGAGGAAGAAGAGGATAGCA
TAAGTTTGCTGTCAGATGGCCCGACCCACCAGGTGCTGCAAGTCG
AGGCAGACATTCACGGGCCGCCCTCTGTATCTAGCTCATCCTGGT
CCATTCCTCATGCATCCGACTTTGATGTGGACAGTTTATCCATAC
TTGACACCCTGGAGGGAGCTAGCGTGACCAGCGGGGCAACGTCAG
CCGAGACTAACTCTTACTTCGCAAAGAGTATGGAGTTTCTGGCGC
GACCGGTGCCTGCGCCTCGAACAGTATTCAGGAACCCTCCACATC
CCGCTCCGCGCACAAGAACACCGTCACTTGCACCCAGCAGGGCCT
GCTCGAGAACCAGCCTAGTTTCCACCCCGCCAGGCGTGAATAGGG
TGATCACTAGAGAGGAGCTCGAGGCGCTTACCCCGTCACGCACTC
CTAGCAGGTCGGTCTCGAGAACCAGCCTGGTCTCCAACCCGCCAG
GCGTAAATAGGGTGATTACAAGAGAGGAGTTTGAGGCGTTCGTAG
CACAACAACAATGACGGTTTGATGCGGGTGCATACATCTTTTCCT
CCGACACCGGTCAAGGGCATTTACAACAAAAATCAGTAAGGCAAA
CGGTGCTATCCGAAGTGGTGTTGGAGAGGACCGAATTGGAGATTT
CGTATGCCCCGCGCCTCGACCAAGAAAAAGAAGAATTACTACGCA
AGAAATTACAGTTAAATCCCACACCTGCTAACAGAAGCAGATACC
AGTCCAGGAAGGTGGAGAACATGAAAGCCATAACAGCTAGACGTA
TTCTGCAAGGCCTAGGGCATTATTTGAAGGCAGAAGGAAAAGTGG
AGTGCTACCGAACCCTGCATCCTGTTCCTTTGTATTCATCTAGTG
TGAACCGTGCCTTTTCAAGCCCCAAGGTCGCAGTGGAAGCCTGTA
ACGCCATGTTGAAAGAGAACTTTCCGACTGTGGCTTCTTACTGTA
TTATTCCAGAGTACGATGCCTATTTGGACATGGTTGACGGAGCTT
CATGCTGCTTAGACACTGCCAGTTTTTGCCCTGCAAAGCTGCGCA
GCTTTCCAAAGAAACACTCCTATTTGGAACCCACAATACGATCGG
CAGTGCCTTCAGCGATCCAGAACACGCTCCAGAACGTCCTGGCAG
CTGCCACAAAAAGAAATTGCAATGTCACGCAAATGAGAGAATTGC
CCGTATTGGATTCGGCGGCCTTTAATGTGGAATGCTTCAAGAAAT
ATGCGTGTAATAATGAATATTGGGAAACGTTTAAAGAAAACCCCA
TCAGGCTTACTGAAGAAAACGTGGTAAATTACATTACCAAATTAA
AAGGACCAAAAGCTGCTGCTCTTTTTGCGAAGACACATAATTTGA
ATATGTTGCAGGACATACCAATGGACAGGTTTGTAATGGACTTAA
AGAGAGACGTGAAAGTGACTCCAGGAACAAAACATACTGAAGAAC
GGCCCAAGGTACAGGTGATCCAGGCTGCCGATCCGCTAGCAACAG
CGTATCTGTGCGGAATCCACCGAGAGCTGGTTAGGAGATTAAATG
CGGTCCTGCTTCCGAACATTCATACACTGTTTGATATGTCGGCTG
AAGACTTTGACGCTATTATAGCCGAGCACTTCCAGCCTGGGGATT
GTGTTCTGGAAACTGACATCGCGTCGTTTGATAAAAGTGAGGACG
ACGCCATGGCTCTGACCGCGTTAATGATTCTGGAAGACTTAGGTG
TGGACGCAGAGCTGTTGACGCTGATTGAGGCGGCTTTCGGCGAAA
TTTCATCAATACATTTGCCCACTAAAACTAAATTTAAATTCGGAG
CCATGATGAAATCTGGAATGTTCCTCACACTGTTTGTGAACACAG
TCATTAACATTGTAATCGCAAGCAGAGTGTTGAGAGAACGGCTAA
CCGGATCACCATGTGCAGCATTCATTGGAGATGACAATATCGTGA
AAGGAGTCAAATCGGACAAATTAATGGCAGACAGGTGCGCCACCT
GGTTGAATATGGAAGTCAAGATTATAGATGCTGTGGTGGGCGAGA
AAGCGCCTTATTTCTGTGGAGGGTTTATTTTGTGTGACTCCGTGA
CCGGCACAGCGTGCCGTGTGGCAGACCCCCTAAAAAGGCTGTTTA
AGCTTGGCAAACCTCTGGCAGCAGACGATGAACATGATGATGACA
GGAGAAGGGCATTGCATGAAGAGTCAACACGCTGGAACCGAGTGG
GTATTCTTTCAGAGCTGTGCAAGGCAGTAGAATCAAGGTATGAAA
CCGTAGGAACTTCCATCATAGTTATGGCCATGACTACTCTAGCTA
GCAGTGTTAAATCATTCAGCTACCTGAGAGGGGCCCCTATAACTC
TCTACGGCTAACCTGAATGGACTACGACGTATCACGCCCAAACAT
TTACAGCCGCGGTGTCAAAAACCGCGTGGACGTGGTTAACATCCC
TGCTGGGAGGATCAGCCGTAATTATTATAATTGGCTTGGTGCTGG
CTACTATTGTGGCCATGTACGTGCTGACCAACCAGAAACATAATT
GAATACAGCAGCAATTGGCAAGCTGCTTACATAGAACTCGCGGCG
ATTGGCATGCCGCCTTAAAATTTTTATTTTATTTTTCTTTTCTTT
TCCGAATCGGATTTTGTTTTTAATATTTCAAAAAAAAAAAAAAAA
AAAAAAAAAAAAAAAAAAAAAAAAAAAAAAAAAAAAAAAAAAAAA
AAAAAAAAAAAAAAAAAAA
VEE-UbAAY (SEQ ID NO: 14); VEE delivery vector
with MHC class I mouse tumor epitopes
SIINFEKL and AH1-A5 inserted
VEE-Luciferase (SEQ ID NO: 15);
VEE delivery vector with luciferase gene
inserted at 7545
ubiquitin (SEQ ID NO: 38)>UbG76 0-228
Ubiquitin A76 (SEQ ID NO: 39)>UbA76 0-228
HLA-A2 (MHC class I) signal peptide
(SEQ ID NO: 40)>MHC SignalPep 0-78
HLA-A2 (MHC class I) Trans Membrane
domain (SEQ ID NO: 41)>HLA A2 TM Domain 0-201
IgK Leader Seq (SEQ ID NO: 42)>IgK Leader Seq 0-60
Human DC-Lamp (SEQ ID NO: 43)> HumanDCLAMP 0-3178
Mouse LAMP1 (SEQ ID NO: 44)>MouseLamp1 0-1858
Human Lamp1 cDNA (SEQ ID NO: 45)>Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50)>WPRE 0-593
IRES (SEQ ID NO: 51)>eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)
GPGPG linker (SEQ ID NO: 56)
1X (20x1) Cassette Nucleotide Sequence
(SEQ ID NO: 69)
ATGGCTGGCATGACCGAGTACAAACTTGTGGTGGTTGGTGCTGGA
GACGTGGGGAAAAGCGCCCTGACAATTCAGCTTATTCAGGAAACT
TGCTTATTAGACATATTAGATACTGCCGGAAAAGAAGAGTATTCT
GCTATGAGAGATCAGTACATGCGGATGTGCAACAGCAGCTGCATG
GGCGGCATGAACCGCATGCCCATCTTGACTATTATCACCCTGGAG
GATTCCTCCAGTGGAATACATTCGGGAGCCACCACCACTGCTCCT
CCCCTGTCAGGTAAGGGAAATCCTGAAGAAGAAGATGTTAGCGGC
ATTCATTCCGGTGCTACGACAACTGCACCATTTCTGTCTGGTAAA
GGTAATCCAGAGGAGGAGGATGTGGAAATTCTGGATGAGGCCTAC
GTCATGGCCTATGTGATGGCTGGCGTTGGAAGCCCATACGTGAGT
AGACTGCTGATGACAGAATATAAATTAGTGGTAGTAGGAGCAGAT
GGGGTAGGAAAGAGTGCCTTAACAATCCAACTCATTCAGGAGACC
TGTTTGTTGGATATTCTGGATACAGCTGGCCGGGAAGAGTATTCG
GCAATGAGGGACCAATATATGCGATCTTACCTCGACAGCGGAATC
CACTCCGGTGCCACTGCAACAGCGCCGAGCCTGAGTGGTAAGGGG
AACCCTGAGAAGTCAGTAACGTGTACATACAGCCCTGCTTTAAAC
AACATGTTTTGCCAACTTGCAAAAACATGTCCTGTACAGATGACA
GAGTATAAACTTGTAGTGGTGGGGGCAGCTGGTGTAGGTAAAAGT
GCACTTACCATCCAGCTGATTCAAGAGACATGTCTGTTAGACATC
TTGGACACAGCCGGGCTCGAGGAATATAGCGCCATGAGAGACCAG
TACATGCGACTGCGCGTGGAGTATCTGGATGACAGGAACACCTTC
CTGCATTCTGTTGTGGTGCCCTATGAGCCCCCGGAGGTGGATGGA
CAGATCACAGTGGGCCAGAGAATTGGCAGCGTGTCCTTCGGGACT
GTGTACAAGGGCAAGTGGCACGGAATGACGGAATATAAGCTGGTG
GTGGTAGGAGCAGTAGGTGTCGGTAAATCAGCCCTCACCATTCAG
CTTATACAGGAGACTTGTTTGCTGGACATCCTAGACACTGCTGGC
CATGAGGAGTACTCCGCCATGCGCGATCAATACATGAGGTGGCAG
CAGCAGAGCTACCTGGACTCAGGGATCCACTTTGGAGCTACCACA
ACTGCGCCATCACTAAGTGGGAAACACAGCGGCACCGCCAAATCT
GTCACGTGTACTTACTACCCAGCCTTGAACAAGATGTTCTGTCAA
TTAGCAAAGAAAAGTGTCACCTGCACATATTCACCGGCACTGAAT
GAGATGTTCTGCCAGCTGGCTAAGACCTGTCCTGTGCAGATGACT
GAGTACAAACTGGTCGTTGTGGGGGCCTGTGGGGTTGGGAAGAGC
GCTCTGACCATCCAGCTCATCCAGGGACCCGGACCAGGCGCCAAA
TTTGTTGCTGCTTGGACACTGAAAGCTGCTGCTGGGCCCGGACCA
GGCCAGTACATCAAGGCCAACTCTAAGTTTATCGGCATCACCGAA
TTGGGACCTGGACCCGGCTAGTAGTGA
KRAS 2X (8x2) Cassette Nucleotide Sequence
(SEQ ID NO: 70)
ATGGCTGGCATGACTGAATACAAATTAGTTGTCGTCGGTGCATGC
GGGGTAGGCAAGTCGGCCTTAACGATACAACTTATTCAAGAGACA
TGTCTACTGGATATCCTTGACACTGCCGGTCACGAAGAATATAGT
GCAATGAGGGACCAGTACATGCGTATGACGGAATATAAGTTAGTA
GTAGTTGGCGCAGGCGACGTAGGGAAATCCGCTCTAACTATCCAA
CTAATTCAAGAAACGTGCCTACTAGATATATTAGACACAGCGGGA
CGTGAGGAGTATAGCGCCATGCGTGACCAGTATATGCGGATGACC
GAGTATAAGCTAGTCGTAGTCGGCGCGGATGGGGTTGGTAAGAGC
GCCTTGACCATCCAGCTCATACAAGAAACTTGCCTTCTGGACATC
CTAGACACCGCGGGTCTCGAAGAGTACAGTGCGATGAGAGATCAG
TACATGAGGATGACAGAATACAAGCTCGTTGTTGTTGGTGCGGTC
GGTGTTGGAAAGAGTGCGCTAACCATTCAGCTTATCCAGGAAACC
TGTCTGTTAGACATCTTAGATACCGCAGGTAAAGAGGAATATTCG
GCCATGAGGGATCAATATATGCGAATGACAGAGTATAAATTGGTA
GTGGTAGGGGCTTGCGGAGTGGGGAAAAGCGCATTGACTATACAA
TTGATTCAGGAAACATGCCTATTGGACATACTCGACACGGCCGGG
AAAGAAGAGTATTCCGCGATGCGAGATCAATACATGCGCATGACC
GAATATAAACTTGTCGTTGTCGGAGCGGGTGATGTAGGTAAATCG
GCGCTCACAATCCAATTAATCCAAGAGACGTGCTTGCTAGACATT
CTGGATACAGCTGGGCACGAGGAGTACTCAGCTATGCGCGATCAG
TATATGAGGATGACTGAGTACAAGTTGGTCGTCGTAGGAGCGGTT
GGTGTCGGAAAATCTGCGTTGACAATTCAACTGATACAAGAGACT
TGTTTGTTAGATATTCTCGATACTGCGGGTCGGGAAGAATACTCG
GCTATGAGAGACCAATATATGAGAATGACGGAGTACAAACTCGTA
GTTGTAGGTGCGGACGGTGTAGGAAAGTCTGCGCTTACGATTCAG
TTGATACAGGAGACCTGTTTGCTCGATATCTTGGATACGGCGGGT
TTGGAGGAATACAGCGCAATGCGGGACCAATACATGAGAGGACCC
GGACCAGGCGCCAAATTTGTTGCTGCTTGGACACTGAAAGCTGCT
GCTGGGCCCGGACCAGGCCAGTACATCAAGGCCAACTCTAAGTTT
ATCGGCATCACCGAATTGGGACCTGGACCCGGCTAG
KRAS 4X (4x4) Cassette Nucleotide Sequence
(SEQ ID NO: 71)
ATGGCTGGCATGACCGAGTATAAACTAGTAGTTGTGGGAGCGTGT
GGTGTAGGCAAGTCGGCACTTACAATTCAGTTGATACAAATGACG
GAATATAAGCTCGTAGTAGTCGGAGCAGACGGCGTGGGGAAATCA
GCGTTGACTATCCAGTTAATACAGGAAACTTGCCTATTAGACATC
TTGGATACGGCAGGTCATGAGGAATATTCCGCTATGAGAGATCAG
TATATGCGCATGACGGAGTATAAGCTTGTGGTTGTCGGGGCCGAC
GGGGTAGGTAAGTCAGCGCTCACGATACAATTAATTCAAATGACC
GAATACAAGTTGGTCGTGGTGGGGGCAGTTGGGGTCGGTAAATCC
GCGTTAACGATCCAACTTATCCAAATGACAGAATATAAACTCGTT
GTTGTAGGTGCATGTGGCGTAGGAAAAAGCGCATTGACCATCCAG
CTAATTCAGGAGACGTGTCTCCTTGATATCCTAGACACGGCGGGG
CACGAAGAATACTCGGCTATGCGCGACCAGTACATGAGAATGACG
GAATACAAACTTGTTGTCGTGGGTGCGGATGGAGTAGGGAAAAGT
GCTCTAACAATACAACTCATTCAGATGACAGAGTACAAATTGGTA
GTCGTCGGTGCGGTAGGAGTTGGGAAGTCTGCACTAACTATTCAG
CTCATACAGATGACCGAGTACAAGCTGGTGGTGGTAGGCGCTTGC
GGTGTGGGTAAGAGTGCATTAACCATACAGCTTATACAAGAGACA
TGTCTGCTAGATATATTAGATACCGCCGGGCATGAAGAGTACTCT
GCCATGCGAGACCAATACATGCGTATGACAGAGTATAAATTAGTA
GTGGTTGGGGCGGACGGTGTTGGCAAGAGCGCCTTAACTATACAG
TTGATCCAGATGACGGAGTACAAACTGGTCGTCGTTGGTGCAGTG
GGAGTGGGAAAATCTGCGCTGACGATTCAACTAATCCAAGAAACA
TGTTTACTTGACATCCTCGACACTGCGGGTCACGAGGAGTATTCG
GCGATGCGTGATCAATATATGAGGATGACTGAGTATAAGTTAGTC
GTAGTTGGAGCGGTCGGTGTCGGAAAGTCCGCGCTAACCATTCAA
TTGATTCAAATGACTGAATACAAGCTAGTGGTAGTAGGAGCATGC
GGCGTCGGCAAATCGGCTTTAACAATCCAACTGATACAGGGACCC
GGACCAGGCGCCAAATTTGTTGCTGCTTGGACACTGAAAGCTGCT
GCTGGGCCCGGACCAGGCCAGTACATCAAGGCCAACTCTAAGTTT
ATCGGCATCACCGAATTGGGACCTGGACCCGGCTAG
KRAS 4x1 Cassette Nucleotide Sequence
(SEQ ID NO: 72)
ATGGCTGGCATGACCGAGTATAAACTAGTAGTTGTGGGAGCGTGT
GGTGTAGGCAAGTCGGCACTTACAATTCAGTTGATACAAATGACG
GAATATAAGCTCGTAGTAGTCGGAGCAGACGGCGTGGGGAAATCA
GCGTTGACTATCCAGTTAATACAGGAAACTTGCCTATTAGACATC
TTGGATACGGCAGGTCATGAGGAATATTCCGCTATGAGAGATCAG
TATATGCGCATGACCGAATACAAGTTGGTCGTGGTGGGGGCAGTT
GGGGTCGGTAAATCCGCGTTAACGATCCAACTTATCCAAGGACCC
GGACCAGGCGCCAAATTTGTTGCTGCTTGGACACTGAAAGCTGCT
GCTGGGCCCGGACCAGGCCAGTACATCAAGGCCAACTCTAAGTTT
ATCGGCATCACCGAATTGGGACCTGGACCCGGCTAG
KRAS 4x1 + R213L Cassette Nucleotide Sequence
(SEQ ID NO: 73)
ATGGCTGGCATGACCGAGTATAAACTAGTAGTTGTGGGAGCGTGT
GGTGTAGGCAAGTCGGCACTTACAATTCAGTTGATACAAATGACG
GAATATAAGCTCGTAGTAGTCGGAGCAGACGGCGTGGGGAAATCA
GCGTTGACTATCCAGTTAATACAGGAAACTTGCCTATTAGACATC
TTGGATACGGCAGGTCATGAGGAATATTCCGCTATGAGAGATCAG
TATATGCGCATGACCGAATACAAGTTGGTCGTGGTGGGGGCAGTT
GGGGTCGGTAAATCCGCGTTAACGATCCAACTTATCCAACTGCGC
GTGGAGTATCTGGATGACAGGAACACCTTCCTGCATTCTGTTGTG
GTGCCCTATGAGCCCCCGGAGGTGGGACCCGGACCAGGCGCCAAA
TTTGTTGCTGCTTGGACACTGAAAGCTGCTGCTGGGCCCGGACCA
GGCCAGTACATCAAGGCCAACTCTAAGTTTATCGGCATCACCGAA
TTGGGACCTGGACCCGGCTAG
KRAS 4x1 + S127Y Cassette Nucleotide
Sequence (SEQ ID NO: 74)
ATGGCTGGCATGACCGAGTATAAACTAGTAGTTGTGGGAGCGTGT
GGTGTAGGCAAGTCGGCACTTACAATTCAGTTGATACAAATGACG
GAATATAAGCTCGTAGTAGTCGGAGCAGACGGCGTGGGGAAATCA
GCGTTGACTATCCAGTTAATACAGGAAACTTGCCTATTAGACATC
TTGGATACGGCAGGTCATGAGGAATATTCCGCTATGAGAGATCAG
TATATGCGCATGACCGAATACAAGTTGGTCGTGGTGGGGGCAGTT
GGGGTCGGTAAATCCGCGTTAACGATCCAACTTATCCAACACAGC
GGCACCGCCAAATCTGTCACGTGTACTTACTACCCAGCCTTGAAC
AAGATGTTCTGTCAATTAGCAAAGGGACCCGGACCAGGCGCCAAA
TTTGTTGCTGCTTGGACACTGAAAGCTGCTGCTGGGCCCGGACCA
GGCCAGTACATCAAGGCCAACTCTAAGTTTATCGGCATCACCGAA
TTGGGACCTGGACCCGGCTAG
KRAS G12C Neoepitope (SEQ ID NO: 75)
VVVGACGVGK
KRAS G12C Neoepitope (SEQ ID NO: 76)
KLVVVGACGV
KRAS G12D Neoepitope (SEQ ID NO: 77)
VVGADGVGK
KRAS G12D Neoepitope (SEQ ID NO: 78)
VVVGADGVGK
KRAS G12V Neoepitope (SEQ ID NO: 79)
VVGAVGVGK
KRAS G12V Neoepitope (SEQ ID NO: 80)
AVGVGKSAL
KRAS G12V Neoepitope (SEQ ID NO: 81)
VVVGAVGVGK
KRAS Q61H Neoepitope (SEQ ID NO: 82)
ILDTAGHEEY
ChAdV68-Empty-E4deleted (SEQ ID NO: 83)
CATCTTCAATAATATACCTCAAACTTTTTGTGCGCGTTAATATGC
AAATGAGGCGTTTGAATTTGGGGAGGAAGGGCGGTGATTGGTCGA
GGGATGAGCGACCGTTAGGGGCGGGGCGAGTGACGTTTTGATGAC
GTGGTTGCGAGGAGGAGCCAGTTTGCAAGTTCTCGTGGGAAAAGT
GACGTCAAACGAGGTGTGGTTTGAACACGGAAATACTCAATTTTC
CCGCGCTCTCTGACAGGAAATGAGGTGTTTCTGGGCGGATGCAAG
TGAAAACGGGCCATTTTCGCGCGAAAACTGAATGAGGAAGTGAAA
ATCTGAGTAATTTCGCGTTTATGGCAGGGAGGAGTATTTGCCGAG
GGCCGAGTAGACTTTGACCGATTACGTGGGGGTTTCGATTACCGT
GTTTTTCACCTAAATTTCCGCGTACGGTGTCAAAGTCCGGTGTTT
TTACGTAGGTGTCAGCTGATCGCCAGGGTATTTAAACCTGCGCTC
TCCAGTCAAGAGGCCACTCTTGAGTGCCAGCGAGAAGAGTTTTCT
CCTCCGCGCCGCGAGTCAGATCTACACTTTGAAAGTAGGGataaG
GTAGCGAGTGAGTAGTGTTCTGGGGCGGGGGAGGACCTGCATGAG
GGCCAGAATAACTGAAATCTGTGCTTTTCTGTGTGTTGCAGCAGC
ATGAGCGGAAGCGGCTCCTTTGAGGGAGGGGTATTCAGCCCTTAT
CTGACGGGGCGTCTCCCCTCCTGGGCGGGAGTGCGTCAGAATGTG
ATGGGATCCACGGTGGACGGCCGGCCCGTGCAGCCCGCGAACTCT
TCAACCCTGACCTATGCAACCCTGAGCTCTTCGTCGTTGGACGCA
GCTGCCGCCGCAGCTGCTGCATCTGCCGCCAGCGCCGTGCGCGGA
ATGGCCATGGGCGCCGGCTACTACGGCACTCTGGTGGCCAACTCG
AGTTCCACCAATAATCCCGCCAGCCTGAACGAGGAGAAGCTGTTG
CTGCTGATGGCCCAGCTCGAGGCCTTGACCCAGCGCCTGGGCGAG
CTGACCCAGCAGGTGGCTCAGCTGCAGGAGCAGACGCGGGCCGCG
GTTGCCACGGTGAAATCCAAATAAAAAATGAATCAATAAATAAAC
GGAGACGGTTGTTGATTTTAACACAGAGTCTGAATCTTTATTTGA
TTTTTCGCGCGCGGTAGGCCCTGGACCACCGGTCTCGATCATTGA
GCACCCGGTGGATCTTTTCCAGGACCCGGTAGAGGTGGGCTTGGA
TGTTGAGGTACATGGGCATGAGCCCGTCCCGGGGGTGGAGGTAGC
TCCATTGCAGGGCCTCGTGCTCGGGGGTGGTGTTGTAAATCACCC
AGTCATAGCAGGGGCGCAGGGCATGGTGTTGCACAATATCTTTGA
GGAGGAGACTGATGGCCACGGGCAGCCCTTTGGTGTAGGTGTTTA
CAAATCTGTTGAGCTGGGAGGGATGCATGCGGGGGGAGATGAGGT
GCATCTTGGCCTGGATCTTGAGATTGGCGATGTTACCGCCCAGAT
CCCGCCTGGGGTTCATGTTGTGCAGGACCACCAGCACGGTGTATC
CGGTGCACTTGGGGAATTTATCATGCAACTTGGAAGGGAAGGCGT
GAAAGAATTTGGCGACGCCTTTGTGCCCGCCCAGGTTTTCCATGC
ACTCATCCATGATGATGGCGATGGGCCCGTGGGCGGCGGCCTGGG
CAAAGACGTTTCGGGGGTCGGACACATCATAGTTGTGGTCCTGGG
TGAGGTCATCATAGGCCATTTTAATGAATTTGGGGCGGAGGGTGC
CGGACTGGGGGACAAAGGTACCCTCGATCCCGGGGGCGTAGTTCC
CCTCACAGATCTGCATCTCCCAGGCTTTGAGCTCGGAGGGGGGGA
TCATGTCCACCTGCGGGGCGATAAAGAACACGGTTTCCGGGGCGG
GGGAGATGAGCTGGGCCGAAAGCAAGTTCCGGAGCAGCTGGGACT
TGCCGCAGCCGGTGGGGCCGTAGATGACCCCGATGACCGGCTGCA
GGTGGTAGTTGAGGGAGAGACAGCTGCCGTCCTCCCGGAGGAGGG
GGGCCACCTCGTTCATCATCTCGCGCACGTGCATGTTCTCGCGCA
CCAGTTCCGCCAGGAGGCGCTCTCCCCCCAGGGATAGGAGCTCCT
GGAGCGAGGCGAAGTTTTTCAGCGGCTTGAGTCCGTCGGCCATGG
GCATTTTGGAGAGGGTTTGTTGCAAGAGTTCCAGGCGGTCCCAGA
GCTCGGTGATGTGCTCTACGGCATCTCGATCCAGCAGACCTCCTC
GTTTCGCGGGTTGGGACGGCTGCGGGAGTAGGGCACCAGACGATG
GGCGTCCAGCGCAGCCAGGGTCCGGTCCTTCCAGGGTCGCAGCGT
CCGCGTCAGGGTGGTCTCCGTCACGGTGAAGGGGTGCGCGCCGGG
CTGGGCGCTTGCGAGGGTGCGCTTCAGGCTCATCCGGCTGGTCGA
AAACCGCTCCCGATCGGCGCCCTGCGCGTCGGCCAGGTAGCAATT
GACCATGAGTTCGTAGTTGAGCGCCTCGGCCGCGTGGCCTTTGGC
GCGGAGCTTACCTTTGGAAGTCTGCCCGCAGGCGGGACAGAGGAG
GGACTTGAGGGCGTAGAGCTTGGGGGCGAGGAAGACGGACTCGGG
GGCGTAGGCGTCCGCGCCGCAGTGGGCGCAGACGGTCTCGCACTC
CACGAGCCAGGTGAGGTCGGGCTGGTCGGGGTCAAAAACCAGTTT
CCCGCCGTTCTTTTTGATGCGTTTCTTACCTTTGGTCTCCATGAG
CTCGTGTCCCCGCTGGGTGACAAAGAGGCTGTCCGTGTCCCCGTA
GACCGACTTTATGGGCCGGTCCTCGAGCGGTGTGCCGCGGTCCTC
CTCGTAGAGGAACCCCGCCCACTCCGAGACGAAAGCCCGGGTCCA
GGCCAGCACGAAGGAGGCCACGTGGGACGGGTAGCGGTCGTTGTC
CACCAGCGGGTCCACCTTTTCCAGGGTATGCAAACACATGTCCCC
CTCGTCCACATCCAGGAAGGTGATTGGCTTGTAAGTGTAGGCCAC
GTGACCGGGGGTCCCGGCCGGGGGGGTATAAAAGGGTGCGGGTCC
CTGCTCGTCCTCACTGTCTTCCGGATCGCTGTCCAGGAGCGCCAG
CTGTTGGGGTAGGTATTCCCTCTCGAAGGCGGGCATGACCTCGGC
ACTCAGGTTGTCAGTTTCTAGAAACGAGGAGGATTTGATATTGAC
GGTGCCGGCGGAGATGCCTTTCAAGAGCCCCTCGTCCATCTGGTC
AGAAAAGACGATCTTTTTGTTGTCGAGCTTGGTGGCGAAGGAGCC
GTAGAGGGCGTTGGAGAGGAGCTTGGCGATGGAGCGCATGGTCTG
GTTTTTTTCCTTGTCGGCGCGCTCCTTGGCGGCGATGTTGAGCTG
CACGTACTCGCGCGCCACGCACTTCCATTCGGGGAAGACGGTGGT
CAGCTCGTCGGGCACGATTCTGACCTGCCAGCCCCGATTATGCAG
GGTGATGAGGTCCACACTGGTGGCCACCTCGCCGCGCAGGGGCTC
ATTAGTCCAGCAGAGGCGTCCGCCCTTGCGCGAGCAGAAGGGGGG
CAGGGGGTCCAGCATGACCTCGTCGGGGGGGTCGGCATCGATGGT
GAAGATGCCGGGCAGGAGGTCGGGGTCAAAGTAGCTGATGGAAGT
GGCCAGATCGTCCAGGGCAGCTTGCCATTCGCGCACGGCCAGCGC
GCGCTCGTAGGGACTGAGGGGCGTGCCCCAGGGCATGGGATGGGT
AAGCGCGGAGGCGTACATGCCGCAGATGTCGTAGACGTAGAGGGG
CTCCTCGAGGATGCCGATGTAGGTGGGGTAGCAGCGCCCCCCGCG
GATGCTGGCGCGCACGTAGTCATACAGCTCGTGCGAGGGGGCGAG
GAGCCCCGGGCCCAGGTTGGTGCGACTGGGCTTTTCGGCGCGGTA
GACGATCTGGCGGAAAATGGCATGCGAGTTGGAGGAGATGGTGGG
CCTTTGGAAGATGTTGAAGTGGGCGTGGGGCAGTCCGACCGAGTC
GCGGATGAAGTGGGCGTAGGAGTCTTGCAGCTTGGCGACGAGCTC
GGCGGTGACTAGGACGTCCAGAGCGCAGTAGTCGAGGGTCTCCTG
GATGATGTCATACTTGAGCTGTCCCTTTTGTTTCCACAGCTCGCG
GTTGAGAAGGAACTCTTCGCGGTCCTTCCAGTACTCTTCGAGGGG
GAACCCGTCCTGATCTGCACGGTAAGAGCCTAGCATGTAGAACTG
GTTGACGGCCTTGTAGGCGCAGCAGCCCTTCTCCACGGGGAGGGC
GTAGGCCTGGGCGGCCTTGCGCAGGGAGGTGTGCGTGAGGGCGAA
AGTGTCCCTGACCATGACCTTGAGGAACTGGTGCTTGAAGTCGAT
ATCGTCGCAGCCCCCCTGCTCCCAGAGCTGGAAGTCCGTGCGCTT
CTTGTAGGCGGGGTTGGGCAAAGCGAAAGTAACATCGTTGAAGAG
GATCTTGCCCGCGCGGGGCATAAAGTTGCGAGTGATGCGGAAAGG
TTGGGGCACCTCGGCCCGGTTGTTGATGACCTGGGCGGCGAGCAC
GATCTCGTCGAAGCCGTTGATGTTGTGGCCCACGATGTAGAGTTC
CACGAATCGCGGACGGCCCTTGACGTGGGGCAGTTTCTTGAGCTC
CTCGTAGGTGAGCTCGTCGGGGTCGCTGAGCCCGTGCTGCTCGAG
CGCCCAGTCGGCGAGATGGGGGTTGGCGCGGAGGAAGGAAGTCCA
GAGATCCACGGCCAGGGCGGTTTGCAGACGGTCCCGGTACTGACG
GAACTGCTGCCCGACGGCCATTTTTTCGGGGGTGACGCAGTAGAA
GGTGCGGGGGTCCCCGTGCCAGCGATCCCATTTGAGCTGGAGGGC
GAGATCGAGGGCGAGCTCGACGAGCCGGTCGTCCCCGGAGAGTTT
CATGACCAGCATGAAGGGGACGAGCTGCTTGCCGAAGGACCCCAT
CCAGGTGTAGGTTTCCACATCGTAGGTGAGGAAGAGCCTTTCGGT
GCGAGGATGCGAGCCGATGGGGAAGAACTGGATCTCCTGCCACCA
ATTGGAGGAATGGCTGTTGATGTGATGGAAGTAGAAATGCCGACG
GCGCGCCGAACACTCGTGCTTGTGTTTATACAAGCGGCCACAGTG
CTCGCAACGCTGCACGGGATGCACGTGCTGCACGAGCTGTACCTG
AGTTCCTTTGACGAGGAATTTCAGTGGGAAGTGGAGTCGTGGCGC
CTGCATCTCGTGCTGTACTACGTCGTGGTGGTCGGCCTGGCCCTC
TTCTGCCTCGATGGTGGTCATGCTGACGAGCCCGCGCGGGAGGCA
GGTCCAGACCTCGGCGCGAGCGGGTCGGAGAGCGAGGACGAGGGC
GCGCAGGCCGGAGCTGTCCAGGGTCCTGAGACGCTGCGGAGTCAG
GTCAGTGGGCAGCGGCGGCGCGCGGTTGACTTGCAGGAGTTTTTC
CAGGGCGCGCGGGAGGTCCAGATGGTACTTGATCTCCACCGCGCC
ATTGGTGGCGACGTCGATGGCTTGCAGGGTCCCGTGCCCCTGGGG
TGTGACCACCGTCCCCCGTTTCTTCTTGGGCGGCTGGGGCGACGG
GGGCGGTGCCTCTTCCATGGTTAGAAGCGGCGGCGAGGACGCGCG
CCGGGCGGCAGGGGCGGCTCGGGGCCCGGAGGCAGGGGCGGCAGG
GGCACGTCGGCGCCGCGCGCGGGTAGGTTCTGGTACTGCGCCCGG
AGAAGACTGGCGTGAGCGACGACGCGACGGTTGACGTCCTGGATC
TGACGCCTCTGGGTGAAGGCCACGGGACCCGTGAGTTTGAACCTG
AAAGAGAGTTCGACAGAATCAATCTCGGTATCGTTGACGGCGGCC
TGCCGCAGGATCTCTTGCACGTCGCCCGAGTTGTCCTGGTAGGCG
ATCTCGGTCATGAACTGCTCGATCTCCTCCTCTTGAAGGTCTCCG
CGGCCGGCGCGCTCCACGGTGGCCGCGAGGTCGTTGGAGATGCGG
CCCATGAGCTGCGAGAAGGCGTTCATGCCCGCCTCGTTCCAGACG
CGGCTGTAGACCACGACGCCCTCGGGATCGCgGGCGCGCATGACC
ACCTGGGCGAGGTTGAGCTCCACGTGGCGCGTGAAGACCGCGTAG
TTGCAGAGGCGCTGGTAGAGGTAGTTGAGCGTGGTGGCGATGTGC
TCGGTGACGAAGAAATACATGATCCAGCGGCGGAGCGGCATCTCG
CTGACGTCGCCCAGCGCCTCCAAACGTTCCATGGCCTCGTAAAAG
TCCACGGCGAAGTTGAAAAACTGGGAGTTGCGCGCCGAGACGGTC
AACTCCTCCTCCAGAAGACGGATGAGCTCGGCGATGGTGGCGCGC
ACCTCGCGCTCGAAGGCCCCCGGGAGTTCCTCCACTTCCTCTTCT
TCCTCCTCCACTAACATCTCTTCTACTTCCTCCTCAGGCGGCAGT
GGTGGCGGGGGAGGGGGCCTGCGTCGCCGGCGGCGCACGGGCAGA
CGGTCGATGAAGCGCTCGATGGTCTCGCCGCGCCGGCGTCGCATG
GTCTCGGTGACGGCGCGCCCGTCCTCGCGGGGCCGCAGCGTGAAG
ACGCCGCCGCGCATCTCCAGGTGGCCGGGGGGGTCCCCGTTGGGC
AGGGAGAGGGCGCTGACGATGCATCTTATCAATTGCCCCGTAGGG
ACTCCGCGCAAGGACCTGAGCGTCTCGAGATCCACGGGATCTGAA
AACCGCTGAACGAAGGCTTCGAGCCAGTCGCAGTCGCAAGGTAGG
CTGAGCACGGTTTCTTCTGGCGGGTCATGTTGGTTGGGAGCGGGG
GGGGCGATGCTGCTGGTGATGAAGTTGAAATAGGCGGTTCTGAGA
CGGCGGATGGTGGCGAGGAGCACCAGGTCTTTGGGCCCGGCTTGC
TGGATGCGCAGACGGTCGGCCATGCCCCAGGCGTGGTCCTGACAC
CTGGCCAGGTCCTTGTAGTAGTCCTGCATGAGCCGCTCCACGGGC
ACCTCCTCCTCGCCCGCGCGGCCGTGCATGCGCGTGAGCCCGAAG
CCGCGCTGGGGCTGGACGAGCGCCAGGTCGGCGACGACGCGCTCG
GCGAGGATGGCTTGCTGGATCTGGGTGAGGGTGGTCTGGAAGTCA
TCAAAGTCGACGAAGCGGTGGTAGGCTCCGGTGTTGATGGTGTAG
GAGCAGTTGGCCATGACGGACCAGTTGACGGTCTGGTGGCCCGGA
CGCACGAGCTCGTGGTACTTGAGGCGCGAGTAGGCGCGCGTGTCG
AAGATGTAGTCGTTGCAGGTGCGCACCAGGTACTGGTAGCCGATG
AGGAAGTGCGGCGGCGGCTGGCGGTAGAGCGGCCATCGCTCGGTG
GCGGGGGCGCCGGGCGCGAGGTCCTCGAGCATGGTGCGGTGGTAG
CCGTAGATGTACCTGGACATCCAGGTGATGCCGGCGGCGGTGGTG
GAGGCGCGCGGGAACTCGCGGACGCGGTTCCAGATGTTGCGCAGC
GGCAGGAAGTAGTTCATGGTGGGCACGGTCTGGCCCGTGAGGCGC
GCGCAGTCGTGGATGCTCTATACGGGCAAAAACGAAAGCGGTCAG
CGGCTCGACTCCGTGGCCTGGAGGCTAAGCGAACGGGTTGGGCTG
CGCGTGTACCCCGGTTCGAATCTCGAATCAGGCTGGAGCCGCAGC
TAACGTGGTATTGGCACTCCCGTCTCGACCCAAGCCTGCACCAAC
CCTCCAGGATACGGAGGCGGGTCGTTTTGCAACTTTTTTTTGGAG
GCCGGATGAGACTAGTAAGCGCGGAAAGCGGCCGACCGCGATGGC
TCGCTGCCGTAGTCTGGAGAAGAATCGCCAGGGTTGCGTTGCGGT
GTGCCCCGGTTCGAGGCCGGCCGGATTCCGCGGCTAACGAGGGCG
TGGCTGCCCCGTCGTTTCCAAGACCCCATAGCCAGCCGACTTCTC
CAGTTACGGAGCGAGCCCCTCTTTTGTTTTGTTTGTTTTTGCCAG
ATGCATCCCGTACTGCGGCAGATGCGCCCCCACCACCCTCCACCG
CAACAACAGCCCCCTCCACAGCCGGCGCTTCTGCCCCCGCCCCAG
CAGCAACTTCCAGCCACGACCGCCGCGGCCGCCGTGAGCGGGGCT
GGACAGAGTTATGATCACCAGCTGGCCTTGGAAGAGGGCGAGGGG
CTGGCGCGCCTGGGGGCGTCGTCGCCGGAGCGGCACCCGCGCGTG
CAGATGAAAAGGGACGCTCGCGAGGCCTACGTGCCCAAGCAGAAC
CTGTTCAGAGACAGGAGCGGCGAGGAGCCCGAGGAGATGCGCGCG
GCCCGGTTCCACGCGGGGGGGGAGCTGCGGCGCGGCCTGGACCGA
AAGAGGGTGCTGAGGGACGAGGATTTCGAGGCGGACGAGCTGACG
GGGATCAGCCCCGCGCGCGCGCACGTGGCCGCGGCCAACCTGGTC
ACGGCGTACGAGCAGACCGTGAAGGAGGAGAGCAACTTCCAAAAA
TCCTTCAACAACCACGTGCGCACCCTGATCGCGCGCGAGGAGGTG
ACCCTGGGCCTGATGCACCTGTGGGACCTGCTGGAGGCCATCGTG
CAGAACCCCACCAGCAAGCCGCTGACGGCGCAGCTGTTCCTGGTG
GTGCAGCATAGTCGGGACAACGAAGCGTTCAGGGAGGCGCTGCTG
AATATCACCGAGCCCGAGGGCCGCTGGCTCCTGGACCTGGTGAAC
ATTCTGCAGAGCATCGTGGTGCAGGAGCGCGGGCTGCCGCTGTCC
GAGAAGCTGGCGGCCATCAACTTCTCGGTGCTGAGTTTGGGCAAG
TACTACGCTAGGAAGATCTACAAGACCCCGTACGTGCCCATAGAC
AAGGAGGTGAAGATCGACGGGTTTTACATGCGCATGACCCTGAAA
GTGCTGACCCTGAGCGACGATCTGGGGGTGTACCGCAACGACAGG
ATGCACCGTGCGGTGAGCGCCAGCAGGCGGCGCGAGCTGAGCGAC
CAGGAGCTGATGCATAGTCTGCAGCGGGCCCTGACCGGGGCCGGG
ACCGAGGGGGAGAGCTACTTTGACATGGGCGCGGACCTGCACTGG
CAGCCCAGCCGCCGGGCCTTGGAGGCGGCGGCAGGACCCTACGTA
GAAGAGGTGGACGATGAGGTGGACGAGGAGGGCGAGTACCTGGAA
GACTGATGGCGCGACCGTATTTTTGCTAGATGCAACAACAACAGC
CACCTCCTGATCCCGCGATGCGGGCGGCGCTGCAGAGCCAGCCGT
CCGGCATTAACTCCTCGGACGATTGGACCCAGGCCATGCAACGCA
TCATGGCGCTGACGACCCGCAACCCCGAAGCCTTTAGACAGCAGC
CCCAGGCCAACCGGCTCTCGGCCATCCTGGAGGCCGTGGTGCCCT
CGCGCTCCAACCCCACGCACGAGAAGGTCCTGGCCATCGTGAACG
CGCTGGTGGAGAACAAGGCCATCCGCGGCGACGAGGCCGGCCTGG
TGTACAACGCGCTGCTGGAGCGCGTGGCCCGCTACAACAGCACCA
ACGTGCAGACCAACCTGGACCGCATGGTGACCGACGTGCGCGAGG
CCGTGGCCCAGCGCGAGCGGTTCCACCGCGAGTCCAACCTGGGAT
CCATGGTGGCGCTGAACGCCTTCCTCAGCACCCAGCCCGCCAACG
TGCCCCGGGGCCAGGAGGACTACACCAACTTCATCAGCGCCCTGC
GCCTGATGGTGACCGAGGTGCCCCAGAGCGAGGTGTACCAGTCCG
GGCCGGACTACTTCTTCCAGACCAGTCGCCAGGGCTTGCAGACCG
TGAACCTGAGCCAGGCTTTCAAGAACTTGCAGGGCCTGTGGGGCG
TGCAGGCCCCGGTCGGGGACCGCGCGACGGTGTCGAGCCTGCTGA
CGCCGAACTCGCGCCTGCTGCTGCTGCTGGTGGCCCCCTTCACGG
ACAGCGGCAGCATCAACCGCAACTCGTACCTGGGCTACCTGATTA
ACCTGTACCGCGAGGCCATCGGCCAGGCGCACGTGGACGAGCAGA
CCTACCAGGAGATCACCCACGTGAGCCGCGCCCTGGGCCAGGACG
ACCCGGGCAACCTGGAAGCCACCCTGAACTTTTTGCTGACCAACC
GGTCGCAGAAGATCCCGCCCCAGTACGCGCTCAGCACCGAGGAGG
AGCGCATCCTGCGTTACGTGCAGCAGAGCGTGGGCCTGTTCCTGA
TGCAGGAGGGGGCCACCCCCAGCGCCGCGCTCGACATGACCGCGC
GCAACATGGAGCCCAGCATGTACGCCAGCAACCGCCCGTTCATCA
ATAAACTGATGGACTACTTGCATCGGGCGGCCGCCATGAACTCTG
ACTATTTCACCAACGCCATCCTGAATCCCCACTGGCTCCCGCCGC
CGGGGTTCTACACGGGCGAGTACGACATGCCCGACCCCAATGACG
GGTTCCTGTGGGACGATGTGGACAGCAGCGTGTTCTCCCCCCGAC
CGGGTGCTAACGAGCGCCCCTTGTGGAAGAAGGAAGGCAGCGACC
GACGCCCGTCCTCGGCGCTGTCCGGCCGCGAGGGTGCTGCCGCGG
CGGTGCCCGAGGCCGCCAGTCCTTTCCCGAGCTTGCCCTTCTCGC
TGAACAGTATCCGCAGCAGCGAGCTGGGCAGGATCACGCGCCCGC
GCTTGCTGGGCGAAGAGGAGTACTTGAATGACTCGCTGTTGAGAC
CCGAGCGGGAGAAGAACTTCCCCAATAACGGGATAGAAAGCCTGG
TGGACAAGATGAGCCGCTGGAAGACGTATGCGCAGGAGCACAGGG
ACGATCCCCGGGCGTCGCAGGGGGCCACGAGCCGGGGCAGCGCCG
CCCGTAAACGCCGGTGGCACGACAGGCAGCGGGGACAGATGTGGG
ACGATGAGGACTCCGCCGACGACAGCAGCGTGTTGGACTTGGGTG
GGAGTGGTAACCCGTTCGCTCACCTGCGCCCCCGTATCGGGCGCA
TGATGTAAGAGAAACCGAAAATAAATGATACTCACCAAGGCCATG
GCGACCAGCGTGCGTTCGTTTCTTCTCTGTTGTTGTTGTATCTAG
TATGATGAGGCGTGCGTACCCGGAGGGTCCTCCTCCCTCGTACGA
GAGCGTGATGCAGCAGGCGATGGCGGCGGCGGCGATGCAGCCCCC
GCTGGAGGCTCCTTACGTGCCCCCGCGGTACCTGGCGCCTACGGA
GGGGCGGAACAGCATTCGTTACTCGGAGCTGGCACCCTTGTACGA
TACCACCCGGTTGTACCTGGTGGACAACAAGTCGGCGGACATCGC
CTCGCTGAACTACCAGAACGACCACAGCAACTTCCTGACCACCGT
GGTGCAGAACAATGACTTCACCCCCACGGAGGCCAGCACCCAGAC
CATCAACTTTGACGAGCGCTCGCGGTGGGGCGGCCAGCTGAAAAC
CATCATGCACACCAACATGCCCAACGTGAACGAGTTCATGTACAG
CAACAAGTTCAAGGCGCGGGTGATGGTCTCCCGCAAGACCCCCAA
TGGGGTGACAGTGACAGAGGATTATGATGGTAGTCAGGATGAGCT
GAAGTATGAATGGGTGGAATTTGAGCTGCCCGAAGGCAACTTCTC
GGTGACCATGACCATCGACCTGATGAACAACGCCATCATCGACAA
TTACTTGGCGGTGGGGCGGCAGAACGGGGTGCTGGAGAGCGACAT
CGGCGTGAAGTTCGACACTAGGAACTTCAGGCTGGGCTGGGACCC
CGTGACCGAGCTGGTCATGCCCGGGGTGTACACCAACGAGGCTTT
CCATCCCGATATTGTCTTGCTGCCCGGCTGCGGGGTGGACTTCAC
CGAGAGCCGCCTCAGCAACCTGCTGGGCATTCGCAAGAGGCAGCC
CTTCCAGGAAGGCTTCCAGATCATGTACGAGGATCTGGAGGGGGG
CAACATCCCCGCGCTCCTGGATGTCGACGCCTATGAGAAAAGCAA
GGAGGATGCAGCAGCTGAAGCAACTGCAGCCGTAGCTACCGCCTC
TACCGAGGTCAGGGGCGATAATTTTGCAAGCGCCGCAGCAGTGGC
AGCGGCCGAGGCGGCTGAAACCGAAAGTAAGATAGTCATTCAGCC
GGTGGAGAAGGATAGCAAGAACAGGAGCTACAACGTACTACCGGA
CAAGATAAACACCGCCTACCGCAGCTGGTACCTAGCCTACAACTA
TGGCGACCCCGAGAAGGGCGTGCGCTCCTGGACGCTGCTCACCAC
CTCGGACGTCACCTGCGGCGTGGAGCAAGTCTACTGGTCGCTGCC
CGACATGATGCAAGACCCGGTCACCTTCCGCTCCACGCGTCAAGT
TAGCAACTACCCGGTGGTGGGCGCCGAGCTCCTGCCCGTCTACTC
CAAGAGCTTCTTCAACGAGCAGGCCGTCTACTCGCAGCAGCTGCG
CGCCTTCACCTCGCTTACGCACGTCTTCAACCGCTTCCCCGAGAA
CCAGATCCTCGTCCGCCCGCCCGCGCCCACCATTACCACCGTCAG
TGAAAACGTTCCTGCTCTCACAGATCACGGGACCCTGCCGCTGCG
CAGCAGTATCCGGGGAGTCCAGCGCGTGACCGTTACTGACGCCAG
ACGCCGCACCTGCCCCTACGTCTACAAGGCCCTGGGCATAGTCGC
GCCGCGCGTCCTCTCGAGCCGCACCTTCTAAATGTCCATTCTCAT
CTCGCCCAGTAATAACACCGGTTGGGGCCTGCGCGCGCCCAGCAA
GATGTACGGAGGCGCTCGCCAACGCTCCACGCAACACCCCGTGCG
CGTGCGCGGGCACTTCCGCGCTCCCTGGGGCGCCCTCAAGGGCCG
CGTGCGGTCGCGCACCACCGTCGACGACGTGATCGACCAGGTGGT
GGCCGACGCGCGCAACTACACCCCCGCCGCCGCGCCCGTCTCCAC
CGTGGACGCCGTCATCGACAGCGTGGTGGCcGACGCGCGCCGGTA
CGCCCGCGCCAAGAGCCGGCGGCGGCGCATCGCCCGGCGGCACCG
GAGCACCCCCGCCATGCGCGCGGCGCGAGCCTTGCTGCGCAGGGC
CAGGCGCACGGGACGCAGGGCCATGCTCAGGGCGGCCAGACGCGC
GGCTTCAGGCGCCAGCGCCGGCAGGACCCGGAGACGCGCGGCCAC
GGCGGCGGCAGCGGCCATCGCCAGCATGTCCCGCCCGCGGCGAGG
GAACGTGTACTGGGTGCGCGACGCCGCCACCGGTGTGCGCGTGCC
CGTGCGCACCCGCCCCCCTCGCACTTGAAGATGTTCACTTCGCGA
TGTTGATGTGTCCCAGCGGCGAGGAGGATGTCCAAGCGCAAATTC
AAGGAAGAGATGCTCCAGGTCATCGCGCCTGAGATCTACGGCCCT
GCGGTGGTGAAGGAGGAAAGAAAGCCCCGCAAAATCAAGCGGGTC
AAAAAGGACAAAAAGGAAGAAGAAAGTGATGTGGACGGATTGGTG
GAGTTTGTGCGCGAGTTCGCCCCCCGGCGGCGCGTGCAGTGGCGC
GGGCGGAAGGTGCAACCGGTGCTGAGACCCGGCACCACCGTGGTC
TTCACGCCCGGCGAGCGCTCCGGCACCGCTTCCAAGCGCTCCTAC
GACGAGGTGTACGGGGATGATGATATTCTGGAGCAGGCGGCCGAG
CGCCTGGGCGAGTTTGCTTACGGCAAGCGCAGCCGTTCCGCACCG
AAGGAAGAGGCGGTGTCCATCCCGCTGGACCACGGCAACCCCACG
CCGAGCCTCAAGCCCGTGACCTTGCAGCAGGTGCTGCCGACCGCG
GCGCCGCGCCGGGGGTTCAAGCGCGAGGGCGAGGATCTGTACCCC
ACCATGCAGCTGATGGTGCCCAAGCGCCAGAAGCTGGAAGACGTG
CTGGAGACCATGAAGGTGGACCCGGACGTGCAGCCCGAGGTCAAG
GTGCGGCCCATCAAGCAGGTGGCCCCGGGCCTGGGCGTGCAGACC
GTGGACATCAAGATTCCCACGGAGCCCATGGAAACGCAGACCGAG
CCCATGATCAAGCCCAGCACCAGCACCATGGAGGTGCAGACGGAT
CCCTGGATGCCATCGGCTCCTAGTCGAAGACCCCGGCGCAAGTAC
GGCGCGGCCAGCCTGCTGATGCCCAACTACGCGCTGCATCCTTCC
ATCATCCCCACGCCGGGCTACCGCGGCACGCGCTTCTACCGCGGT
CATACCAGCAGCCGCCGCCGCAAGACCACCACTCGCCGCCGCCGT
CGCCGCACCGCCGCTGCAACCACCCCTGCCGCCCTGGTGCGGAGA
GTGTACCGCCGCGGCCGCGCACCTCTGACCCTGCCGCGCGCGCGC
TACCACCCGAGCATCGCCATTTAAACTTTCGCCtGCTTTGCAGAT
CAATGGCCCTCACATGCCGCCTTCGCGTTCCCATTACGGGCTACC
GAGGAAGAAAACCGCGCCGTAGAAGGCTGGCGGGGAACGGGATGC
GTCGCCACCACCACCGGCGGCGGCGCGCCATCAGCAAGCGGTTGG
GGGGAGGCTTCCTGCCCGCGCTGATCCCCATCATCGCCGCGGCGA
TCGGGGCGATCCCCGGCATTGCTTCCGTGGCGGTGCAGGCCTCTC
AGCGCCACTGAGACACACTTGGAAACATCTTGTAATAAACCaATG
GACTCTGACGCTCCTGGTCCTGTGATGTGTTTTCGTAGACAGATG
GAAGACATCAATTTTTCGTCCCTGGCTCCGCGACACGGCACGCGG
CCGTTCATGGGCACCTGGAGCGACATCGGCACCAGCCAACTGAAC
GGGGGCGCCTTCAATTGGAGCAGTCTCTGGAGCGGGCTTAAGAAT
TTCGGGTCCACGCTTAAAACCTATGGCAGCAAGGCGTGGAACAGC
ACCACAGGGCAGGCGCTGAGGGATAAGCTGAAAGAGCAGAACTTC
CAGCAGAAGGTGGTCGATGGGCTCGCCTCGGGCATCAACGGGGTG
GTGGACCTGGCCAACCAGGCCGTGCAGCGGCAGATCAACAGCCGC
CTGGACCCGGTGCCGCCCGCCGGCTCCGTGGAGATGCCGCAGGTG
GAGGAGGAGCTGCCTCCCCTGGACAAGCGGGGCGAGAAGCGACCC
CGCCCCGATGCGGAGGAGACGCTGCTGACGCACACGGACGAGCCG
CCCCCGTACGAGGAGGCGGTGAAACTGGGTCTGCCCACCACGCGG
CCCATCGCGCCCCTGGCCACCGGGGTGCTGAAACCCGAAAAGCCC
GCGACCCTGGACTTGCCTCCTCCCCAGCCTTCCCGCCCCTCTACA
GTGGCTAAGCCCCTGCCGCCGGTGGCCGTGGCCCGCGCGCGACCC
GGGGGCACCGCCCGCCCTCATGCGAACTGGCAGAGCACTCTGAAC
AGCATCGTGGGTCTGGGAGTGCAGAGTGTGAAGCGCCGCCGCTGC
TATTAAACCTACCGTAGCGCTTAACTTGCTTGTCTGTGTGTGTAT
GTATTATGTCGCCGCCGCCGCTGTCCACCAGAAGGAGGAGTGAAG
AGGCGCGTCGCCGAGTTGCAAGATGGCCACCCCATCGATGCTGCC
CCAGTGGGCGTACATGCACATCGCCGGACAGGACGCTTCGGAGTA
CCTGAGTCCGGGTCTGGTGCAGTTTGCCCGCGCCACAGACACCTA
CTTCAGTCTGGGGAACAAGTTTAGGAACCCCACGGTGGCGCCCAC
GCACGATGTGACCACCGACCGCAGCCAGCGGCTGACGCTGCGCTT
CGTGCCCGTGGACCGCGAGGACAACACCTACTCGTACAAAGTGCG
CTACACGCTGGCCGTGGGCGACAACCGCGTGCTGGACATGGCCAG
CACCTACTTTGACATCCGCGGCGTGCTGGATCGGGGCCCTAGCTT
CAAACCCTACTCCGGCACCGCCTACAACAGTCTGGCCCCCAAGGG
AGCACCCAACACTTGTCAGTGGACATATAAAGCCGATGGTGAAAC
TGCCACAGAAAAAACCTATACATATGGAAATGCACCCGTGCAGGG
CATTAACATCACAAAAGATGGTATTCAACTTGGAACTGACACCGA
TGATCAGCCAATCTACGCAGATAAAACCTATCAGCCTGAACCTCA
AGTGGGTGATGCTGAATGGCATGACATCACTGGTACTGATGAAAA
GTATGGAGGCAGAGCTCTTAAGCCTGATACCAAAATGAAGCCTTG
TTATGGTTCTTTTGCCAAGCCTACTAATAAAGAAGGAGGTCAGGC
AAATGTGAAAACAGGAACAGGCACTACTAAAGAATATGACATAGA
CATGGCTTTCTTTGACAACAGAAGTGCGGCTGCTGCTGGCCTAGC
TCCAGAAATTGTTTTGTATACTGAAAATGTGGATTTGGAAACTCC
AGATACCCATATTGTATACAAAGCAGGCACAGATGACAGCAGCTC
TTCTATTAATTTGGGTCAGCAAGCCATGCCCAACAGACCTAACTA
CATTGGTTTCAGAGACAACTTTATCGGGCTCATGTACTACAACAG
CACTGGCAATATGGGGGTGCTGGCCGGTCAGGCTTCTCAGCTGAA
TGCTGTGGTTGACTTGCAAGACAGAAACACCGAGCTGTCCTACCA
GCTCTTGCTTGACTCTCTGGGTGACAGAACCCGGTATTTCAGTAT
GTGGAATCAGGCGGTGGACAGCTATGATCCTGATGTGCGCATTAT
TGAAAATCATGGTGTGGAGGATGAACTTCCCAACTATTGTTTCCC
TCTGGATGCTGTTGGCAGAACAGATACTTATCAGGGAATTAAGGC
TAATGGAACTGATCAAACCACATGGACCAAAGATGACAGTGTCAA
TGATGCTAATGAGATAGGCAAGGGTAATCCATTCGCCATGGAAAT
CAACATCCAAGCCAACCTGTGGAGGAACTTCCTCTACGCCAACGT
GGCCCTGTACCTGCCCGACTCTTACAAGTACACGCCGGCCAATGT
TACCCTGCCCACCAACACCAACACCTACGATTACATGAACGGCCG
GGTGGTGGCGCCCTCGCTGGTGGACTCCTACATCAACATCGGGGC
GCGCTGGTCGCTGGATCCCATGGACAACGTGAACCCCTTCAACCA
CCACCGCAATGCGGGGCTGCGCTACCGCTCCATGCTCCTGGGCAA
CGGGCGCTACGTGCCCTTCCACATCCAGGTGCCCCAGAAATTTTT
CGCCATCAAGAGCCTCCTGCTCCTGCCCGGGTCCTACACCTACGA
GTGGAACTTCCGCAAGGACGTCAACATGATCCTGCAGAGCTCCCT
CGGCAACGACCTGCGCACGGACGGGGCCTCCATCTCCTTCACCAG
CATCAACCTCTACGCCACCTTCTTCCCCATGGCGCACAACACGGC
CTCCACGCTCGAGGCCATGCTGCGCAACGACACCAACGACCAGTC
CTTCAACGACTACCTCTCGGCGGCCAACATGCTCTACCCCATCCC
GGCCAACGCCACCAACGTGCCCATCTCCATCCCCTCGCGCAACTG
GGCCGCCTTCCGCGGCTGGTCCTTCACGCGTCTCAAGACCAAGGA
GACGCCCTCGCTGGGCTCCGGGTTCGACCCCTACTTCGTCTACTC
GGGCTCCATCCCCTACCTCGACGGCACCTTCTACCTCAACCACAC
CTTCAAGAAGGTCTCCATCACCTTCGACTCCTCCGTCAGCTGGCC
CGGCAACGACCGGCTCCTGACGCCCAACGAGTTCGAAATCAAGCG
CACCGTCGACGGCGAGGGCTACAACGTGGCCCAGTGCAACATGAC
CAAGGACTGGTTCCTGGTCCAGATGCTGGCCCACTACAACATCGG
CTACCAGGGCTTCTACGTGCCCGAGGGCTACAAGGACCGCATGTA
CTCCTTCTTCCGCAACTTCCAGCCCATGAGCCGCCAGGTGGTGGA
CGAGGTCAACTACAAGGACTACCAGGCCGTCACCCTGGCCTACCA
GCACAACAACTCGGGCTTCGTCGGCTACCTCGCGCCCACCATGCG
CCAGGGCCAGCCCTACCCCGCCAACTACCCCTACCCGCTCATCGG
CAAGAGCGCCGTCACCAGCGTCACCCAGAAAAAGTTCCTCTGCGA
CAGGGTCATGTGGCGCATCCCCTTCTCCAGCAACTTCATGTCCAT
GGGCGCGCTCACCGACCTCGGCCAGAACATGCTCTATGCCAACTC
CGCCCACGCGCTAGACATGAATTTCGAAGTCGACCCCATGGATGA
GTCCACCCTTCTCTATGTTGTCTTCGAAGTCTTCGACGTCGTCCG
AGTGCACCAGCCCCACCGCGGCGTCATCGAGGCCGTCTACCTGCG
CACCCCCTTCTCGGCCGGTAACGCCACCACCTAAGCTCTTGCTTC
TTGCAAGCCATGGCCGCGGGCTCCGGCGAGCAGGAGCTCAGGGCC
ATCATCCGCGACCTGGGCTGCGGGCCCTACTTCCTGGGCACCTTC
GATAAGCGCTTCCCGGGATTCATGGCCCCGCACAAGCTGGCCTGC
GCCATCGTCAACACGGCCGGCCGCGAGACCGGGGGCGAGCACTGG
CTGGCCTTCGCCTGGAACCCGCGCTCGAACACCTGCTACCTCTTC
GACCCCTTCGGGTTCTCGGACGAGCGCCTCAAGCAGATCTACCAG
TTCGAGTACGAGGGCCTGCTGCGCCGCAGCGCCCTGGCCACCGAG
GACCGCTGCGTCACCCTGGAAAAGTCCACCCAGACCGTGCAGGGT
CCGCGCTCGGCCGCCTGCGGGCTCTTCTGCTGCATGTTCCTGCAC
GCCTTCGTGCACTGGCCCGACCGCCCCATGGACAAGAACCCCACC
ATGAACTTGCTGACGGGGGTGCCCAACGGCATGCTCCAGTCGCCC
CAGGTGGAACCCACCCTGCGCCGCAACCAGGAGGCGCTCTACCGC
TTCCTCAACTCCCACTCCGCCTACTTTCGCTCCCACCGCGCGCGC
ATCGAGAAGGCCACCGCCTTCGACCGCATGAATCAAGACATGTAA
ACCGTGTGTGTATGTTAAATGTCTTTAATAAACAGCACTTTCATG
TTACACATGCATCTGAGATGATTTATTTAGAAATCGAAAGGGTTC
TGCCGGGTCTCGGCATGGCCCGCGGGCAGGGACACGTTGCGGAAC
TGGTACTTGGCCAGCCACTTGAACTCGGGGATCAGCAGTTTGGGC
AGCGGGGTGTCGGGGAAGGAGTCGGTCCACAGCTTCCGCGTCAGT
TGCAGGGCGCCCAGCAGGTCGGGCGCGGAGATCTTGAAATCGCAG
TTGGGACCCGCGTTCTGCGCGCGGGAGTTGCGGTACACGGGGTTG
CAGCACTGGAACACCATCAGGGCCGGGTGCTTCACGCTCGCCAGC
ACCGTCGCGTCGGTGATGCTCTCCACGTCGAGGTCCTCGGCGTTG
GCCATCCCGAAGGGGGTCATCTTGCAGGTCTGCCTTCCCATGGTG
GGCACGCACCCGGGCTTGTGGTTGCAATCGCAGTGCAGGGGGATC
AGCATCATCTGGGCCTGGTCGGCGTTCATCCCCGGGTACATGGCC
TTCATGAAAGCCTCCAATTGCCTGAACGCCTGCTGGGCCTTGGCT
CCCTCGGTGAAGAAGACCCCGCAGGACTTGCTAGAGAACTGGTTG
GTGGCGCACCCGGCGTCGTGCACGCAGCAGCGCGCGTCGTTGTTG
GCCAGCTGCACCACGCTGCGCCCCCAGCGGTTCTGGGTGATCTTG
GCCCGGTCGGGGTTCTCCTTCAGCGCGCGCTGCCCGTTCTCGCTC
GCCACATCCATCTCGATCATGTGCTCCTTCTGGATCATGGTGGTC
CCGTGCAGGCACCGCAGCTTGCCCTCGGCCTCGGTGCACCCGTGC
AGCCACAGCGCGCACCCGGTGCACTCCCAGTTCTTGTGGGCGATC
TGGGAATGCGCGTGCACGAAGCCCTGCAGGAAGCGGCCCATCATG
GTGGTCAGGGTCTTGTTGCTAGTGAAGGTCAGCGGAATGCCGCGG
TGCTCCTCGTTGATGTACAGGTGGCAGATGCGGCGGTACACCTCG
CCCTGCTCGGGCATCAGCTGGAAGTTGGCTTTCAGGTCGGTCTCC
ACGCGGTAGCGGTCCATCAGCATAGTCATGATTTCCATACCCTTC
TCCCAGGCCGAGACGATGGGCAGGCTCATAGGGTTCTTCACCATC
ATCTTAGCGCTAGCAGCCGCGGCCAGGGGGTCGCTCTCGTCCAGG
GTCTCAAAGCTCCGCTTGCCGTCCTTCTCGGTGATCCGCACCGGG
GGGTAGCTGAAGCCCACGGCCGCCAGCTCCTCCTCGGCCTGTCTT
TCGTCCTCGCTGTCCTGGCTGACGTCCTGCAGGACCACATGCTTG
GTCTTGCGGGGTTTCTTCTTGGGCGGCAGCGGCGGCGGAGATGTT
GGAGATGGCGAGGGGGAGCGCGAGTTCTCGCTCACCACTACTATC
TCTTCCTCTTCTTGGTCCGAGGCCACGCGGCGGTAGGTATGTCTC
TTCGGGGGCAGAGGCGGAGGCGACGGGCTCTCGCCGCCGCGACTT
GGCGGATGGCTGGCAGAGCCCCTTCCGCGTTCGGGGGTGCGCTCC
CGGCGGCGCTCTGACTGACTTCCTCCGCGGCCGGCCATTGTGTTC
TCCTAGGGAGGAACAACAAGCATGGAGACTCAGCCATCGCCAACC
TCGCCATCTGCCCCCACCGCCGACGAGAAGCAGCAGCAGCAGAAT
GAAAGCTTAACCGCCCCGCCGCCCAGCCCCGCCACCTCCGACGCG
GCCGTCCCAGACATGCAAGAGATGGAGGAATCCATCGAGATTGAC
CTGGGCTATGTGACGCCCGCGGAGCACGAGGAGGAGCTGGCAGTG
CGCTTTTCACAAGAAGAGATACACCAAGAACAGCCAGAGCAGGAA
GCAGAGAATGAGCAGAGTCAGGCTGGGCTCGAGCATGACGGCGAC
TACCTCCACCTGAGCGGGGGGGAGGACGCGCTCATCAAGCATCTG
GCCCGGCAGGCCACCATCGTCAAGGATGCGCTGCTCGACCGCACC
GAGGTGCCCCTCAGCGTGGAGGAGCTCAGCCGCGCCTACGAGTTG
AACCTCTTCTCGCCGCGCGTGCCCCCCAAGCGCCAGCCCAATGGC
ACCTGCGAGCCCAACCCGCGCCTCAACTTCTACCCGGTCTTCGCG
GTGCCCGAGGCCCTGGCCACCTACCACATCTTTTTCAAGAACCAA
AAGATCCCCGTCTCCTGCCGCGCCAACCGCACCCGCGCCGACGCC
CTTTTCAACCTGGGTCCCGGCGCCCGCCTACCTGATATCGCCTCC
TTGGAAGAGGTTCCCAAGATCTTCGAGGGTCTGGGCAGCGACGAG
ACTCGGGCCGCGAACGCTCTGCAAGGAGAAGGAGGAGAGCATGAG
CACCACAGCGCCCTGGTCGAGTTGGAAGGCGACAACGCGCGGCTG
GCGGTGCTCAAACGCACGGTCGAGCTGACCCATTTCGCCTACCCG
GCTCTGAACCTGCCCCCCAAAGTCATGAGCGCGGTCATGGACCAG
GTGCTCATCAAGCGCGCGTCGCCCATCTCCGAGGACGAGGGCATG
CAAGACTCCGAGGAGGGCAAGCCCGTGGTCAGCGACGAGCAGCTG
GCCCGGTGGCTGGGTCCTAATGCTAGTCCCCAGAGTTTGGAAGAG
CGGCGCAAACTCATGATGGCCGTGGTCCTGGTGACCGTGGAGCTG
GAGTGCCTGCGCCGCTTCTTCGCCGACGCGGAGACCCTGCGCAAG
GTCGAGGAGAACCTGCACTACCTCTTCAGGCACGGGTTCGTGCGC
CAGGCCTGCAAGATCTCCAACGTGGAGCTGACCAACCTGGTCTCC
TACATGGGCATCTTGCACGAGAACCGCCTGGGGCAGAACGTGCTG
CACACCACCCTGCGCGGGGAGGCCCGGCGCGACTACATCCGCGAC
TGCGTCTACCTCTACCTCTGCCACACCTGGCAGACGGGCATGGGC
GTGTGGCAGCAGTGTCTGGAGGAGCAGAACCTGAAAGAGCTCTGC
AAGCTCCTGCAGAAGAACCTCAAGGGTCTGTGGACCGGGTTCGAC
GAGCGCACCACCGCCTCGGACCTGGCCGACCTCATTTTCCCCGAG
CGCCTCAGGCTGACGCTGCGCAACGGCCTGCCCGACTTTATGAGC
CAAAGCATGTTGCAAAACTTTCGCTCTTTCATCCTCGAACGCTCC
GGAATCCTGCCCGCCACCTGCTCCGCGCTGCCCTCGGACTTCGTG
CCGCTGACCTTCCGCGAGTGCCCCCCGCCGCTGTGGAGCCACTGC
TACCTGCTGCGCCTGGCCAACTACCTGGCCTACCACTCGGACGTG
ATCGAGGACGTCAGCGGCGAGGGCCTGCTCGAGTGCCACTGCCGC
TGCAACCTCTGCACGCCGCACCGCTCCCTGGCCTGCAACCCCCAG
CTGCTGAGCGAGACCCAGATCATCGGCACCTTCGAGTTGCAAGGG
CCCAGCGAAGGCGAGGGTTCAGCCGCCAAGGGGGGTCTGAAACTC
ACCCCGGGGCTGTGGACCTCGGCCTACTTGCGCAAGTTCGTGCCC
GAGGACTACCATCCCTTCGAGATCAGGTTCTACGAGGACCAATCC
CATCCGCCCAAGGCCGAGCTGTCGGCCTGCGTCATCACCCAGGGG
GCGATCCTGGCCCAATTGCAAGCCATCCAGAAATCCCGCCAAGAA
TTCTTGCTGAAAAAGGGCCGCGGGGTCTACCTCGACCCCCAGACC
GGTGAGGAGCTCAACCCCGGCTTCCCCCAGGATGCCCCGAGGAAA
CAAGAAGCTGAAAGTGGAGCTGCCGCCCGTGGAGGATTTGGAGGA
AGACTGGGAGAACAGCAGTCAGGCAGAGGAGGAGGAGATGGAGGA
AGACTGGGACAGCACTCAGGCAGAGGAGGACAGCCTGCAAGACAG
TCTGGAGGAAGACGAGGAGGAGGCAGAGGAGGAGGTGGAAGAAGC
AGCCGCCGCCAGACCGTCGTCCTCGGCGGGGGAGAAAGCAAGCAG
CACGGATACCATCTCCGCTCCGGGTCGGGGTCCCGCTCGACCACA
CAGTAGATGGGACGAGACCGGACGATTCCCGAACCCCACCACCCA
GACCGGTAAGAAGGAGCGGCAGGGATACAAGTCCTGGCGGGGGCA
CAAAAACGCCATCGTCTCCTGCTTGCAGGCCTGCGGGGGCAACAT
CTCCTTCACCCGGCGCTACCTGCTCTTCCACCGCGGGGTGAACTT
TCCCCGCAACATCTTGCATTACTACCGTCACCTCCACAGCCCCTA
CTACTTCCAAGAAGAGGCAGCAGCAGCAGAAAAAGACCAGCAGAA
AACCAGCAGCTAGAAAATCCACAGCGGCGGCAGCAGGTGGACTGA
GGATCGCGGCGAACGAGCCGGCGCAAACCCGGGAGCTGAGGAACC
GGATCTTTCCCACCCTCTATGCCATCTTCCAGCAGAGTCGGGGGC
AGGAGCAGGAACTGAAAGTCAAGAACCGTTCTCTGCGCTCGCTCA
CCCGCAGTTGTCTGTATCACAAGAGCGAAGACCAACTTCAGCGCA
CTCTCGAGGACGCCGAGGCTCTCTTCAACAAGTACTGCGCGCTCA
CTCTTAAAGAGTAGCCCGCGCCCGCCCAGTCGCAGAAAAAGGCGG
GAATTACGTCACCTGTGCCCTTCGCCCTAGCCGCCTCCACCCATC
ATCATGAGCAAAGAGATTCCCACGCCTTACATGTGGAGCTACCAG
CCCCAGATGGGCCTGGCCGCCGGTGCCGCCCAGGACTACTCCACC
CGCATGAATTGGCTCAGCGCCGGGCCCGCGATGATCTCACGGGTG
AATGACATCCGCGCCCACCGAAACCAGATACTCCTAGAACAGTCA
GCGCTCACCGCCACGCCCCGCAATCACCTCAATCCGCGTAATTGG
CCCGCCGCCCTGGTGTACCAGGAAATTCCCCAGCCCACGACCGTA
CTACTTCCGCGAGACGCCCAGGCCGAAGTCCAGCTGACTAACTCA
GGTGTCCAGCTGGCGGGCGGCGCCACCCTGTGTCGTCACCGCCCC
GCTCAGGGTATAAAGCGGCTGGTGATCCGGGGCAGAGGCACACAG
CTCAACGACGAGGTGGTGAGCTCTTCGCTGGGTCTGCGACCTGAC
GGAGTCTTCCAACTCGCCGGATCGGGGAGATCTTCCTTCACGCCT
CGTCAGGCCGTCCTGACTTTGGAGAGTTCGTCCTCGCAGCCCCGC
TCGGGTGGCATCGGCACTCTCCAGTTCGTGGAGGAGTTCACTCCC
TCGGTCTACTTCAACCCCTTCTCCGGCTCCCCCGGCCACTACCCG
GACGAGTTCATCCCGAACTTCGACGCCATCAGCGAGTCGGTGGAC
GGCTACGATTGAAACTAATCACCCCCTTATCCAGTGAAATAAAGA
TCATATTGATGATGATTTTACAGAAATAAAAAATAATCATTTGAT
TTGAAATAAAGATACAATCATATTGATGATTTGAGTTTAACAAAA
AAATAAAGAATCACTTACTTGAAATCTGATACCAGGTCTCTGTCC
ATGTTTTCTGCCAACACCACTTCACTCCCCTCTTCCCAGCTCTGG
TACTGCAGGCCCCGGCGGGCTGCAAACTTCCTCCACACGCTGAAG
GGGATGTCAAATTCCTCCTGTCCCTCAATCTTCATTTTATCTTCT
ATCAGATGTCCAAAAAGCGCGTCCGGGTGGATGATGACTTCGACC
CCGTCTACCCCTACGATGCAGACAACGCACCGACCGTGCCCTTCA
TCAACCCCCCCTTCGTCTCTTCAGATGGATTCCAAGAGAAGCCCC
TGGGGGTGTTGTCCCTGCGACTGGCCGACCCCGTCACCACCAAGA
ACGGGGAAATCACCCTCAAGCTGGGAGAGGGGGTGGACCTCGATT
CCTCGGGAAAACTCATCTCCAACACGGCCACCAAGGCCGCCGCCC
CTCTCAGTTTTTCCAACAACACCATTTCCCTTAACATGGATCACC
CCTTTTACACTAAAGATGGAAAATTATCCTTACAAGTTTCTCCAC
CATTAAATATACTGAGAACAAGCATTCTAAACACACTAGCTTTAG
GTTTTGGATCAGGTTTAGGACTCCGTGGCTCTGCCTTGGCAGTAC
AGTTAGTCTCTCCACTTACATTTGATACTGATGGAAACATAAAGC
TTACCTTAGACAGAGGTTTGCATGTTACAACAGGAGATGCAATTG
AAAGCAACATAAGCTGGGCTAAAGGTTTAAAATTTGAAGATGGAG
CCATAGCAACCAACATTGGAAATGGGTTAGAGTTTGGAAGCAGTA
GTACAGAAACAGGTGTTGATGATGCTTACCCAATCCAAGTTAAAC
TTGGATCTGGCCTTAGCTTTGACAGTACAGGAGCCATAATGGCTG
GTAACAAAGAAGACGATAAACTCACTTTGTGGACAACACCTGATC
CATCACCAAACTGTCAAATACTCGCAGAAAATGATGCAAAACTAA
CACTTTGCTTGACTAAATGTGGTAGTCAAATACTGGCCACTGTGT
CAGTCTTAGTTGTAGGAAGTGGAAACCTAAACCCCATTACTGGCA
CCGTAAGCAGTGCTCAGGTGTTTCTACGTTTTGATGCAAACGGTG
TTCTTTTAACAGAACATTCTACACTAAAAAAATACTGGGGGTATA
GGCAGGGAGATAGCATAGATGGCACTCCATATACCAATGCTGTAG
GATTCATGCCCAATTTAAAAGCTTATCCAAAGTCACAAAGTTCTA
CTACTAAAAATAATATAGTAGGGCAAGTATACATGAATGGAGATG
TTTCAAAACCTATGCTTCTCACTATAACCCTCAATGGTACTGATG
ACAGCAACAGTACATATTCAATGTCATTTTCATACACCTGGACTA
ATGGAAGCTATGTTGGAGCAACATTTGGGGCTAACTCTTATACCT
TCTCATACATCGCCCAAGAATGAACACTGTATCCCACCCTGCATG
CCAACCCTTCCCACCCCACTCTGTGGAACAAACTCTGAAACACAA
AATAAAATAAAGTTCAAGTGTTTTATTGATTCAACAGTTTTACAG
GATTCGAGCAGTTATTTTTCCTCCACCCTCCCAGGACATGGAATA
CACCACCCTCTCCCCCCGCACAGCCTTGAACATCTGAATGCCATT
GGTGATGGACATGCTTTTGGTCTCCACGTTCCACACAGTTTCAGA
GCGAGCCAGTCTCGGGTCGGTCAGGGAGATGAAACCCTCCGGGCA
CTCCCGCATCTGCACCTCACAGCTCAACAGCTGAGGATTGTCCTC
GGTGGTCGGGATCACGGTTATCTGGAAGAAGCAGAAGAGCGGCGG
TGGGAATCATAGTCCGCGAACGGGATCGGCCGGTGGTGTCGCATC
AGGCCCCGCAGCAGTCGCTGCCGCCGCCGCTCCGTCAAGCTGCTG
CTCAGGGGGTCCGGGTCCAGGGACTCCCTCAGCATGATGCCCACG
GCCCTCAGCATCAGTCGTCTGGTGCGGCGGGCGCAGCAGCGCATG
CGGATCTCGCTCAGGTCGCTGCAGTACGTGCAACACAGAACCACC
AGGTTGTTCAACAGTCCATAGTTCAACACGCTCCAGCCGAAACTC
ATCGCGGGAAGGATGCTACCCACGTGGCCGTCGTACCAGATCCTC
AGGTAAATCAAGTGGTGCCCCCTCCAGAACACGCTGCCCACGTAC
ATGATCTCCTTGGGCATGTGGCGGTTCACCACCTCCCGGTACCAC
ATCACCCTCTGGTTGAACATGCAGCCCCGGATGATCCTGCGGAAC
CACAGGGCCAGCACCGCCCCGCCCGCCATGCAGCGAAGAGACCCC
GGGTCCCGGCAATGGCAATGGAGGACCCACCGCTCGTACCCGTGG
ATCATCTGGGAGCTGAACAAGTCTATGTTGGCACAGCACAGGCAT
ATGCTCATGCATCTCTTCAGCACTCTCAACTCCTCGGGGGTCAAA
ACCATATCCCAGGGCACGGGGAACTCTTGCAGGACAGCGAACCCC
GCAGAACAGGGCAATCCTCGCACAGAACTTACATTGTGCATGGAC
AGGGTATCGCAATCAGGCAGCACCGGGTGATCCTCCACCAGAGAA
GCGCGGGTCTCGGTCTCCTCACAGCGTGGTAAGGGGGCCGGCCGA
TACGGGTGATGGCGGGACGCGGCTGATCGTGTTCGCGACCGTGTC
ATGATGCAGTTGCTTTCGGACATTTTCGTACTTGCTGTAGCAGAA
CCTGGTCCGGGCGCTGCACACCGATCGCCGGCGGCGGTCTCGGCG
CTTGGAACGCTCGGTGTTGAAATTGTAAAACAGCCACTCTCTCAG
ACCGTGCAGCAGATCTAGGGCCTCAGGAGTGATGAAGATCCCATC
ATGCCTGATGGCTCTGATCACATCGACCACCGTGGAATGGGCCAG
ACCCAGCCAGATGATGCAATTTTGTTGGGTTTCGGTGACGGCGAG
CCTCGGGAACAACGATGAAGTAAATGCAAGCGGTGCGTTCCAGCA
TGGTTAGTTAGCTGATCTGTAGAAAAAACAAAAATGAACATTAAA
CCATGCTAGCCTGGCGAACAGGTGGGTAAATCGTTCTCTCCAGCA
CCAGGCAGGCCACGGGGTCTCCGGCGCGACCCTCGTAAAAATTGT
CGCTATGATTGAAAACCATCACAGAGAGACGTTCCCGGTGGCCGG
CGTGAATGATTCGACAAGATGAATACACCCCCGGAACATTGGCGT
CCGCGAGTGAAAAAAAGCGCCCGAGGAAGCAATAAGGCACTACAA
TGCTCAGTCTCAAGTCCAGCAAAGCGATGCCATGCGGATGAAGCA
CAAAATTCTCAGGTGCGTACAAAATGTAATTACTCCCCTCCTGCA
CAGGCAGCAAAGCCCCCGATCCCTCCAGGTACACATACAAAGCCT
CAGCGTCCATAGCTTACCGAGCAGCAGCACACAACAGGCGCAAGA
GTCAGAGAAAGGCTGAGCTCTAACCTGTCCACCCGCTCTCTGCTC
AATATATAGCCCAGATCTACACTGACGTAAAGGCCAAAGTCTAAA
AATACCCGCCAAATAATCACACACGCCCAGCACACGCCCAGAAAC
CGGTGACACACTCAAAAAAATACGCGCACTTCCTCAAACGCCCAA
AACTGCCGTCATTTCCGGGTTCCCACGCTACGTCATCAAAACACG
ACTTTCAAATTCCGTCGACCGTTAAAAACGTCACCCGCCCCGCCC
CTAACGGTCGCCCGTCTCTCAGCCAATCAGCGCCCCGCATCCCCA
AATTCAAACACCTCATTTGCATATTAACGCGCACAAAAAGTTTGA
GGTATATTATTGATGATG

### REFERENCES

1. Desrichard, A., Snyder, A. & Chan, T. A. Cancer Neoantigens and Applications for Immunotherapy. Clin. Cancer Res. Off J. Am. Assoc. Cancer Res. (2015). doi:10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J. Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637—COMPOSITIONS AND METHODS OF IDENTIFYING TUMOR SPECIFIC NEOANTIGENS. (A1). at <http://appft1.uspto.gov/netacgi/nph-Parser?Sect1=PTO1&Sect2=HITOFF&d=PG01&p=1&u=/netahtml/PTO/srchnum.html&r=1&f=G&l=50&s1=20110293637.PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. patent application Ser. No. 01/202,08706—OPTIMIZATION OF MULTIGENE ANALYSIS OF TUMOR SAMPLES. (A1). at <http://appft1.uspto.gov/netacgi/nph-Parser?Sect1=PTO1&Sect2=HITOFF&d=PG01&p=1&u=/netahtml/PTO/srchnum.html&r=1&f=G&l=50&s1=20120208706.PGNR.>
17. Target Capture for NextGen Sequencing—IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf Engl. (2011). doi:10.1093/bioinformatics/btr355
36. Vitting-Seerup, K., Porse, B. T., Sandelin, A. & Waage, J. splice R: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf Engl. (2015). doi:10.1093/bioinformatics/btv639
46. Jørgensen, K. W., Rasmussen, M., Buus, S. & Nielsen, M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Ke mir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books @Amazon.com. at <http://www.amazon.com/Janeways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss M J. A deeper look into Comet—implementation and features. J Am Soc Mass Spectrom. 2015 November; 26(11):1865-74. doi: 10.1007/s13361-015-1179-x. Epub 2015 Jun. 27.
63. Lukas Kall, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Kall, John D. Storey, Michael J. MacCoss and William Stafford Noble. Assigning confidence measures to peptides identified by tandem mass spectrometry. Journal of Proteome Research, 7(1):29-34, January 2008
65. Lukas Kall, John D. Storey and William Stafford Noble. Nonparametric estimation of posterior error probabilities associated with peptides identified by tandem mass spectrometry. Bioinformatics, 24(16):i42-i48, August 2008
66. Kinney R M, B J Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frédérique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pasternack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Krüse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Comet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, J H and E G Strauss. The alphaviruses: gene expression, replication, and evolution. Microbiol Rev. 1994 September; 58(3): 491-562.
84. Rhême C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. II, and Wilfred Vermerris. Recent Advances in Nanomaterials for Gene Delivery—A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a referenfe genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Côté, Sylvie Mader, Sébastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-DR (HLA-DR) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrörs, Fulvia Vascotto, John C. Castle, Arbel D. Tadmor, Stephen P. Schoenberger, Christoph Huber, Özlem Türeci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. NN-align. An artificial neural network-based alignment algorithm for MHC class II peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS DB: de novo sequencing assisted database search for sensitive and accurate peptide identification. Molecular & Cellular Proteomics. 11(4):1-8. Jan. 2, 2012.
98. Jensen, Kamilla Kjaergaard, et al. “Improved Methods for Prediting Peptide Binding Affinity to MHC Class II Molecules.” Immunology, 2018, doi:10.1111/imm.12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Veeriah, S., Van Loo, P., Herrero, J., et al. (2017). Allele-Specific HLA Loss and Immune Escape in Lung Cancer Evolution. Cell 171, 1259-1271.e11.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
104. MacLean, B., Tomazela, D. M., Shulman, N., Chambers, M., Finney, G. L., Frewen, B., Kern, R., Tabb, D. L., Liebler, D. C., MacCoss, M. J., (2010). Skyline: an open source document editor for creating and analyzing targeted proteomics experiments. Bioinformatics 26(7), 966-8.

What is claimed is:

1. A method for treating a subject with a disease, wherein the disease is cancer comprising (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:
(Eₓ-(E^{N}ₙ)_{y})_{z}
wherein,
E represents a nucleotide sequence a distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and
at least one of the distinct epitope-encoding nucleic acid sequences comprising the at least two iterations encodes the KRAS-associated MHC class I neoepitope.

2. A method for treating a subject with a disease, wherein the disease is cancer comprising (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen expression system, comprising:
the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) an epitope-encoding nucleic acid sequence encoding the KRAS-associated MHC class I neoepitope, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence, and
wherein the at least one antigen-encoding nucleic acid sequence comprises at least two iterations of the epitope-encoding nucleic acid sequence encoding the KRAS-associated MHC class I neoepitope.

3. The method of any one of the above claims, wherein the epitope-encoding nucleic acid sequence is derived from a tumor of the subject with cancer or from a cell or sample of the infected subject.

4. The method any one of the above claims, wherein the epitope-encoding nucleic acid sequence are not derived from a tumor of the subject with cancer or from a cell or sample of the infected subject.

5. A method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:
(Eₓ-(E^{N}ₙ)_{y})_{z}
wherein,
E represents a nucleotide sequence comprising a distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and
at least one of the distinct epitope-encoding nucleic acid sequences comprising the at least two iterations encodes the KRAS-associated MHC class I neoepitope.

6. A method for stimulating an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising the method comprising administering to the subject an antigen-based vaccine to the subject, wherein the antigen-based vaccine comprises:
an antigen expression system, comprising:
the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) an epitope-encoding nucleic acid sequence encoding the KRAS-associated MHC class I neoepitope, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence, and
wherein the at least one antigen-encoding nucleic acid sequence comprises at least two iterations of at least one of the epitope-encoding nucleic acid sequences encoding the KRAS-associated MHC class I neoepitope.

7. The method of any one of the above claims, wherein the subject expresses at least one HLA allele predicted or known to present the at least one epitope sequence, optionally wherein the at least one epitope sequence predicted or known to be presented comprises the KRAS-associated MHC class I neoepitope.

8. The method of any one of the above claims, wherein the subject expresses at least one HLA allele predicted or known to present the at least one epitope sequence, and wherein the at least one epitope sequence comprises an epitope known or suspected to be presented by MHC class I on a surface of a cell, optionally wherein the at least one epitope sequence predicted or known to be presented comprises the KRAS-associated MHC class I neoepitope.

9. The method of claim 8, wherein the surface of the cell is a tumor cell surface.

10. A method for inducing an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises an antigen-encoding cassette, or a polypeptide sequence encoded by the cassette, wherein the antigen-encoding cassette comprises at least one antigen-encoding nucleic acid sequence described, from 5′ to 3′, by the formula:
(Eₓ-(E^{N}ₙ)_{y})_{z}
wherein,
E represents a nucleotide sequence comprising a distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof, and
at least one of the distinct epitope-encoding nucleic acid sequences comprising the at least two iterations encodes a KRAS-associated MHC class I neoepitope.

11. A method for inducing an immune response in a subject with cancer, wherein the cancer comprises (1) a solid tumor expressing a KRAS-associated MHC class I neoepitope, (2) colorectal cancer (CRC), (3) non-small cell lung cancer (NSCLC), and/or (4) pancreatic ductal adenocarcinoma (PDA), the method comprising administering to the subject an antigen-based vaccine to the subject, wherein the antigen-based vaccine comprises:
an antigen expression system, comprising:
the antigen expression system,
wherein the antigen expression system comprises one or more vectors,
the one or more vectors comprising:
(a) a vector backbone, wherein the backbone comprises:
(i) at least one promoter nucleotide sequence, and
(ii) optionally, at least one polyadenylation (poly(A)) sequence; and
(b) a cassette, wherein the cassette comprises:
(i) at least one antigen-encoding nucleic acid sequence, comprising:
(I) an epitope-encoding nucleic acid sequence encoding the KRAS-associated MHC class I neoepitope, and
wherein each of the epitope-encoding nucleic acid sequences comprises;
(A) optionally, a 5′ linker sequence, and
(B) optionally, a 3′ linker sequence;
(ii) optionally, a second promoter nucleotide sequence operably linked to the antigen-encoding nucleic acid sequence; and
(iii) optionally, at least one MHC class II epitope-encoding nucleic acid sequence;
(iv) optionally, at least one nucleic acid sequence encoding a GPGPG amino acid linker sequence (SEQ ID NO:56); and
(v) optionally, at least one second poly(A) sequence, wherein the second poly(A) sequence is a native poly(A) sequence or an exogenous poly(A) sequence to the vector backbone,
wherein if the second promoter nucleotide sequence is absent, the antigen-encoding nucleic acid sequence is operably linked to the at least one promoter nucleotide sequence,
wherein the at least one antigen-encoding nucleic acid sequence comprises at least two iterations of the epitope-encoding nucleic acid sequence encoding the KRAS-associated MHC class I neoepitope, and
wherein the subject expresses at least one HLA allele predicted or known to present the at least one KRAS-associated MHC class I neoepitope.

12. The method of any one of the above claims, wherein the antigen-based vaccine is administered as a priming dose.

13. The method of any one of the above claims, wherein the antigen-based vaccine is administered as one or more boosting doses.

14. The method of claim 13, wherein the boosting dose is different than the priming dose.

15. The method of claim 14, wherein:
a) the priming dose comprises a chimpanzee adenovirus vector and the boosting dose comprises an alphavirus vector; or
b) the priming dose comprises an alphavirus vector and the boosting dose comprises a chimpanzee adenovirus vector.

16. The method of claim 13, wherein the boosting dose is the same as the priming dose.

17. The method of any one of claims 13-16, wherein the injection site of the one or more boosting doses is as close as possible to the injection site of the priming dose.

18. The method of any one of the above method claims, further comprising determining or having determined the HLA-haplotype of the subject.

19. The method of any one of the above method claims, wherein the antigen-based vaccine is administered intramuscularly (IM), intradermally (ID), subcutaneously (SC), or intravenously (IV).

20. The method of any one of the above method claims, wherein the antigen-based vaccine is administered intramuscularly (IM).

21. The method of claim 20, wherein the IM administration is administered at separate injection sites.

22. The method of claim 21, wherein the separate injection sites are in opposing deltoid muscles.

23. The method of claim 22, wherein the separate injection sites are in gluteus or rectus femoris sites on each side.

24. The method of any of the above claims, wherein the KRAS-associated MHC class I neoepitope or the KRAS mutation comprises a KRAS G12C mutation, a KRAS G12V mutation, a KRAS G12D mutation, or a KRAS Q61H mutation.

25. The method of any of the above claims, wherein the KRAS-associated MHC class I neoepitope or the KRAS mutation comprises any one of the amino acid sequence shown in SEQ ID NOs: 75-82.

26. The method of any of the above claims, wherein the antigen-encoding cassette comprises each of the amino acid sequence shown in SEQ ID NOs: 75-82.

27. The method or composition of any of the above claims, wherein the antigen-encoding cassette comprises two or more iterations of each of the amino acid sequence shown in SEQ ID NOs: 75-82, optionally comprising 4 iterations of each of the amino acid sequence shown in SEQ ID NOs: 75-82.

28. The method of any of the above claims, wherein the KRAS-associated MHC class I neoepitope or the KRAS mutation comprises the amino acid sequence shown in SEQ ID NO: 57, SEQ ID NO: 58, SEQ ID NO: 59, or SEQ ID NO: 60.

29. The method of any of the above claims, wherein the epitope-encoding nucleic acid sequences comprises two or more distinct epitope-encoding nucleic acid sequences independently encoding a distinct KRAS-associated MHC class I neoepitope or a distinct KRAS mutation.

30. The method of any of the above claims, wherein each of the epitope-encoding nucleic acid sequences independently encodes a distinct KRAS-associated MHC class I neoepitope or a distinct KRAS mutation.

31. The method of any of the above claims, wherein the epitope-encoding nucleic acid sequences comprises two or more distinct epitope-encoding nucleic acid sequences independently encoding a KRAS G12C mutation, a KRAS G12V mutation, a KRAS G12D mutation, or a KRAS Q61H mutation.

32. The method of any of the above claims, wherein the epitope-encoding nucleic acid sequences independently encodes each of a KRAS G12C mutation, a KRAS G12V mutation, and a KRAS G12D mutation, and optionally a KRAS Q61H mutation.

33. The method of any of the above claims, wherein the antigen-encoding nucleic acid sequence encodes a peptide comprising the amino acid sequence shown in SEQ ID NO: 64 or SEQ ID NO: 65.

34. The method of any of the above claims, wherein the cassette does not encode an immunodominant MHC class I epitope that:
(1) stimulates a 5-fold or greater immune response when administered in a vaccine composition to a subject relative to another MHC class I epitope encoded in the cassette and capable of stimulating an immune response in the subject, and/or
(2) reduces an immune response to another MHC class I epitope encoded in the cassette when administered in a vaccine composition to a subject relative to an immune response when the other MHC class I epitope is administered in the absence of the immunodominant MHC class I epitope, optionally wherein the immune response is reduced to below a limit of detection and/or wherein the immune response is not a therapeutically effective response.

35. Any one of the above method claims, wherein the cancer comprises a solid tumor expressing a KRAS-associated and/or a NRAS-associated MHC class I neoepitope.

36. The method of claim 35, wherein the KRAS-associated and/or the NRAS-associated MHC class I neoepitope comprises a mutation selected from the group consisting of: KRAS_G12C, NRAS_G12C, KRAS_G12D, NRAS_G12D, KRAS_G12V, NRAS_G12V, KRAS_Q61H, and NRAS_Q61H.

37. Any one of the above method claims, wherein the cancer comprises colorectal cancer (CRC).

38. Any one of the above method claims, wherein the cancer comprises non-small cell lung cancer (NSCLC).

39. Any one of the above method claims, wherein the cancer comprises pancreatic ductal adenocarcinoma (PDA).

40. Any one of the above method claims, wherein the antigen-based vaccine or the one or more boosting doses is administered every 4 weeks (Q4W).

41. Any one of the above method claims, wherein the antigen-based vaccine or the one or more boosting doses is administered every 8 weeks (Q8W).

42. Any one of the above method claims, wherein the antigen-based vaccine or the one or more boosting doses is administered monthly.

43. Any one of the above method claims, wherein the antigen-based vaccine or the one or more boosting doses is administered every two months.

44. Any one of the above method claims, wherein the method comprises administering to the subject a composition for delivery of a self-replicating alphavirus-based expression system and administering to the subject a composition for delivery of a chimpanzee adenovirus (ChAdV)-based expression system, and
wherein the composition for delivery of the ChAdV-based expression system is administered as a priming dose and the composition for delivery of the self-replicating alphavirus-based expression system is administered as one or more boosting doses.

45. The method of any one of the above claims, wherein two or more boosting doses are administered.

46. The method of any one of the above claims, wherein 1, 2, 3, 4, 5, 6, 7, or 8 boosting doses are administered.

47. The method of any one of the above claims, wherein the ChAdV-based expression system is further administered as a boosting dose.

48. The method of claim 47, wherein the ChAdV-based boosting dose is only administered as a single boosting dose.

49. The method of claim 47 or 48, wherein the ChAdV-based expression system is administered as the boosting dose on or about day 140 after the priming dose of the ChAdV-based expression system.

50. The method of claim 47 or 48, wherein the ChAdV-based expression system is administered as the boosting dose on or about week 20 after the priming dose of the ChAdV-based expression system.

51. The method of claim 47 or 48, wherein the ChAdV-based expression system is administered as the boosting dose on or about month 5 after the priming dose of the ChAdV-based expression system.

52. The method of claim 47 or 48, wherein the ChAdV-based expression system is administered as the boosting dose on or after day 140 after the priming dose of the ChAdV-based expression system.

53. The method of claim 47 or 48, wherein the ChAdV-based expression system is administered as the boosting dose on or after week 20 after the priming dose of the ChAdV-based expression system.

54. The method of claim 47 or 48, wherein the ChAdV-based expression system is administered as the boosting dose on or after month 5 after the priming dose of the ChAdV-based expression system.

55. The method of any one of the above claims, wherein the self-replicating alphavirus-based expression system is administered as at least two boosting doses.

56. The method of any one of the above claims, wherein the self-replicating alphavirus-based expression system is administered as at least two or more boosting doses at least 28 days apart.

57. The method of any one of the above claims, wherein the self-replicating alphavirus-based expression system is administered as at least two or more boosting doses at least 4 weeks (Q4W) apart.

58. The method of any one of the above claims, wherein the self-replicating alphavirus-based expression system is administered as at least two or more boosting doses at least one month apart.

59. The method of any one of the above claims, wherein the self-replicating alphavirus-based expression system is administered as at least two or more boosting doses at least 56 days apart.

60. The method of any one of the above claims, wherein the self-replicating alphavirus-based expression system is administered as at least two or more boosting doses at least 8 weeks (Q8W) apart.

61. The method of any one of the above claims, wherein the self-replicating alphavirus-based expression system is administered as at least two or more boosting doses at least 2 months apart.

62. The method of any one of the above claims, wherein the self-replicating alphavirus-based expression system is administered as at least two boosting doses on or about days 28 and 84 after the priming dose of the ChAdV-based expression system.

63. The method of any one of the above claims, wherein the self-replicating alphavirus-based expression system is administered as at least two boosting doses on or about weeks 4 and 12 after the priming dose of the ChAdV-based expression system.

64. The method of any one of the above claims, wherein the self-replicating alphavirus-based expression system is administered as at least two boosting doses on or about months 1 and 3 after the priming dose of the ChAdV-based expression system.

65. The method of any one of the above claims, wherein the self-replicating alphavirus-based expression system is administered as at least four boosting doses.

66. The method of claim 65, wherein the self-replicating alphavirus-based expression system is administered on or about days 28, 84, 196, and 252 relative to the priming dose of the ChAdV-based expression system.

67. The method of claim 65, wherein the self-replicating alphavirus-based expression system is administered on or about weeks 4, 12, 28, and 40 relative to the priming dose of the ChAdV-based expression system.

68. The method of claim 65, wherein the self-replicating alphavirus-based expression system is administered on or about months 1, 3, 7, and 10 relative to the priming dose of the ChAdV-based expression system.

69. The method of any one of the above claims, the method further comprising administration of one or more immune modulators, optionally wherein the immune modulator is administered before, concurrently with, or after administration of the composition or pharmaceutical composition.

70. The method of claim 55, wherein the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

71. The method of claim 56 or 57, wherein the immune modulator is administered intravenously (IV), intramuscularly (IM), intradermally (ID), or subcutaneously (SC).

72. The method of claim 58, wherein the subcutaneous administration is near the site of the composition or pharmaceutical composition administration or in close proximity to one or more vector or composition draining lymph nodes.

73. The method of any one claims 56-59, wherein the method comprises administration of an anti-CTLA-4 antibody or an antigen-binding fragment thereof only with the priming dose and the first boosting dose.

74. The method of claim 60, wherein the anti-CTLA-4 antibody comprises ipilimumab.

75. The method of claim 61, wherein the ipilimumab is administered at a dose of 30 mg subcutaneously.

76. The method of any one of claims 56-62, wherein the method comprises administration of an anti-PD-L1 antibody or an antigen-binding fragment thereof every 4 weeks (Q4W), optionally comprising.

77. The method of claim 63, wherein the anti-PD-L1 antibody comprises atezolizumab or nivolumab.

78. The method of claim 64, wherein the atezolizumab is administered at a dose of 1680 mg intravenously or the nivolumab is administered at a dose of 480 mg intravenously.

79. The method of any one of claims 56-65, wherein the method comprises at least 2, 3, 4, 5, 6, 7, 8, 9, 10, 11, 12, or 13 administrations of one or more of the immune modulators.

80. The method of any one of claims 56-65, wherein the method comprises at least 13 administrations of the anti-PD-L1 antibody.

81. The method of any one of claims 56-67, wherein the one or more immune modulators are selected from the group consisting of: atezolizumab, nivolumamb, cemiplimab, and ipilimumab.

82. Any one of the above method or composition claims, wherein
the KRAS-associated MHC class I neoepitope comprising a KRAS G12C mutation is selected from the group consisting of: VVVGACGVGK (SEQ ID NO: 75), KLVVVGACGV (SEQ ID NO: 76), and GACGVGKSAL (SEQ ID NO: 93);
the KRAS-associated MHC class I neoepitope comprising a KRAS G12D mutation is selected from the group consisting of: VVGADGVGK (SEQ ID NO: 77), VVVGADGVGK (SEQ ID NO: 78), KLVVVGADGV (SEQ ID NO: 94), and GADGVGKSAL (SEQ ID NO: 95);
the KRAS-associated MHC class I neoepitope comprising a KRAS G12V mutation is selected from the group consisting of: VVGAVGVGK (SEQ ID NO: 79), VVVGAVGVGK (SEQ ID NO: 81), AVGVGKSAL (SEQ ID NO: 80), and GAVGVGKSAL (SEQ ID NO: 96).

83. Any one of the above method or composition claims, wherein the KRAS-associated MHC class I neoepitope comprising a KRAS G12C mutation is selected from the group consisting of: VVVGACGVGK (SEQ ID NO: 75), KLVVVGACGV (SEQ ID NO: 76), and GACGVGKSAL (SEQ ID NO: 93).

84. Any one of the above method or composition claims, wherein the KRAS-associated MHC class I neoepitope comprising a KRAS G12D mutation is selected from the group consisting of: VVGADGVGK (SEQ ID NO: 77), VVVGADGVGK (SEQ ID NO: 78), KLVVVGADGV (SEQ ID NO: 94), and GADGVGKSAL (SEQ ID NO: 95).

85. Any one of the above method or composition claims, wherein the KRAS-associated MHC class I neoepitope comprising a KRAS G12V mutation is selected from the group consisting of: VVGAVGVGK (SEQ ID NO: 79), VVVGAVGVGK (SEQ ID NO: 81), AVGVGKSAL (SEQ ID NO: 80), and GAVGVGKSAL (SEQ ID NO: 96).

86. Any one of the above method or composition claims, wherein the stimulating the immune response comprises stimulating a molecular response.

87. The method or composition of claim 73, wherein the molecular response comprises a reduction in ctDNA.

88. The method or composition of claim 74, wherein the reduction in ctDNA is at least a 20%, at least a 30%, at least a 40%, or at least a 50% reduction in ctDNA.

89. The method or composition of claim 74, wherein the reduction in ctDNA is at least a 30% reduction in ctDNA.


SEQUENCE LISTING (100 sequences)

SEQ ID NO: 1-2 - skipped (2 entries)

SEQ ID NO: 3 - RNA, 11447 nt, Venezuelan equine encephalitis virus
  source 1..11447: mol_type = other RNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaaggagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactccatacatctcgcccgtgtcacattgtggatgggcacggttatttcctgcttgccaggtgcccggcaggggactccatcaccatggaatttaagaaagattccgtcacacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgttgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataacctttggtttcagatcagtgtcactgaaactgcaccctaagaatcccacatatctaaccacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaattaactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctcagatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 4 - skipped

SEQ ID NO: 5 - RNA, 11446 nt, Venezuelan equine encephalitis virus
  source 1..11446: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttc

SEQ ID NO: 6 - DNA, 7895 bp, Venezuelan equine encephalitis virus
  source 1..7895: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 11446 bp, Venezuelan equine encephalitis virus
  source 1..11446: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtcagacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgctgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgaatgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttc

SEQ ID NO: 8 - DNA, 7914 bp, Venezuelan equine encephalitis virus
  source 1..7914: mol_type = other DNA
taatacgactcactataggatgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 7894 bp, Venezuelan equine encephalitis virus
  source 1..7894: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacgtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10-55 - skipped (46 entries)

SEQ ID NO: 56 - protein, 5 aa, synthetic construct
GPGPG

SEQ ID NO: 57 - protein, 25 aa, synthetic construct
MTEYKLVVVGACGVGKSALTIQLIQ

SEQ ID NO: 58 - protein, 25 aa, synthetic construct
MTEYKLVVVGADGVGKSALTIQLIQ

SEQ ID NO: 59 - protein, 25 aa, synthetic construct
MTEYKLVVVGAVGVGKSALTIQLIQ

SEQ ID NO: 60 - protein, 25 aa, synthetic construct
ETCLLDILDTAGHEEYSAMRDQYMR

SEQ ID NO: 61 - protein, 25 aa, synthetic construct
LRVEYLDDRNTFLHSVVVPYEPPEV

SEQ ID NO: 62 - protein, 25 aa, synthetic construct
HSGTAKSVTCTYYPALNKMFCQLAK

SEQ ID NO: 63 - protein, 543 aa, synthetic construct
MTEYKLVVVGAGDVGKSALTIQLIQETCLLDILDTAGKEEYSAMRDQYMRMCNSSCMGGMNRMPILTIITLEDSSSGIHSGATTTAPPLSGKGNPEEEDVSGIHSGATTTAPFLSGKGNPEEEDVEILDEAYVMAYVMAGVGSPYVSRLLMTEYKLVVVGADGVGKSALTIQLIQETCLLDILDTAGREEYSAMRDQYMRSYLDSGIHSGATATAPSLSGKGNPEKSVTCTYSPALNNMFCQLAKTCPVQMTEYKLVVVGAAGVGKSALTIQLIQETCLLDILDTAGLEEYSAMRDQYMRLRVEYLDDRNTFLHSVVVPYEPPEVDGQITVGQRIGSVSFGTVYKGKWHGMTEYKLVVVGAVGVGKSALTIQLIQETCLLDILDTAGHEEYSAMRDQYMRWQQQSYLDSGIHFGATTTAPSLSGKHSGTAKSVTCTYYPALNKMFCQLAKKSVTCTYSPALNEMFCQLAKTCPVQMTEYKLVVVGACGVGKSALTIQLIQGPGPGAKFVAAWTLKAAAGPGPGQYIKANSKFIGITELGPGPG

SEQ ID NO: 64 - protein, 443 aa, synthetic construct
MTEYKLVVVGACGVGKSALTIQLIQETCLLDILDTAGHEEYSAMRDQYMRMTEYKLVVVGAGDVGKSALTIQLIQETCLLDILDTAGREEYSAMRDQYMRMTEYKLVVVGADGVGKSALTIQLIQETCLLDILDTAGLEEYSAMRDQYMRMTEYKLVVVGAVGVGKSALTIQLIQETCLLDILDTAGKEEYSAMRDQYMRMTEYKLVVVGACGVGKSALTIQLIQETCLLDILDTAGKEEYSAMRDQYMRMTEYKLVVVGAGDVGKSALTIQLIQETCLLDILDTAGHEEYSAMRDQYMRMTEYKLVVVGAVGVGKSALTIQLIQETCLLDILDTAGREEYSAMRDQYMRMTEYKLVVVGADGVGKSALTIQLIQETCLLDILDTAGLEEYSAMRDQYMRGPGPGAKFVAAWTLKAAAGPGPGQYIKANSKFIGITELGPGPG

SEQ ID NO: 65 - protein, 443 aa, synthetic construct
MTEYKLVVVGACGVGKSALTIQLIQMTEYKLVVVGADGVGKSALTIQLIQETCLLDILDTAGHEEYSAMRDQYMRMTEYKLVVVGADGVGKSALTIQLIQMTEYKLVVVGAVGVGKSALTIQLIQMTEYKLVVVGACGVGKSALTIQLIQETCLLDILDTAGHEEYSAMRDQYMRMTEYKLVVVGADGVGKSALTIQLIQMTEYKLVVVGAVGVGKSALTIQLIQMTEYKLVVVGACGVGKSALTIQLIQETCLLDILDTAGHEEYSAMRDQYMRMTEYKLVVVGADGVGKSALTIQLIQMTEYKLVVVGAVGVGKSALTIQLIQETCLLDILDTAGHEEYSAMRDQYMRMTEYKLVVVGAVGVGKSALTIQLIQMTEYKLVVVGACGVGKSALTIQLIQGPGPGAKFVAAWTLKAAAGPGPGQYIKANSKFIGITELGPGPG

SEQ ID NO: 66 - protein, 143 aa, synthetic construct
MTEYKLVVVGACGVGKSALTIQLIQMTEYKLVVVGADGVGKSALTIQLIQETCLLDILDTAGHEEYSAMRDQYMRMTEYKLVVVGAVGVGKSALTIQLIQGPGPGAKFVAAWTLKAAAGPGPGQYIKANSKFIGITELGPGPG

SEQ ID NO: 67 - protein, 168 aa, synthetic construct
MTEYKLVVVGACGVGKSALTIQLIQMTEYKLVVVGADGVGKSALTIQLIQETCLLDILDTAGHEEYSAMRDQYMRMTEYKLVVVGAVGVGKSALTIQLIQLRVEYLDDRNTFLHSVVVPYEPPEVGPGPGAKFVAAWTLKAAAGPGPGQYIKANSKFIGITELGPGPG

SEQ ID NO: 68 - protein, 168 aa, synthetic construct
MTEYKLVVVGACGVGKSALTIQLIQMTEYKLVVVGADGVGKSALTIQLIQETCLLDILDTAGHEEYSAMRDQYMRMTEYKLVVVGAVGVGKSALTIQLIQHSGTAKSVTCTYYPALNKMFCQLAKGPGPGAKFVAAWTLKAAAGPGPGQYIKANSKFIGITELGPGPG

SEQ ID NO: 69 - DNA, 1647 bp, synthetic construct
  source 1..1647: mol_type = other DNA
atggctggcatgaccgagtacaaacttgtggtggttggtgctggagacgtggggaaaagcgccctgacaattcagcttattcaggaaacttgcttattagacatattagatactgccggaaaagaagagtattctgctatgagagatcagtacatgcggatgtgcaacagcagctgcatgggcggcatgaaccgcatgcccatcttgactattatcaccctggaggattcctccagtggaatacattcgggagccaccaccactgctcctcccctgtcaggtaagggaaatcctgaagaagaagatgttagcggcattcattccggtgctacgacaactgcaccatttctgtctggtaaaggtaatccagaggaggaggatgtggaaattctggatgaggcctacgtcatggcctatgtgatggctggcgttggaagcccatacgtgagtagactgctgatgacagaatataaattagtggtagtaggagcagatggggtaggaaagagtgccttaacaatccaactcattcaggagacctgtttgttggatattctggatacagctggccgggaagagtattcggcaatgagggaccaatatatgcgatcttacctcgacagcggaatccactccggtgccactgcaacagcgccgagcctgagtggtaaggggaaccctgagaagtcagtaacgtgtacatacagccctgctttaaacaacatgttttgccaacttgcaaaaacatgtcctgtacagatgacagagtataaacttgtagtggtgggggcagctggtgtaggtaaaagtgcacttaccatccagctgattcaagagacatgtctgttagacatcttggacacagccgggctcgaggaatatagcgccatgagagaccagtacatgcgactgcgcgtggagtatctggatgacaggaacaccttcctgcattctgttgtggtgccctatgagcccccggaggtggatggacagatcacagtgggccagagaattggcagcgtgtccttcgggactgtgtacaagggcaagtggcacggaatgacggaatataagctggtggtggtaggagcagtaggtgtcggtaaatcagccctcaccattcagcttatacaggagacttgtttgctggacatcctagacactgctggccatgaggagtactccgccatgcgcgatcaatacatgaggtggcagcagcagagctacctggactcagggatccactttggagctaccacaactgcgccatcactaagtgggaaacacagcggcaccgccaaatctgtcacgtgtacttactacccagccttgaacaagatgttctgtcaattagcaaagaaaagtgtcacctgcacatattcaccggcactgaatgagatgttctgccagctggctaagacctgtcctgtgcagatgactgagtacaaactggtcgttgtgggggcctgtggggttgggaagagcgctctgaccatccagctcatccagggacccggaccaggcgccaaatttgttgctgcttggacactgaaagctgctgctgggcccggaccaggccagtacatcaaggccaactctaagtttatcggcatcaccgaattgggacctggacccggctagtagtga

SEQ ID NO: 70 - DNA, 1341 bp, synthetic construct
  source 1..1341: mol_type = other DNA
atggctggcatgactgaatacaaattagttgtcgtcggtgcatgcggggtaggcaagtcggccttaacgatacaacttattcaagagacatgtctactggatatccttgacactgccggtcacgaagaatatagtgcaatgagggaccagtacatgcgtatgacggaatataagttagtagtagttggcgcaggcgacgtagggaaatccgctctaactatccaactaattcaagaaacgtgcctactagatatattagacacagcgggacgtgaggagtatagcgccatgcgtgaccagtatatgcggatgaccgagtataagctagtcgtagtcggcgcggatggggttggtaagagcgccttgaccatccagctcatacaagaaacttgccttctggacatcctagacaccgcgggtctcgaagagtacagtgcgatgagagatcagtacatgaggatgacagaatacaagctcgttgttgttggtgcggtcggtgttggaaagagtgcgctaaccattcagcttatccaggaaacctgtctgttagacatcttagataccgcaggtaaagaggaatattcggccatgagggatcaatatatgcgaatgacagagtataaattggtagtggtaggggcttgcggagtggggaaaagcgcattgactatacaattgattcaggaaacatgcctattggacatactcgacacggccgggaaagaagagtattccgcgatgcgagatcaatacatgcgcatgaccgaatataaacttgtcgttgtcggagcgggtgatgtaggtaaatcggcgctcacaatccaattaatccaagagacgtgcttgctagacattctggatacagctgggcacgaggagtactcagctatgcgcgatcagtatatgaggatgactgagtacaagttggtcgtcgtaggagcggttggtgtcggaaaatctgcgttgacaattcaactgatacaagagacttgtttgttagatattctcgatactgcgggtcgggaagaatactcggctatgagagaccaatatatgagaatgacggagtacaaactcgtagttgtaggtgcggacggtgtaggaaagtctgcgcttacgattcagttgatacaggagacctgtttgctcgatatcttggatacggcgggtttggaggaatacagcgcaatgcgggaccaatacatgagaggacccggaccaggcgccaaatttgttgctgcttggacactgaaagctgctgctgggcccggaccaggccagtacatcaaggccaactctaagtttatcggcatcaccgaattgggacctggacccggctag

SEQ ID NO: 71 - DNA, 1341 bp, synthetic construct
  source 1..1341: mol_type = other DNA
atggctggcatgaccgagtataaactagtagttgtgggagcgtgtggtgtaggcaagtcggcacttacaattcagttgatacaaatgacggaatataagctcgtagtagtcggagcagacggcgtggggaaatcagcgttgactatccagttaatacaggaaacttgcctattagacatcttggatacggcaggtcatgaggaatattccgctatgagagatcagtatatgcgcatgacggagtataagcttgtggttgtcggggccgacggggtaggtaagtcagcgctcacgatacaattaattcaaatgaccgaatacaagttggtcgtggtgggggcagttggggtcggtaaatccgcgttaacgatccaacttatccaaatgacagaatataaactcgttgttgtaggtgcatgtggcgtaggaaaaagcgcattgaccatccagctaattcaggagacgtgtctccttgatatcctagacacggcggggcacgaagaatactcggctatgcgcgaccagtacatgagaatgacggaatacaaacttgttgtcgtgggtgcggatggagtagggaaaagtgctctaacaatacaactcattcagatgacagagtacaaattggtagtcgtcggtgcggtaggagttgggaagtctgcactaactattcagctcatacagatgaccgagtacaagctggtggtggtaggcgcttgcggtgtgggtaagagtgcattaaccatacagcttatacaagagacatgtctgctagatatattagataccgccgggcatgaagagtactctgccatgcgagaccaatacatgcgtatgacagagtataaattagtagtggttggggcggacggtgttggcaagagcgccttaactatacagttgatccagatgacggagtacaaactggtcgtcgttggtgcagtgggagtgggaaaatctgcgctgacgattcaactaatccaagaaacatgtttacttgacatcctcgacactgcgggtcacgaggagtattcggcgatgcgtgatcaatatatgaggatgactgagtataagttagtcgtagttggagcggtcggtgtcggaaagtccgcgctaaccattcaattgattcaaatgactgaatacaagctagtggtagtaggagcatgcggcgtcggcaaatcggctttaacaatccaactgatacagggacccggaccaggcgccaaatttgttgctgcttggacactgaaagctgctgctgggcccggaccaggccagtacatcaaggccaactctaagtttatcggcatcaccgaattgggacctggacccggctag

SEQ ID NO: 72 - DNA, 441 bp, synthetic construct
  source 1..441: mol_type = other DNA
atggctggcatgaccgagtataaactagtagttgtgggagcgtgtggtgtaggcaagtcggcacttacaattcagttgatacaaatgacggaatataagctcgtagtagtcggagcagacggcgtggggaaatcagcgttgactatccagttaatacaggaaacttgcctattagacatcttggatacggcaggtcatgaggaatattccgctatgagagatcagtatatgcgcatgaccgaatacaagttggtcgtggtgggggcagttggggtcggtaaatccgcgttaacgatccaacttatccaaggacccggaccaggcgccaaatttgttgctgcttggacactgaaagctgctgctgggcccggaccaggccagtacatcaaggccaactctaagtttatcggcatcaccgaattgggacctggacccggctag

SEQ ID NO: 73 - DNA, 516 bp, synthetic construct
  source 1..516: mol_type = other DNA
atggctggcatgaccgagtataaactagtagttgtgggagcgtgtggtgtaggcaagtcggcacttacaattcagttgatacaaatgacggaatataagctcgtagtagtcggagcagacggcgtggggaaatcagcgttgactatccagttaatacaggaaacttgcctattagacatcttggatacggcaggtcatgaggaatattccgctatgagagatcagtatatgcgcatgaccgaatacaagttggtcgtggtgggggcagttggggtcggtaaatccgcgttaacgatccaacttatccaactgcgcgtggagtatctggatgacaggaacaccttcctgcattctgttgtggtgccctatgagcccccggaggtgggacccggaccaggcgccaaatttgttgctgcttggacactgaaagctgctgctgggcccggaccaggccagtacatcaaggccaactctaagtttatcggcatcaccgaattgggacctggacccggctag

SEQ ID NO: 74 - DNA, 516 bp, synthetic construct
  source 1..516: mol_type = other DNA
atggctggcatgaccgagtataaactagtagttgtgggagcgtgtggtgtaggcaagtcggcacttacaattcagttgatacaaatgacggaatataagctcgtagtagtcggagcagacggcgtggggaaatcagcgttgactatccagttaatacaggaaacttgcctattagacatcttggatacggcaggtcatgaggaatattccgctatgagagatcagtatatgcgcatgaccgaatacaagttggtcgtggtgggggcagttggggtcggtaaatccgcgttaacgatccaacttatccaacacagcggcaccgccaaatctgtcacgtgtacttactacccagccttgaacaagatgttctgtcaattagcaaagggacccggaccaggcgccaaatttgttgctgcttggacactgaaagctgctgctgggcccggaccaggccagtacatcaaggccaactctaagtttatcggcatcaccgaattgggacctggacccggctag

SEQ ID NO: 75 - protein, 10 aa, synthetic construct
VVVGACGVGK

SEQ ID NO: 76 - protein, 10 aa, synthetic construct
KLVVVGACGV

SEQ ID NO: 77 - protein, 9 aa, synthetic construct
VVGADGVGK

SEQ ID NO: 78 - protein, 10 aa, synthetic construct
VVVGADGVGK

SEQ ID NO: 79 - protein, 9 aa, synthetic construct
VVGAVGVGK

SEQ ID NO: 80 - protein, 9 aa, synthetic construct
AVGVGKSAL

SEQ ID NO: 81 - protein, 10 aa, synthetic construct
VVVGAVGVGK

SEQ ID NO: 82 - protein, 10 aa, synthetic construct
ILDTAGHEEY

SEQ ID NO: 83 - DNA, 28278 bp, synthetic construct
  source 1..28278: mol_type = other DNA
catcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgtaggtgtcagctgatcgccagggtatttaaacctgcgctctccagtcaagaggccactcttgagtgccagcgagaagagttttctcctccgcgccgcgagtcagatctacactttgaaagtagggataaggtagcgagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgcgggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggccgacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgcctgctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaaccaatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaaactaatcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacagaaccaccaggttgttcaacagtccatagttcaacacgctccagccgaaactcatcgcgggaaggatgctacccacgtggccgtcgtaccagatcctcaggtaaatcaagtggtgccccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcaactcctcgggggtcaaaaccatatcccagggcacggggaactcttgcaggacagcgaaccccgcagaacagggcaatcctcgcacagaacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtctcggcgcttggaacgctcggtgttgaaattgtaaaacagccactctctcagaccgtgcagcagatctagggcctcaggagtgatgaagatcccatcatgcctgatggctctgatcacatcgaccaccgtggaatgggccagacccagccagatgatgcaattttgttgggtttcggtgacggcgagcctcgggaacaacgatgaagtaaatgcaagcggtgcgttccagcatggttagttagctgatctgtagaaaaaacaaaaatgaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaattctcaggtgcgtacaaaatgtaattactcccctcctgcacaggcagcaaagcccccgatccctccaggtacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatg

SEQ ID NO: 84 - protein, 189 aa, synthetic construct [sequence omitted]

SEQ ID NO: 85 - protein, 100 aa, synthetic construct
VTNTEMFVTAPDNLGYMYEVQWPGQTQPQIANCSVYDFFVWLHYYSVRDTVTNTEMFVTAPDNLGYMYEVQWPGQTQPQIANCSVYDFFVWLHYYSVRDT

SEQ ID NO: 86 - protein, 25 aa, synthetic construct
VTNTEMFVTAPDNLGYMYEVQWPGQ

SEQ ID NO: 87 - protein, 25 aa, synthetic construct
TQPQIANCSVYDFFVWLHYYSVRDT

SEQ ID NO: 88 - protein, 10 aa, synthetic construct
YVYVADVAAK

SEQ ID NO: 89 - RNA, 17 nt, synthetic construct
  source 1..17: mol_type = other RNA
taatacgactcactata

SEQ ID NO: 90 - skipped

SEQ ID NO: 91 - protein, 9 aa, synthetic construct
VVVGAVGGK

SEQ ID NO: 92 - DNA, 19 bp, synthetic construct
  source 1..19: mol_type = other DNA
tccctatcagtgatagaga

SEQ ID NO: 93 - protein, 10 aa, synthetic construct
GACGVGKSAL

SEQ ID NO: 94 - protein, 10 aa, synthetic construct
KLVVVGADGV

SEQ ID NO: 95 - protein, 10 aa, synthetic construct
GADGVGKSAL

SEQ ID NO: 96 - protein, 10 aa, synthetic construct
GAVGVGKSAL

SEQ ID NO: 97 - protein, 10 aa, synthetic construct
KLVVVGAVGV

SEQ ID NO: 98 - RNA, 20 nt, synthetic construct
  source 1..20: mol_type = other RNA
  misc_feature >20: note = The nucleotides can be repeated 21 to 100 times
aaaaaaaaaaaaaaaaaaaa

SEQ ID NO: 99 - protein, 4 aa, synthetic construct
  REGION >4: note = The amino acid residues can be repeated 5 to 10 times
GGGG

SEQ ID NO: 100 - protein, 4 aa, synthetic construct
  REGION >4: note = The amino acid residues can be repeated 5 to 10 times
AAAA